(19) United States
(12) Patent Application Publication
(10) Pub. No.: US 2025/0057040 A1
(43) Pub. Date: Feb. 13, 2025

(54) COMPOUND, MATERIAL FOR ORGANIC ELECTROLUMINESCENCE DEVICE, ORGANIC ELECTROLUMINESCENCE DEVICE, AND ELECTRONIC APPARATUS

(71) Applicant: IDEMITSU KOSAN CO.,LTD., Tokyo (JP)

(72) Inventors: Ryota TAKAHASHI, Tokyo (JP); Sayaka MIZUTANI, Tokyo (JP); Masato MITANI, Tokyo (JP); Kei YOSHIDA, Tokyo (JP)

(73) Assignee: IDEMITSU KOSAN CO.,LTD., Tokyo (JP)

(21) Appl. No.: 18/719,475

(22) PCT Filed: Dec. 13, 2022
(86) PCT No.: PCT/JP2022/045841

§ 371 Date: Jun. 13, 2024

(30) Foreign Application Priority Data
Dec. 14, 2021 (JP) 2021-202532

(51) Int. Cl. H10K 85/60; C07D 239/26; C07D 251/24; C07D 403/10; C07D 405/10; H10K 50/16

(52) U.S. Cl. CPC H10K 85/654; C07D 239/26; C07D 251/24; C07D 403/10; C07D 405/10; H10K 85/626; H10K 85/6572; H10K 85/6574; H10K 50/166

(57) ABSTRACT

A compound represented by the following formula (1).

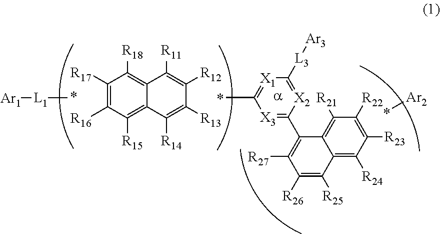

### TECHNICAL FIELD

The present invention relates to a novel compound, a material for an organic electroluminescence device, an organic electroluminescence device, and an electronic apparatus.

### BACKGROUND ART

When voltage is applied to an organic electroluminescence device (hereinafter, also referred to as an organic EL device), holes and electrons are injected into an emitting layer from an anode and a cathode, respectively. Then, thus injected holes and electrons are recombined in the emitting layer, and excitons are formed therein.

Conventional organic EL devices have not yet had sufficient device performance. Although materials used for the organic EL device are gradually improved to enhance the device performance, further performance enhancement is required.

Patent Document 1 discloses that a fused ring-containing compound having a specific structure is used in the emitting layer of an organic EL device.

### RELATED ART DOCUMENTS

### Patent Documents

[Patent Document 1] WO 2005/112519 A1

### Summary of the Invention

It is an object of the present invention to provide a compound that can achieve a high-performance organic EL device and to provide an organic EL device having such performance.

The inventors of the present invention have made extensive investigations with a view to achieving the above-mentioned object, and as a result, have found that the use of a compound having a specific structure in an organic layer of an organic EL device provides a long-lifetime organic EL device. Thus, the inventors have completed the present invention.

According to the present invention, the following compound and the like are provided.

### 1. A Compound Represented by the Following Formula (1):

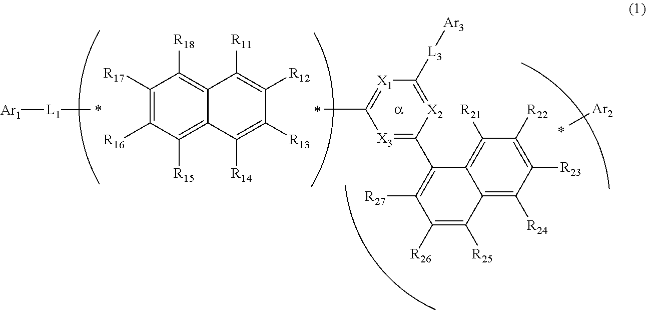

wherein in the formula (1),
X₁ to X₃ are independently C(R₁) or N, and at least two of X₁ to X₃ are N,
R₁ is a hydrogen atom or a substituent R,
Ar₁ and Ar₃ are independently
a substituted or unsubstituted aryl group having 6 to 50 ring carbon atoms, or
a substituted or unsubstituted monovalent heterocyclic group having 5 to 50 ring atoms,
Ar₂ is
a substituted or unsubstituted aryl group having 6 to 50 ring carbon atoms,
L₁ and L₃ are
a single bond,
a substituted or unsubstituted arylene group having 6 to 30 ring carbon atoms,
a substituted or unsubstituted divalent heterocyclic group having 5 to 30 ring atoms, or
a divalent group obtained by combining two or three of a substituted or unsubstituted arylene group having 6 to 30 ring carbon atoms, and a substituted or unsubstituted divalent heterocyclic group having 5 to 30 ring atoms,
one or more sets of adjacent two or more of R₁₁ to R₁₈ do not bond with each other,
any one of R₁₁ to R₁₈ represents a bond with a six-membered ring α,
any one of R₁₁ to R₁₈ which do not represent bonds with the six-membered ring α represents a bond with L₁, and when L₁ is a single bond, any one of R₁₁ to R₁₈ which do not represent bonds with the six-membered ring α is directly bonded with Ar₁,
R₁₁ to R₁₈ which do not represent bonds with the six-membered ring α and which do not represent bonds with L₁ are independently a hydrogen atom or a substituent R,
one or more sets of adjacent two or more of R₂₁ to R₂₇ do not bond with each other,
any one of R₂₁ to R₂₇ represents a bond with Ar₂,
R₂₁ to R₂₇ which do not represent bonds with Ar₂ are independently a hydrogen atom or a substituent R,
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇):
   wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms, and when two or more of each of R₉₀₁ to R₉₀₇ are present, the two or more of each of R₉₀₁ to R₉₀₇ may be the same as or different from each other;
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms, and
when the two or more substituents R are present, the two or more substituents R may be the same as or different from each other.

### 2. An Organic Electroluminescence Device Comprising

a cathode,
an anode, and
one or more organic layers arranged between the cathode and the anode,
wherein at least one layer of the one or more organic layers comprises the compound according to 1.

### 3. An Electronic Apparatus Comprising the Organic Electroluminescence Device According to 2.

According to the present invention, there can be provided the compound that can achieve a long-lifetime organic EL device and the long-lifetime organic EL device.

### BRIEF DESCRIPTION OF DRAWING

FIG. 1 is a view for illustrating the schematic configuration of an organic EL device according to one embodiment of the present invention.

### MODE FOR CARRYING OUT THE INVENTION

### [Definition]

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group“is a” ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”.). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

### Unsubstituted Aryl Group (Specific Example Group G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by any of the following general formulas (TEMP-1) to (TEMP-15).

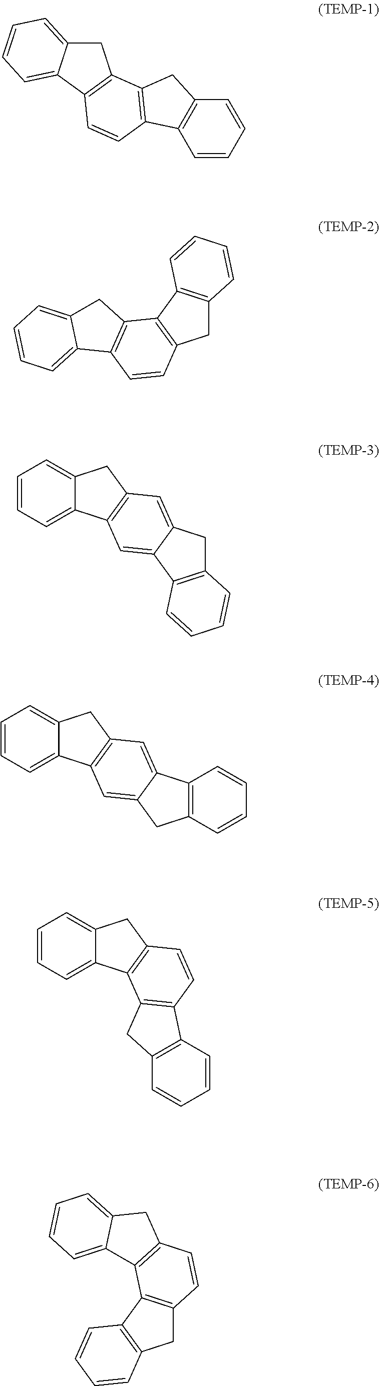

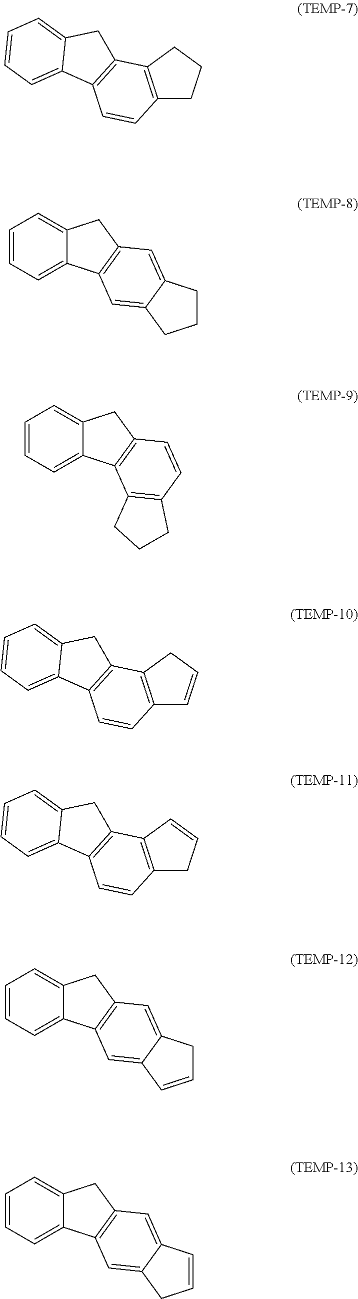

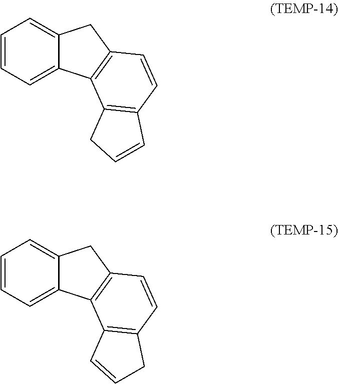

### Substituted Aryl Group (Specific Example Group G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by any of the general formulas (TEMP-1) to (TEMP-15) are substituted by a substituent.

### “Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group“is a” heterocyclic group unsubstituted by a substituent”, and the substituted heterocyclic group refers to the case where the “substituted or unsubstituted heterocyclic group“is a” heterocyclic group substituted by a substituent”.). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

### Unsubstituted Heterocyclic Group Containing a Nitrogen Atom (Specific Example Group G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### Unsubstituted Heterocyclic Group Containing an Oxygen Atom (Specific Example Group G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing a Sulfur Atom (Specific Example Group G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
   Monovalent Heterocyclic Group Derived by Removing One Hydrogen Atom from the Ring Structures Represented by any of the Following General Formulas (TEMP-16) to (TEMP-33) (Specific Example Group G2A4):

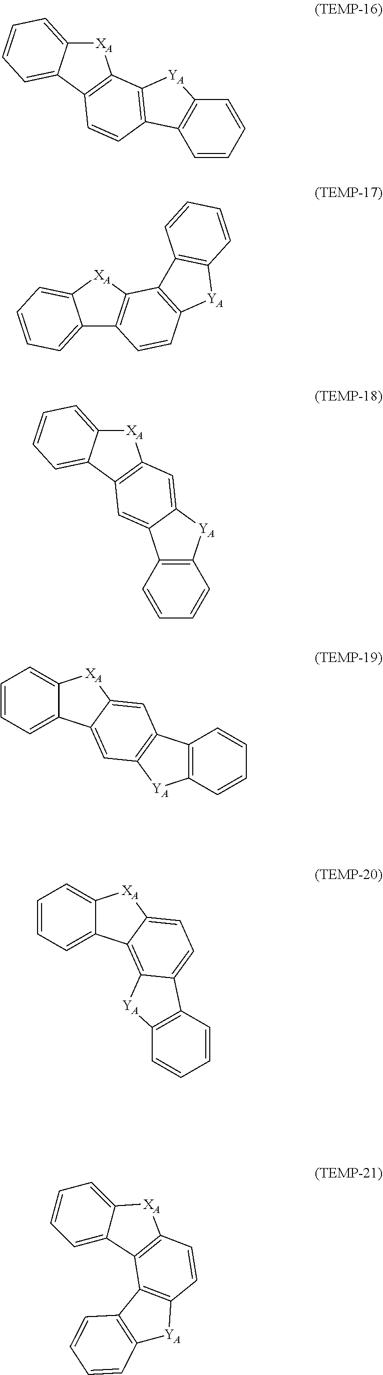

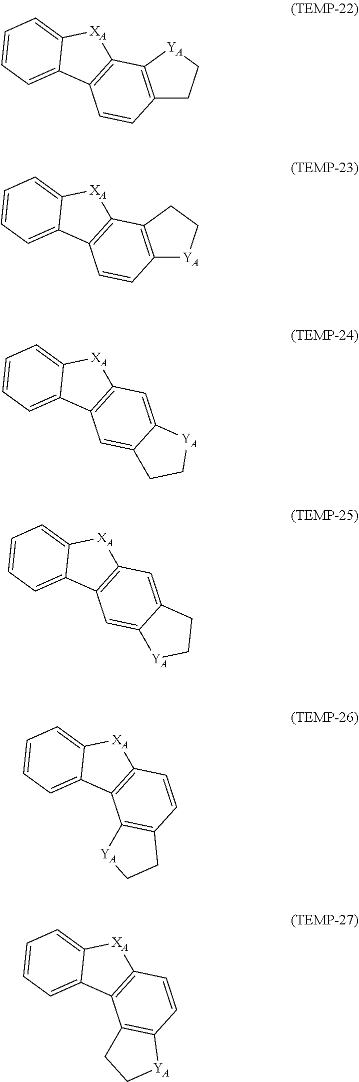

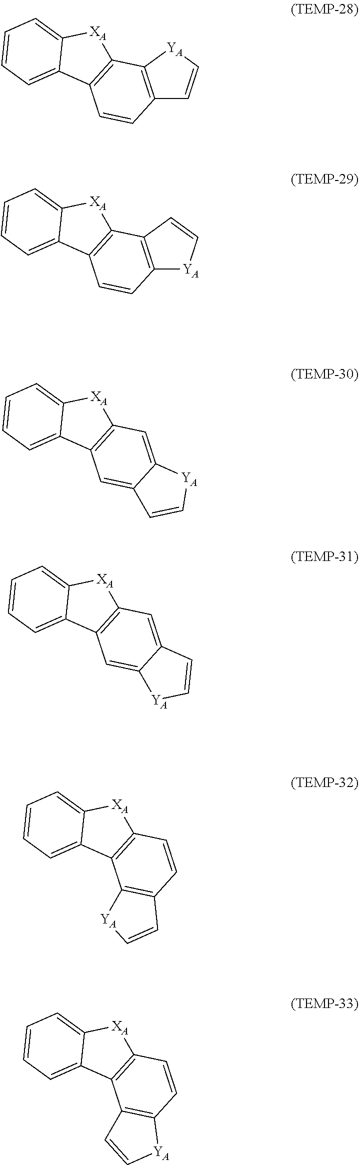

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.

### Substituted Heterocyclic Group Containing a Nitrogen Atom (Specific Example Group G2B1):

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

### Substituted Heterocyclic Group Containing an Oxygen Atom (Specific Example Group G2B2):

a phenyldibenzofuranyl group,
a methyldibenzofuranyl group,
a t-butyldibenzofuranyl group, and
a monovalent residue of spiro[9H-xanthene-9,9′-[9H]fluorene].

### Substituted Heterocyclic Group Containing a Sulfur Atom (Specific Example Group G2B3):

a phenyldibenzothiophenyl group,
a methyldibenzothiophenyl group,
a t-butyldibenzothiophenyl group, and
a monovalent residue of spiro[9H-thioxanthene-9,9′-[9H]fluorene].

Group in which one or more hydrogen atoms of the monovalent heterocyclic group derived from the ring structures represented by any of the following general formulas (TEMP-16) to (TEMP-33) are substituted by a substituent (specific example group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

### “Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”.). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

### Unsubstituted Alkyl Group (Specific Example Group G3A):

a methyl group,
an ethyl group,
a n-propyl group,
an isopropyl group,
a n-butyl group,
an isobutyl group,
a s-butyl group, and
a t-butyl group.

### Substituted Alkyl Group (Specific Example Group G3B):

a heptafluoropropyl group (including isomers),
a pentafluoroethyl group,
a 2,2,2-trifluoroethyl group, and
a trifluoromethyl group.

### “Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group“is a” alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

### Unsubstituted Alkenyl Group (Specific Example Group G4A):

a vinyl group,
an allyl group,
a 1-butenyl group,
a 2-butenyl group, and
a 3-butenyl group.

### Substituted Alkenyl Group (Specific Example Group G4B):

a 1,3-butanedienyl group,
a 1-methylvinyl group,
a 1-methylallyl group,
a 1,1-dimethylallyl group,
a 2-methylally group, and
a 1,2-dimethylallyl group.

### “Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”.). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

### Unsubstituted Alkynyl Group (Specific Example Group G5A):

an ethynyl group.

### “Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group“is a” cycloalkyl group unsubstituted by a substituent”, and the substituted cycloalkyl group refers to the case where the “substituted or unsubstituted cycloalkyl group” is a “cycloalkyl group substituted by a substituent”.). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

### Unsubstituted Cycloalkyl Group (Specific Example Group G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Specific Example Group G6B):

a 4-methylcyclohexyl group.
   “Group Represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
Plural G1's in —Si(G1)(G1)(G1) are the same or different.
Plural G2's in —Si(G1)(G2)(G2) are the same or different.
Plural G1's in —Si(G1)(G1)(G2) are the same or different.
Plural G2's in —Si(G2)(G2)(G2) are be the same or different.
Plural G3's in —Si(G3)(G3)(G3) are the same or different.
Plural G6's in —Si(G6)(G6)(G6) are be the same or different.

### “Group Represented by —O—(R₉₀₄)”

Specific examples of the group represented by —O—(R₉₀₄) in this specification (specific example group G8) include:
—O(G1),
—O(G2),
—O(G3), and
—O(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.

### “Group Represented by —S—(R₉₀₅)”

Specific examples of the group represented by —S—(R₉₀₅) in this specification (specific example group G9) include:
—S(G1),
—S(G2),
—S(G3), and
—S(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
   “Group Represented by —N(R₉₀₆)(R₉₀₇)”

Specific examples of the group represented by —N(R₉₀₆)(R₉₀₇) in this specification (specific example group G10) include:
—N(G1)(G1),
—N(G2)(G2),
—N(G1)(G2),
—N(G3)(G3), and
—N(G6)(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
Plural G1's in —N(G1)(G1) are the same or different.
Plural G2's in —N(G2)(G2) are the same or different.
Plural G3's in —N(G3)(G3) are the same or different.
Plural G6's in —N(G6)(G6) are the same or different.

### “Halogen Atom”

Specific examples of the “halogen atom” described in this specification (specific example group G11) include a fluorine atom, a chlorine atom, a bromine atom, an iodine atom, and the like.

### “Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### “Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### “Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### “Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

### “Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### “Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### “Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

### “Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

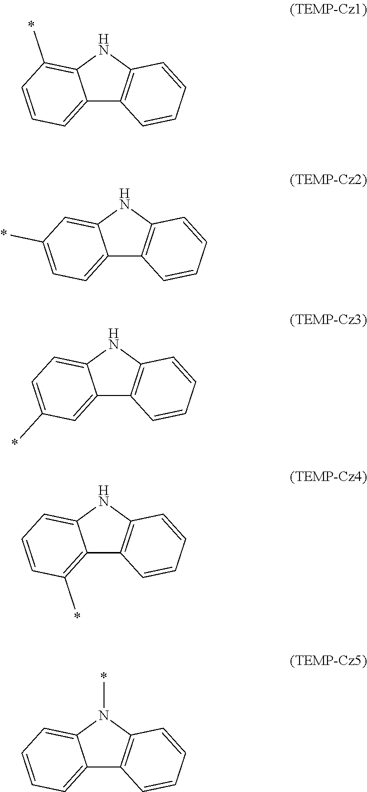

In this specification, the (9-phenyl)carbazolyl group is specifically any of the following groups, unless otherwise specified in this specification.

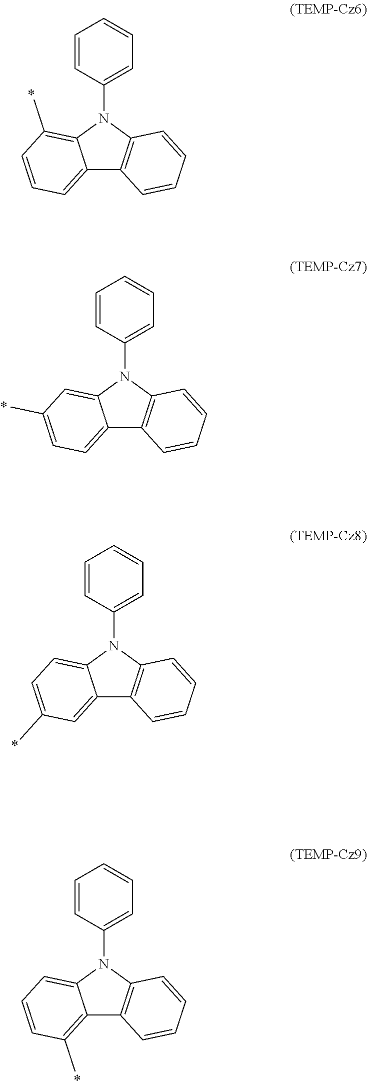 [SMILES: Cc1cccc2c3ccccc3n(-c3ccccc3)c12 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

In the general formulas (TEMP-Cz1) to (TEMP-Cz9),*represents a bonding site.

In this specification, the dibenzofuranyl group and the dibenzothiophenyl group are specifically any of the following groups, unless otherwise specified in this specification.

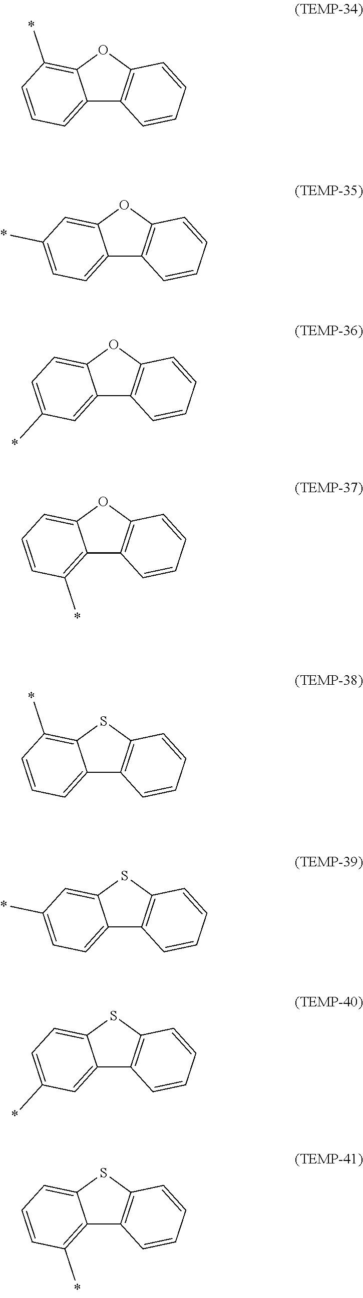

In the general formulas (TEMP-34) to (TEMP-41),*represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### “Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

### “Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### “Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

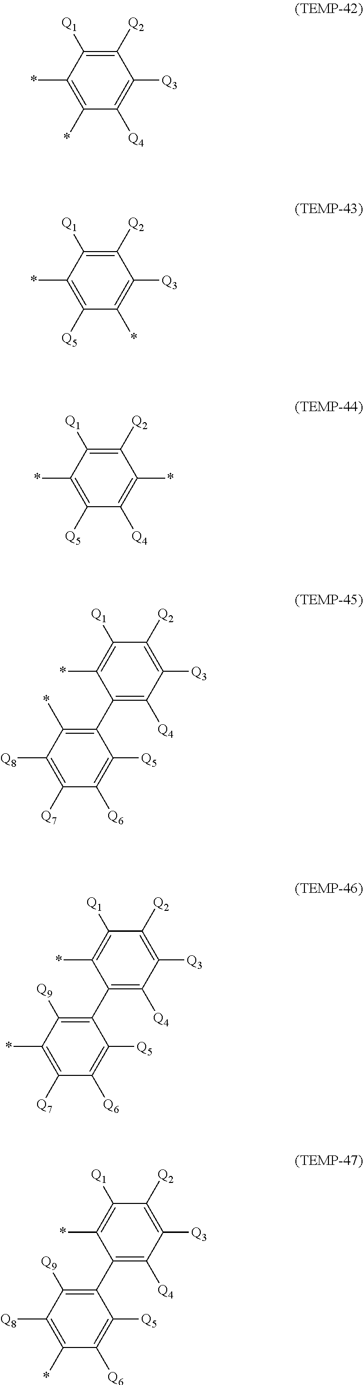

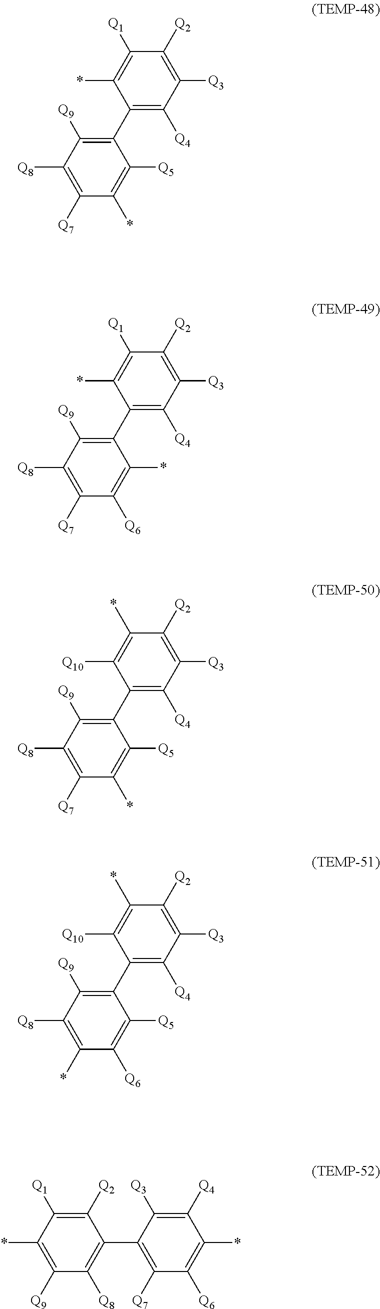

In the general formulas (TEMP-42) to (TEMP-52), Q₁ to Q₁₀ are independently a hydrogen atom or a substituent.

In the general formulas (TEMP-42) to (TEMP-52),*represents a bonding site.

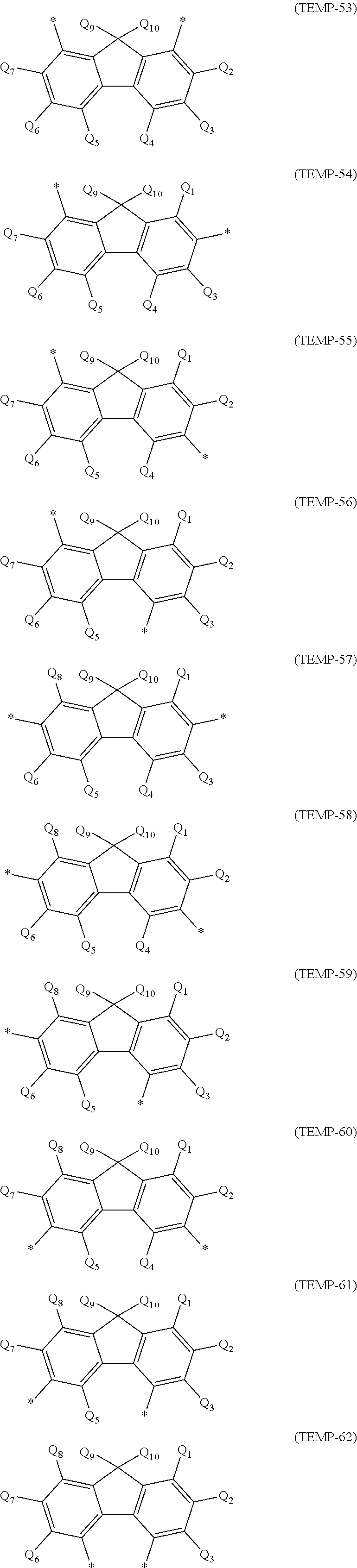

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62),*represents a bonding site.

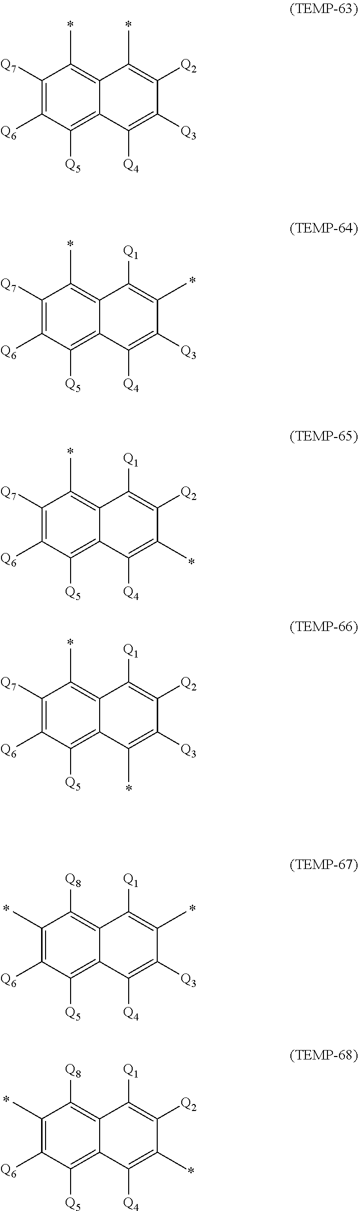

In the general formulas (TEMP-63) to (TEMP-68), Q₁ to Q₈ are independently a hydrogen atom or a substituent.

In the general formulas (TEMP-63) to (TEMP-68),*represents a bonding site.

The substituted or unsubstituted divalent heterocyclic group described in this specification is preferably any group of the following general formulas (TEMP-69) to (TEMP-102), unless otherwise specified in this specification.

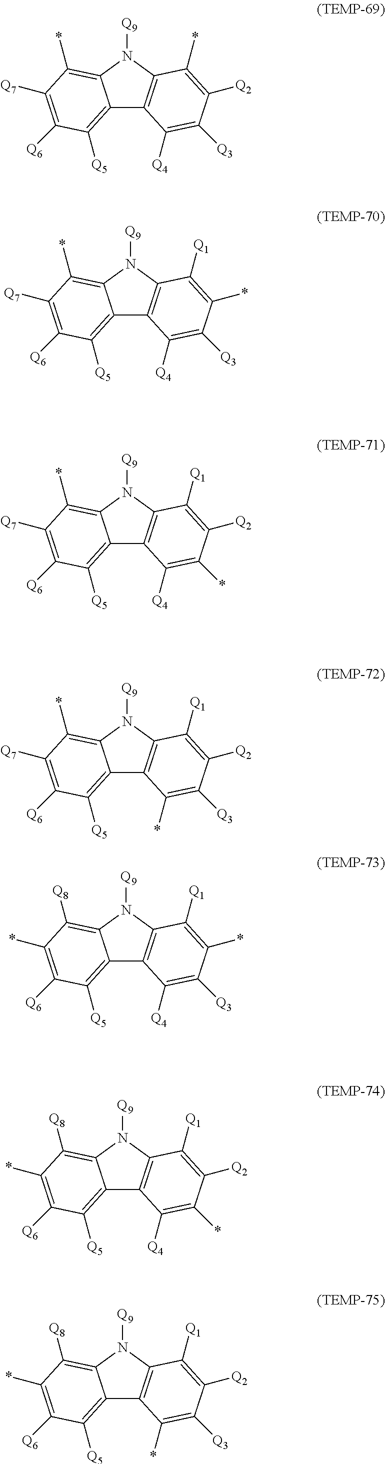

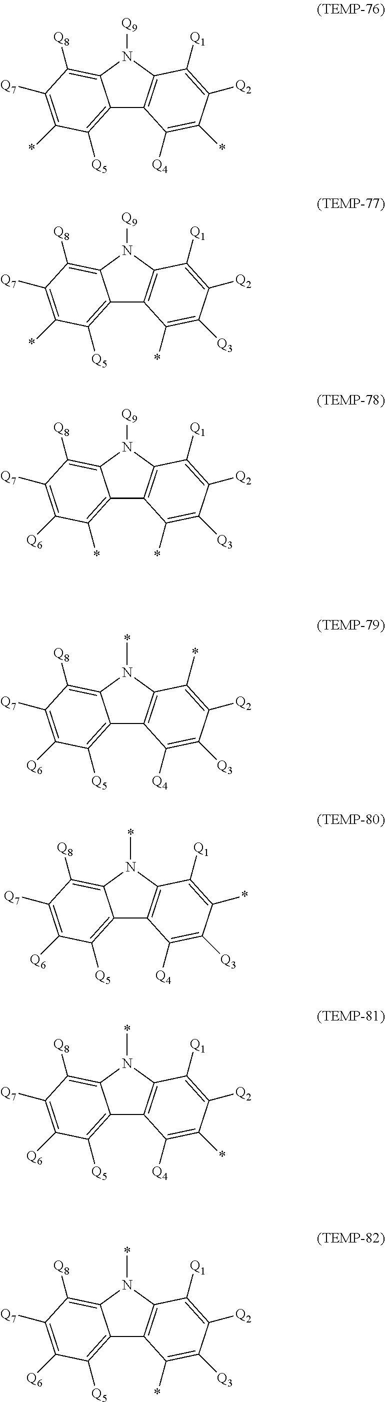

In the general formulas (TEMP-69) to (TEMP-82), Q₁ to Q₉ are independently a hydrogen atom or a substituent.

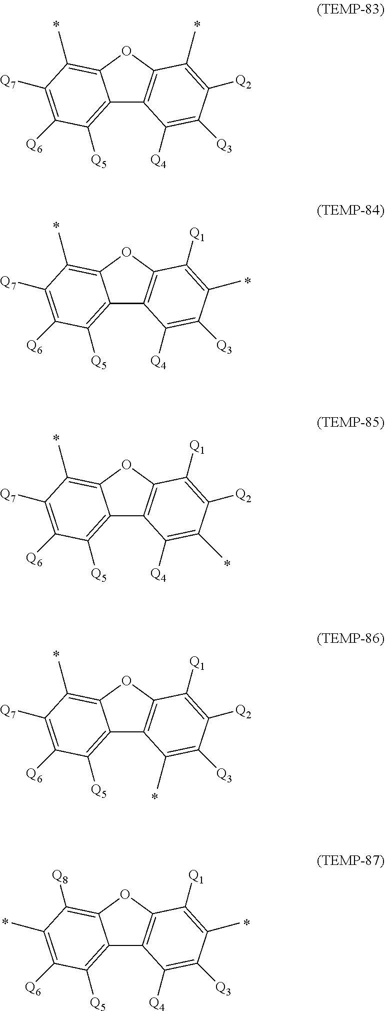

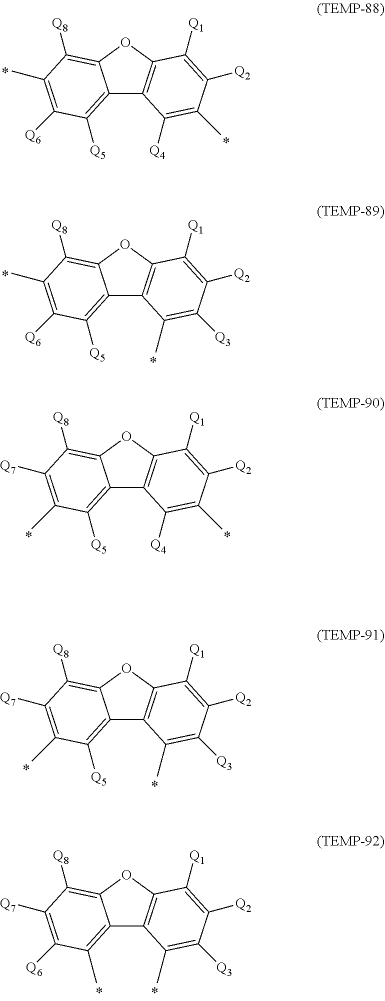

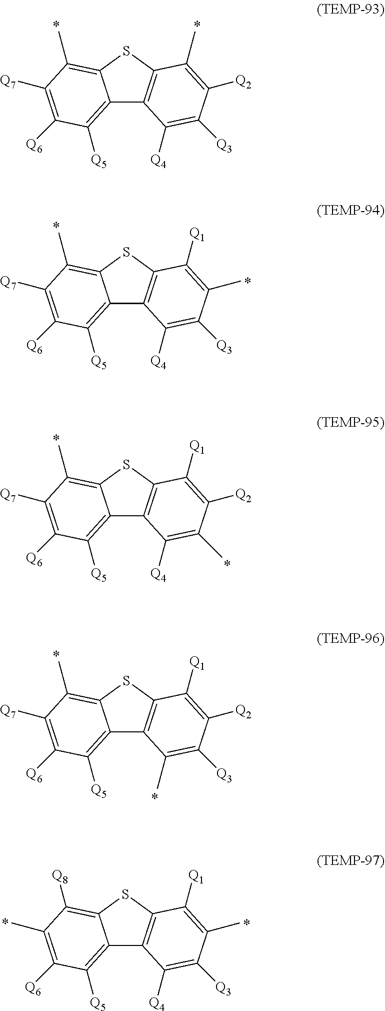

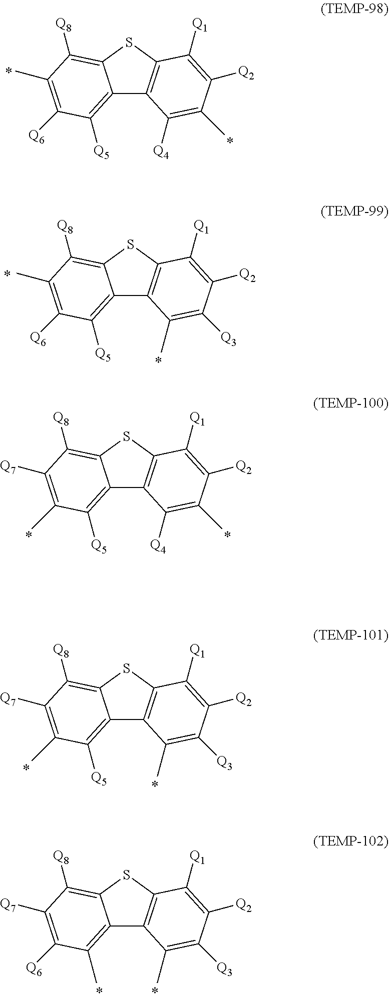

In the general formulas (TEMP-83) to (TEMP-102), Q₁ to Q₈ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

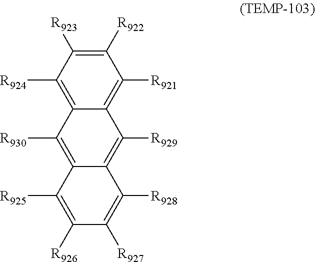

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

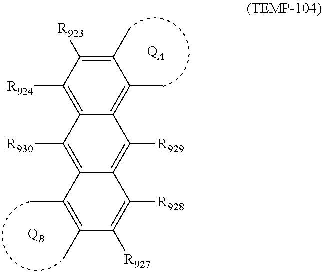

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

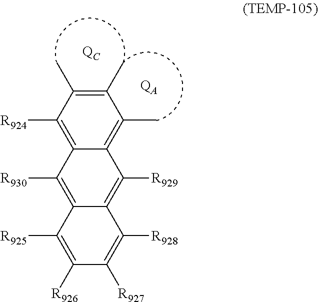

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other”(the case where “forming a ring by bonding with each other”).

Substituent in the case of “substituted or unsubstituted” In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Compound]

A compound according to an aspect of the present invention is represented by the following formula (1):

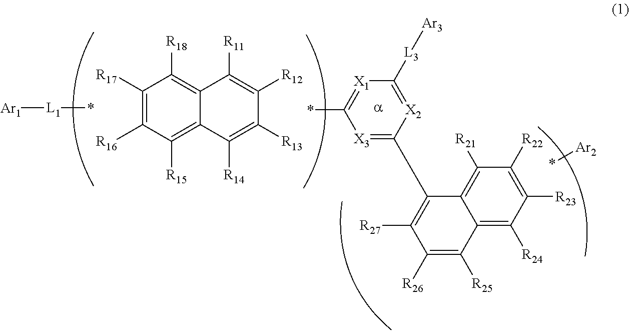

wherein in the formula (1),
X₁ to X₃ are independently C(R₁) or N, and at least two of X₁ to X₃ are N,
R₁ is a hydrogen atom or a substituent R,
Ar₁ and Ar₃ are independently
a substituted or unsubstituted aryl group having 6 to 50 ring carbon atoms, or
a substituted or unsubstituted monovalent heterocyclic group having 5 to 50 ring atoms,
Ar₂ is
a substituted or unsubstituted aryl group having 6 to 50 ring carbon atoms,
L₁ and L₃ are
a single bond,
a substituted or unsubstituted arylene group having 6 to 30 ring carbon atoms,
a substituted or unsubstituted divalent heterocyclic group having 5 to 30 ring atoms, or
a divalent group obtained by combining two or three of a substituted or unsubstituted arylene group having 6 to 30 ring carbon atoms, and a substituted or unsubstituted divalent heterocyclic group having 5 to 30 ring atoms,
one or more sets of adjacent two or more of R₁₁ to R₁₈ do not bond with each other,
any one of R₁₁ to R₁₈ represents a bond with a six-membered ring α,
any one of R₁₁ to R₁₈ which do not represent bonds with the six-membered ring α represents a bond with L₁, and when L₁ is a single bond, any one of R₁₁ to R₁₈ which do not represent bonds with the six-membered ring α is directly bonded with Ar₁,
R₁₁ to R₁₈ which do not represent bonds with the six-membered ring α and which do not represent bonds with L₁ are independently a hydrogen atom or a substituent R,
one or more sets of adjacent two or more of R₂₁ to R₂₇ do not bond with each other,
any one of R₂₁ to R₂₇ represents a bond with Ar₂,
R₂₁ to R₂₇ which do not represent bonds with Ar₂ are independently a hydrogen atom or a substituent R,
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇):
   wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms, and when two or more of each of R₉₀₁ to R₉₀₇ are present, the two or more of each of R₉₀₁ to R₉₀₇ may be the same as or different from each other;
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms, and
when the two or more substituents R are present, the two or more substituents R may be the same as or different from each other.

The compound according to an aspect of the present invention has the above-mentioned structure, and hence can improve the performance of an organic EL device when used in the device.

Although the reason for the foregoing is not necessarily clear, pyrimidine or triazine that is the six-membered ring α of the formula (1) is a moiety contributing to electron injection or electron transportation. Accordingly, it is conceivable that when a plurality of naphthyl groups that is an aromatic ring having a moderate conjugation length is bonded with the moiety, the amount of electrons to be injected and transported, the durability of the compound, and the like are controlled to contribute to the lengthening of the lifetime of the device.

At least two of X₁ to X₃ in the formula (1) are N. A case in which X₁ and X₂ are N, and X₃ is C(R₁) is permitted. A case in which X₂ and X₃ are N, and X₁ is C(R₁) is also permitted. A case in which X₃ and X₁ are N, and X₂ is C(R₁) is also permitted. A case in which X₁, X₂, and X₃ are N is also permitted.

The expressions “any one of R₁₁ to R₁₈ represents a bond with the six-membered ring α”, “any one of R₁₁ to R₁₈ which do not represent bonds with the six-membered ring α represents a bond with L₁”, and “any one of R₂₁ to R₂₇ represents a bond with Ar₂” are described.

In, for example, the case where R₁₁ represents a bond with the six-membered ring α, R₁₄ represents a bond with L₁, and R₂₅ represents a bond with Ar₂, the compound represented by the formula (1) is represented by the following formula (Ex1).

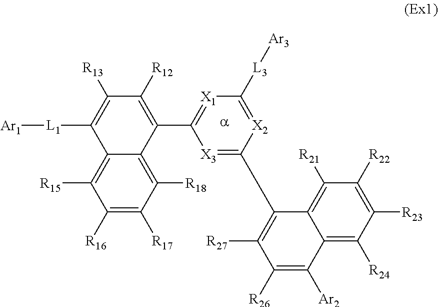

In addition, the expression “when L₁ is a single bond, any one of R₁₁ to R₁₈ which do not represent bonds with the six-membered ring α is directly bonded with Ar₁” is described by using the formula (Ex1). When L₁ in the compound represented by the formula (Ex1) is a single bond, the compound is represented by the following formula (Ex2). That is, R₁₄ in the formula (1) is directly bonded with Ar₁.

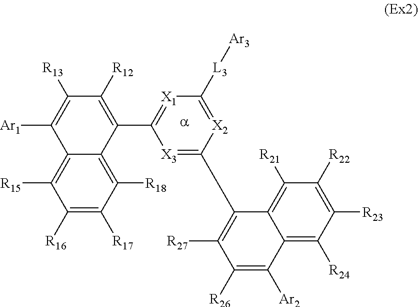

In one embodiment, R₁₁ represents a bond with the six-membered ring α.

In one embodiment, R₁₂ represents a bond with the six-membered ring α.

In one embodiment, the compound represented by the formula (1) is a compound represented by the following formula (1-1) or (1-2):

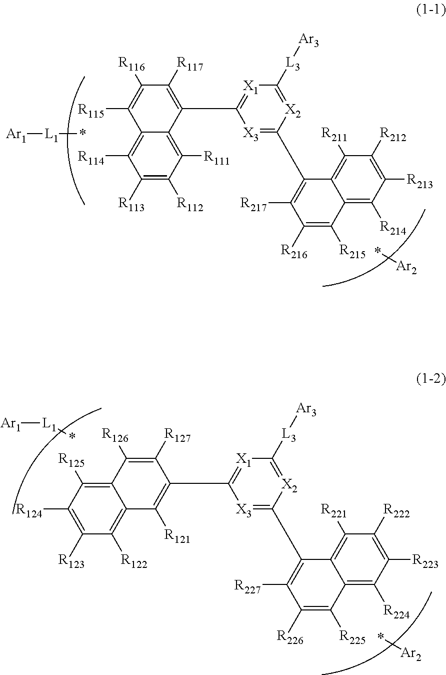

wherein in the formulas (1-1) to (1-2), X₁ to X₃, Ar₁ to Ar₃, L₁, and L₃ are the same as defined in the formula (1),
in the formula (1-1),
one or more sets of adjacent two or more of R₁₁₁ to R₁₁₇ and R₂₁₁ to R₂₁₇ do not bond with each other,
any one of R₁₁₁ to R₁₁₇ represents a bond with L₁, and when L₁ is a single bond, any one of R₁₁₁ to R₁₁₇ is directly bonded with Ar₁,
R₁₁₁ to R₁₁₇ which do not represent bonds with L₁ are independently a hydrogen atom or a substituent R,
any one of R₂₁₁ to R₂₁₇ represents a bond with Ar₂, and
R₂₁₁ to R₂₁₇ which do not represent bonds with Ar₂ are independently a hydrogen atom or a substituent R, and
in the formula (1-2),
one or more sets of adjacent two or more of R₁₂₁ to R₁₂₇ and R₂₂₁ to R₂₂₇ do not bond with each other,
any one of R₁₂₁ to R₁₂₇ represents a bond with L₁, and when L₁ is a single bond, any one of R₁₂₁ to R₁₂₇ is directly bonded with Ar₁,
R₁₂₁ to R₁₂₇ which do not represent bonds with L₁ are independently a hydrogen atom or a substituent R,
any one of R₂₂₁ to R₂₂₇ represents a bond with Ar₂,
R₂₂₁ to R₂₂₇ which do not represent bonds with Ar₂ are independently a hydrogen atom or a substituent R, and
the substituent R is the same as defined in the formula (1).

In one embodiment, two of X₁ to X₃ are N, and another one thereof is C(R₁).

In one embodiment, X₁ and X₂ are N, and X₃ is C(R₁).

In one embodiment, R₁ is a hydrogen atom.

In one embodiment, R₁₁₅ represents a bond with L₁.

In one embodiment, the compound represented by the formula (1) is selected from the group consisting of compounds represented by the following formulas (1-11) to (1-31):

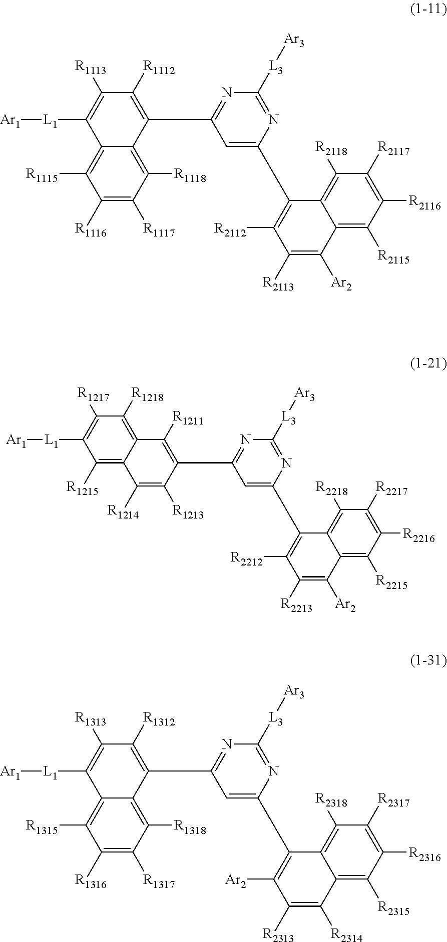

wherein in the formulas (1-11) to (1-31), Ar₁ to Ar₃, L₁, and L₃ are the same as defined in the formula (1),
one or more sets of adjacent two or more of R₁₁₁₂, R₁₁₁₃, R₁₁₁₅ to R₁₁₁₈, R₂₁₁₂, R₂₁₁₃, R₂₁₁₅ to R₂₁₁₈, R₁₂₁₁, R₁₂₁₃ to R₁₂₁₅, R₁₂₁₇ to R₁₂₁₈, R₂₂₁₂ to R₂₂₁₃, R₂₂₁₅ to R₂₂₁₈, R₁₃₁₂ to R₁₃₁₃, R₁₃₁₅ to R₁₃₁₈, and R₂₃₁₃ to R₂₃₁₈ do not bond with each other,
R₁₁₁₂, R₁₁₁₃, R₁₁₁₅ to R₁₁₁₈, R₂₁₁₂, R₂₁₁₃, R₂₁₁₅ to R₂₁₁₈, R₁₂₁₁, R₁₂₁₃ to R₁₂₁₅, R₁₂₁₇ to R₁₂₁₈, R₂₂₁₂ to R₂₂₁₃, R₂₂₁₅ to R₂₂₁₈, R₁₃₁₂ to R₁₃₁₃, R₁₃₁₅ to R₁₃₁₈, and R₂₃₁₃ to R₂₃₁₈ are independently a hydrogen atom or a substituent R, and
the substituent R is the same as defined in the formula (1).

In one embodiment, Ar₁ is a group represented by the following formula (1A):

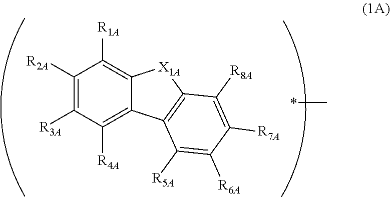

wherein in the formula (1A),
X_{1A} is O, S, N(R_{9A}), or C(R_{10A})(R_{11A}),
any one of R_{9A} in the case where X_{1A} is N(R_{9A}), and R_{1A} to R_{8A} represents a bond with L₁ with which Ar₁ is bonded, and when L₁ is a single bond, any one of R_{9A} in the case where X_{1A} is N(R_{9A}), and R_{1A} to R_{8A} is directly bonded with a naphthalene skeleton with which Ar₁ is bonded,
one or more sets of adjacent two or more of R_{10A} and R_{11A} in the case where X_{1A} is C(R_{10A})(R_{11A}), and R_{1A} to R_{8A} which do not represent bonds with L₁
form a substituted or unsubstituted single ring by bonding with each other,
form a substituted or unsubstituted fused ring by bonding with each other, or
do not bond with each other,
R_{9A} which does not represent a bond with L₁, R_{10A} and R_{11A} which do not form the substituted or unsubstituted single ring and which do not form the substituted or unsubstituted fused ring, and R_{1A} to R_{8A} which do not represent bonds with L₁, which do not form the substituted or unsubstituted single ring, and which do not form the substituted or unsubstituted fused ring are independently a hydrogen atom or a substituent R, and
the substituent R is the same as defined in the formula (1).

The expression “any one of R_{9A} in the case where X_{1A} is N(R_{9A}), and R_{1A} to R_{8A} represents a bond with L₁ with which Ar₁ is bonded” is described by using the formula (Ex2) described above.

In, for example, the case where in the formula (Ex2) described above, Ar₁ is a group represented by the formula (1A) and R_{3A} represents a bond with L₁ with which Ar₁ is bonded, the compound represented by the formula (Ex2) is represented by the following formula (Ex3).

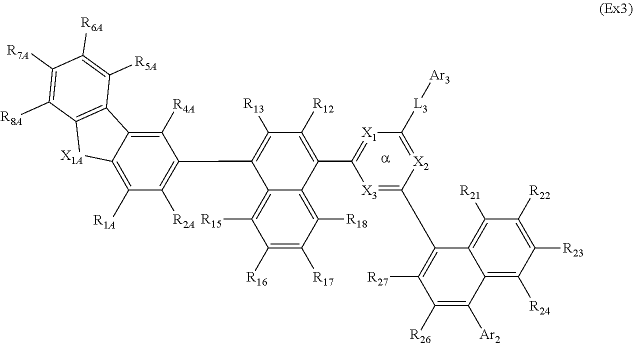

In one embodiment, one or more sets of the adjacent two or more of R_{1A} to R_{8A} which do not represent bonds with L₁ do not bond with each other.

In one embodiment, R_{10A} and R_{11A} in the case where X_{1A} is C(R_{10A})(R_{11A}) do not bond with each other.

In one embodiment, X_{1A} is O, S, or N(R_{9A}).

In one embodiment, X_{1A} is O or N(R_{9A}).

In one embodiment, X_{1A} is N(R_{9A}).

In one embodiment, R_{9A} represents a bond with L₁ with which Ar₁ is bonded.

In one embodiment, X_{1A} is O.

In one embodiment, R_{3A} represents a bond with L₁ with which Ar₁ is bonded.

In one embodiment, R_{1A} to R_{8A} which do not represent bonds with L₁, which do not form the substituted or unsubstituted single ring, and which do not form the substituted or unsubstituted fused ring are hydrogen atoms.

In one embodiment, R_{9A} which does not represent a bond with L₁ is the substituent R.

In one embodiment, R_{10A} and R_{11A} which do not form the substituted or unsubstituted single ring, and which do not form the substituted or unsubstituted fused ring are the substituent R.

In one embodiment, An is
a substituted or unsubstituted phenyl group,
a substituted or unsubstituted 1-naphthyl group, or
a substituted or unsubstituted 2-naphthyl group.

In one embodiment, Ar₂ is
a substituted or unsubstituted phenyl group,
a substituted or unsubstituted 1-naphthyl group, or
a substituted or unsubstituted 2-naphthyl group.

In one embodiment, Ar₃ is a substituted or unsubstituted aryl group having 6 to 50 ring carbon atoms.

In one embodiment, Ar₃ is
a substituted or unsubstituted phenyl group,
a substituted or unsubstituted 1-naphthyl group, or
a substituted or unsubstituted 2-naphthyl group.

In one embodiment, L₃ is a single bond.

In one embodiment, L₁ is a single bond, or a substituted or unsubstituted arylene group having 6 to 30 ring carbon atoms.

In one embodiment, in the formula (1), R₁₁ to R₁₈ which do not represent bonds with the six-membered ring α and which do not represent bonds with Ar₁, and R₂₁ to R₂₇ which do not represent bonds with Ar₂ are independently
a hydrogen atom,
a substituted or unsubstituted alkyl group having 1 to 18 carbon atoms, or
a substituted or unsubstituted aryl group having 6 to 18 ring carbon atoms.

In one embodiment, in the formula (1), R₁₁ to R₁₈ which do not represent bonds with the six-membered ring α and which do not represent bonds with Ar₁, and R₂₁ to R₂₇ which do not represent bonds with Ar₂ are hydrogen atoms.

In one embodiment, a substituent in the case of “substituted or unsubstituted” is selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇): wherein R₉₀₁ to R₉₀₇ are independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms, and when two or more of each of R₉₀₁ to R₉₀₇ are present, the two or more of each of R₉₀₁ to R₉₀₇ may be the same as or different from each other;
a halogen atom, a cyano group, a nitro group,
an unsubstituted aryl group having 6 to 50 ring carbon atoms, and
an unsubstituted heterocyclic group having 5 to 50 ring atoms.

In one embodiment, the substituent in the case of “substituted or unsubstituted” is selected from the group consisting of
an alkyl group having 1 to 18 carbon atoms,
an aryl group having 6 to 18 ring carbon atoms, and
a monovalent heterocyclic group having 5 to 18 ring atoms.

As defined in the definition, the “hydrogen atom” as used herein includes a protium atom, a deuterium atom, and a tritium atom. Accordingly, the inventive compounds may contain naturally derived deuterium atoms.

In addition, deuterium atoms may be intentionally introduced into the inventive compound by using a deuterated compound as a part or all of raw material compounds. Accordingly, in one embodiment of the present invention, the compound represented by the formula (1) includes at least one deuterium atom. That is, the compound of the present embodiment may be the compound represented by the formula (1), wherein at least one of hydrogen atoms contained in the compound is a deuterium atom.

In the compound represented by the formula (1), at least one hydrogen atom selected from
R₁ which is a hydrogen atom when X₁ to X₃ are C(R₁),
a hydrogen atom possessed by R₁ which is a substituent when X₁ to X₃ are C(R₁) (including a hydrogen atom in a substituent in the case where the substituent R has the substituent),
R₁₁ to R₁₈ which do not represent bonds with the six-membered ring α, which do not represent bonds with L₁, and which are hydrogen atoms,
hydrogen atoms possessed by R₁₁ to R₁₈ which do not represent bonds with the six-membered ring α, which do not represent bonds with L₁, and which are the substituent R (including a hydrogen atom in a substituent in the case where the substituent R has the substituent),
R₂₁ to R₂₇ which do not represent bonds with Ar₂ and which are hydrogen atoms, hydrogen atoms possessed by R₂₁ to R₂₇ which do not represent bonds with Ar₂ and which are the substituent R (including a hydrogen atom in a substituent in the case where the substituent R has the substituent),
a hydrogen atom possessed by L₁,
a hydrogen atom possessed by L₃,
a hydrogen atom possessed by Ar₁,
a hydrogen atom possessed by Ar₂, and
a hydrogen atom possessed by Ar₃
may be a deuterium atom:

The deuteration rate of the compound depends on the deuteration rate of the raw material compounds used. Even if a raw material having a predetermined deuteration rate is used, a protium atom isotope may be included at a certain proportion derived naturally. Accordingly, an aspect of the deuteration rate includes a proportion in which a trace amount of naturally derived isotopes is considered, based on a proportion obtained by simply counting the number of deuterium atoms represented by the chemical formula.

In one embodiment, the deuteration rate of the compound is preferably 1% or more, more preferably 3% or more, still more preferably 5% or more, still further more preferably 10% or more, yet still further more preferably 50% or more.

The compound (compound represented by the formula (1)) according to an aspect of the present invention may be synthesized by using a known alternative reaction or raw material suited for the intended product in imitation of Examples.

Specific examples of the compound (compound represented by the formula (1)) according to an aspect of the present invention are represented below. However, the specific examples are merely examples, and the compound according to an aspect of the present invention is not limited to the following specific examples.

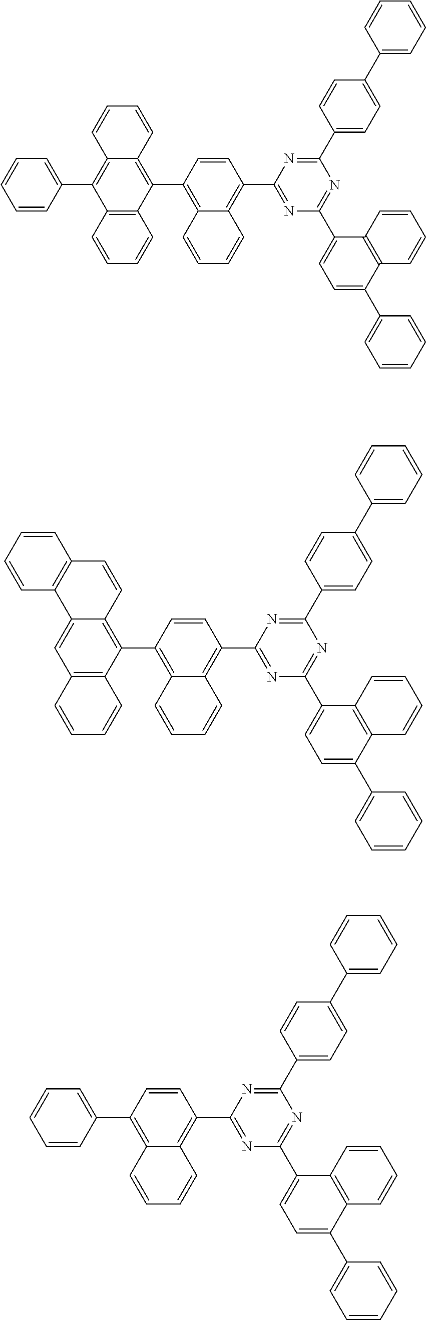 [SMILES: c1ccc(-c2ccc(-c3nc(-c4ccc(-c5ccccc5)c5ccccc45)nc(-c4ccc(-c5c6ccccc6c(-c6ccccc6)c6ccccc56)c5ccccc45)n3)cc2)cc1 ; c1ccc(-c2ccc(-c3nc(-c4ccc(-c5ccccc5)c5ccccc45)nc(-c4ccc(-c5c6ccccc6cc6c5ccc5ccccc56)c5ccccc45)n3)cc2)cc1 ; c1ccc(-c2ccc(-c3nc(-c4ccc(-c5ccccc5)c5ccccc45)nc(-c4ccc(-c5ccccc5)c5ccccc45)n3)cc2)cc1]

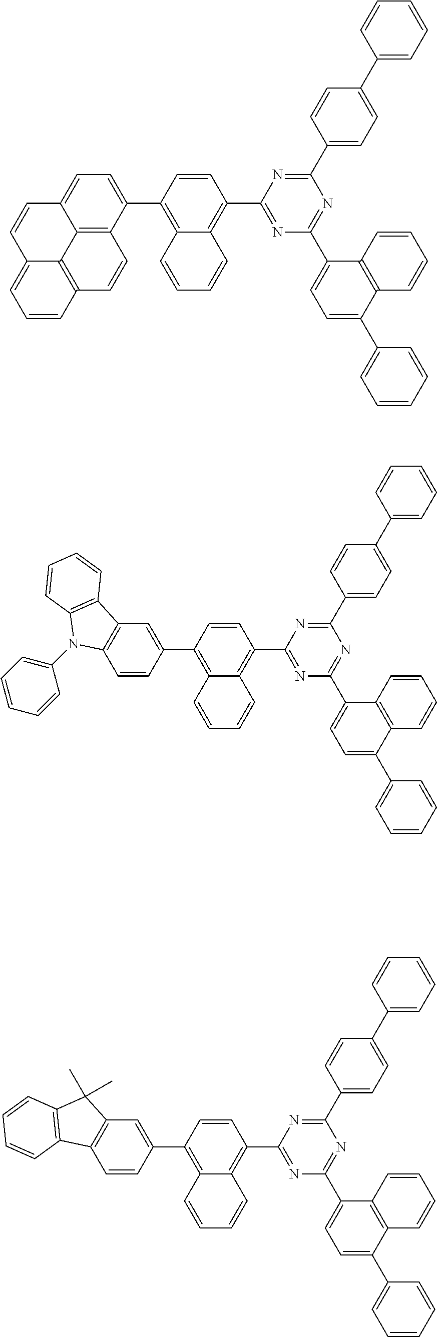 [SMILES: c1ccc(-c2ccc(-c3nc(-c4ccc(-c5ccccc5)c5ccccc45)nc(-c4ccc(-c5ccc6ccc7cccc8ccc5c6c78)c5ccccc45)n3)cc2)cc1 ; c1ccc(-c2ccc(-c3nc(-c4ccc(-c5ccccc5)c5ccccc45)nc(-c4ccc(-c5ccc6c(c5)c5ccccc5n6-c5ccccc5)c5ccccc45)n3)cc2)cc1 ; CC1(C)c2ccccc2-c2ccc(-c3ccc(-c4nc(-c5ccc(-c6ccccc6)cc5)nc(-c5ccc(-c6ccccc6)c6ccccc56)n4)c4ccccc34)cc21]

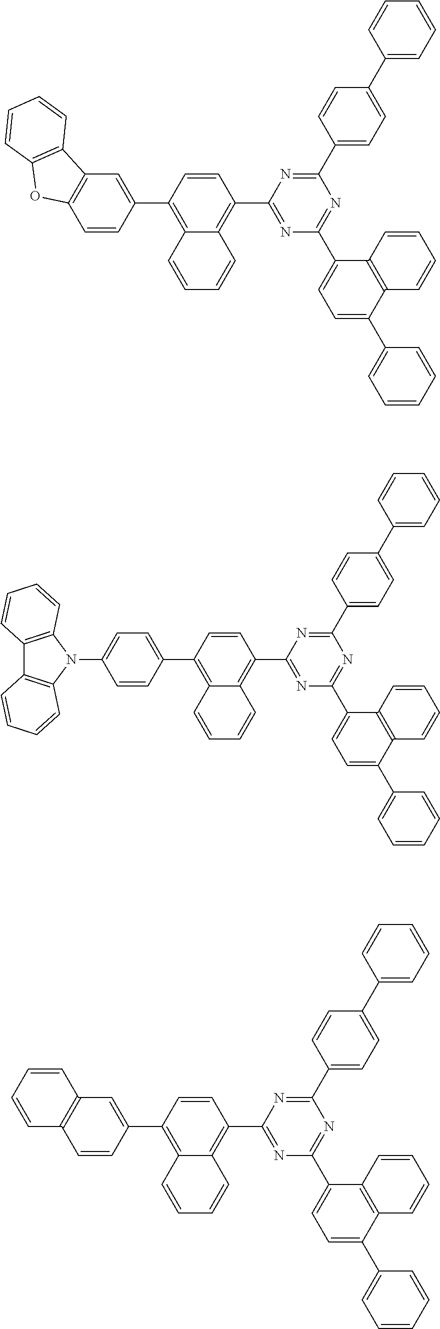 [SMILES: c1ccc(-c2ccc(-c3nc(-c4ccc(-c5ccccc5)c5ccccc45)nc(-c4ccc(-c5ccc6oc7ccccc7c6c5)c5ccccc45)n3)cc2)cc1 ; c1ccc(-c2ccc(-c3nc(-c4ccc(-c5ccccc5)c5ccccc45)nc(-c4ccc(-c5ccc(-n6c7ccccc7c7ccccc76)cc5)c5ccccc45)n3)cc2)cc1 ; c1ccc(-c2ccc(-c3nc(-c4ccc(-c5ccccc5)c5ccccc45)nc(-c4ccc(-c5ccc6ccccc6c5)c5ccccc45)n3)cc2)cc1]

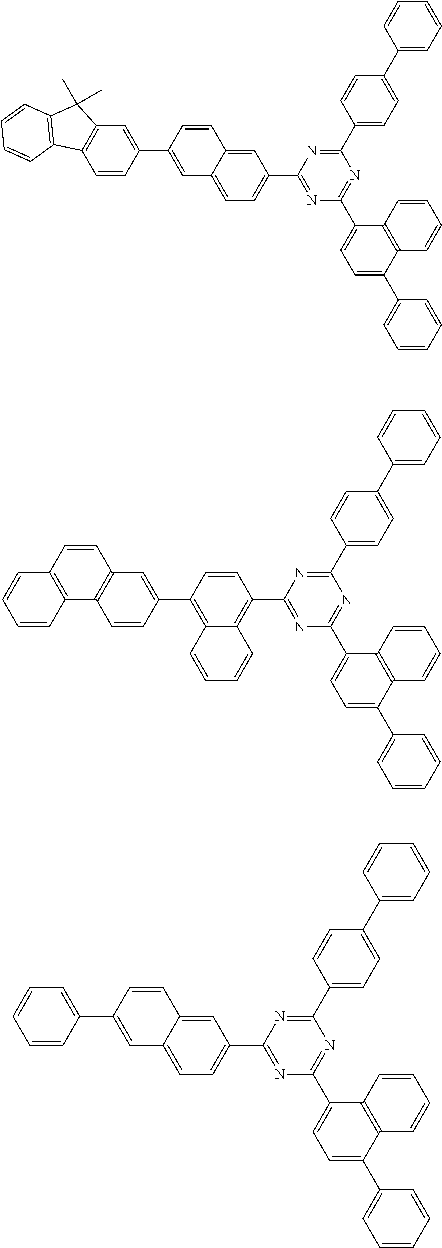 [SMILES: CC1(C)c2ccccc2-c2ccc(-c3ccc4cc(-c5nc(-c6ccc(-c7ccccc7)cc6)nc(-c6ccc(-c7ccccc7)c7ccccc67)n5)ccc4c3)cc21 ; c1ccc(-c2ccc(-c3nc(-c4ccc(-c5ccccc5)c5ccccc45)nc(-c4ccc(-c5ccc6c(ccc7ccccc76)c5)c5ccccc45)n3)cc2)cc1 ; c1ccc(-c2ccc(-c3nc(-c4ccc5cc(-c6ccccc6)ccc5c4)nc(-c4ccc(-c5ccccc5)c5ccccc45)n3)cc2)cc1]

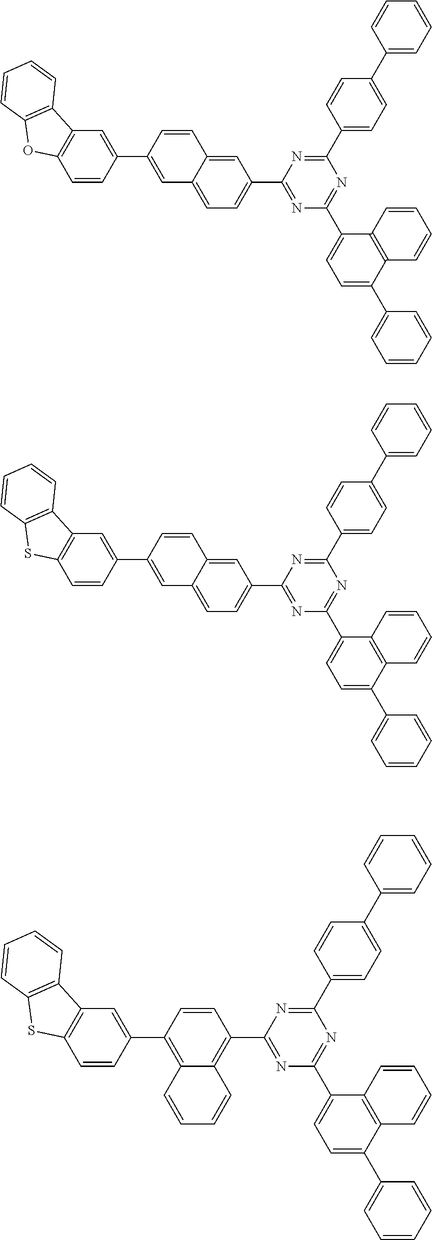

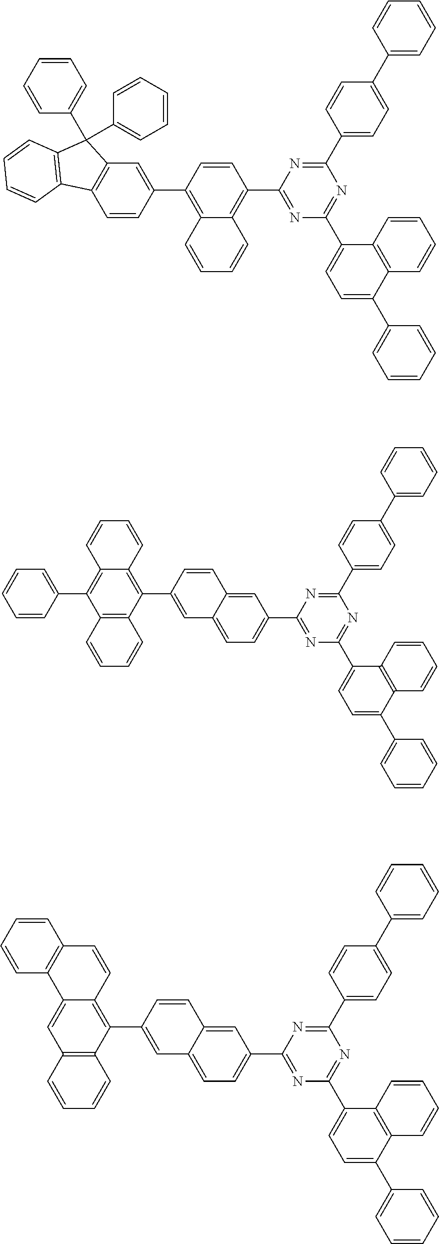 [SMILES: c1ccc(-c2ccc(-c3nc(-c4ccc(-c5ccccc5)c5ccccc45)nc(-c4ccc(-c5ccc6c(c5)C(c5ccccc5)(c5ccccc5)c5ccccc5-6)c5ccccc45)n3)cc2)cc1 ; c1ccc(-c2ccc(-c3nc(-c4ccc5cc(-c6c7ccccc7c(-c7ccccc7)c7ccccc67)ccc5c4)nc(-c4ccc(-c5ccccc5)c5ccccc45)n3)cc2)cc1 ; c1ccc(-c2ccc(-c3nc(-c4ccc5cc(-c6c7ccccc7cc7c6ccc6ccccc67)ccc5c4)nc(-c4ccc(-c5ccccc5)c5ccccc45)n3)cc2)cc1]

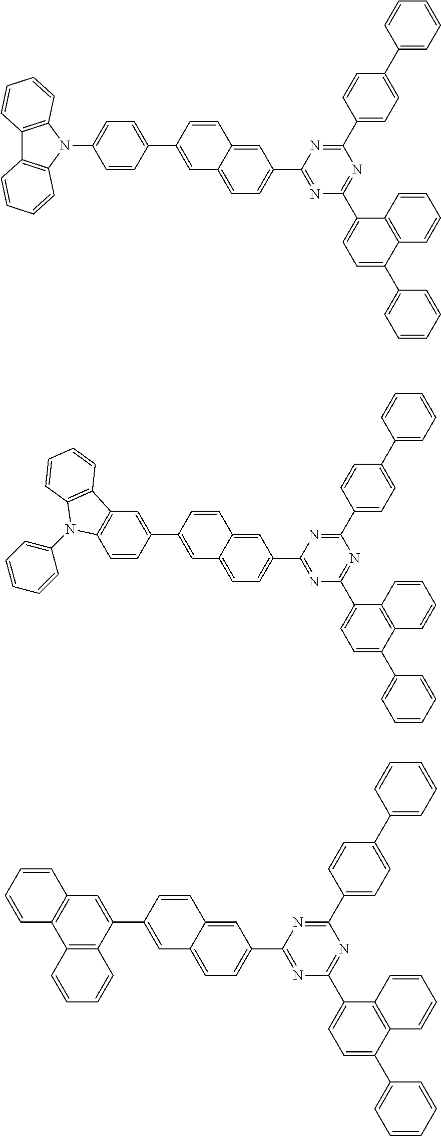 [SMILES: c1ccc(-c2ccc(-c3nc(-c4ccc5cc(-c6ccc(-n7c8ccccc8c8ccccc87)cc6)ccc5c4)nc(-c4ccc(-c5ccccc5)c5ccccc45)n3)cc2)cc1 ; c1ccc(-c2ccc(-c3nc(-c4ccc5cc(-c6ccc7c(c6)c6ccccc6n7-c6ccccc6)ccc5c4)nc(-c4ccc(-c5ccccc5)c5ccccc45)n3)cc2)cc1 ; c1ccc(-c2ccc(-c3nc(-c4ccc5cc(-c6cc7ccccc7c7ccccc67)ccc5c4)nc(-c4ccc(-c5ccccc5)c5ccccc45)n3)cc2)cc1]

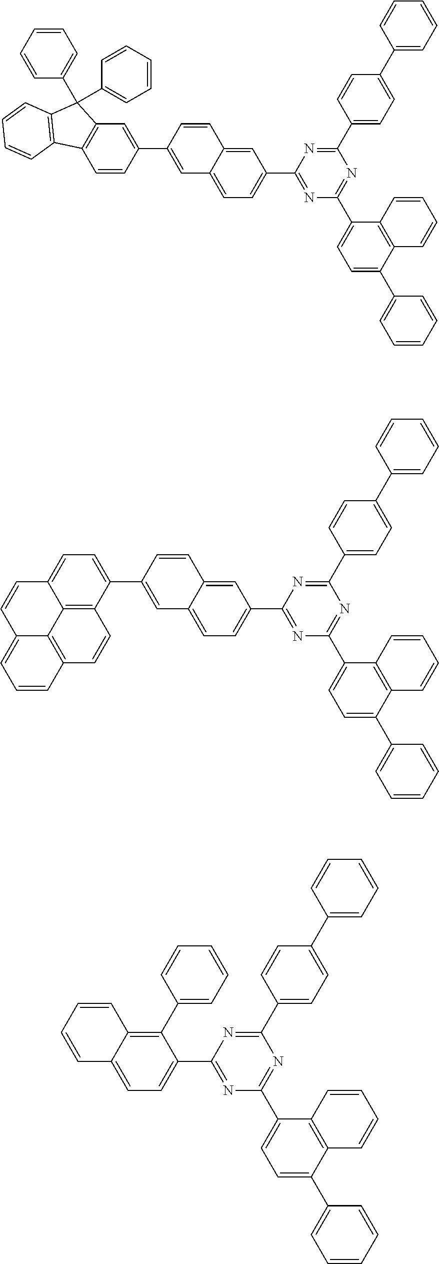 [SMILES: c1ccc(-c2ccc(-c3nc(-c4ccc5cc(-c6ccc7c(c6)C(c6ccccc6)(c6ccccc6)c6ccccc6-7)ccc5c4)nc(-c4ccc(-c5ccccc5)c5ccccc45)n3)cc2)cc1 ; c1ccc(-c2ccc(-c3nc(-c4ccc5cc(-c6ccc7ccc8cccc9ccc6c7c89)ccc5c4)nc(-c4ccc(-c5ccccc5)c5ccccc45)n3)cc2)cc1 ; c1ccc(-c2ccc(-c3nc(-c4ccc5ccccc5c4-c4ccccc4)nc(-c4ccc(-c5ccccc5)c5ccccc45)n3)cc2)cc1]

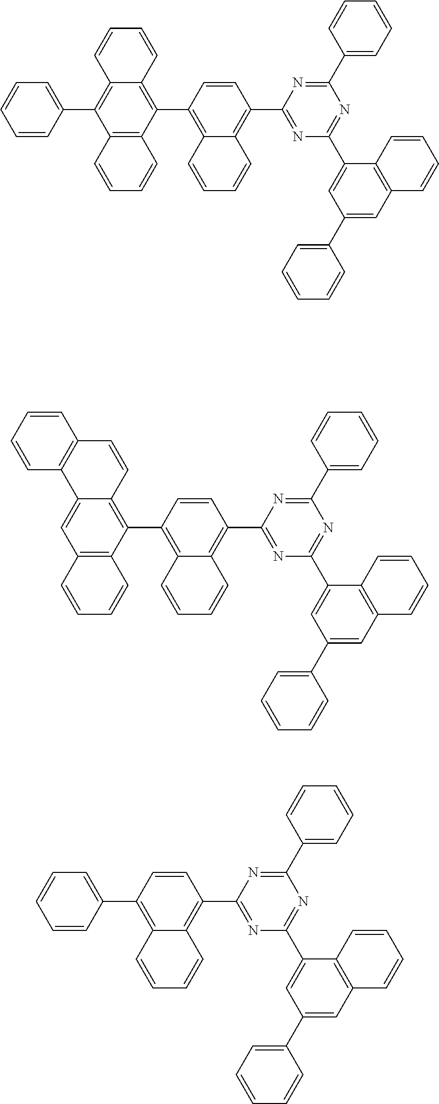 [SMILES: c1ccc(-c2cc(-c3nc(-c4ccccc4)nc(-c4ccc(-c5c6ccccc6c(-c6ccccc6)c6ccccc56)c5ccccc45)n3)c3ccccc3c2)cc1 ; c1ccc(-c2cc(-c3nc(-c4ccccc4)nc(-c4ccc(-c5c6ccccc6cc6c5ccc5ccccc56)c5ccccc45)n3)c3ccccc3c2)cc1 ; c1ccc(-c2cc(-c3nc(-c4ccccc4)nc(-c4ccc(-c5ccccc5)c5ccccc45)n3)c3ccccc3c2)cc1]

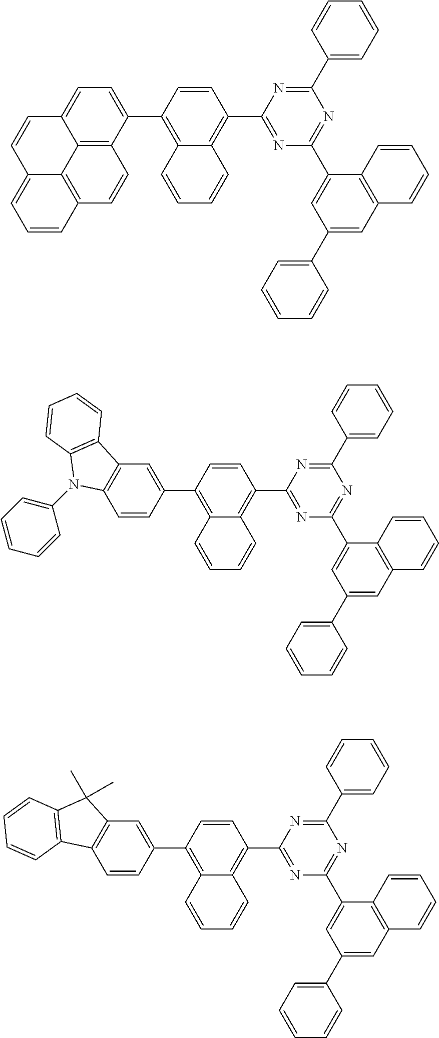 [SMILES: c1ccc(-c2cc(-c3nc(-c4ccccc4)nc(-c4ccc(-c5ccc6ccc7cccc8ccc5c6c78)c5ccccc45)n3)c3ccccc3c2)cc1 ; c1ccc(-c2cc(-c3nc(-c4ccccc4)nc(-c4ccc(-c5ccc6c(c5)c5ccccc5n6-c5ccccc5)c5ccccc45)n3)c3ccccc3c2)cc1 ; CC1(C)c2ccccc2-c2ccc(-c3ccc(-c4nc(-c5ccccc5)nc(-c5cc(-c6ccccc6)cc6ccccc56)n4)c4ccccc34)cc21]

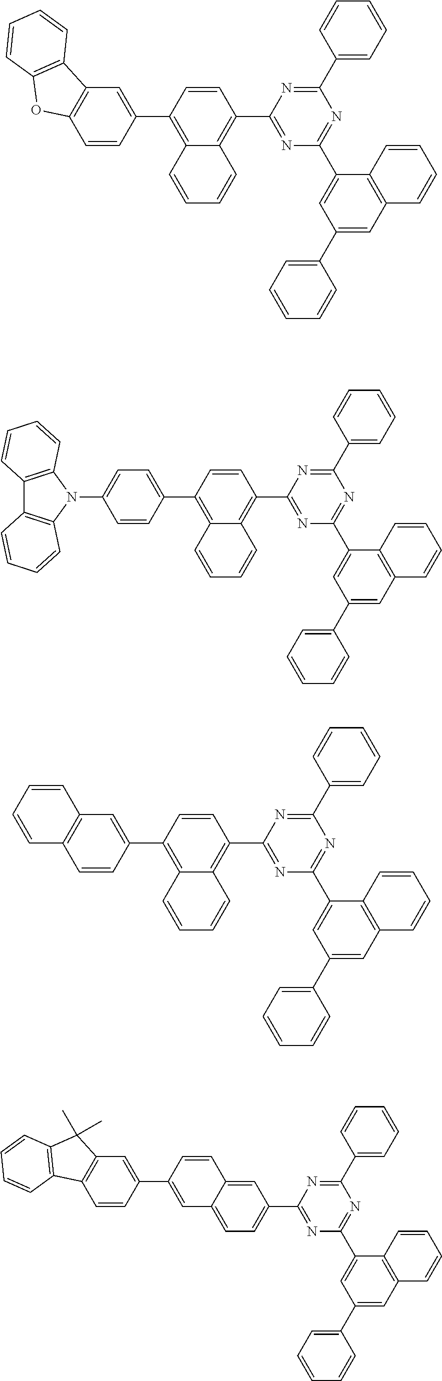 [SMILES: c1ccc(-c2cc(-c3nc(-c4ccccc4)nc(-c4ccc(-c5ccc6oc7ccccc7c6c5)c5ccccc45)n3)c3ccccc3c2)cc1 ; c1ccc(-c2cc(-c3nc(-c4ccccc4)nc(-c4ccc(-c5ccc(-n6c7ccccc7c7ccccc76)cc5)c5ccccc45)n3)c3ccccc3c2)cc1 ; c1ccc(-c2cc(-c3nc(-c4ccccc4)nc(-c4ccc(-c5ccc6ccccc6c5)c5ccccc45)n3)c3ccccc3c2)cc1 ; CC1(C)c2ccccc2-c2ccc(-c3ccc4cc(-c5nc(-c6ccccc6)nc(-c6cc(-c7ccccc7)cc7ccccc67)n5)ccc4c3)cc21]

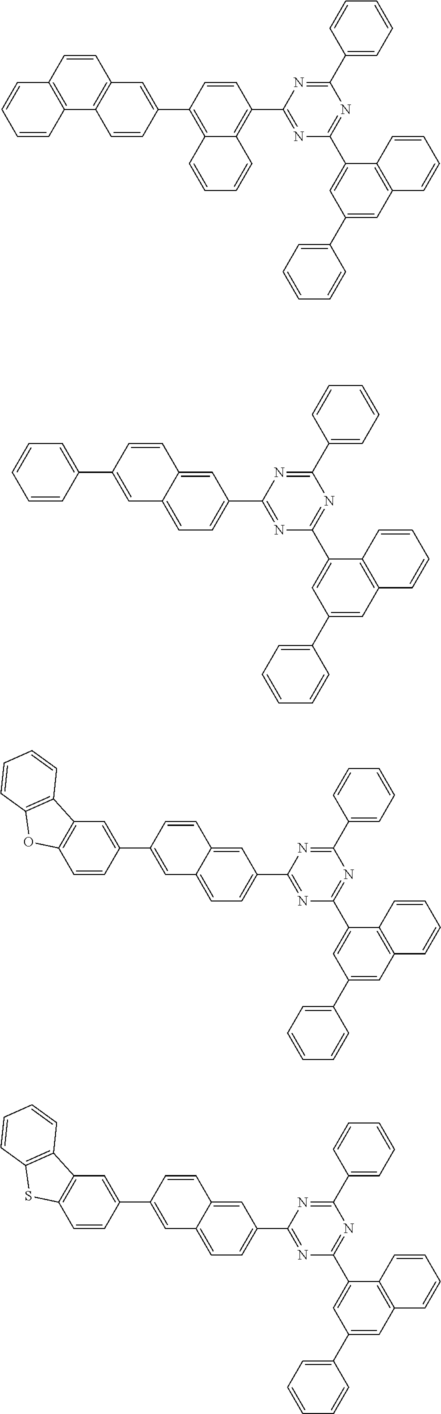 [SMILES: c1ccc(-c2cc(-c3nc(-c4ccccc4)nc(-c4ccc(-c5ccc6c(ccc7ccccc76)c5)c5ccccc45)n3)c3ccccc3c2)cc1 ; c1ccc(-c2ccc3cc(-c4nc(-c5ccccc5)nc(-c5cc(-c6ccccc6)cc6ccccc56)n4)ccc3c2)cc1 ; c1ccc(-c2cc(-c3nc(-c4ccccc4)nc(-c4ccc5cc(-c6ccc7oc8ccccc8c7c6)ccc5c4)n3)c3ccccc3c2)cc1 ; c1ccc(-c2cc(-c3nc(-c4ccccc4)nc(-c4ccc5cc(-c6ccc7sc8ccccc8c7c6)ccc5c4)n3)c3ccccc3c2)cc1]

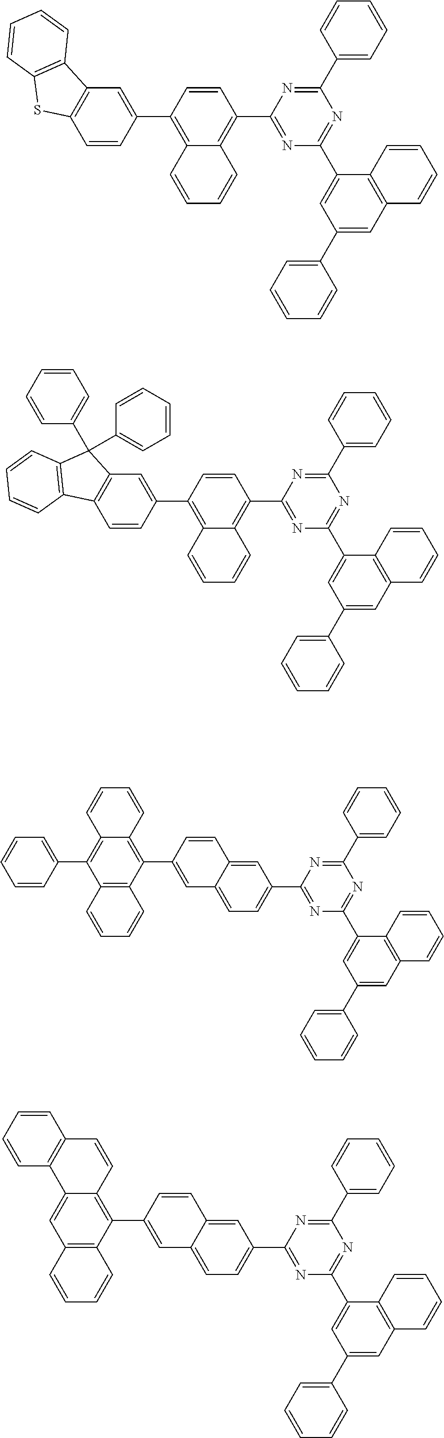 [SMILES: c1ccc(-c2cc(-c3nc(-c4ccccc4)nc(-c4ccc(-c5ccc6sc7ccccc7c6c5)c5ccccc45)n3)c3ccccc3c2)cc1 ; c1ccc(-c2cc(-c3nc(-c4ccccc4)nc(-c4ccc(-c5ccc6c(c5)C(c5ccccc5)(c5ccccc5)c5ccccc5-6)c5ccccc45)n3)c3ccccc3c2)cc1 ; c1ccc(-c2cc(-c3nc(-c4ccccc4)nc(-c4ccc5cc(-c6c7ccccc7c(-c7ccccc7)c7ccccc67)ccc5c4)n3)c3ccccc3c2)cc1 ; c1ccc(-c2cc(-c3nc(-c4ccccc4)nc(-c4ccc5cc(-c6c7ccccc7cc7c6ccc6ccccc67)ccc5c4)n3)c3ccccc3c2)cc1]

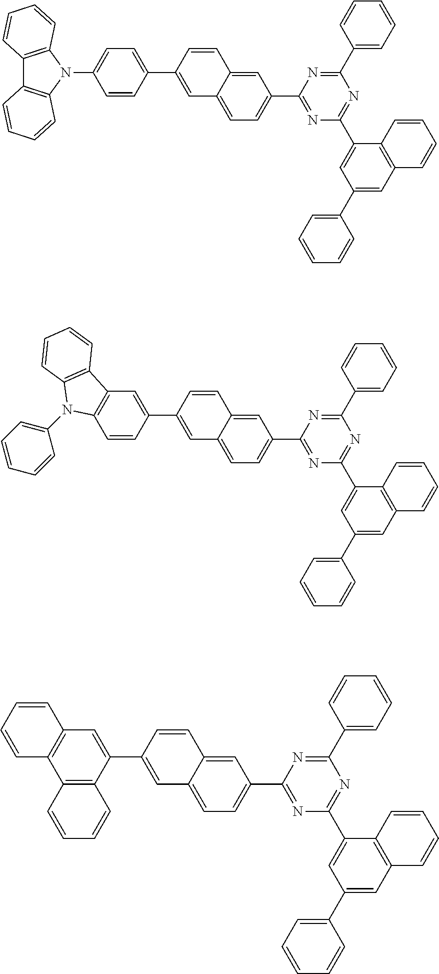 [SMILES: c1ccc(-c2cc(-c3nc(-c4ccccc4)nc(-c4ccc5cc(-c6ccc(-n7c8ccccc8c8ccccc87)cc6)ccc5c4)n3)c3ccccc3c2)cc1 ; c1ccc(-c2cc(-c3nc(-c4ccccc4)nc(-c4ccc5cc(-c6ccc7c(c6)c6ccccc6n7-c6ccccc6)ccc5c4)n3)c3ccccc3c2)cc1 ; c1ccc(-c2cc(-c3nc(-c4ccccc4)nc(-c4ccc5cc(-c6cc7ccccc7c7ccccc67)ccc5c4)n3)c3ccccc3c2)cc1]

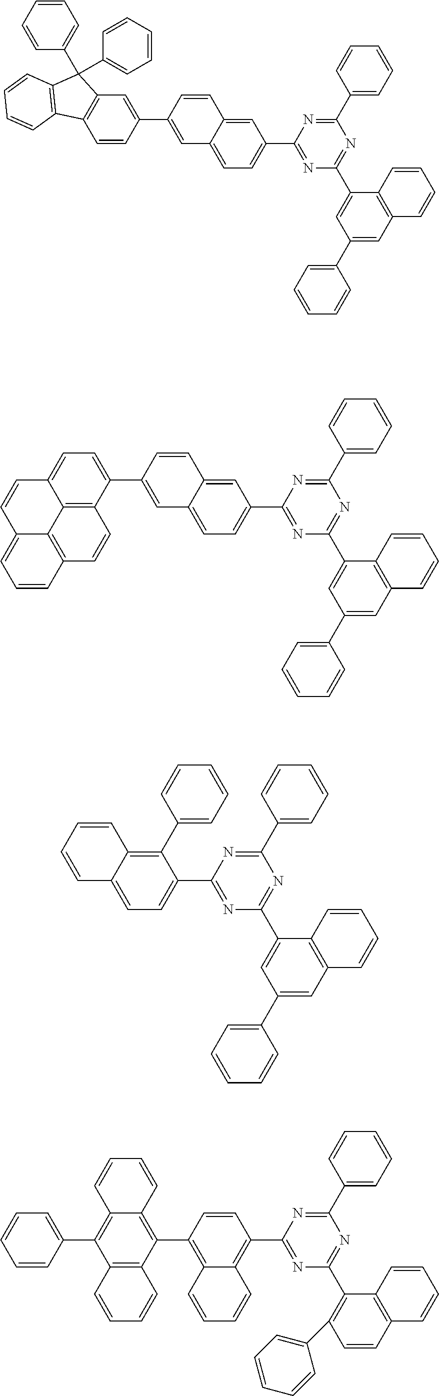 [SMILES: c1ccc(-c2cc(-c3nc(-c4ccccc4)nc(-c4ccc5cc(-c6ccc7c(c6)C(c6ccccc6)(c6ccccc6)c6ccccc6-7)ccc5c4)n3)c3ccccc3c2)cc1 ; c1ccc(-c2cc(-c3nc(-c4ccccc4)nc(-c4ccc5cc(-c6ccc7ccc8cccc9ccc6c7c89)ccc5c4)n3)c3ccccc3c2)cc1 ; c1ccc(-c2cc(-c3nc(-c4ccccc4)nc(-c4ccc5ccccc5c4-c4ccccc4)n3)c3ccccc3c2)cc1 ; c1ccc(-c2nc(-c3c(-c4ccccc4)ccc4ccccc34)nc(-c3ccc(-c4c5ccccc5c(-c5ccccc5)c5ccccc45)c4ccccc34)n2)cc1]

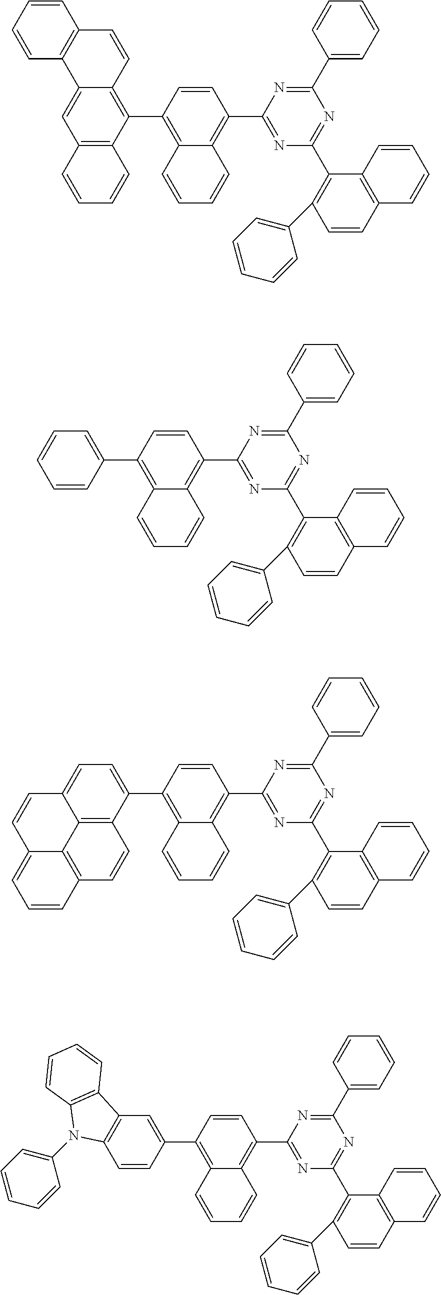 [SMILES: c1ccc(-c2nc(-c3c(-c4ccccc4)ccc4ccccc34)nc(-c3ccc(-c4c5ccccc5cc5c4ccc4ccccc45)c4ccccc34)n2)cc1 ; c1ccc(-c2nc(-c3c(-c4ccccc4)ccc4ccccc34)nc(-c3ccc(-c4ccccc4)c4ccccc34)n2)cc1 ; c1ccc(-c2nc(-c3c(-c4ccccc4)ccc4ccccc34)nc(-c3ccc(-c4ccc5ccc6cccc7ccc4c5c67)c4ccccc34)n2)cc1 ; c1ccc(-c2nc(-c3c(-c4ccccc4)ccc4ccccc34)nc(-c3ccc(-c4ccc5c(c4)c4ccccc4n5-c4ccccc4)c4ccccc34)n2)cc1]

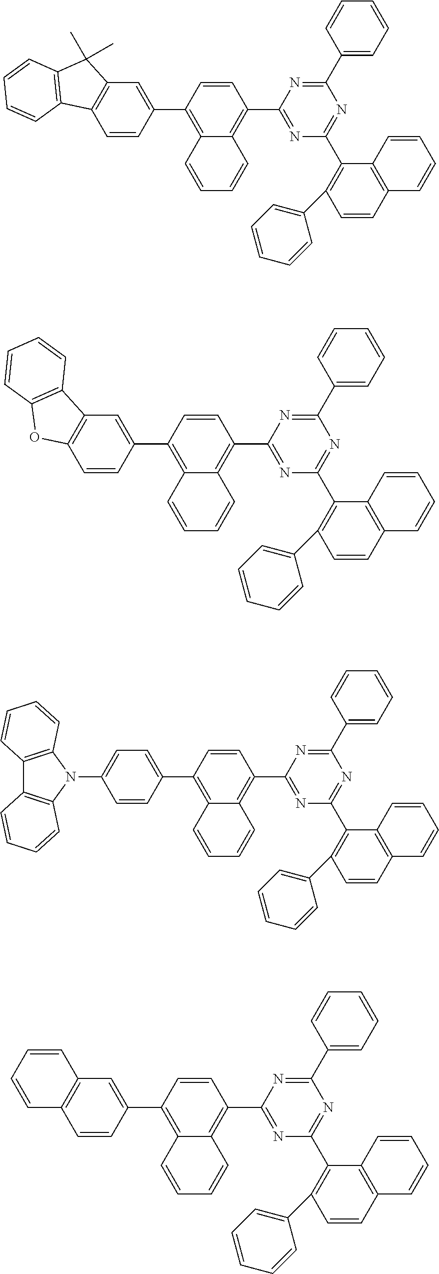 [SMILES: CC1(C)c2ccccc2-c2ccc(-c3ccc(-c4nc(-c5ccccc5)nc(-c5c(-c6ccccc6)ccc6ccccc56)n4)c4ccccc34)cc21 ; c1ccc(-c2nc(-c3c(-c4ccccc4)ccc4ccccc34)nc(-c3ccc(-c4ccc5oc6ccccc6c5c4)c4ccccc34)n2)cc1 ; c1ccc(-c2nc(-c3c(-c4ccccc4)ccc4ccccc34)nc(-c3ccc(-c4ccc(-n5c6ccccc6c6ccccc65)cc4)c4ccccc34)n2)cc1 ; c1ccc(-c2nc(-c3c(-c4ccccc4)ccc4ccccc34)nc(-c3ccc(-c4ccc5ccccc5c4)c4ccccc34)n2)cc1]

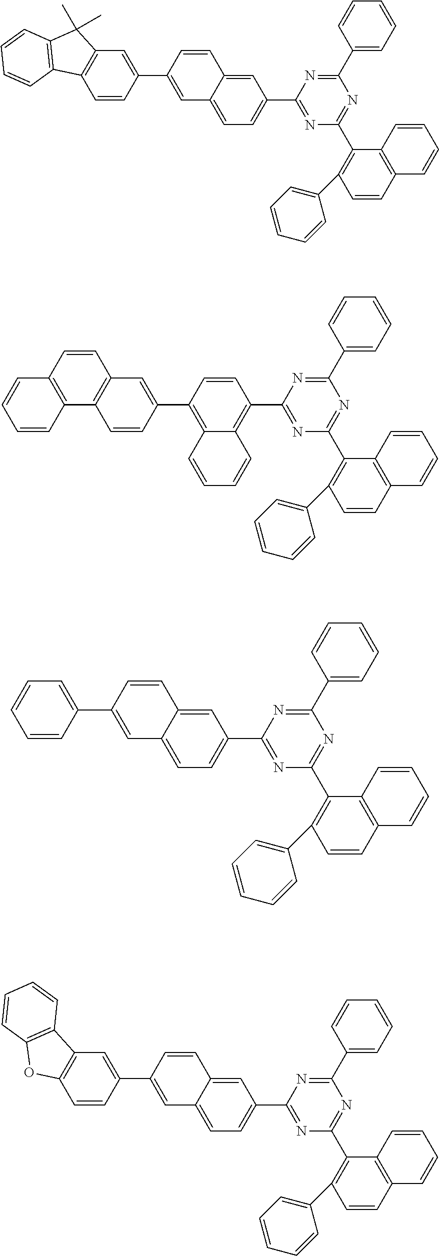 [SMILES: CC1(C)c2ccccc2-c2ccc(-c3ccc4cc(-c5nc(-c6ccccc6)nc(-c6c(-c7ccccc7)ccc7ccccc67)n5)ccc4c3)cc21 ; c1ccc(-c2nc(-c3c(-c4ccccc4)ccc4ccccc34)nc(-c3ccc(-c4ccc5c(ccc6ccccc65)c4)c4ccccc34)n2)cc1 ; c1ccc(-c2ccc3cc(-c4nc(-c5ccccc5)nc(-c5c(-c6ccccc6)ccc6ccccc56)n4)ccc3c2)cc1 ; c1ccc(-c2nc(-c3ccc4cc(-c5ccc6oc7ccccc7c6c5)ccc4c3)nc(-c3c(-c4ccccc4)ccc4ccccc34)n2)cc1]

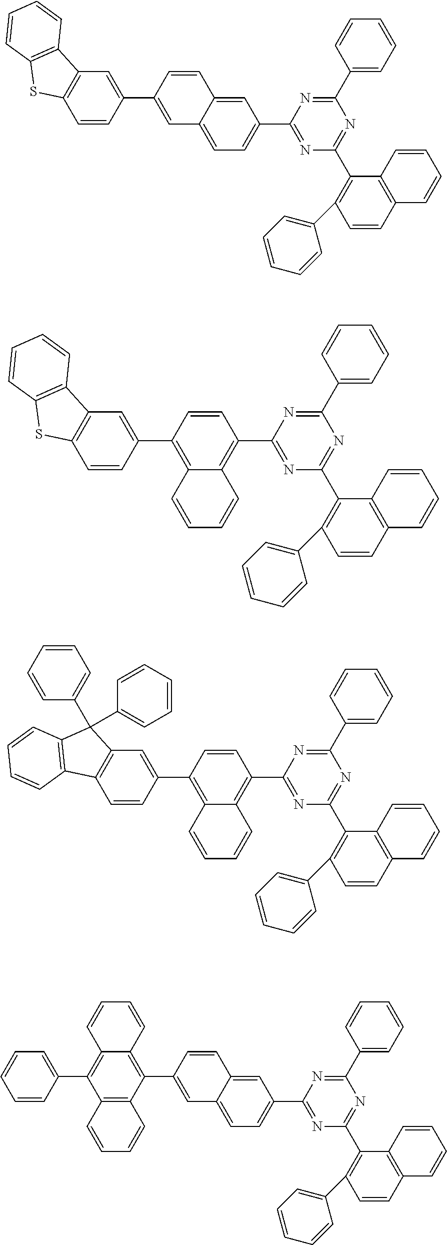 [SMILES: c1ccc(-c2nc(-c3ccc4cc(-c5ccc6sc7ccccc7c6c5)ccc4c3)nc(-c3c(-c4ccccc4)ccc4ccccc34)n2)cc1 ; c1ccc(-c2nc(-c3c(-c4ccccc4)ccc4ccccc34)nc(-c3ccc(-c4ccc5sc6ccccc6c5c4)c4ccccc34)n2)cc1 ; c1ccc(-c2nc(-c3c(-c4ccccc4)ccc4ccccc34)nc(-c3ccc(-c4ccc5c(c4)C(c4ccccc4)(c4ccccc4)c4ccccc4-5)c4ccccc34)n2)cc1 ; c1ccc(-c2nc(-c3ccc4cc(-c5c6ccccc6c(-c6ccccc6)c6ccccc56)ccc4c3)nc(-c3c(-c4ccccc4)ccc4ccccc34)n2)cc1]

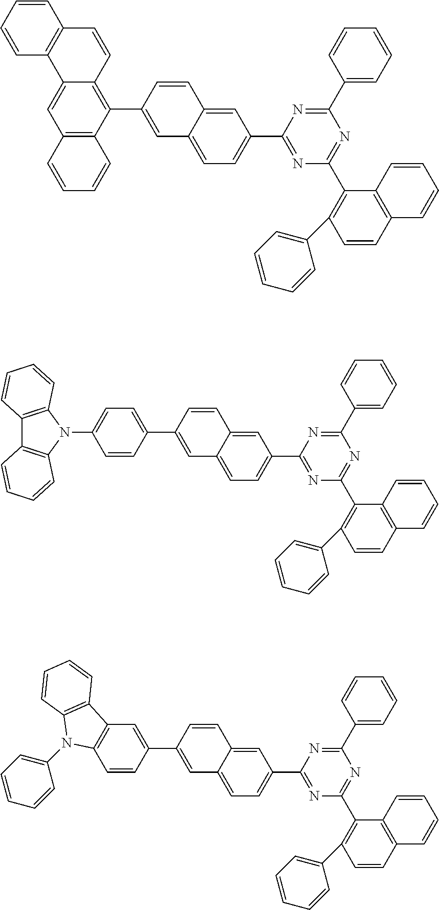 [SMILES: c1ccc(-c2nc(-c3ccc4cc(-c5c6ccccc6cc6c5ccc5ccccc56)ccc4c3)nc(-c3c(-c4ccccc4)ccc4ccccc34)n2)cc1 ; c1ccc(-c2nc(-c3ccc4cc(-c5ccc(-n6c7ccccc7c7ccccc76)cc5)ccc4c3)nc(-c3c(-c4ccccc4)ccc4ccccc34)n2)cc1 ; c1ccc(-c2nc(-c3ccc4cc(-c5ccc6c(c5)c5ccccc5n6-c5ccccc5)ccc4c3)nc(-c3c(-c4ccccc4)ccc4ccccc34)n2)cc1]

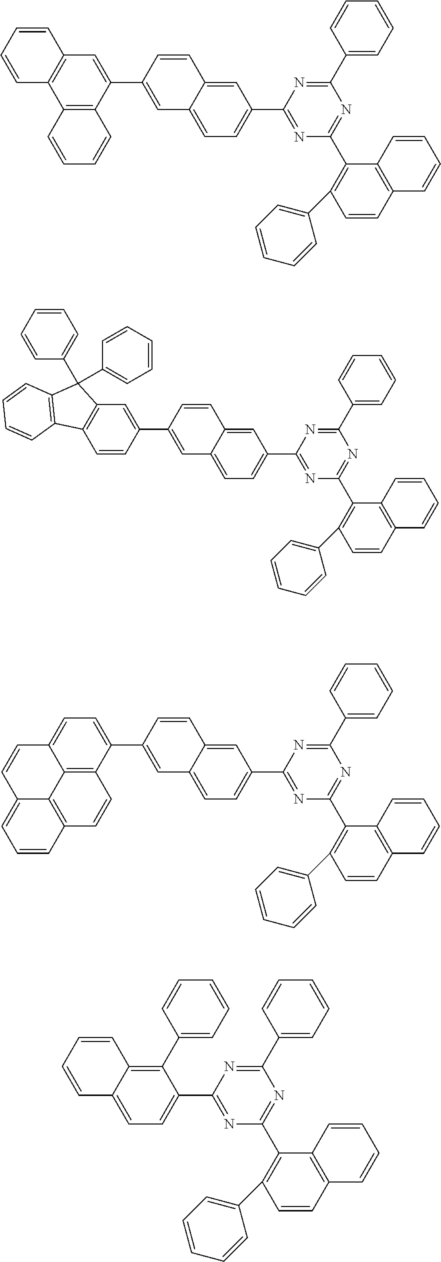 [SMILES: c1ccc(-c2nc(-c3ccc4cc(-c5cc6ccccc6c6ccccc56)ccc4c3)nc(-c3c(-c4ccccc4)ccc4ccccc34)n2)cc1 ; c1ccc(-c2nc(-c3ccc4cc(-c5ccc6c(c5)C(c5ccccc5)(c5ccccc5)c5ccccc5-6)ccc4c3)nc(-c3c(-c4ccccc4)ccc4ccccc34)n2)cc1 ; c1ccc(-c2nc(-c3ccc4cc(-c5ccc6ccc7cccc8ccc5c6c78)ccc4c3)nc(-c3c(-c4ccccc4)ccc4ccccc34)n2)cc1 ; c1ccc(-c2nc(-c3ccc4ccccc4c3-c3ccccc3)nc(-c3c(-c4ccccc4)ccc4ccccc34)n2)cc1]

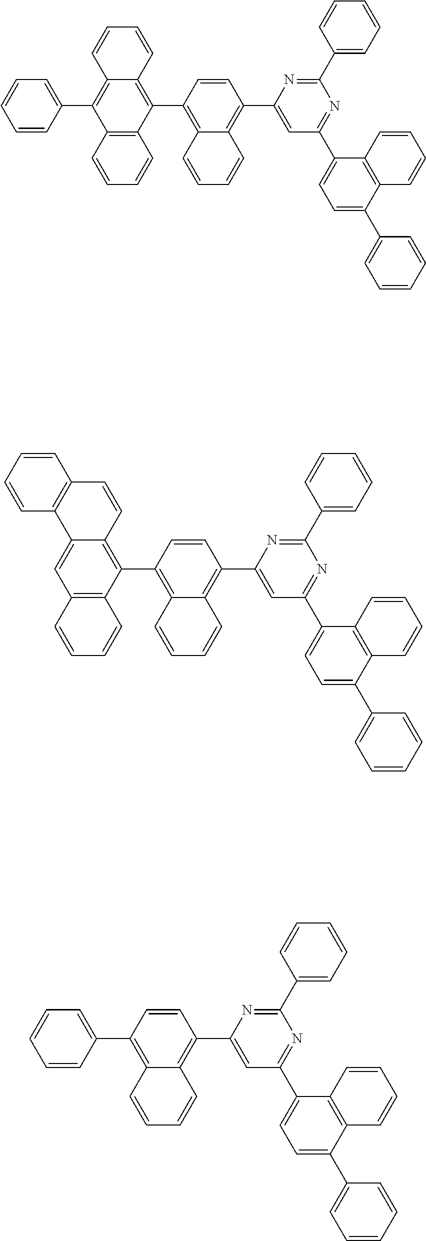 [SMILES: c1ccc(-c2nc(-c3ccc(-c4ccccc4)c4ccccc34)cc(-c3ccc(-c4c5ccccc5c(-c5ccccc5)c5ccccc45)c4ccccc34)n2)cc1 ; c1ccc(-c2nc(-c3ccc(-c4ccccc4)c4ccccc34)cc(-c3ccc(-c4c5ccccc5cc5c4ccc4ccccc45)c4ccccc34)n2)cc1 ; c1ccc(-c2nc(-c3ccc(-c4ccccc4)c4ccccc34)cc(-c3ccc(-c4ccccc4)c4ccccc34)n2)cc1]

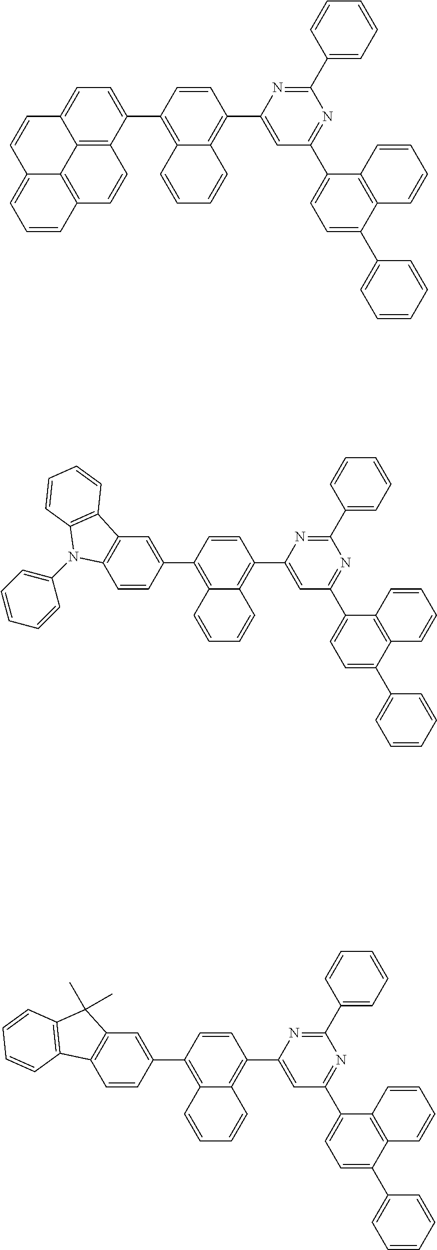 [SMILES: c1ccc(-c2nc(-c3ccc(-c4ccccc4)c4ccccc34)cc(-c3ccc(-c4ccc5ccc6cccc7ccc4c5c67)c4ccccc34)n2)cc1 ; c1ccc(-c2nc(-c3ccc(-c4ccccc4)c4ccccc34)cc(-c3ccc(-c4ccc5c(c4)c4ccccc4n5-c4ccccc4)c4ccccc34)n2)cc1 ; CC1(C)c2ccccc2-c2ccc(-c3ccc(-c4cc(-c5ccc(-c6ccccc6)c6ccccc56)nc(-c5ccccc5)n4)c4ccccc34)cc21]

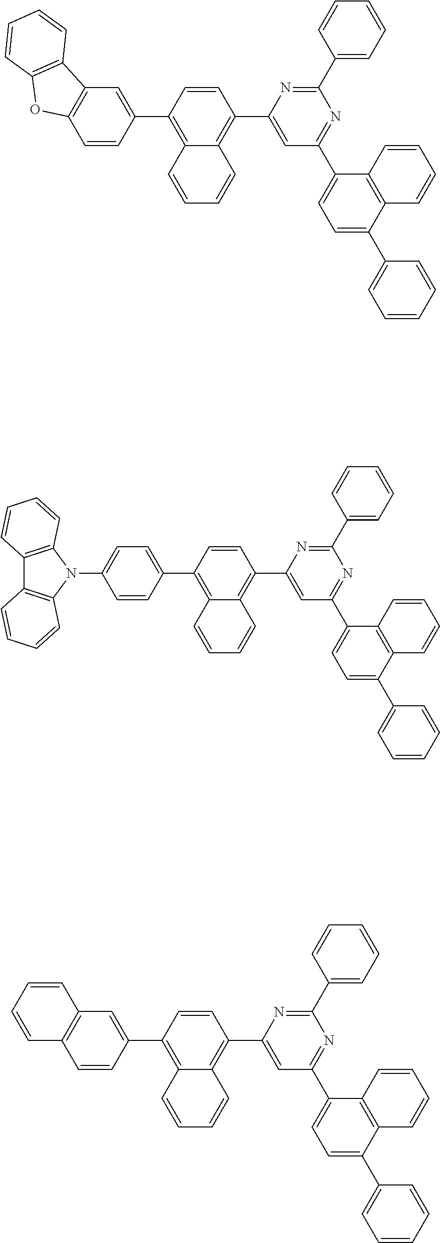 [SMILES: c1ccc(-c2nc(-c3ccc(-c4ccccc4)c4ccccc34)cc(-c3ccc(-c4ccc5oc6ccccc6c5c4)c4ccccc34)n2)cc1 ; c1ccc(-c2nc(-c3ccc(-c4ccccc4)c4ccccc34)cc(-c3ccc(-c4ccc(-n5c6ccccc6c6ccccc65)cc4)c4ccccc34)n2)cc1 ; c1ccc(-c2nc(-c3ccc(-c4ccccc4)c4ccccc34)cc(-c3ccc(-c4ccc5ccccc5c4)c4ccccc34)n2)cc1]

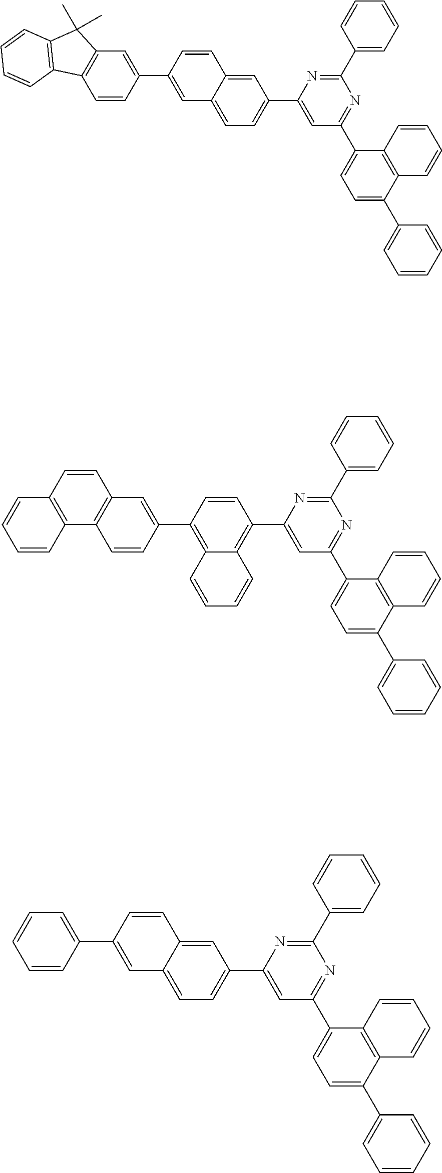 [SMILES: CC1(C)c2ccccc2-c2ccc(-c3ccc4cc(-c5cc(-c6ccc(-c7ccccc7)c7ccccc67)nc(-c6ccccc6)n5)ccc4c3)cc21 ; c1ccc(-c2nc(-c3ccc(-c4ccccc4)c4ccccc34)cc(-c3ccc(-c4ccc5c(ccc6ccccc65)c4)c4ccccc34)n2)cc1 ; c1ccc(-c2ccc3cc(-c4cc(-c5ccc(-c6ccccc6)c6ccccc56)nc(-c5ccccc5)n4)ccc3c2)cc1]

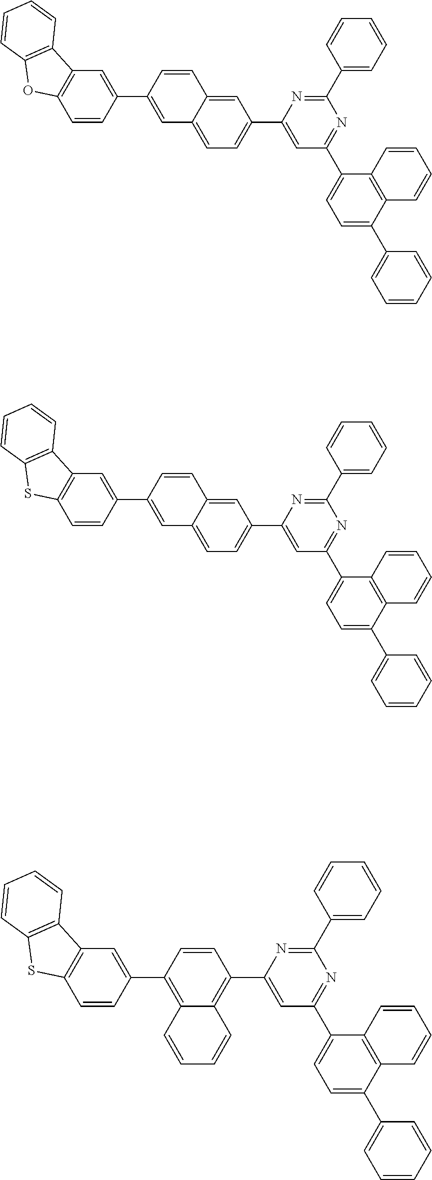 [SMILES: c1ccc(-c2nc(-c3ccc4cc(-c5ccc6oc7ccccc7c6c5)ccc4c3)cc(-c3ccc(-c4ccccc4)c4ccccc34)n2)cc1 ; c1ccc(-c2nc(-c3ccc4cc(-c5ccc6sc7ccccc7c6c5)ccc4c3)cc(-c3ccc(-c4ccccc4)c4ccccc34)n2)cc1 ; c1ccc(-c2nc(-c3ccc(-c4ccccc4)c4ccccc34)cc(-c3ccc(-c4ccc5sc6ccccc6c5c4)c4ccccc34)n2)cc1]

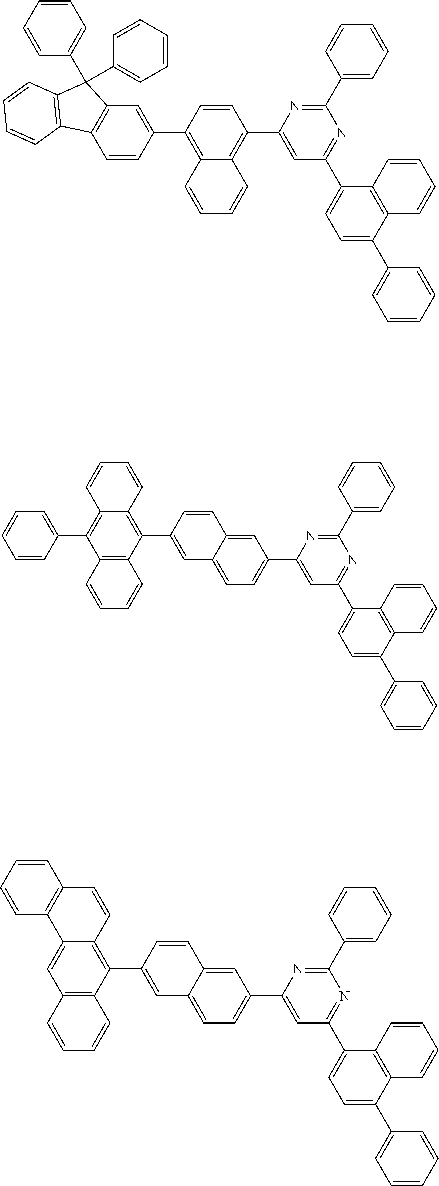 [SMILES: c1ccc(-c2nc(-c3ccc(-c4ccccc4)c4ccccc34)cc(-c3ccc(-c4ccc5c(c4)C(c4ccccc4)(c4ccccc4)c4ccccc4-5)c4ccccc34)n2)cc1 ; c1ccc(-c2nc(-c3ccc4cc(-c5c6ccccc6c(-c6ccccc6)c6ccccc56)ccc4c3)cc(-c3ccc(-c4ccccc4)c4ccccc34)n2)cc1 ; c1ccc(-c2nc(-c3ccc4cc(-c5c6ccccc6cc6c5ccc5ccccc56)ccc4c3)cc(-c3ccc(-c4ccccc4)c4ccccc34)n2)cc1]

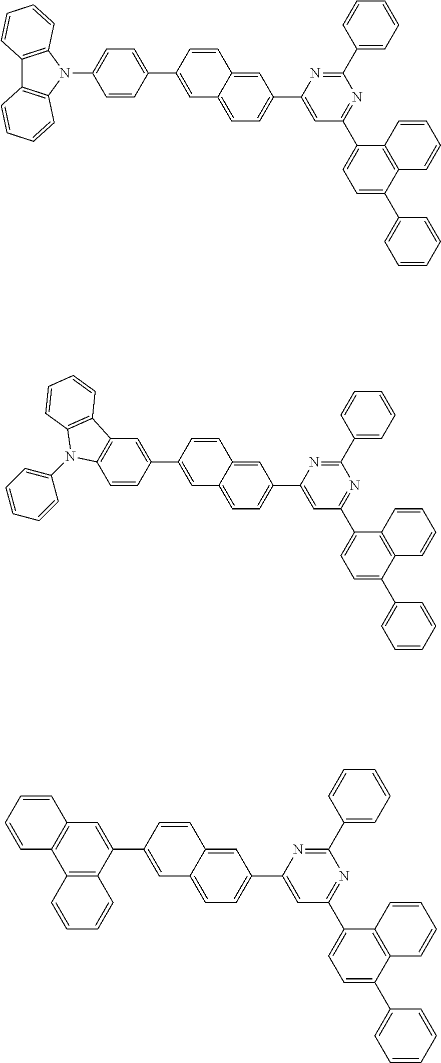 [SMILES: c1ccc(-c2nc(-c3ccc4cc(-c5ccc(-n6c7ccccc7c7ccccc76)cc5)ccc4c3)cc(-c3ccc(-c4ccccc4)c4ccccc34)n2)cc1 ; c1ccc(-c2nc(-c3ccc4cc(-c5ccc6c(c5)c5ccccc5n6-c5ccccc5)ccc4c3)cc(-c3ccc(-c4ccccc4)c4ccccc34)n2)cc1 ; c1ccc(-c2nc(-c3ccc4cc(-c5cc6ccccc6c6ccccc56)ccc4c3)cc(-c3ccc(-c4ccccc4)c4ccccc34)n2)cc1]

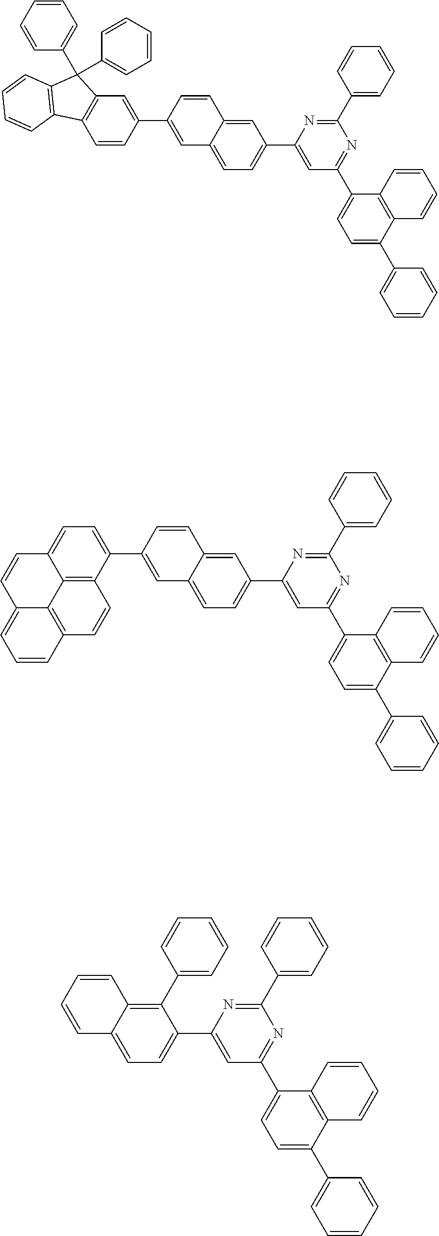 [SMILES: c1ccc(-c2nc(-c3ccc4cc(-c5ccc6c(c5)C(c5ccccc5)(c5ccccc5)c5ccccc5-6)ccc4c3)cc(-c3ccc(-c4ccccc4)c4ccccc34)n2)cc1 ; c1ccc(-c2nc(-c3ccc4cc(-c5ccc6ccc7cccc8ccc5c6c78)ccc4c3)cc(-c3ccc(-c4ccccc4)c4ccccc34)n2)cc1 ; c1ccc(-c2nc(-c3ccc4ccccc4c3-c3ccccc3)cc(-c3ccc(-c4ccccc4)c4ccccc34)n2)cc1]

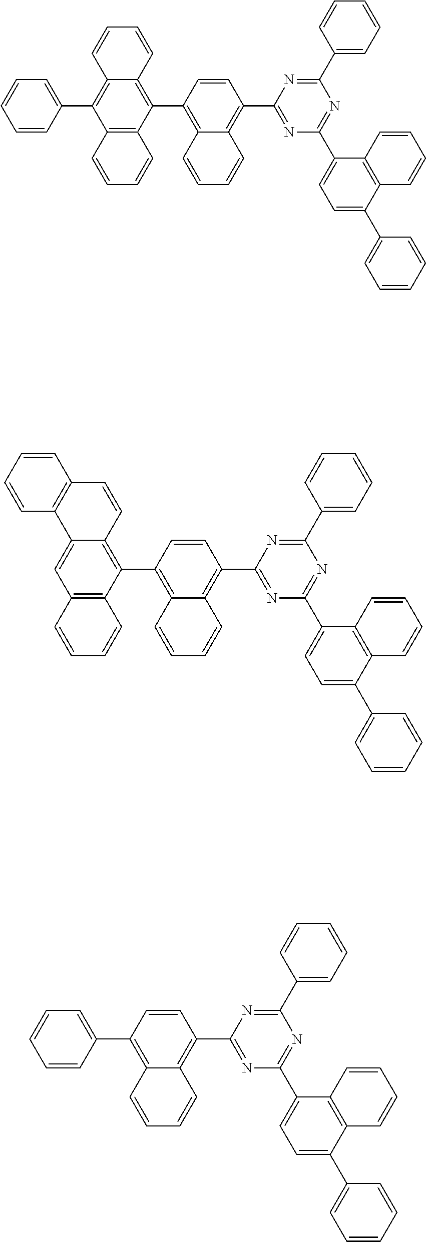 [SMILES: c1ccc(-c2nc(-c3ccc(-c4ccccc4)c4ccccc34)nc(-c3ccc(-c4c5ccccc5c(-c5ccccc5)c5ccccc45)c4ccccc34)n2)cc1 ; c1ccc(-c2nc(-c3ccc(-c4ccccc4)c4ccccc34)nc(-c3ccc(-c4c5ccccc5cc5c4ccc4ccccc45)c4ccccc34)n2)cc1 ; c1ccc(-c2nc(-c3ccc(-c4ccccc4)c4ccccc34)nc(-c3ccc(-c4ccccc4)c4ccccc34)n2)cc1]

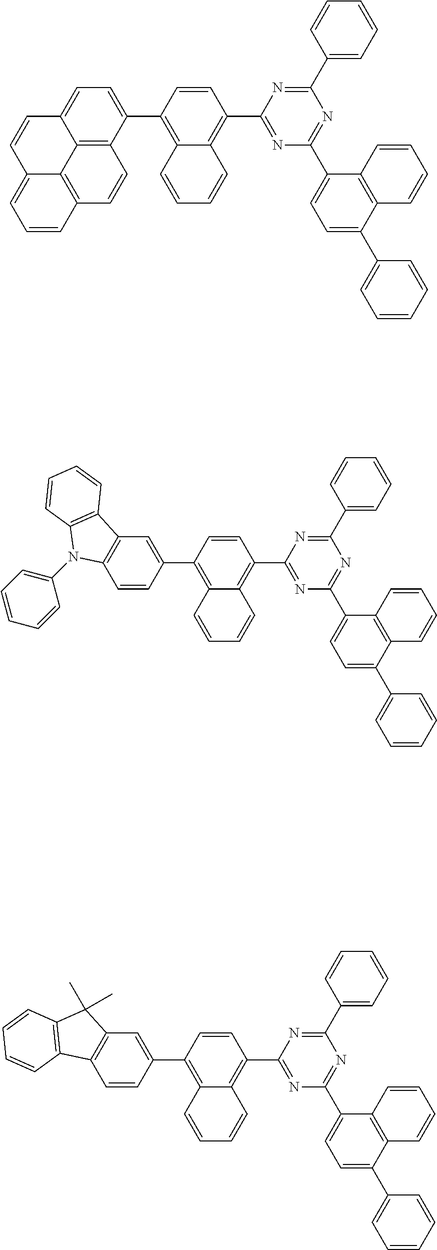 [SMILES: c1ccc(-c2nc(-c3ccc(-c4ccccc4)c4ccccc34)nc(-c3ccc(-c4ccc5ccc6cccc7ccc4c5c67)c4ccccc34)n2)cc1 ; c1ccc(-c2nc(-c3ccc(-c4ccccc4)c4ccccc34)nc(-c3ccc(-c4ccc5c(c4)c4ccccc4n5-c4ccccc4)c4ccccc34)n2)cc1 ; CC1(C)c2ccccc2-c2ccc(-c3ccc(-c4nc(-c5ccccc5)nc(-c5ccc(-c6ccccc6)c6ccccc56)n4)c4ccccc34)cc21]

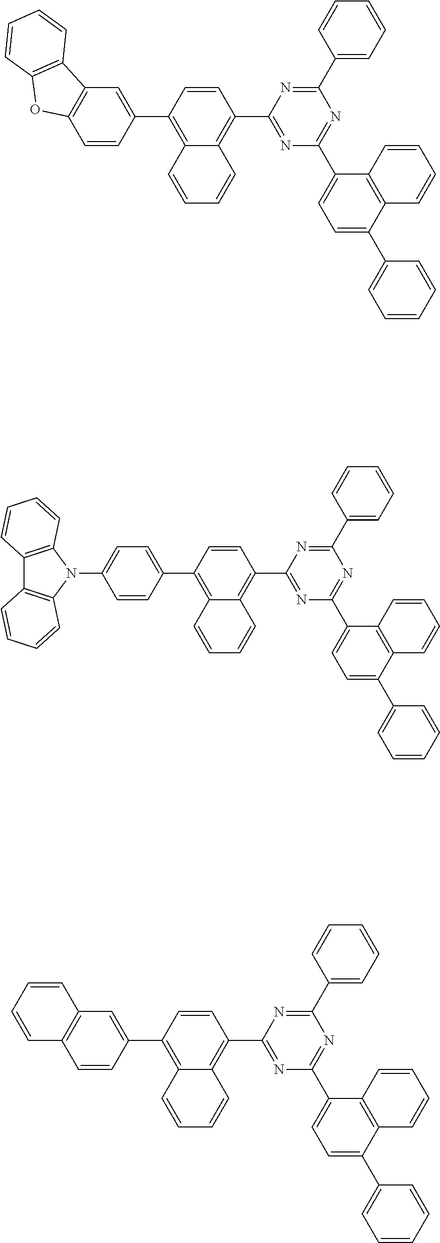 [SMILES: c1ccc(-c2nc(-c3ccc(-c4ccccc4)c4ccccc34)nc(-c3ccc(-c4ccc5oc6ccccc6c5c4)c4ccccc34)n2)cc1 ; c1ccc(-c2nc(-c3ccc(-c4ccccc4)c4ccccc34)nc(-c3ccc(-c4ccc(-n5c6ccccc6c6ccccc65)cc4)c4ccccc34)n2)cc1 ; c1ccc(-c2nc(-c3ccc(-c4ccccc4)c4ccccc34)nc(-c3ccc(-c4ccc5ccccc5c4)c4ccccc34)n2)cc1]

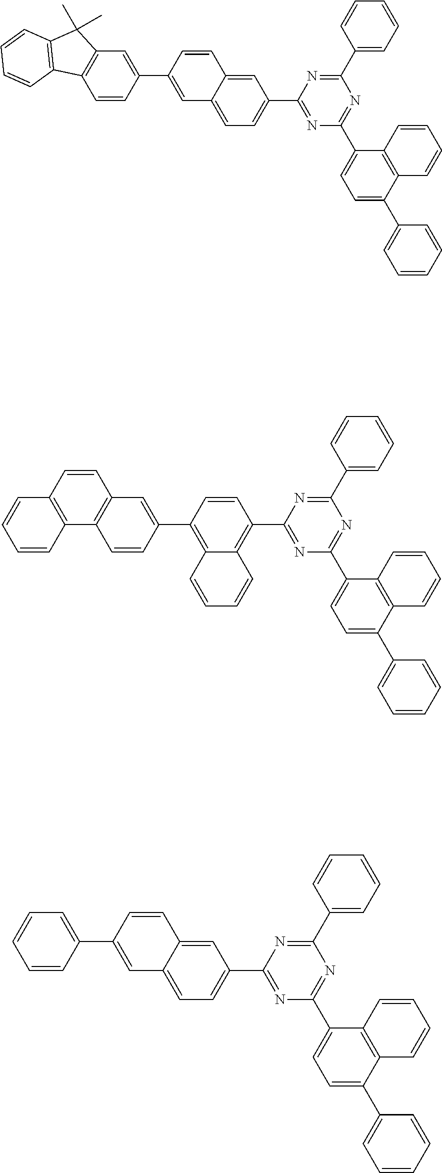 [SMILES: CC1(C)c2ccccc2-c2ccc(-c3ccc4cc(-c5nc(-c6ccccc6)nc(-c6ccc(-c7ccccc7)c7ccccc67)n5)ccc4c3)cc21 ; c1ccc(-c2nc(-c3ccc(-c4ccccc4)c4ccccc34)nc(-c3ccc(-c4ccc5c(ccc6ccccc65)c4)c4ccccc34)n2)cc1 ; c1ccc(-c2ccc3cc(-c4nc(-c5ccccc5)nc(-c5ccc(-c6ccccc6)c6ccccc56)n4)ccc3c2)cc1]

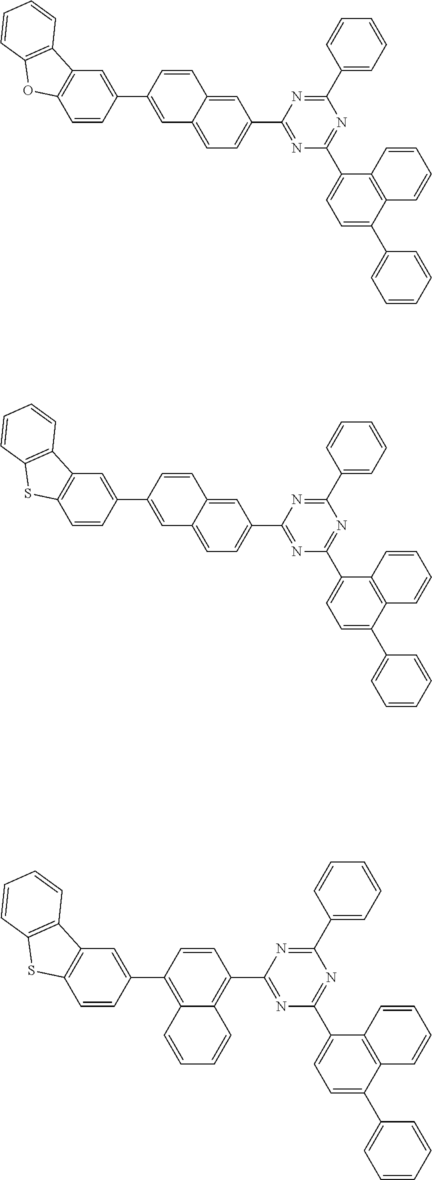 [SMILES: c1ccc(-c2nc(-c3ccc4cc(-c5ccc6oc7ccccc7c6c5)ccc4c3)nc(-c3ccc(-c4ccccc4)c4ccccc34)n2)cc1 ; c1ccc(-c2nc(-c3ccc4cc(-c5ccc6sc7ccccc7c6c5)ccc4c3)nc(-c3ccc(-c4ccccc4)c4ccccc34)n2)cc1 ; c1ccc(-c2nc(-c3ccc(-c4ccccc4)c4ccccc34)nc(-c3ccc(-c4ccc5sc6ccccc6c5c4)c4ccccc34)n2)cc1]

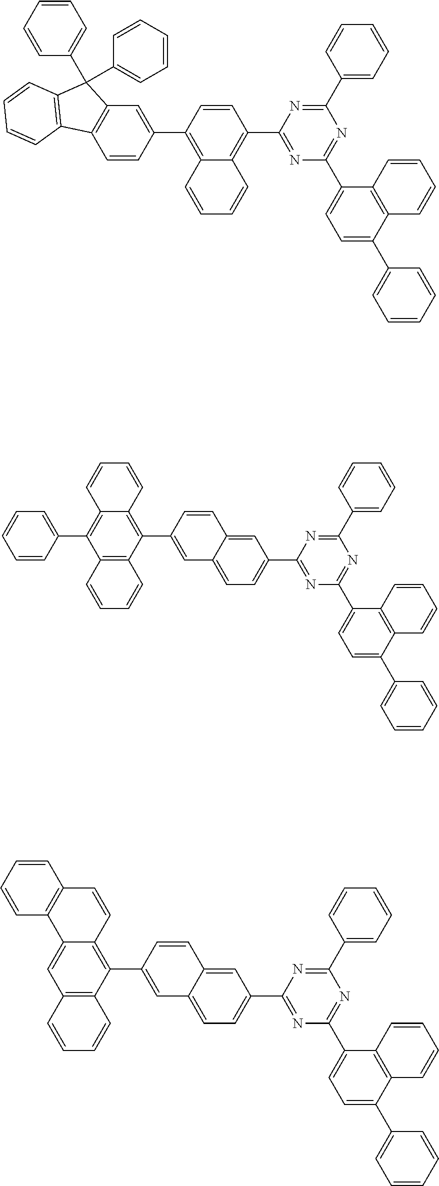 [SMILES: c1ccc(-c2nc(-c3ccc(-c4ccccc4)c4ccccc34)nc(-c3ccc(-c4ccc5c(c4)C(c4ccccc4)(c4ccccc4)c4ccccc4-5)c4ccccc34)n2)cc1 ; c1ccc(-c2nc(-c3ccc4cc(-c5c6ccccc6c(-c6ccccc6)c6ccccc56)ccc4c3)nc(-c3ccc(-c4ccccc4)c4ccccc34)n2)cc1 ; c1ccc(-c2nc(-c3ccc4cc(-c5c6ccccc6cc6c5ccc5ccccc56)ccc4c3)nc(-c3ccc(-c4ccccc4)c4ccccc34)n2)cc1]

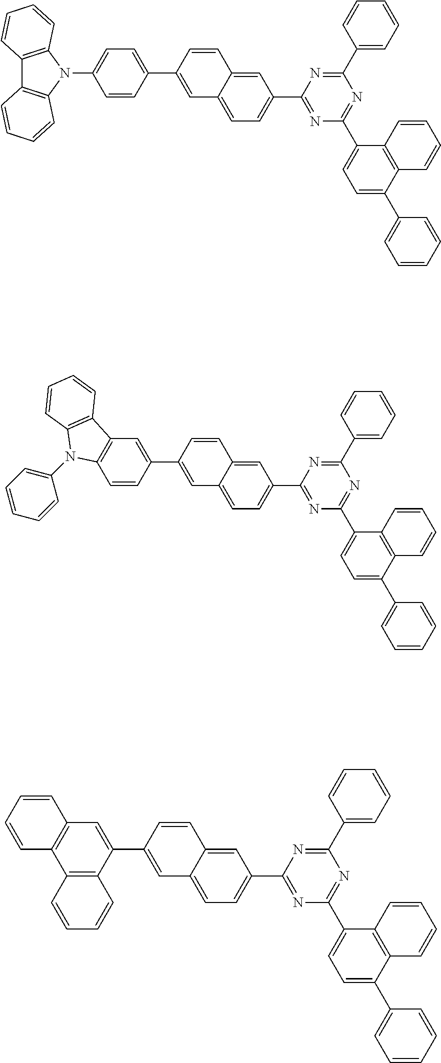 [SMILES: c1ccc(-c2nc(-c3ccc4cc(-c5ccc(-n6c7ccccc7c7ccccc76)cc5)ccc4c3)nc(-c3ccc(-c4ccccc4)c4ccccc34)n2)cc1 ; c1ccc(-c2nc(-c3ccc4cc(-c5ccc6c(c5)c5ccccc5n6-c5ccccc5)ccc4c3)nc(-c3ccc(-c4ccccc4)c4ccccc34)n2)cc1 ; c1ccc(-c2nc(-c3ccc4cc(-c5cc6ccccc6c6ccccc56)ccc4c3)nc(-c3ccc(-c4ccccc4)c4ccccc34)n2)cc1]

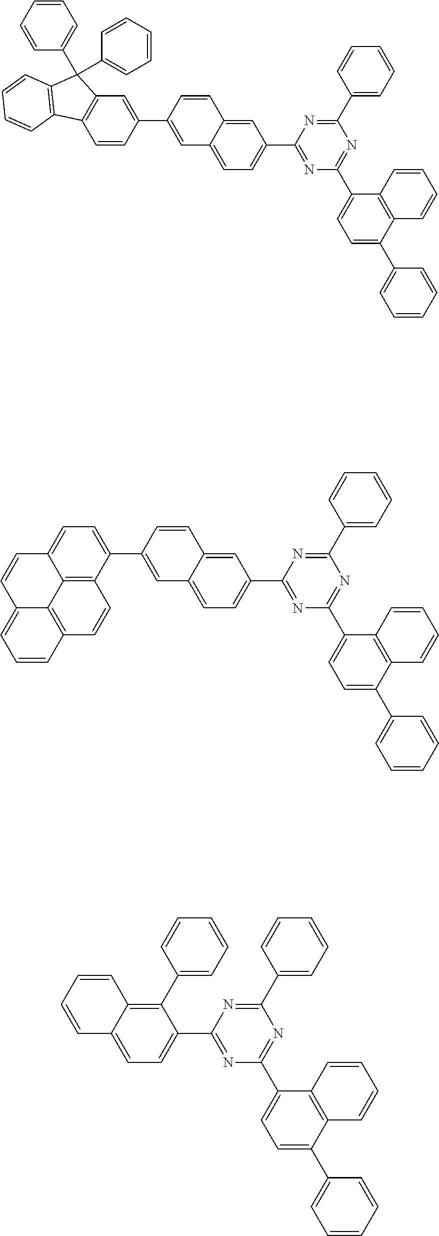 [SMILES: c1ccc(-c2nc(-c3ccc4cc(-c5ccc6c(c5)C(c5ccccc5)(c5ccccc5)c5ccccc5-6)ccc4c3)nc(-c3ccc(-c4ccccc4)c4ccccc34)n2)cc1 ; c1ccc(-c2nc(-c3ccc4cc(-c5ccc6ccc7cccc8ccc5c6c78)ccc4c3)nc(-c3ccc(-c4ccccc4)c4ccccc34)n2)cc1 ; c1ccc(-c2nc(-c3ccc4ccccc4c3-c3ccccc3)nc(-c3ccc(-c4ccccc4)c4ccccc34)n2)cc1]

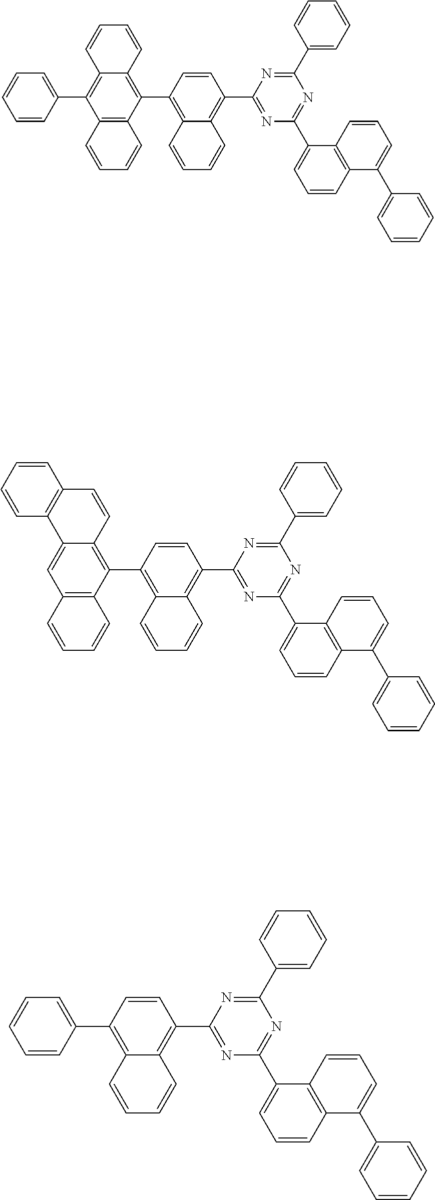 [SMILES: c1ccc(-c2nc(-c3cccc4c(-c5ccccc5)cccc34)nc(-c3ccc(-c4c5ccccc5c(-c5ccccc5)c5ccccc45)c4ccccc34)n2)cc1 ; c1ccc(-c2nc(-c3cccc4c(-c5ccccc5)cccc34)nc(-c3ccc(-c4c5ccccc5cc5c4ccc4ccccc45)c4ccccc34)n2)cc1 ; c1ccc(-c2nc(-c3cccc4c(-c5ccccc5)cccc34)nc(-c3ccc(-c4ccccc4)c4ccccc34)n2)cc1]

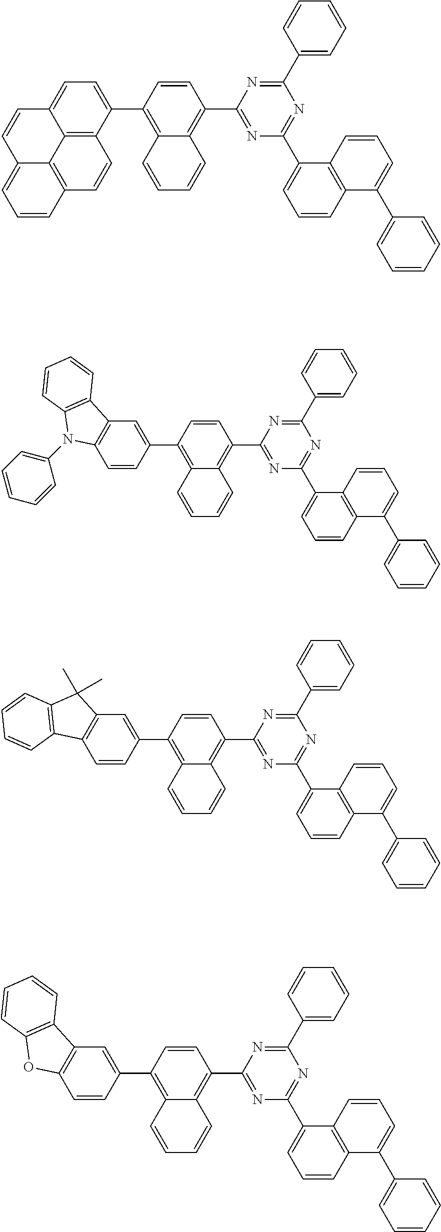 [SMILES: c1ccc(-c2nc(-c3cccc4c(-c5ccccc5)cccc34)nc(-c3ccc(-c4ccc5ccc6cccc7ccc4c5c67)c4ccccc34)n2)cc1 ; c1ccc(-c2nc(-c3cccc4c(-c5ccccc5)cccc34)nc(-c3ccc(-c4ccc5c(c4)c4ccccc4n5-c4ccccc4)c4ccccc34)n2)cc1 ; CC1(C)c2ccccc2-c2ccc(-c3ccc(-c4nc(-c5ccccc5)nc(-c5cccc6c(-c7ccccc7)cccc56)n4)c4ccccc34)cc21 ; c1ccc(-c2nc(-c3cccc4c(-c5ccccc5)cccc34)nc(-c3ccc(-c4ccc5oc6ccccc6c5c4)c4ccccc34)n2)cc1]

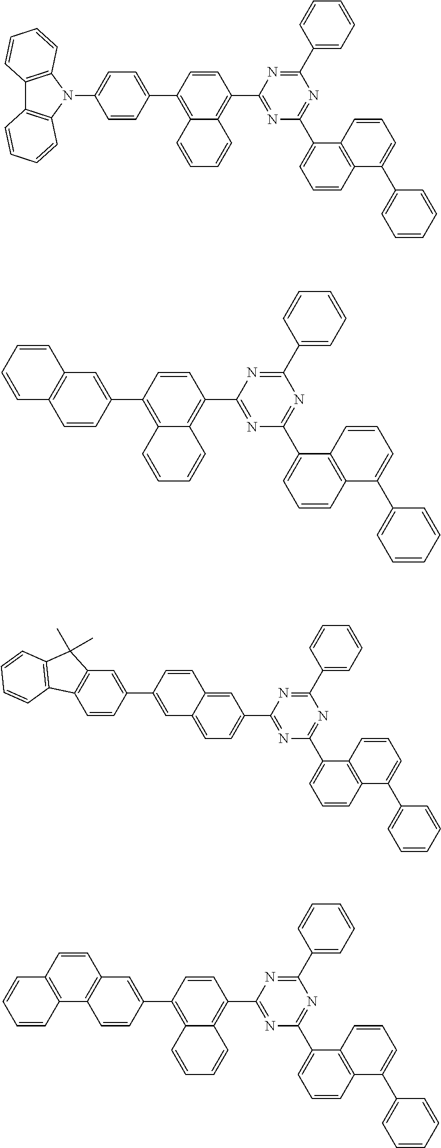 [SMILES: c1ccc(-c2nc(-c3cccc4c(-c5ccccc5)cccc34)nc(-c3ccc(-c4ccc(-n5c6ccccc6c6ccccc65)cc4)c4ccccc34)n2)cc1 ; c1ccc(-c2nc(-c3cccc4c(-c5ccccc5)cccc34)nc(-c3ccc(-c4ccc5ccccc5c4)c4ccccc34)n2)cc1 ; CC1(C)c2ccccc2-c2ccc(-c3ccc4cc(-c5nc(-c6ccccc6)nc(-c6cccc7c(-c8ccccc8)cccc67)n5)ccc4c3)cc21 ; c1ccc(-c2nc(-c3cccc4c(-c5ccccc5)cccc34)nc(-c3ccc(-c4ccc5c(ccc6ccccc65)c4)c4ccccc34)n2)cc1]

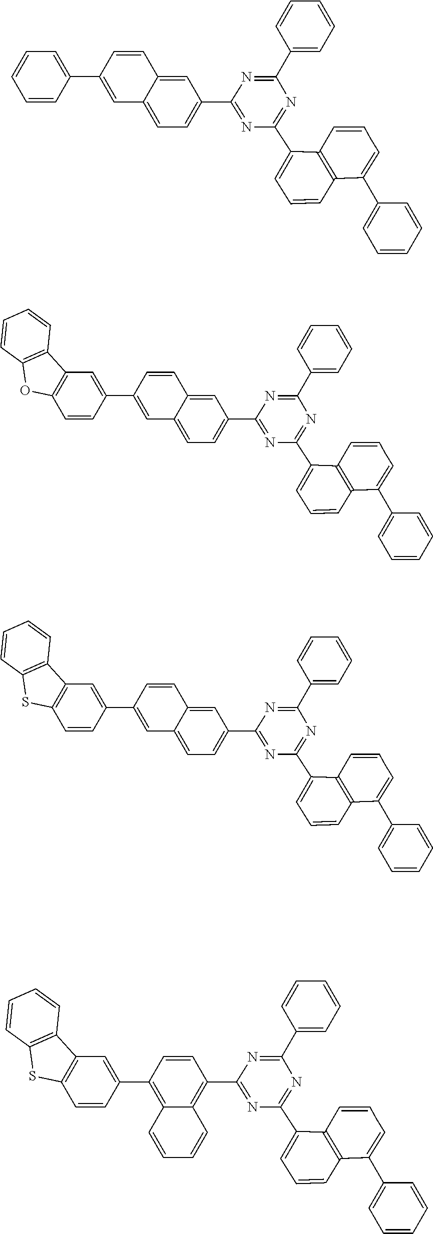 [SMILES: C1=Cc2c(cccc2-c2nc(-c3ccccc3)nc(-c3ccc4cc(-c5ccccc5)ccc4c3)n2)C(c2ccccc2)=CC1 ; C1=Cc2c(cccc2-c2nc(-c3ccccc3)nc(-c3ccc4cc(-c5ccc6oc7ccccc7c6c5)ccc4c3)n2)C(c2ccccc2)=CC1 ; C1=Cc2c(cccc2-c2nc(-c3ccccc3)nc(-c3ccc4cc(-c5ccc6sc7ccccc7c6c5)ccc4c3)n2)C(c2ccccc2)=CC1 ; C1=Cc2c(cccc2-c2nc(-c3ccccc3)nc(-c3ccc(-c4ccc5sc6ccccc6c5c4)c4ccccc34)n2)C(c2ccccc2)=CC1]

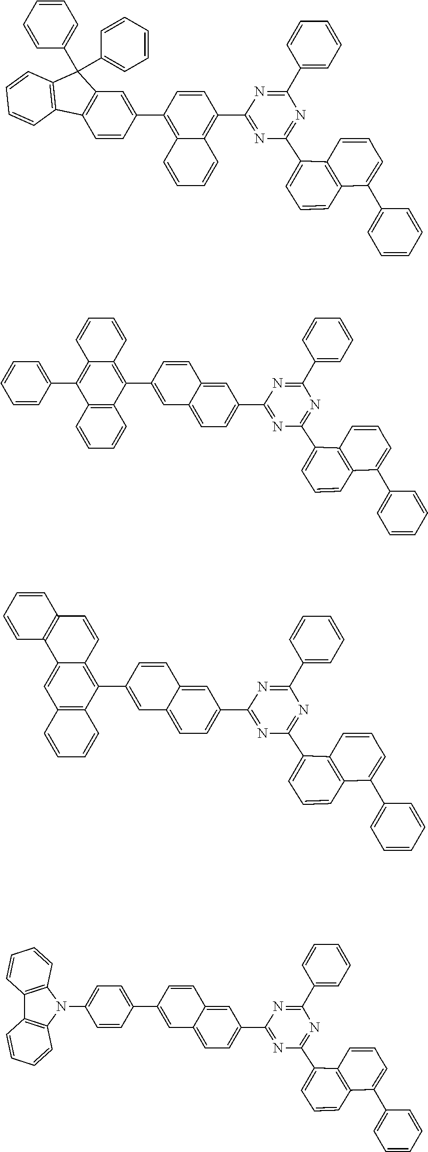 [SMILES: C1=Cc2c(cccc2-c2nc(-c3ccccc3)nc(-c3ccc(-c4ccc5c(c4)C(c4ccccc4)(c4ccccc4)c4ccccc4-5)c4ccccc34)n2)C(c2ccccc2)=CC1 ; C1=Cc2c(cccc2-c2nc(-c3ccccc3)nc(-c3ccc4cc(-c5c6ccccc6c(-c6ccccc6)c6ccccc56)ccc4c3)n2)C(c2ccccc2)=CC1 ; C1=Cc2c(cccc2-c2nc(-c3ccccc3)nc(-c3ccc4cc(-c5c6ccccc6cc6c5ccc5ccccc56)ccc4c3)n2)C(c2ccccc2)=CC1 ; C1=Cc2c(cccc2-c2nc(-c3ccccc3)nc(-c3ccc4cc(-c5ccc(-n6c7ccccc7c7ccccc76)cc5)ccc4c3)n2)C(c2ccccc2)=CC1]

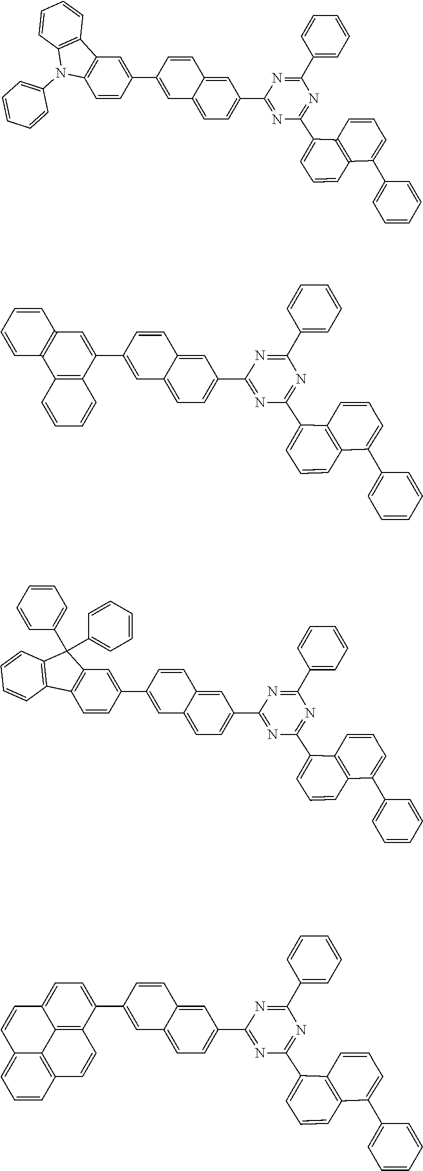 [SMILES: C1=Cc2c(cccc2-c2nc(-c3ccccc3)nc(-c3ccc4cc(-c5ccc6c(c5)c5ccccc5n6-c5ccccc5)ccc4c3)n2)C(c2ccccc2)=CC1 ; C1=Cc2c(cccc2-c2nc(-c3ccccc3)nc(-c3ccc4cc(-c5cc6ccccc6c6ccccc56)ccc4c3)n2)C(c2ccccc2)=CC1 ; C1=Cc2c(cccc2-c2nc(-c3ccccc3)nc(-c3ccc4cc(-c5ccc6c(c5)C(c5ccccc5)(c5ccccc5)c5ccccc5-6)ccc4c3)n2)C(c2ccccc2)=CC1 ; C1=Cc2c(cccc2-c2nc(-c3ccccc3)nc(-c3ccc4cc(-c5ccc6ccc7cccc8ccc5c6c78)ccc4c3)n2)C(c2ccccc2)=CC1]

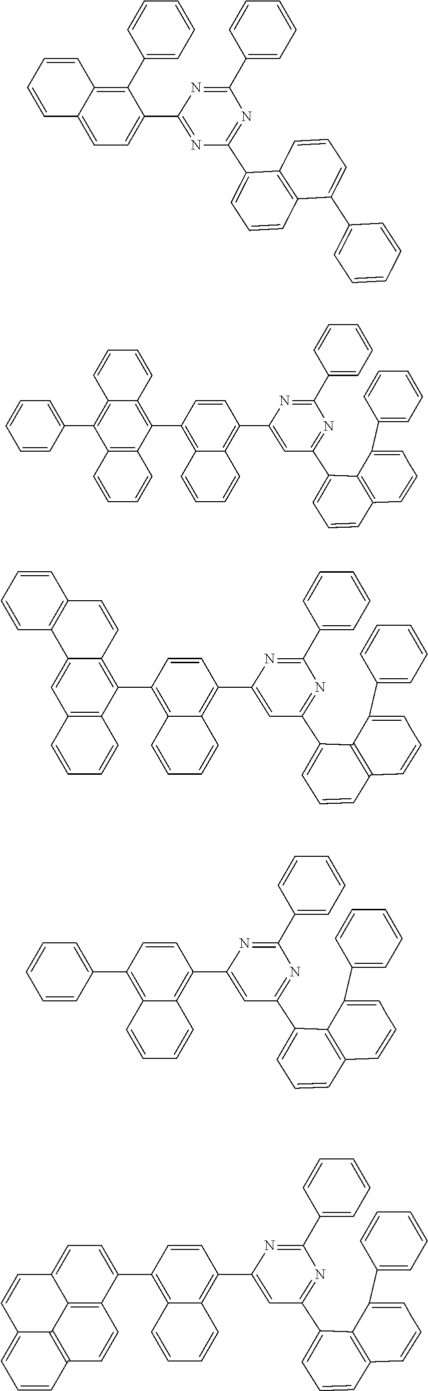

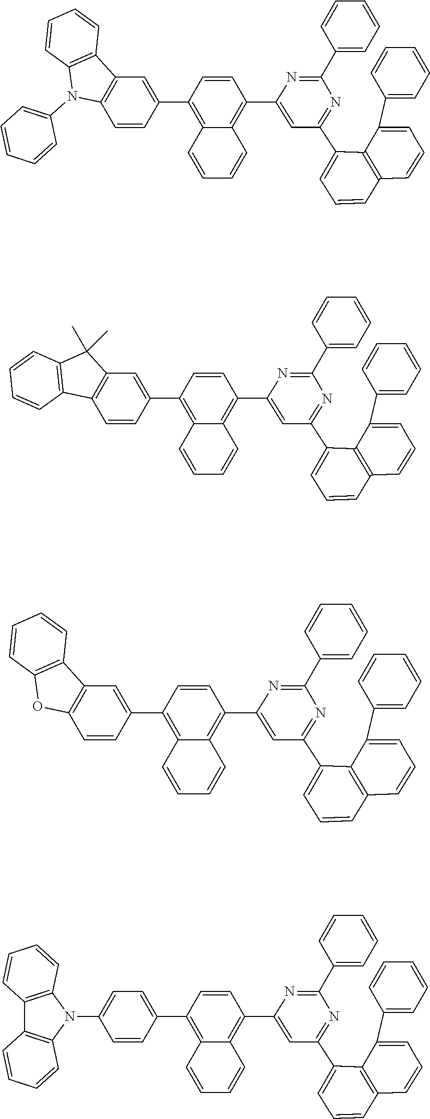 [SMILES: C1=Cc2cccc(-c3cc(-c4ccc(-c5ccc6c(c5)c5ccccc5n6-c5ccccc5)c5ccccc45)nc(-c4ccccc4)n3)c2C(c2ccccc2)=CC1 ; CC1(C)c2ccccc2-c2ccc(-c3ccc(-c4cc(-c5cccc6c5C(c5ccccc5)=CCC=C6)nc(-c5ccccc5)n4)c4ccccc34)cc21 ; C1=Cc2cccc(-c3cc(-c4ccc(-c5ccc6oc7ccccc7c6c5)c5ccccc45)nc(-c4ccccc4)n3)c2C(c2ccccc2)=CC1 ; C1=Cc2cccc(-c3cc(-c4ccc(-c5ccc(-n6c7ccccc7c7ccccc76)cc5)c5ccccc45)nc(-c4ccccc4)n3)c2C(c2ccccc2)=CC1]

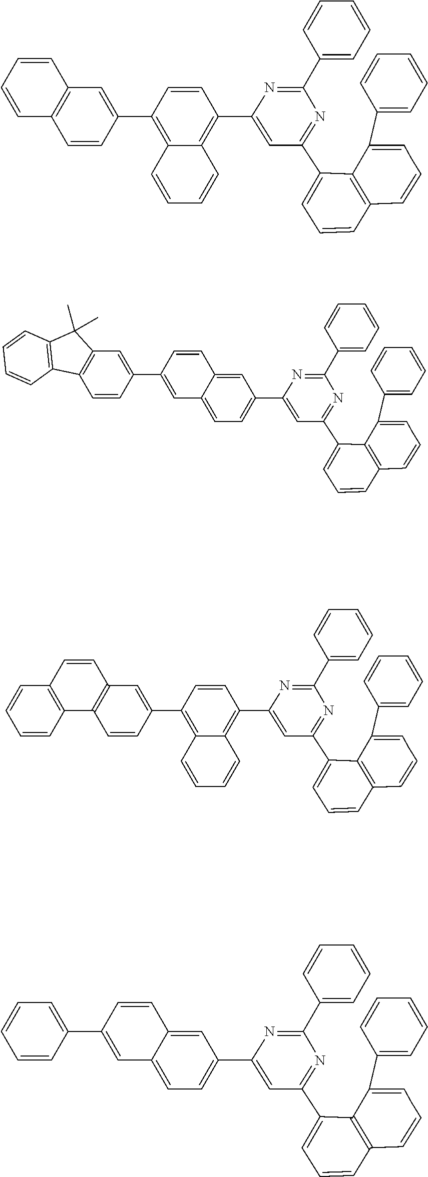 [SMILES: C1=Cc2cccc(-c3cc(-c4ccc(-c5ccc6ccccc6c5)c5ccccc45)nc(-c4ccccc4)n3)c2C(c2ccccc2)=CC1 ; CC1(C)c2ccccc2-c2ccc(-c3ccc4cc(-c5cc(-c6cccc7c6C(c6ccccc6)=CCC=C7)nc(-c6ccccc6)n5)ccc4c3)cc21 ; C1=Cc2cccc(-c3cc(-c4ccc(-c5ccc6c(ccc7ccccc76)c5)c5ccccc45)nc(-c4ccccc4)n3)c2C(c2ccccc2)=CC1 ; C1=Cc2cccc(-c3cc(-c4ccc5cc(-c6ccccc6)ccc5c4)nc(-c4ccccc4)n3)c2C(c2ccccc2)=CC1]

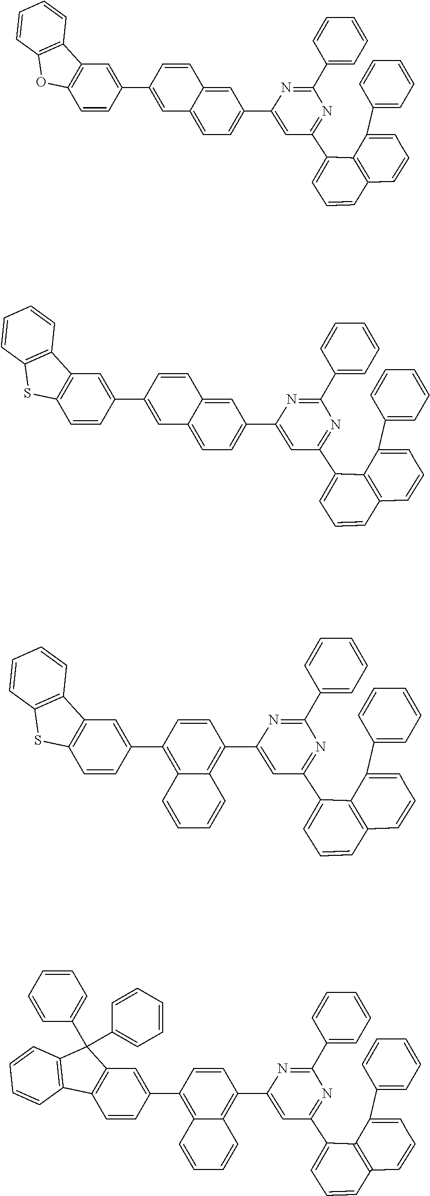 [SMILES: C1=Cc2cccc(-c3cc(-c4ccc5cc(-c6ccc7oc8ccccc8c7c6)ccc5c4)nc(-c4ccccc4)n3)c2C(c2ccccc2)=CC1 ; C1=Cc2cccc(-c3cc(-c4ccc5cc(-c6ccc7sc8ccccc8c7c6)ccc5c4)nc(-c4ccccc4)n3)c2C(c2ccccc2)=CC1 ; C1=Cc2cccc(-c3cc(-c4ccc(-c5ccc6sc7ccccc7c6c5)c5ccccc45)nc(-c4ccccc4)n3)c2C(c2ccccc2)=CC1 ; C1=Cc2cccc(-c3cc(-c4ccc(-c5ccc6c(c5)C(c5ccccc5)(c5ccccc5)c5ccccc5-6)c5ccccc45)nc(-c4ccccc4)n3)c2C(c2ccccc2)=CC1]

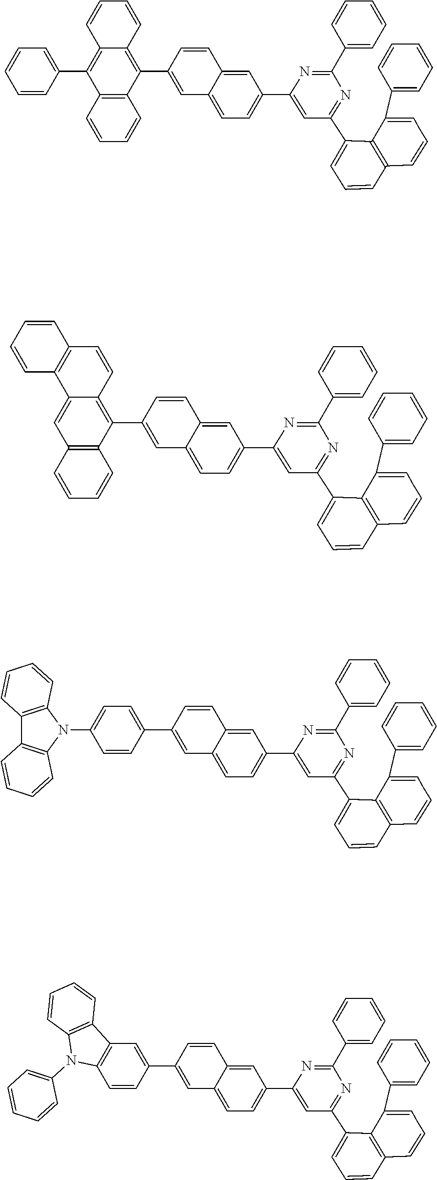 [SMILES: C1=Cc2cccc(-c3cc(-c4ccc5cc(-c6c7ccccc7c(-c7ccccc7)c7ccccc67)ccc5c4)nc(-c4ccccc4)n3)c2C(c2ccccc2)=CC1 ; C1=Cc2cccc(-c3cc(-c4ccc5cc(-c6c7ccccc7cc7c6ccc6ccccc67)ccc5c4)nc(-c4ccccc4)n3)c2C(c2ccccc2)=CC1 ; C1=Cc2cccc(-c3cc(-c4ccc5cc(-c6ccc(-n7c8ccccc8c8ccccc87)cc6)ccc5c4)nc(-c4ccccc4)n3)c2C(c2ccccc2)=CC1 ; C1=Cc2cccc(-c3cc(-c4ccc5cc(-c6ccc7c(c6)c6ccccc6n7-c6ccccc6)ccc5c4)nc(-c4ccccc4)n3)c2C(c2ccccc2)=CC1]

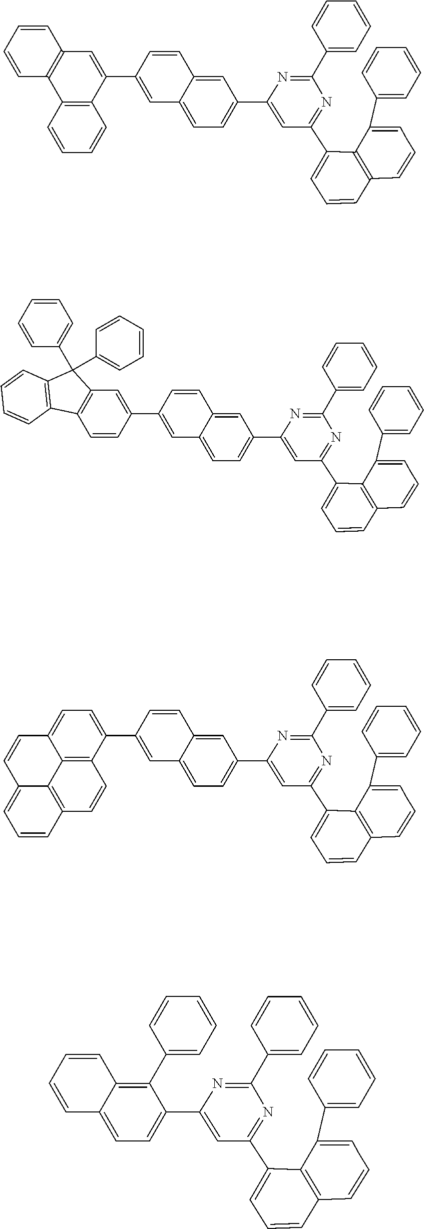 [SMILES: C1=Cc2cccc(-c3cc(-c4ccc5cc(-c6cc7ccccc7c7ccccc67)ccc5c4)nc(-c4ccccc4)n3)c2C(c2ccccc2)=CC1 ; C1=Cc2cccc(-c3cc(-c4ccc5cc(-c6ccc7c(c6)C(c6ccccc6)(c6ccccc6)c6ccccc6-7)ccc5c4)nc(-c4ccccc4)n3)c2C(c2ccccc2)=CC1 ; C1=Cc2cccc(-c3cc(-c4ccc5cc(-c6ccc7ccc8cccc9ccc6c7c89)ccc5c4)nc(-c4ccccc4)n3)c2C(c2ccccc2)=CC1 ; C1=Cc2cccc(-c3cc(-c4ccc5ccccc5c4-c4ccccc4)nc(-c4ccccc4)n3)c2C(c2ccccc2)=CC1]

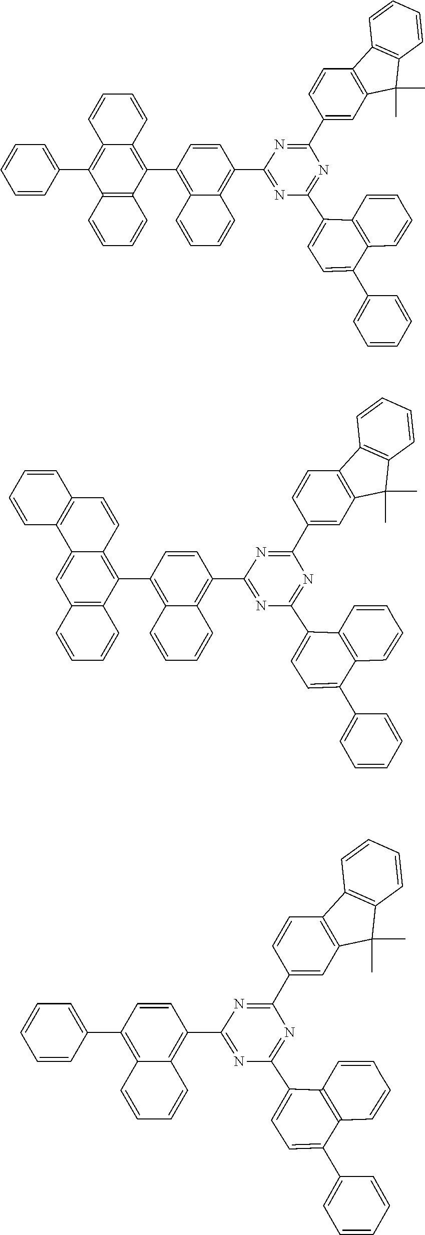 [SMILES: CC1(C)c2ccccc2-c2ccc(-c3nc(-c4ccc(-c5ccccc5)c5c4C=CCC=C5)nc(-c4ccc(-c5c6ccccc6c(-c6ccccc6)c6ccccc56)c5ccccc45)n3)cc21 ; CC1(C)c2ccccc2-c2ccc(-c3nc(-c4ccc(-c5ccccc5)c5c4C=CCC=C5)nc(-c4ccc(-c5c6ccccc6cc6c5ccc5ccccc56)c5ccccc45)n3)cc21 ; CC1(C)c2ccccc2-c2ccc(-c3nc(-c4ccc(-c5ccccc5)c5c4C=CCC=C5)nc(-c4ccc(-c5ccccc5)c5ccccc45)n3)cc21]

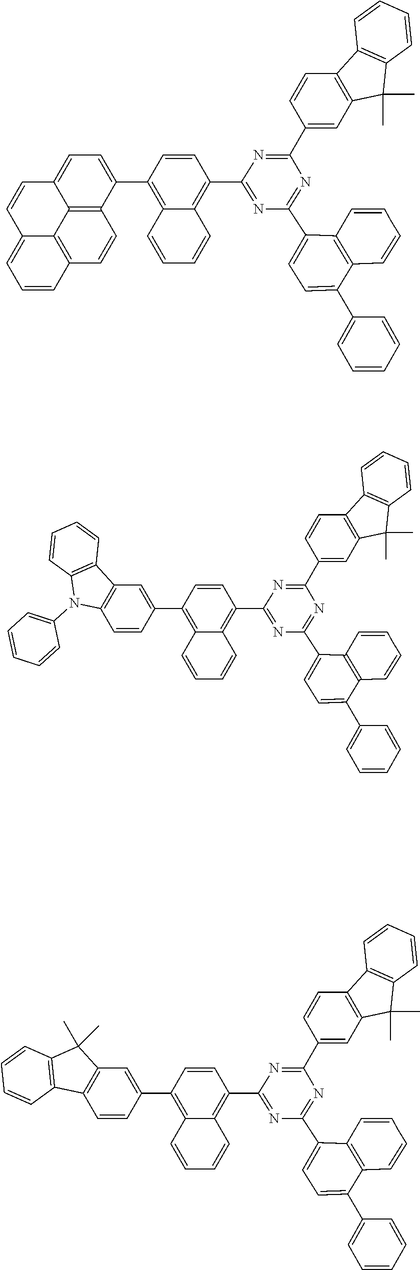 [SMILES: CC1(C)c2ccccc2-c2ccc(-c3nc(-c4ccc(-c5ccccc5)c5c4C=CCC=C5)nc(-c4ccc(-c5ccc6ccc7cccc8ccc5c6c78)c5ccccc45)n3)cc21 ; CC1(C)c2ccccc2-c2ccc(-c3nc(-c4ccc(-c5ccccc5)c5c4C=CCC=C5)nc(-c4ccc(-c5ccc6c(c5)c5ccccc5n6-c5ccccc5)c5ccccc45)n3)cc21 ; CC1(C)c2ccccc2-c2ccc(-c3nc(-c4ccc(-c5ccccc5)c5c4C=CCC=C5)nc(-c4ccc(-c5ccc6c(c5)C(C)(C)c5ccccc5-6)c5ccccc45)n3)cc21]

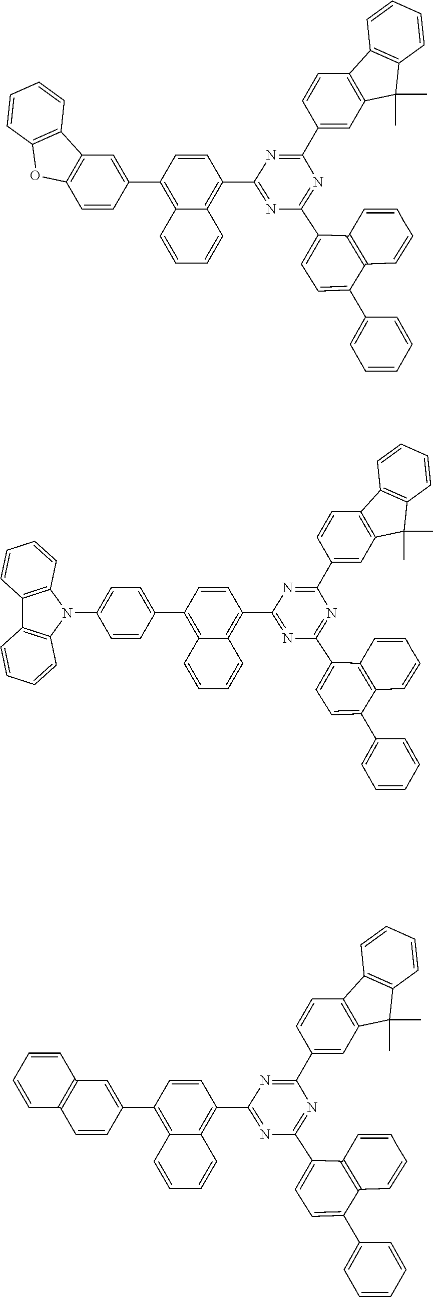 [SMILES: CC1(C)c2ccccc2-c2ccc(-c3nc(-c4ccc(-c5ccccc5)c5c4C=CCC=C5)nc(-c4ccc(-c5ccc6oc7ccccc7c6c5)c5ccccc45)n3)cc21 ; CC1(C)c2ccccc2-c2ccc(-c3nc(-c4ccc(-c5ccccc5)c5c4C=CCC=C5)nc(-c4ccc(-c5ccc(-n6c7ccccc7c7ccccc76)cc5)c5ccccc45)n3)cc21 ; CC1(C)c2ccccc2-c2ccc(-c3nc(-c4ccc(-c5ccccc5)c5c4C=CCC=C5)nc(-c4ccc(-c5ccc6ccccc6c5)c5ccccc45)n3)cc21]

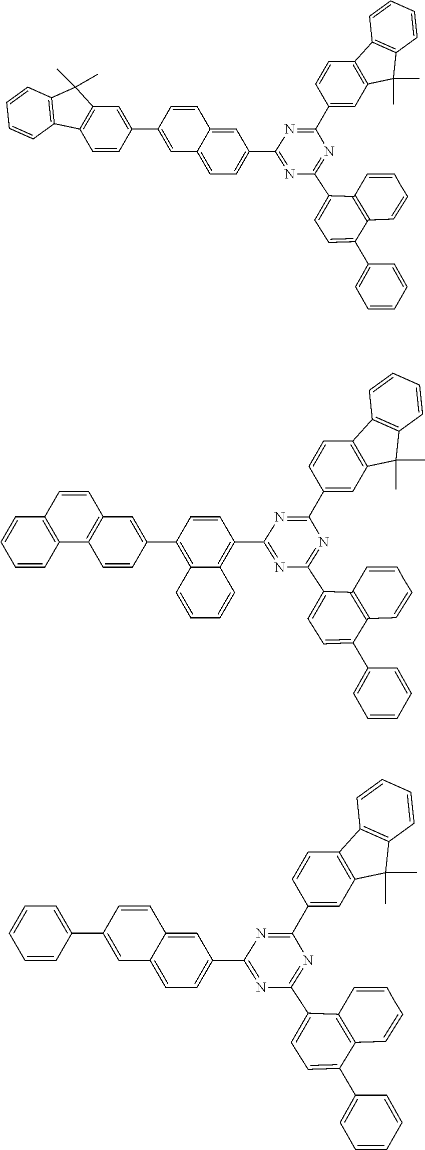 [SMILES: CC1(C)c2ccccc2-c2ccc(-c3ccc4cc(-c5nc(-c6ccc7c(c6)C(C)(C)c6ccccc6-7)nc(-c6ccc(-c7ccccc7)c7c6C=CCC=C7)n5)ccc4c3)cc21 ; CC1(C)c2ccccc2-c2ccc(-c3nc(-c4ccc(-c5ccccc5)c5c4C=CCC=C5)nc(-c4ccc(-c5ccc6c(ccc7ccccc76)c5)c5ccccc45)n3)cc21 ; CC1(C)c2ccccc2-c2ccc(-c3nc(-c4ccc5cc(-c6ccccc6)ccc5c4)nc(-c4ccc(-c5ccccc5)c5c4C=CCC=C5)n3)cc21]

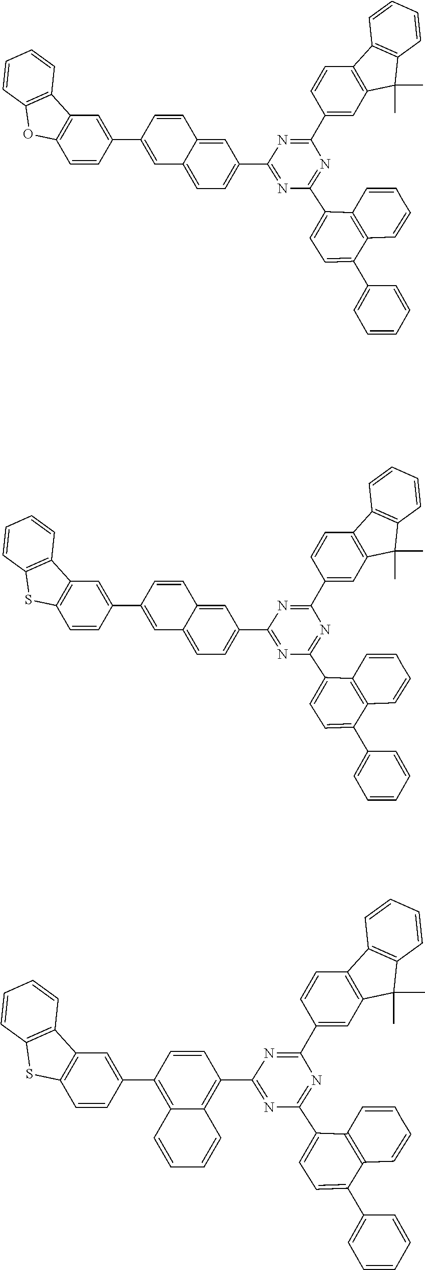 [SMILES: CC1(C)c2ccccc2-c2ccc(-c3nc(-c4ccc5cc(-c6ccc7oc8ccccc8c7c6)ccc5c4)nc(-c4ccc(-c5ccccc5)c5c4C=CCC=C5)n3)cc21 ; CC1(C)c2ccccc2-c2ccc(-c3nc(-c4ccc5cc(-c6ccc7sc8ccccc8c7c6)ccc5c4)nc(-c4ccc(-c5ccccc5)c5c4C=CCC=C5)n3)cc21 ; CC1(C)c2ccccc2-c2ccc(-c3nc(-c4ccc(-c5ccccc5)c5c4C=CCC=C5)nc(-c4ccc(-c5ccc6sc7ccccc7c6c5)c5ccccc45)n3)cc21]

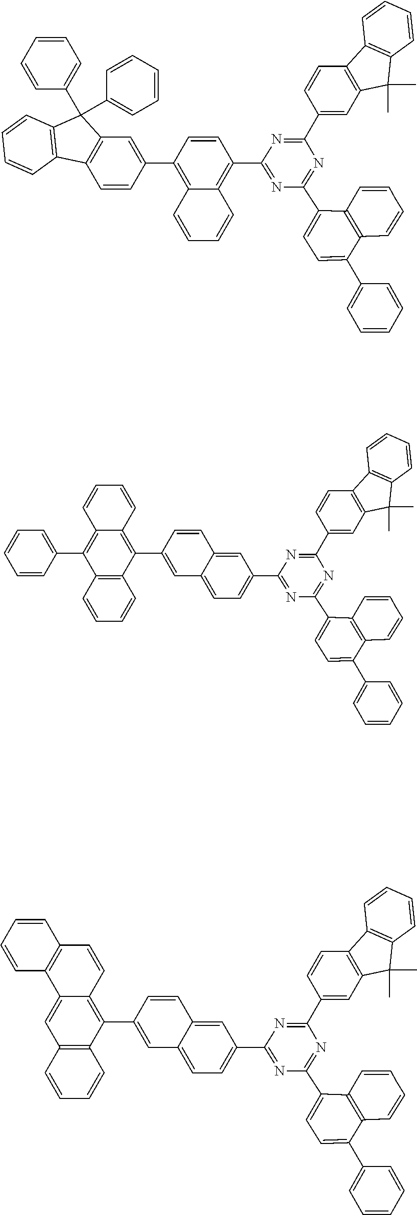 [SMILES: CC1(C)c2ccccc2-c2ccc(-c3nc(-c4ccc(-c5ccccc5)c5c4C=CCC=C5)nc(-c4ccc(-c5ccc6c(c5)C(c5ccccc5)(c5ccccc5)c5ccccc5-6)c5ccccc45)n3)cc21 ; CC1(C)c2ccccc2-c2ccc(-c3nc(-c4ccc5cc(-c6c7ccccc7c(-c7ccccc7)c7ccccc67)ccc5c4)nc(-c4ccc(-c5ccccc5)c5c4C=CCC=C5)n3)cc21 ; CC1(C)c2ccccc2-c2ccc(-c3nc(-c4ccc5cc(-c6c7ccccc7cc7c6ccc6ccccc67)ccc5c4)nc(-c4ccc(-c5ccccc5)c5c4C=CCC=C5)n3)cc21]

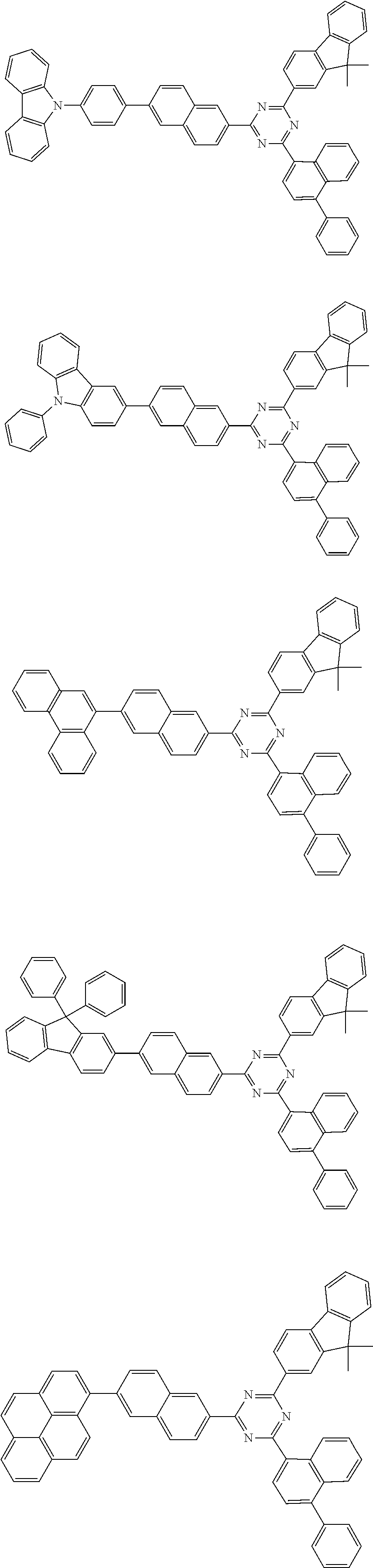

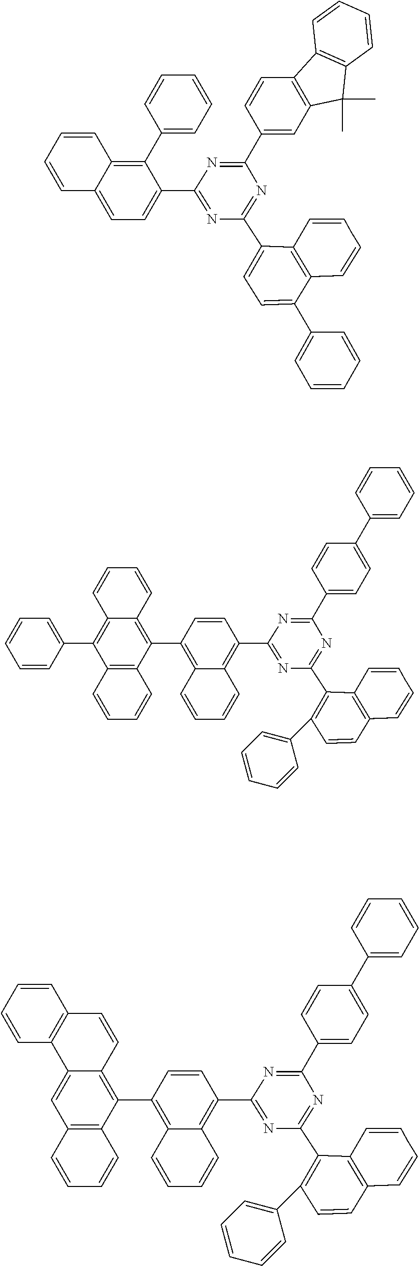 [SMILES: CC1(C)c2ccccc2-c2ccc(-c3nc(-c4ccc(-c5ccccc5)c5c4C=CCC=C5)nc(-c4ccc5ccccc5c4-c4ccccc4)n3)cc21 ; C1=Cc2ccc(-c3ccccc3)c(-c3nc(-c4ccc(-c5ccccc5)cc4)nc(-c4ccc(-c5c6ccccc6c(-c6ccccc6)c6ccccc56)c5ccccc45)n3)c2C=CC1 ; C1=Cc2ccc(-c3ccccc3)c(-c3nc(-c4ccc(-c5ccccc5)cc4)nc(-c4ccc(-c5c6ccccc6cc6c5ccc5ccccc56)c5ccccc45)n3)c2C=CC1]

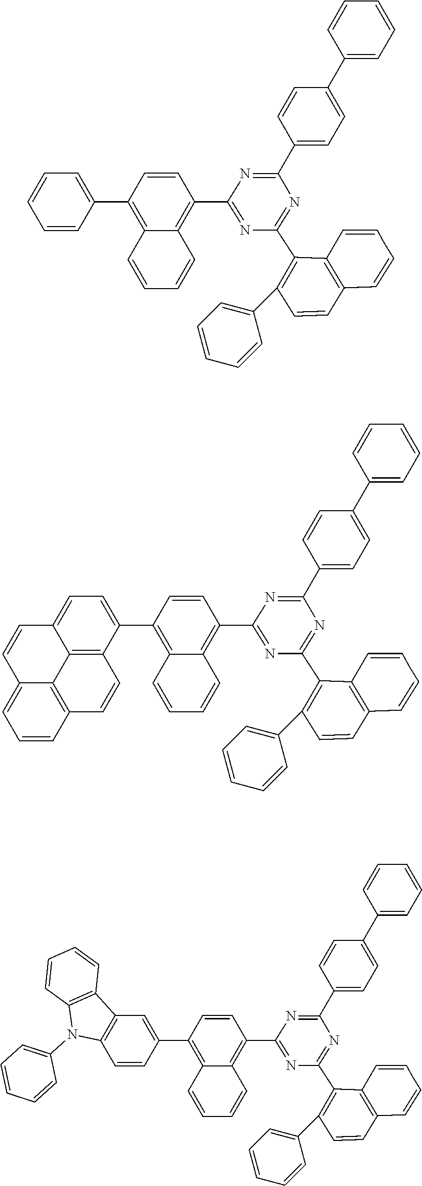 [SMILES: C1=Cc2ccc(-c3ccccc3)c(-c3nc(-c4ccc(-c5ccccc5)cc4)nc(-c4ccc(-c5ccccc5)c5ccccc45)n3)c2C=CC1 ; C1=Cc2ccc(-c3ccccc3)c(-c3nc(-c4ccc(-c5ccccc5)cc4)nc(-c4ccc(-c5ccc6ccc7cccc8ccc5c6c78)c5ccccc45)n3)c2C=CC1 ; C1=Cc2ccc(-c3ccccc3)c(-c3nc(-c4ccc(-c5ccccc5)cc4)nc(-c4ccc(-c5ccc6c(c5)c5ccccc5n6-c5ccccc5)c5ccccc45)n3)c2C=CC1]

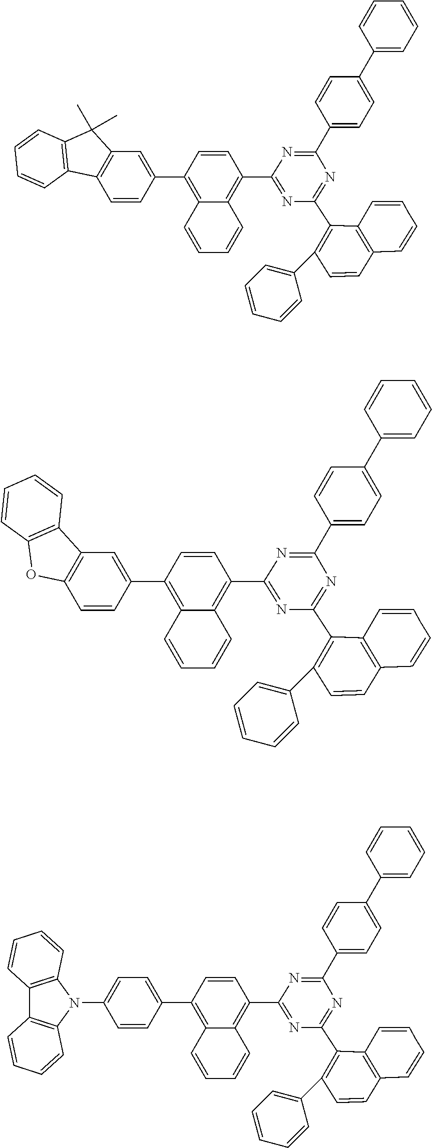 [SMILES: CC1(C)c2ccccc2-c2ccc(-c3ccc(-c4nc(-c5ccc(-c6ccccc6)cc5)nc(-c5c(-c6ccccc6)ccc6c5C=CCC=C6)n4)c4ccccc34)cc21 ; C1=Cc2ccc(-c3ccccc3)c(-c3nc(-c4ccc(-c5ccccc5)cc4)nc(-c4ccc(-c5ccc6oc7ccccc7c6c5)c5ccccc45)n3)c2C=CC1 ; C1=Cc2ccc(-c3ccccc3)c(-c3nc(-c4ccc(-c5ccccc5)cc4)nc(-c4ccc(-c5ccc(-n6c7ccccc7c7ccccc76)cc5)c5ccccc45)n3)c2C=CC1]

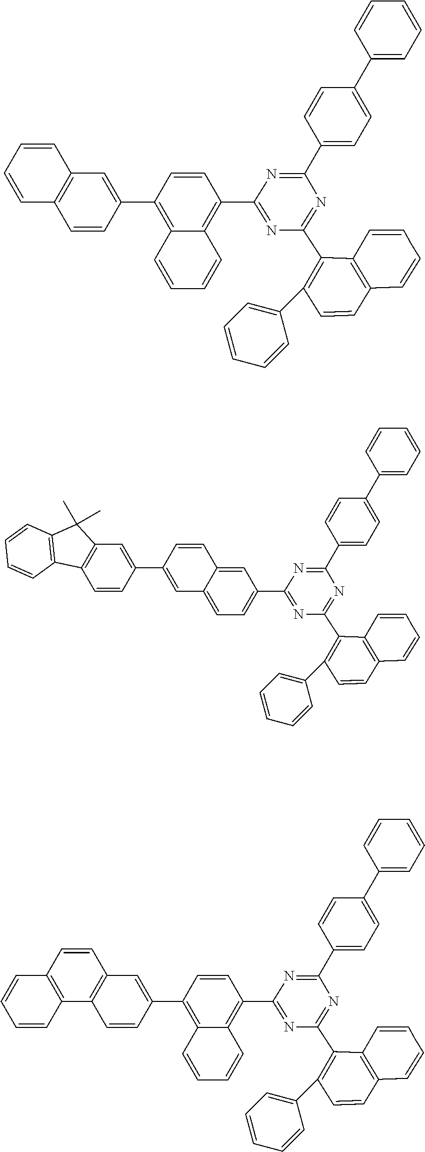 [SMILES: C1=Cc2ccc(-c3ccccc3)c(-c3nc(-c4ccc(-c5ccccc5)cc4)nc(-c4ccc(-c5ccc6ccccc6c5)c5ccccc45)n3)c2C=CC1 ; CC1(C)c2ccccc2-c2ccc(-c3ccc4cc(-c5nc(-c6ccc(-c7ccccc7)cc6)nc(-c6c(-c7ccccc7)ccc7c6C=CCC=C7)n5)ccc4c3)cc21 ; C1=Cc2ccc(-c3ccccc3)c(-c3nc(-c4ccc(-c5ccccc5)cc4)nc(-c4ccc(-c5ccc6c(ccc7ccccc76)c5)c5ccccc45)n3)c2C=CC1]

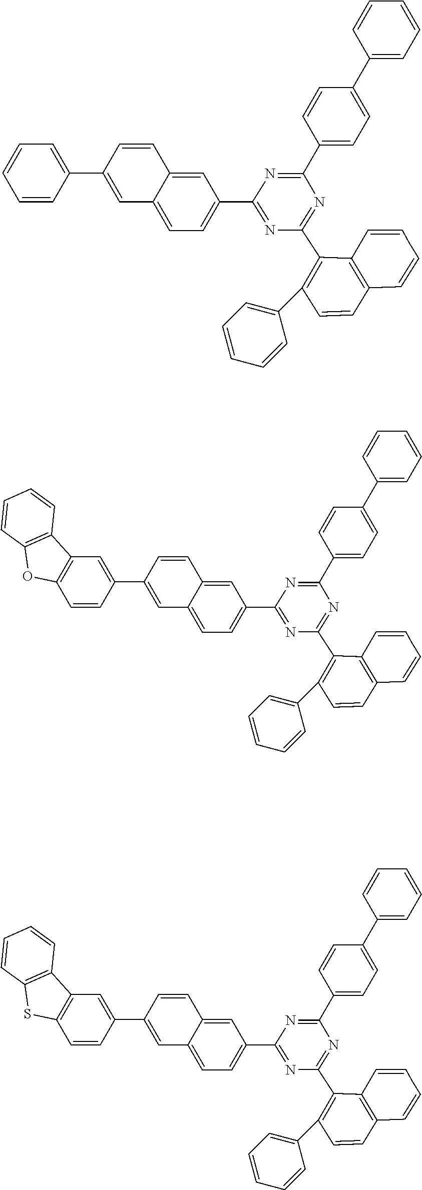 [SMILES: C1=Cc2ccc(-c3ccccc3)c(-c3nc(-c4ccc(-c5ccccc5)cc4)nc(-c4ccc5cc(-c6ccccc6)ccc5c4)n3)c2C=CC1 ; C1=Cc2ccc(-c3ccccc3)c(-c3nc(-c4ccc(-c5ccccc5)cc4)nc(-c4ccc5cc(-c6ccc7oc8ccccc8c7c6)ccc5c4)n3)c2C=CC1 ; C1=Cc2ccc(-c3ccccc3)c(-c3nc(-c4ccc(-c5ccccc5)cc4)nc(-c4ccc5cc(-c6ccc7sc8ccccc8c7c6)ccc5c4)n3)c2C=CC1]

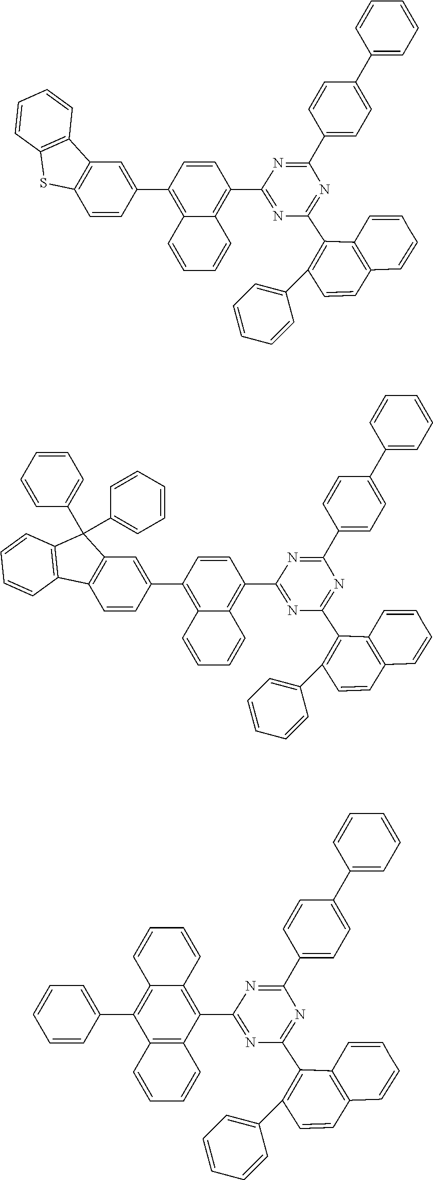 [SMILES: C1=Cc2ccc(-c3ccccc3)c(-c3nc(-c4ccc(-c5ccccc5)cc4)nc(-c4ccc(-c5ccc6sc7ccccc7c6c5)c5ccccc45)n3)c2C=CC1 ; C1=Cc2ccc(-c3ccccc3)c(-c3nc(-c4ccc(-c5ccccc5)cc4)nc(-c4ccc(-c5ccc6c(c5)C(c5ccccc5)(c5ccccc5)c5ccccc5-6)c5ccccc45)n3)c2C=CC1 ; C1=Cc2ccc(-c3ccccc3)c(-c3nc(-c4ccc(-c5ccccc5)cc4)nc(-c4c5ccccc5c(-c5ccccc5)c5ccccc45)n3)c2C=CC1]

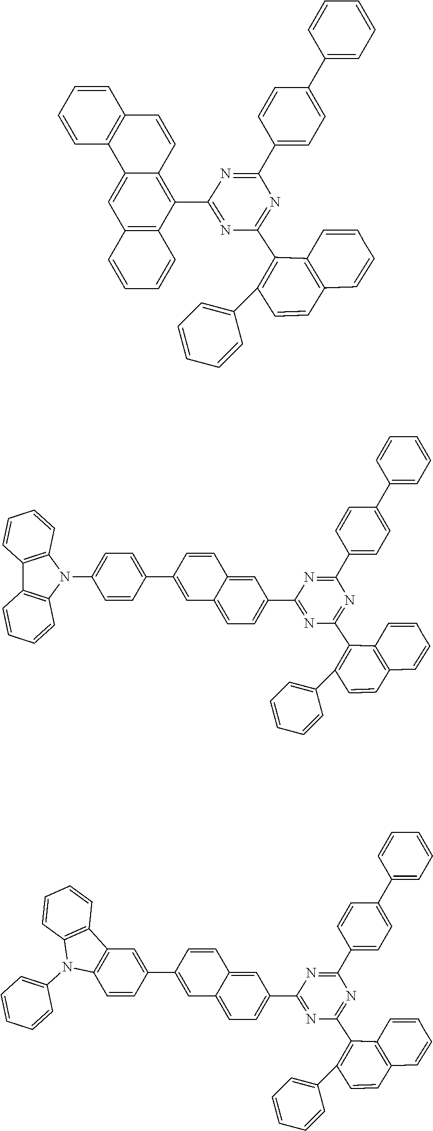 [SMILES: C1=Cc2ccc(-c3ccccc3)c(-c3nc(-c4ccc(-c5ccccc5)cc4)nc(-c4c5ccccc5cc5c4ccc4ccccc45)n3)c2C=CC1 ; C1=Cc2ccc(-c3ccccc3)c(-c3nc(-c4ccc(-c5ccccc5)cc4)nc(-c4ccc5cc(-c6ccc(-n7c8ccccc8c8ccccc87)cc6)ccc5c4)n3)c2C=CC1 ; C1=Cc2ccc(-c3ccccc3)c(-c3nc(-c4ccc(-c5ccccc5)cc4)nc(-c4ccc5cc(-c6ccc7c(c6)c6ccccc6n7-c6ccccc6)ccc5c4)n3)c2C=CC1]

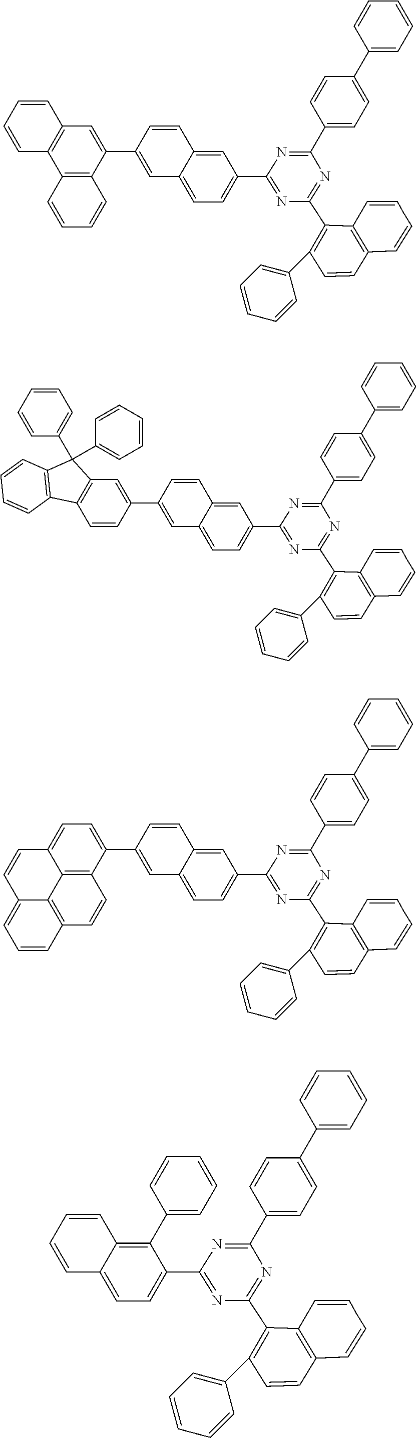 [SMILES: C1=Cc2ccc(-c3ccccc3)c(-c3nc(-c4ccc(-c5ccccc5)cc4)nc(-c4ccc5cc(-c6cc7ccccc7c7ccccc67)ccc5c4)n3)c2C=CC1 ; C1=Cc2ccc(-c3ccccc3)c(-c3nc(-c4ccc(-c5ccccc5)cc4)nc(-c4ccc5cc(-c6ccc7c(c6)C(c6ccccc6)(c6ccccc6)c6ccccc6-7)ccc5c4)n3)c2C=CC1 ; C1=Cc2ccc(-c3ccccc3)c(-c3nc(-c4ccc(-c5ccccc5)cc4)nc(-c4ccc5cc(-c6ccc7ccc8cccc9ccc6c7c89)ccc5c4)n3)c2C=CC1 ; C1=Cc2ccc(-c3ccccc3)c(-c3nc(-c4ccc(-c5ccccc5)cc4)nc(-c4ccc5ccccc5c4-c4ccccc4)n3)c2C=CC1]

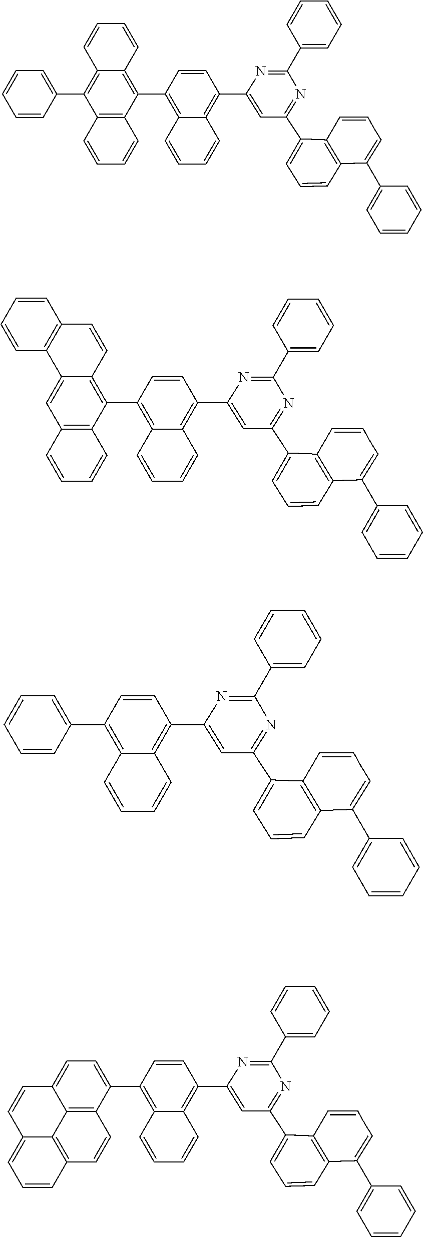 [SMILES: C1=Cc2c(cccc2-c2cc(-c3ccc(-c4c5ccccc5c(-c5ccccc5)c5ccccc45)c4ccccc34)nc(-c3ccccc3)n2)C(c2ccccc2)=CC1 ; C1=Cc2c(cccc2-c2cc(-c3ccc(-c4c5ccccc5cc5c4ccc4ccccc45)c4ccccc34)nc(-c3ccccc3)n2)C(c2ccccc2)=CC1 ; C1=Cc2c(cccc2-c2cc(-c3ccc(-c4ccccc4)c4ccccc34)nc(-c3ccccc3)n2)C(c2ccccc2)=CC1 ; C1=Cc2c(cccc2-c2cc(-c3ccc(-c4ccc5ccc6cccc7ccc4c5c67)c4ccccc34)nc(-c3ccccc3)n2)C(c2ccccc2)=CC1]

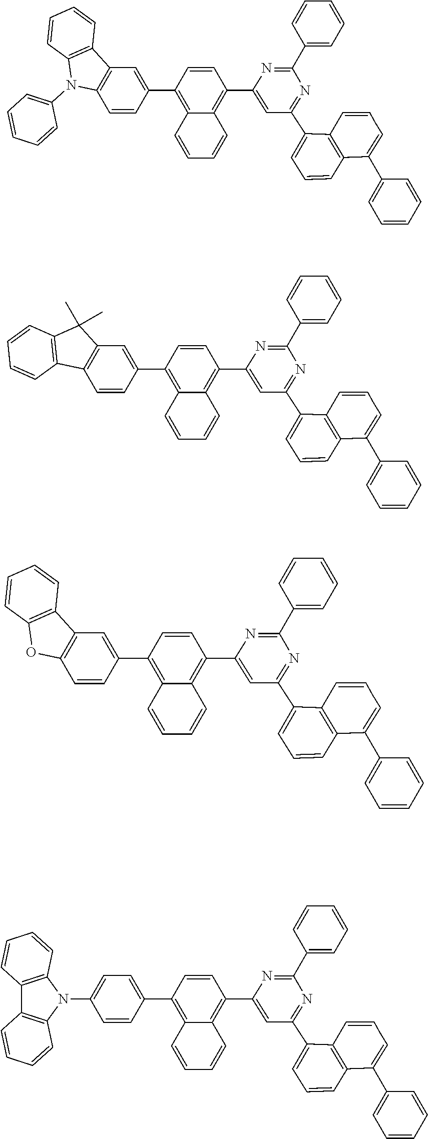 [SMILES: C1=Cc2c(cccc2-c2cc(-c3ccc(-c4ccc5c(c4)c4ccccc4n5-c4ccccc4)c4ccccc34)nc(-c3ccccc3)n2)C(c2ccccc2)=CC1 ; CC1(C)c2ccccc2-c2ccc(-c3ccc(-c4cc(-c5cccc6c5C=CCC=C6c5ccccc5)nc(-c5ccccc5)n4)c4ccccc34)cc21 ; C1=Cc2c(cccc2-c2cc(-c3ccc(-c4ccc5oc6ccccc6c5c4)c4ccccc34)nc(-c3ccccc3)n2)C(c2ccccc2)=CC1 ; C1=Cc2c(cccc2-c2cc(-c3ccc(-c4ccc(-n5c6ccccc6c6ccccc65)cc4)c4ccccc34)nc(-c3ccccc3)n2)C(c2ccccc2)=CC1]

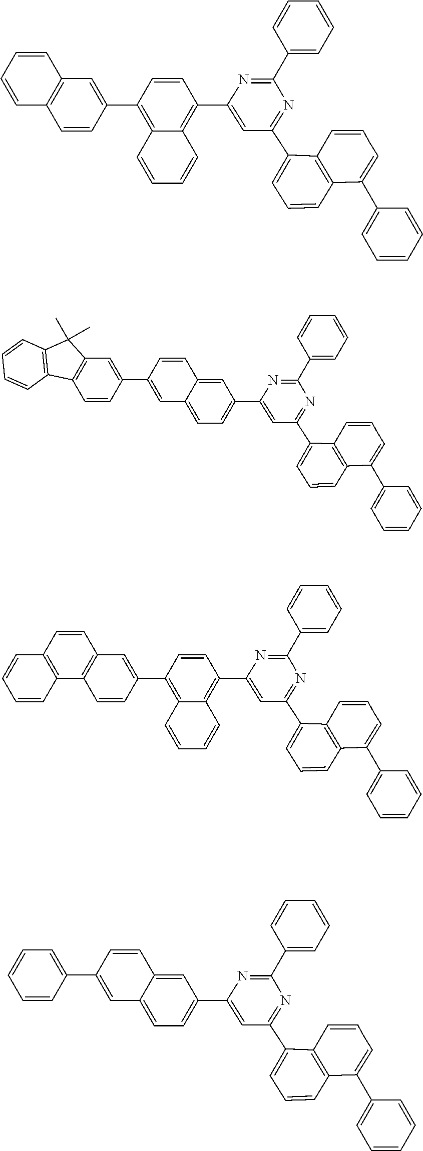 [SMILES: C1=Cc2c(cccc2-c2cc(-c3ccc(-c4ccc5ccccc5c4)c4ccccc34)nc(-c3ccccc3)n2)C(c2ccccc2)=CC1 ; CC1(C)c2ccccc2-c2ccc(-c3ccc4cc(-c5cc(-c6cccc7c6C=CCC=C7c6ccccc6)nc(-c6ccccc6)n5)ccc4c3)cc21 ; C1=Cc2c(cccc2-c2cc(-c3ccc(-c4ccc5c(ccc6ccccc65)c4)c4ccccc34)nc(-c3ccccc3)n2)C(c2ccccc2)=CC1 ; C1=Cc2c(cccc2-c2cc(-c3ccc4cc(-c5ccccc5)ccc4c3)nc(-c3ccccc3)n2)C(c2ccccc2)=CC1]

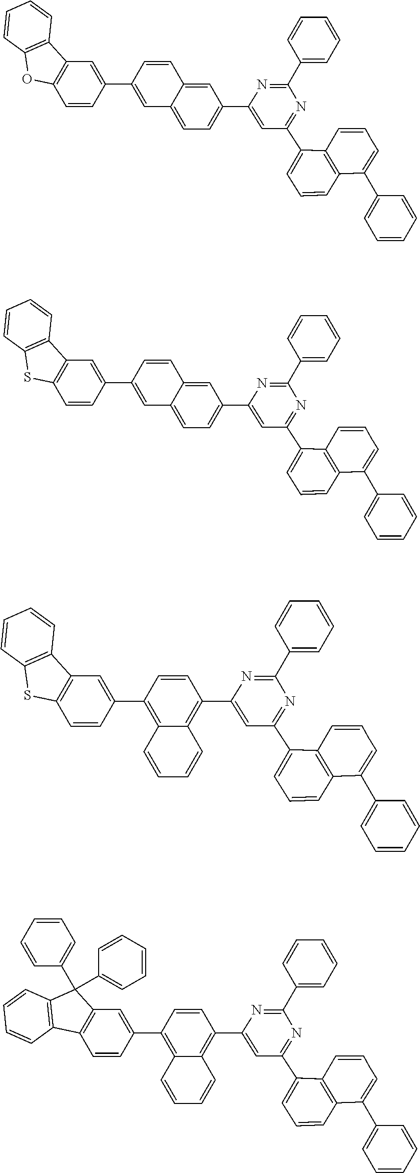 [SMILES: C1=Cc2c(cccc2-c2cc(-c3ccc4cc(-c5ccc6oc7ccccc7c6c5)ccc4c3)nc(-c3ccccc3)n2)C(c2ccccc2)=CC1 ; C1=Cc2c(cccc2-c2cc(-c3ccc4cc(-c5ccc6sc7ccccc7c6c5)ccc4c3)nc(-c3ccccc3)n2)C(c2ccccc2)=CC1 ; C1=Cc2c(cccc2-c2cc(-c3ccc(-c4ccc5sc6ccccc6c5c4)c4ccccc34)nc(-c3ccccc3)n2)C(c2ccccc2)=CC1 ; C1=Cc2c(cccc2-c2cc(-c3ccc(-c4ccc5c(c4)C(c4ccccc4)(c4ccccc4)c4ccccc4-5)c4ccccc34)nc(-c3ccccc3)n2)C(c2ccccc2)=CC1]

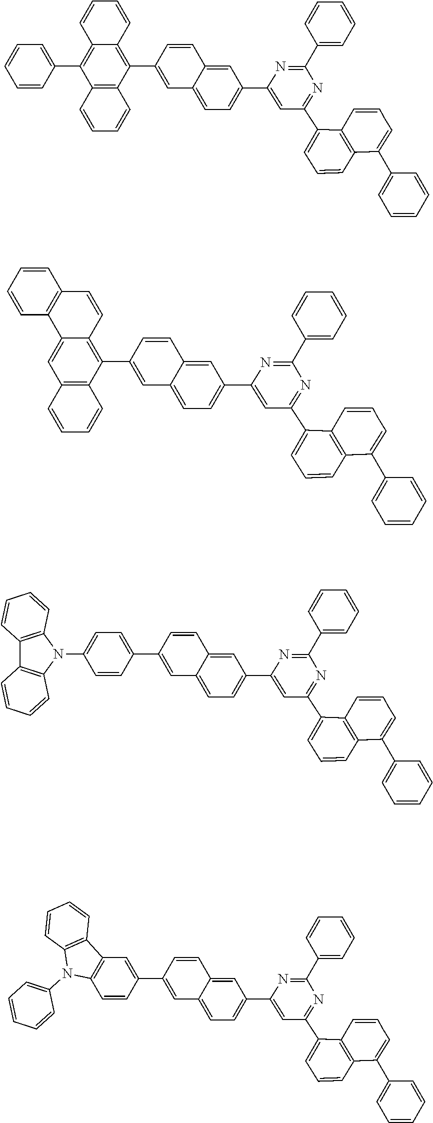 [SMILES: C1=Cc2c(cccc2-c2cc(-c3ccc4cc(-c5c6ccccc6c(-c6ccccc6)c6ccccc56)ccc4c3)nc(-c3ccccc3)n2)C(c2ccccc2)=CC1 ; C1=Cc2c(cccc2-c2cc(-c3ccc4cc(-c5c6ccccc6cc6c5ccc5ccccc56)ccc4c3)nc(-c3ccccc3)n2)C(c2ccccc2)=CC1 ; C1=Cc2c(cccc2-c2cc(-c3ccc4cc(-c5ccc(-n6c7ccccc7c7ccccc76)cc5)ccc4c3)nc(-c3ccccc3)n2)C(c2ccccc2)=CC1 ; C1=Cc2c(cccc2-c2cc(-c3ccc4cc(-c5ccc6c(c5)c5ccccc5n6-c5ccccc5)ccc4c3)nc(-c3ccccc3)n2)C(c2ccccc2)=CC1]

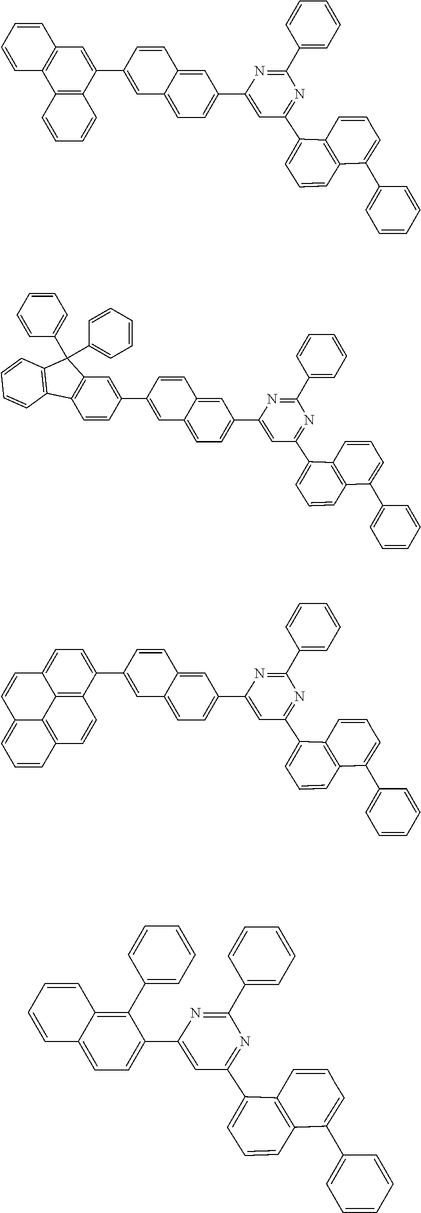 [SMILES: C1=Cc2c(cccc2-c2cc(-c3ccc4cc(-c5cc6ccccc6c6ccccc56)ccc4c3)nc(-c3ccccc3)n2)C(c2ccccc2)=CC1 ; C1=Cc2c(cccc2-c2cc(-c3ccc4cc(-c5ccc6c(c5)C(c5ccccc5)(c5ccccc5)c5ccccc5-6)ccc4c3)nc(-c3ccccc3)n2)C(c2ccccc2)=CC1 ; C1=Cc2c(cccc2-c2cc(-c3ccc4cc(-c5ccc6ccc7cccc8ccc5c6c78)ccc4c3)nc(-c3ccccc3)n2)C(c2ccccc2)=CC1 ; C1=Cc2c(cccc2-c2cc(-c3ccc4ccccc4c3-c3ccccc3)nc(-c3ccccc3)n2)C(c2ccccc2)=CC1]

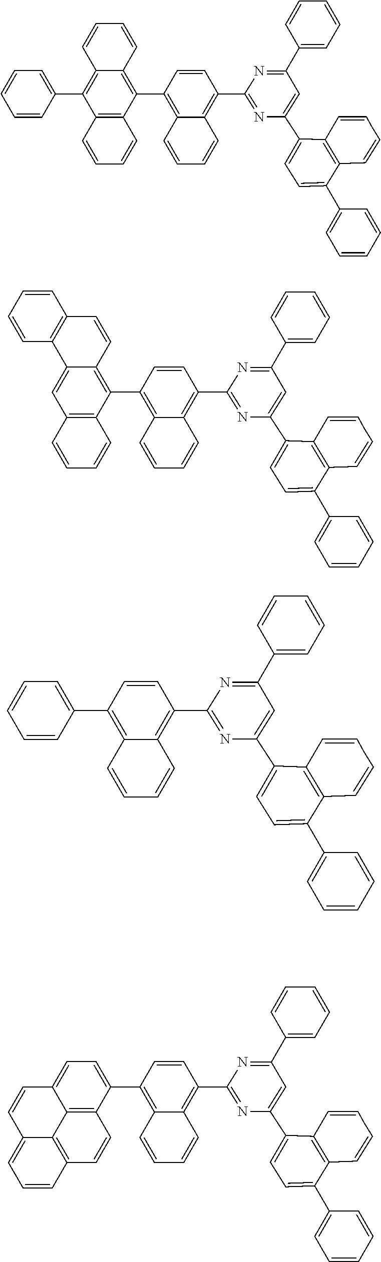

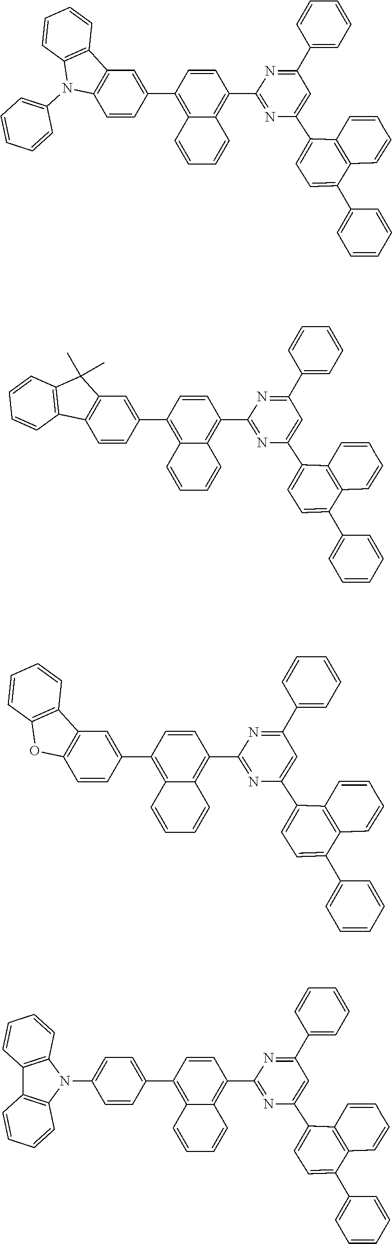 [SMILES: C1=Cc2c(-c3ccccc3)ccc(-c3cc(-c4ccccc4)nc(-c4ccc(-c5ccc6c(c5)c5ccccc5n6-c5ccccc5)c5ccccc45)n3)c2C=CC1 ; CC1(C)c2ccccc2-c2ccc(-c3ccc(-c4nc(-c5ccccc5)cc(-c5ccc(-c6ccccc6)c6c5C=CCC=C6)n4)c4ccccc34)cc21 ; C1=Cc2c(-c3ccccc3)ccc(-c3cc(-c4ccccc4)nc(-c4ccc(-c5ccc6oc7ccccc7c6c5)c5ccccc45)n3)c2C=CC1 ; C1=Cc2c(-c3ccccc3)ccc(-c3cc(-c4ccccc4)nc(-c4ccc(-c5ccc(-n6c7ccccc7c7ccccc76)cc5)c5ccccc45)n3)c2C=CC1]

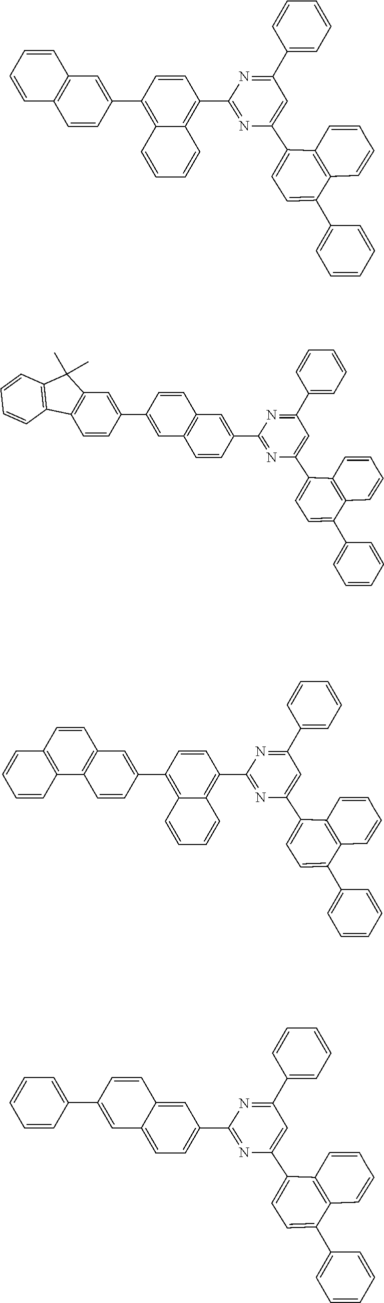 [SMILES: C1=Cc2c(-c3ccccc3)ccc(-c3cc(-c4ccccc4)nc(-c4ccc(-c5ccc6ccccc6c5)c5ccccc45)n3)c2C=CC1 ; CC1(C)c2ccccc2-c2ccc(-c3ccc4cc(-c5nc(-c6ccccc6)cc(-c6ccc(-c7ccccc7)c7c6C=CCC=C7)n5)ccc4c3)cc21 ; C1=Cc2c(-c3ccccc3)ccc(-c3cc(-c4ccccc4)nc(-c4ccc(-c5ccc6c(ccc7ccccc76)c5)c5ccccc45)n3)c2C=CC1 ; C1=Cc2c(-c3ccccc3)ccc(-c3cc(-c4ccccc4)nc(-c4ccc5cc(-c6ccccc6)ccc5c4)n3)c2C=CC1]

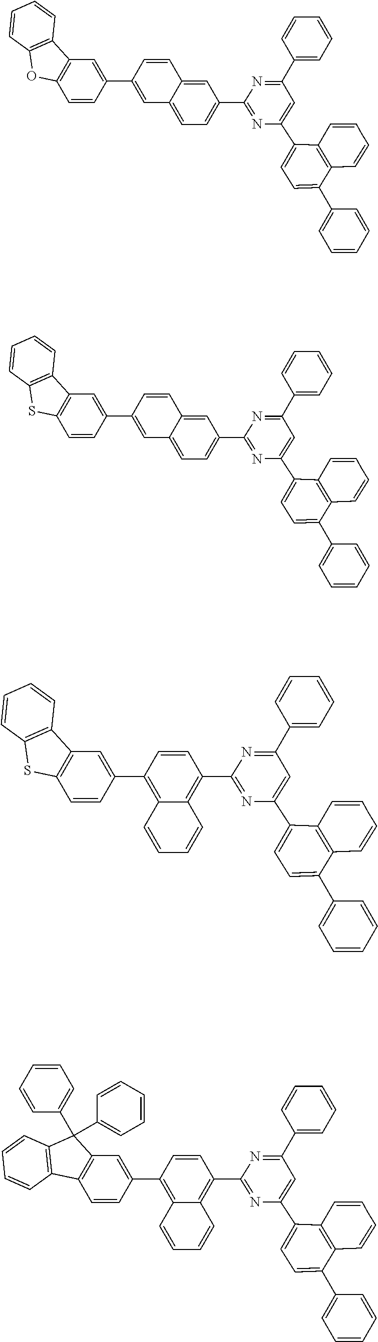 [SMILES: C1=Cc2c(-c3ccccc3)ccc(-c3cc(-c4ccccc4)nc(-c4ccc5cc(-c6ccc7oc8ccccc8c7c6)ccc5c4)n3)c2C=CC1 ; C1=Cc2c(-c3ccccc3)ccc(-c3cc(-c4ccccc4)nc(-c4ccc5cc(-c6ccc7sc8ccccc8c7c6)ccc5c4)n3)c2C=CC1 ; C1=Cc2c(-c3ccccc3)ccc(-c3cc(-c4ccccc4)nc(-c4ccc(-c5ccc6sc7ccccc7c6c5)c5ccccc45)n3)c2C=CC1 ; C1=Cc2c(-c3ccccc3)ccc(-c3cc(-c4ccccc4)nc(-c4ccc(-c5ccc6c(c5)C(c5ccccc5)(c5ccccc5)c5ccccc5-6)c5ccccc45)n3)c2C=CC1]

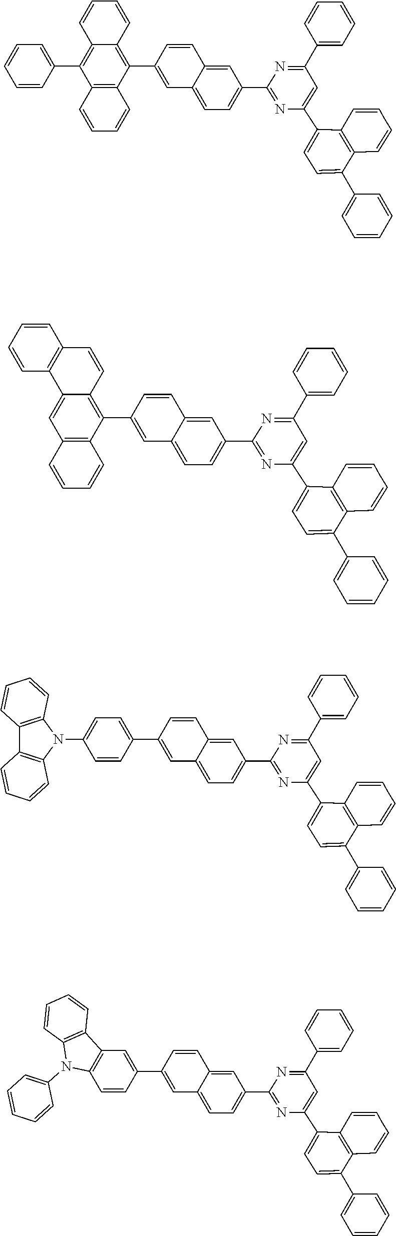 [SMILES: C1=Cc2c(-c3ccccc3)ccc(-c3cc(-c4ccccc4)nc(-c4ccc5cc(-c6c7ccccc7c(-c7ccccc7)c7ccccc67)ccc5c4)n3)c2C=CC1 ; C1=Cc2c(-c3ccccc3)ccc(-c3cc(-c4ccccc4)nc(-c4ccc5cc(-c6c7ccccc7cc7c6ccc6ccccc67)ccc5c4)n3)c2C=CC1 ; C1=Cc2c(-c3ccccc3)ccc(-c3cc(-c4ccccc4)nc(-c4ccc5cc(-c6ccc(-n7c8ccccc8c8ccccc87)cc6)ccc5c4)n3)c2C=CC1 ; C1=Cc2c(-c3ccccc3)ccc(-c3cc(-c4ccccc4)nc(-c4ccc5cc(-c6ccc7c(c6)c6ccccc6n7-c6ccccc6)ccc5c4)n3)c2C=CC1]

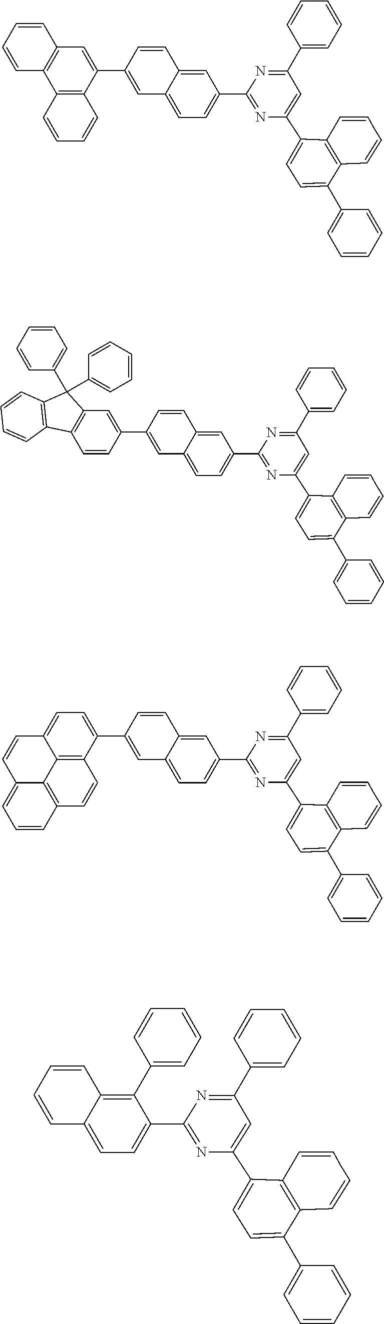 [SMILES: C1=Cc2c(-c3ccccc3)ccc(-c3cc(-c4ccccc4)nc(-c4ccc5cc(-c6cc7ccccc7c7ccccc67)ccc5c4)n3)c2C=CC1 ; C1=Cc2c(-c3ccccc3)ccc(-c3cc(-c4ccccc4)nc(-c4ccc5cc(-c6ccc7c(c6)C(c6ccccc6)(c6ccccc6)c6ccccc6-7)ccc5c4)n3)c2C=CC1 ; C1=Cc2c(-c3ccccc3)ccc(-c3cc(-c4ccccc4)nc(-c4ccc5cc(-c6ccc7ccc8cccc9ccc6c7c89)ccc5c4)n3)c2C=CC1 ; C1=Cc2c(-c3ccccc3)ccc(-c3cc(-c4ccccc4)nc(-c4ccc5ccccc5c4-c4ccccc4)n3)c2C=CC1]

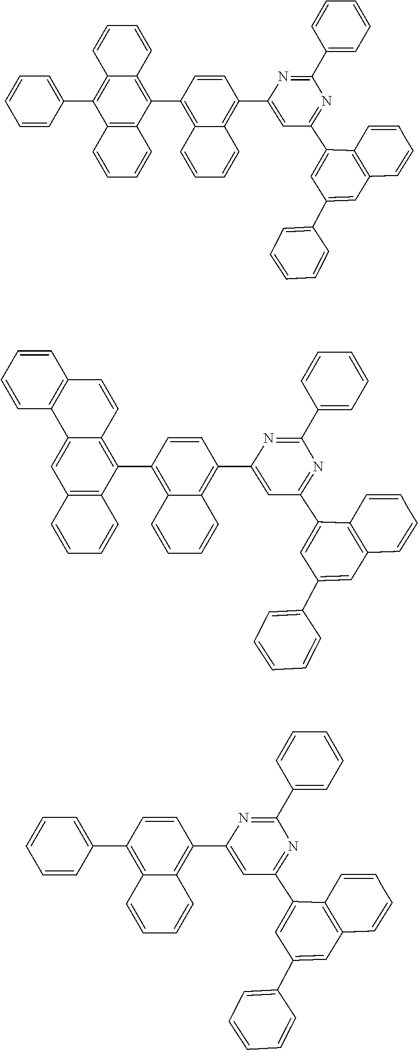 [SMILES: C1=Cc2cc(-c3ccccc3)cc(-c3cc(-c4ccc(-c5c6ccccc6c(-c6ccccc6)c6ccccc56)c5ccccc45)nc(-c4ccccc4)n3)c2C=CC1 ; C1=Cc2cc(-c3ccccc3)cc(-c3cc(-c4ccc(-c5c6ccccc6cc6c5ccc5ccccc56)c5ccccc45)nc(-c4ccccc4)n3)c2C=CC1 ; C1=Cc2cc(-c3ccccc3)cc(-c3cc(-c4ccc(-c5ccccc5)c5ccccc45)nc(-c4ccccc4)n3)c2C=CC1]

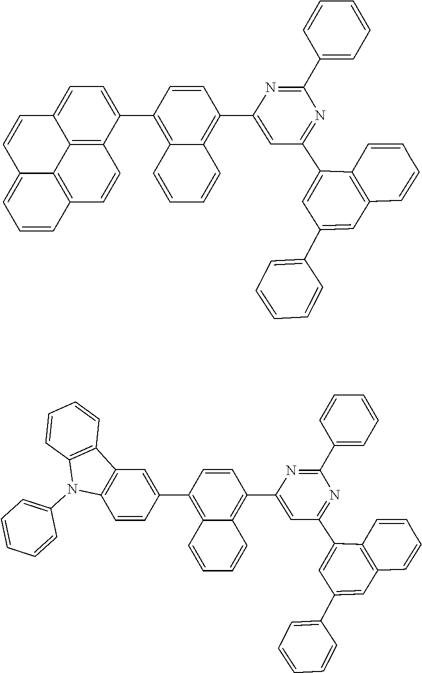 [SMILES: C1=Cc2cc(-c3ccccc3)cc(-c3cc(-c4ccc(-c5ccc6ccc7cccc8ccc5c6c78)c5ccccc45)nc(-c4ccccc4)n3)c2C=CC1 ; C1=Cc2cc(-c3ccccc3)cc(-c3cc(-c4ccc(-c5ccc6c(c5)c5ccccc5n6-c5ccccc5)c5ccccc45)nc(-c4ccccc4)n3)c2C=CC1]

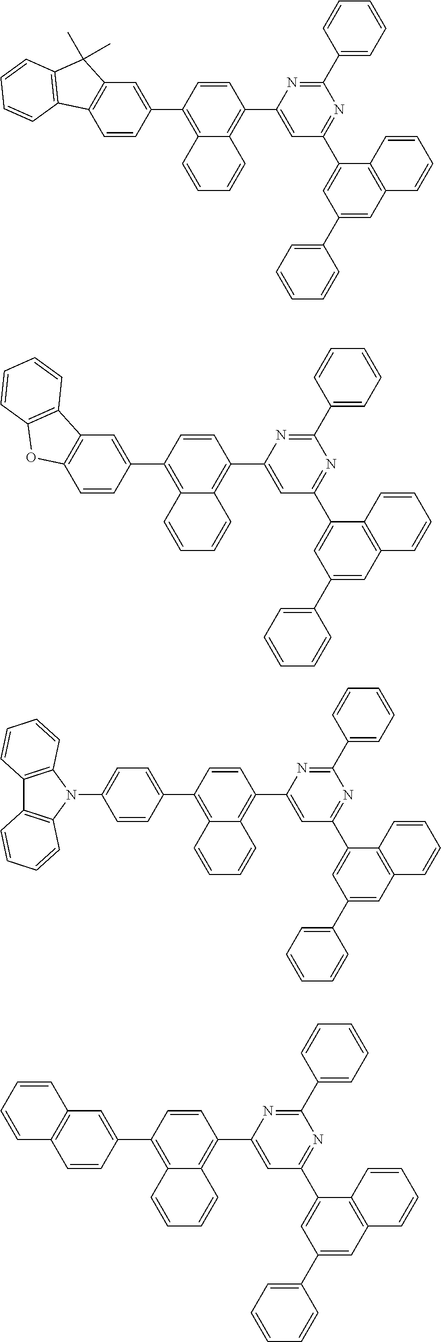

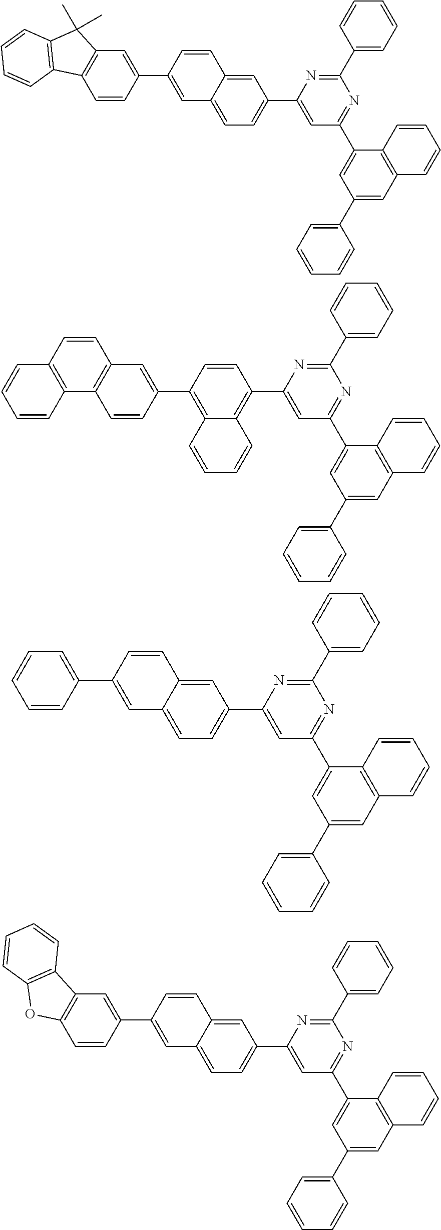

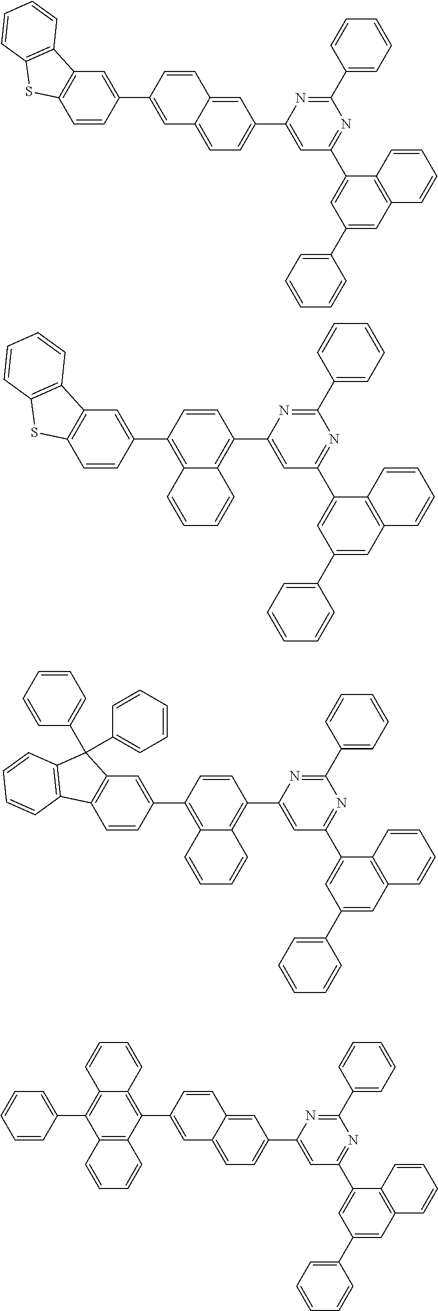

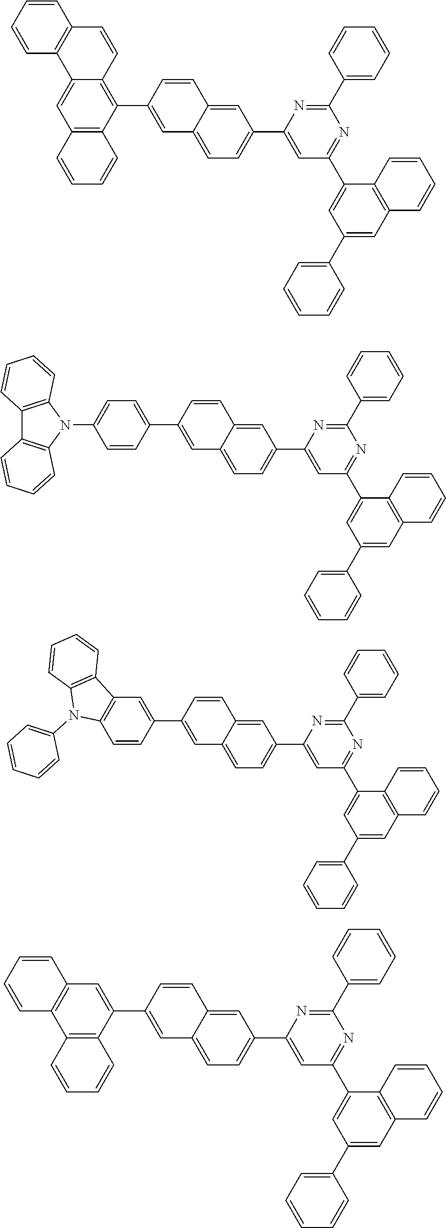

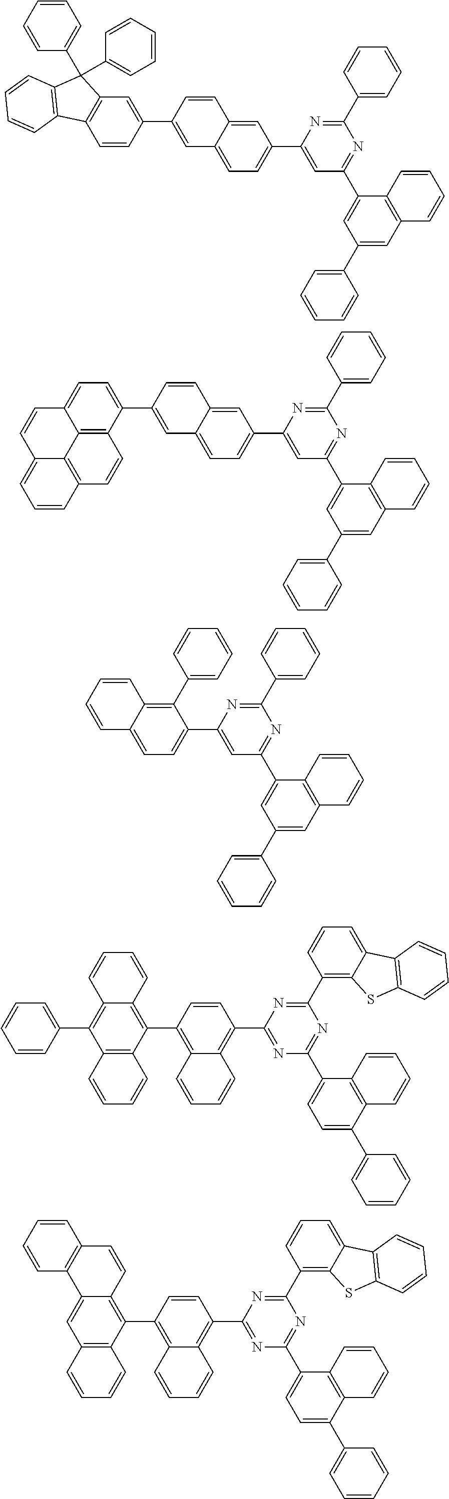

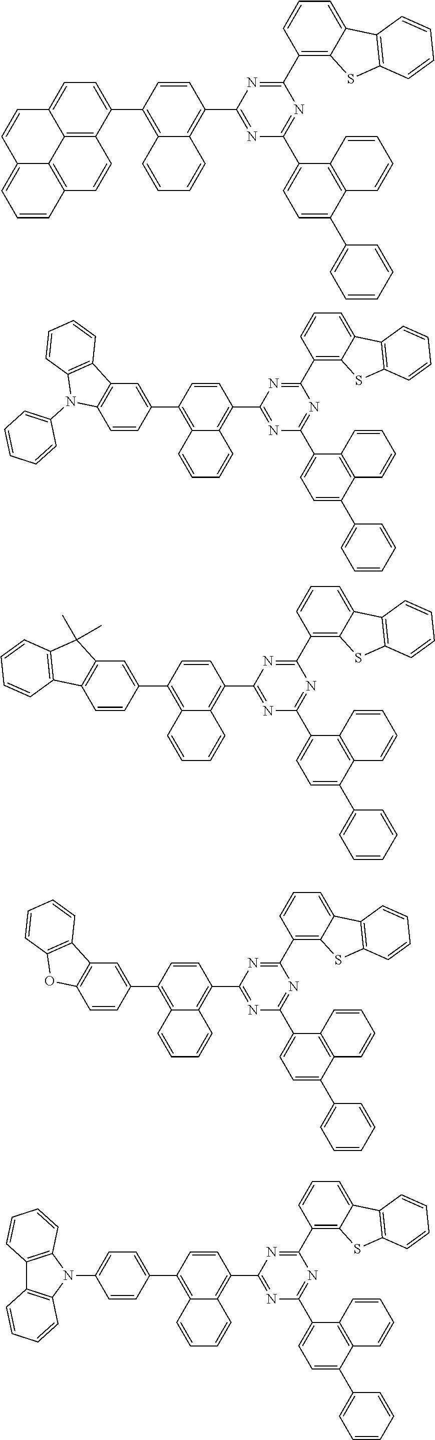

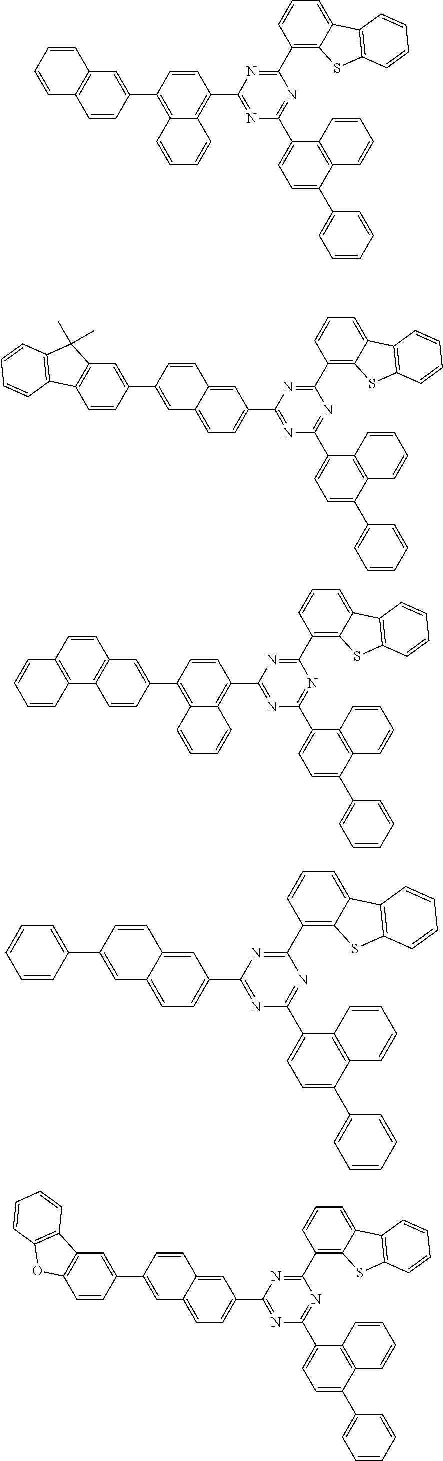

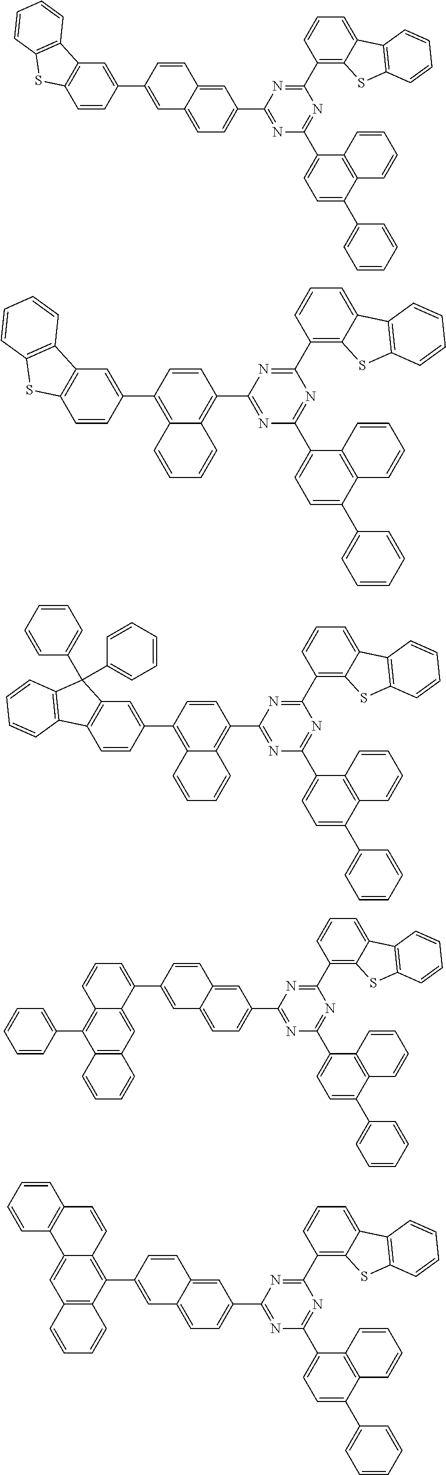

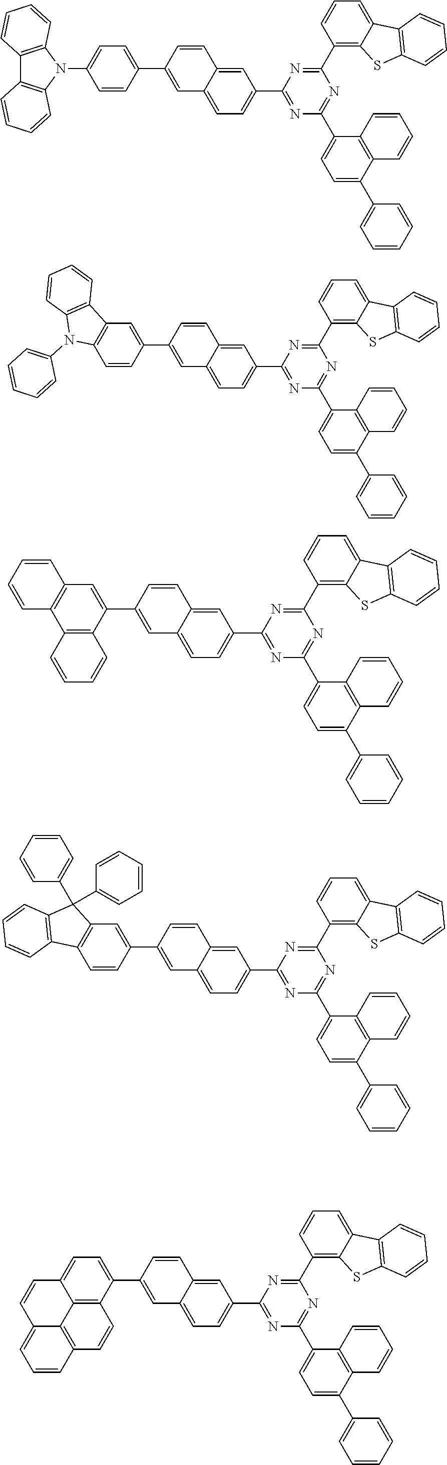

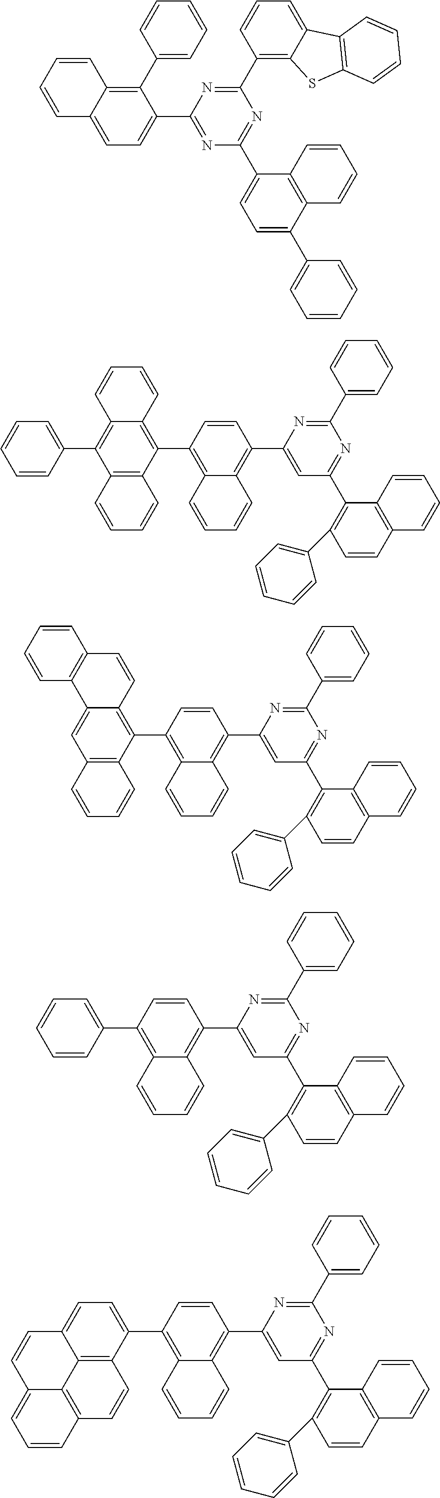

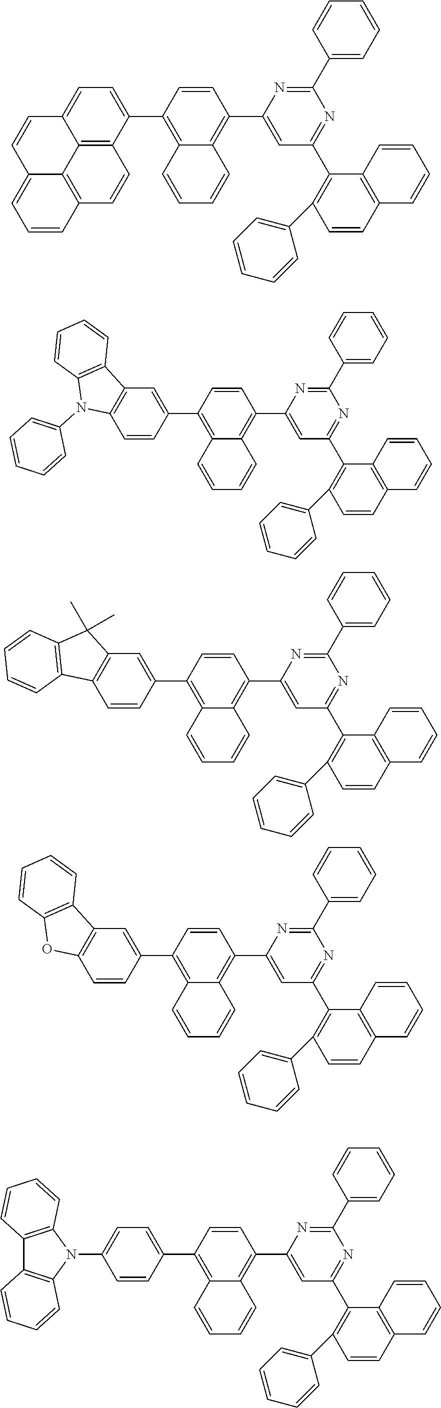

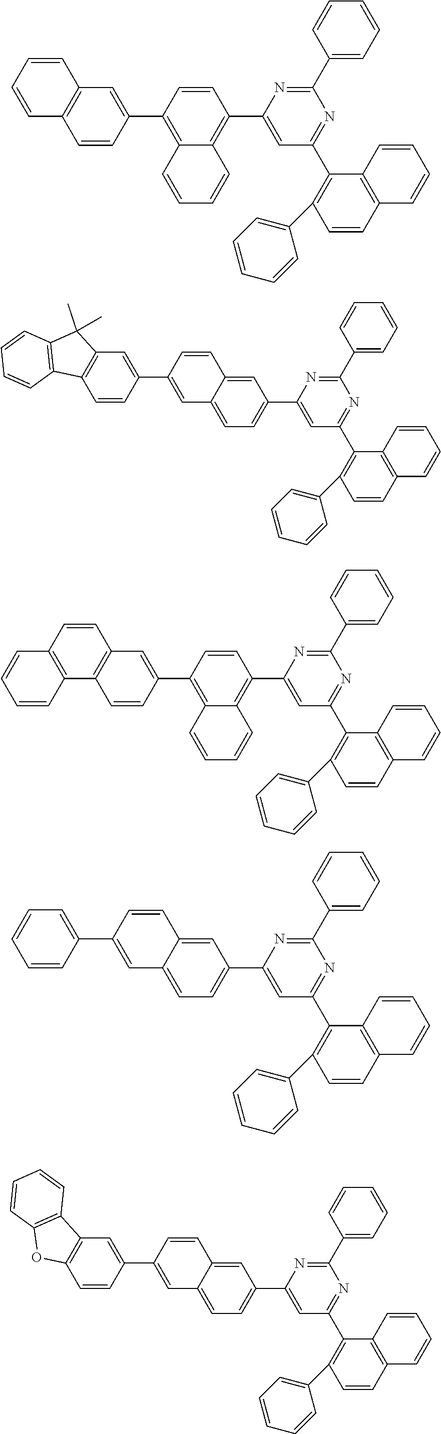

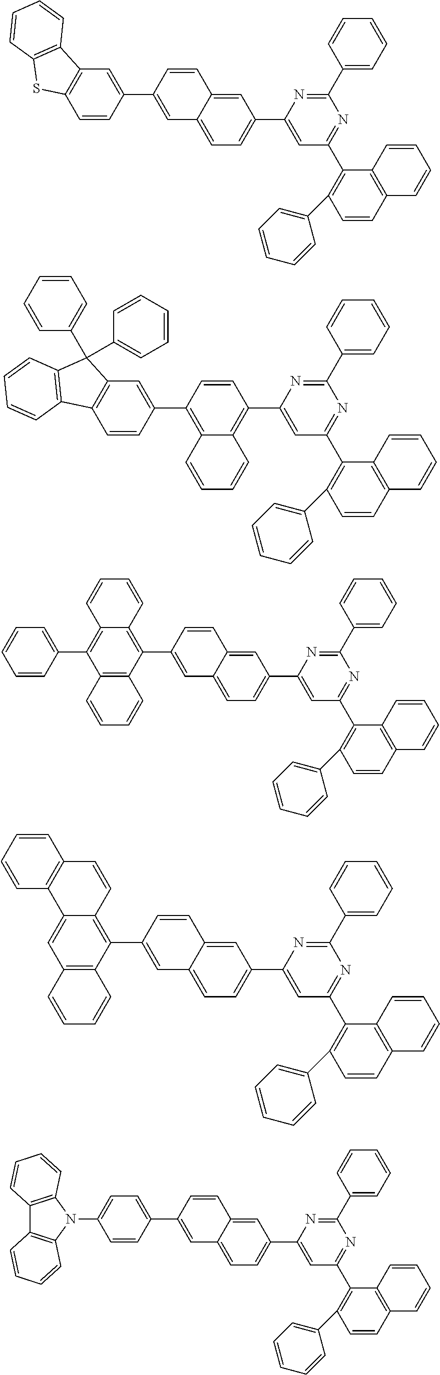 [SMILES: c1ccc(-c2nc(-c3ccc4cc(-c5ccc6sc7ccccc7c6c5)ccc4c3)cc(-c3c(-c4ccccc4)ccc4ccccc34)n2)cc1 ; c1ccc(-c2nc(-c3c(-c4ccccc4)ccc4ccccc34)cc(-c3ccc(-c4ccc5c(c4)C(c4ccccc4)(c4ccccc4)c4ccccc4-5)c4ccccc34)n2)cc1 ; c1ccc(-c2nc(-c3ccc4cc(-c5c6ccccc6c(-c6ccccc6)c6ccccc56)ccc4c3)cc(-c3c(-c4ccccc4)ccc4ccccc34)n2)cc1 ; c1ccc(-c2nc(-c3ccc4cc(-c5c6ccccc6cc6c5ccc5ccccc56)ccc4c3)cc(-c3c(-c4ccccc4)ccc4ccccc34)n2)cc1 ; c1ccc(-c2nc(-c3ccc4cc(-c5ccc(-n6c7ccccc7c7ccccc76)cc5)ccc4c3)cc(-c3c(-c4ccccc4)ccc4ccccc34)n2)cc1]

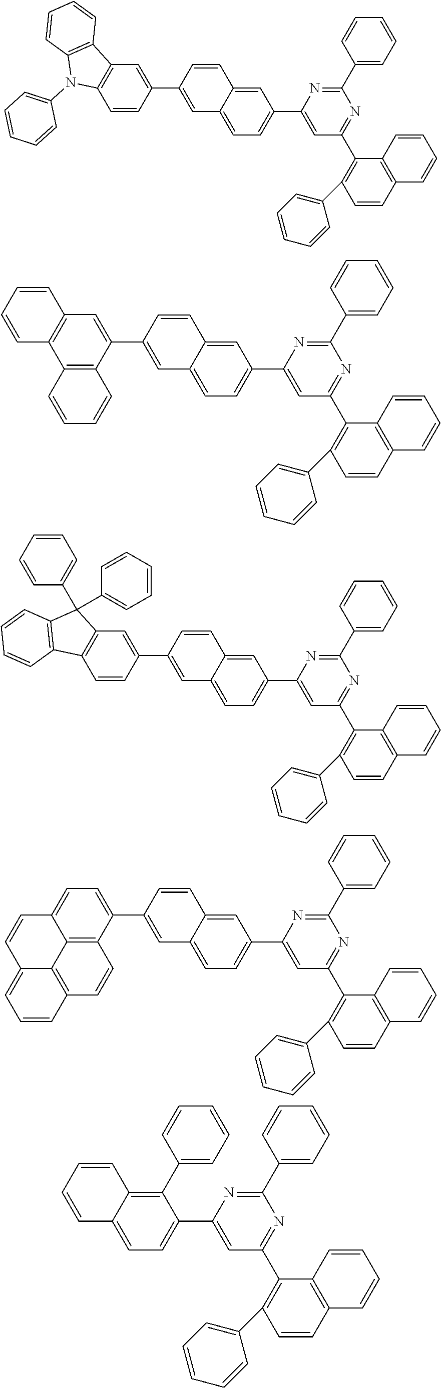

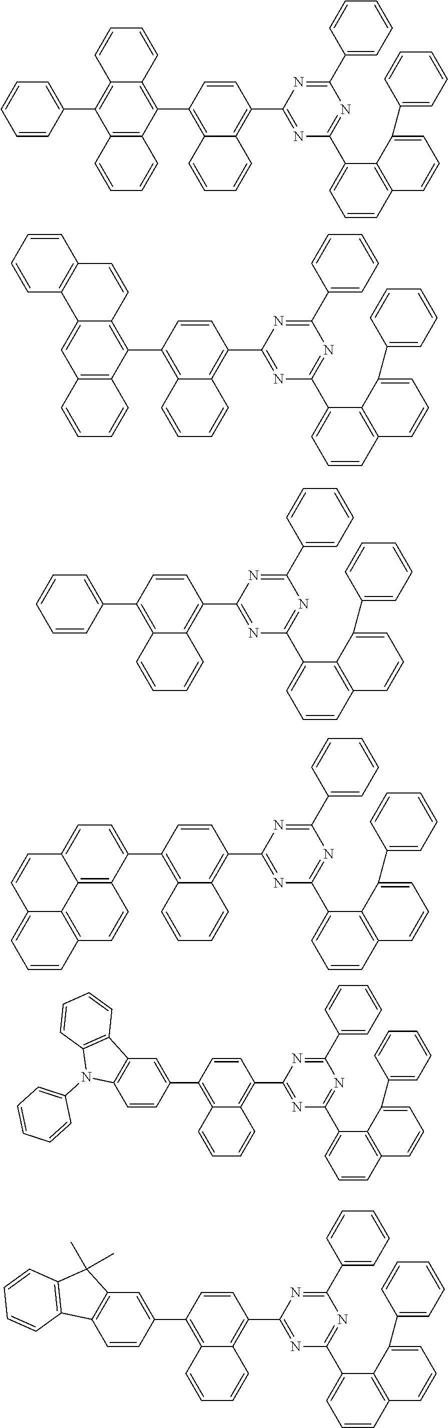

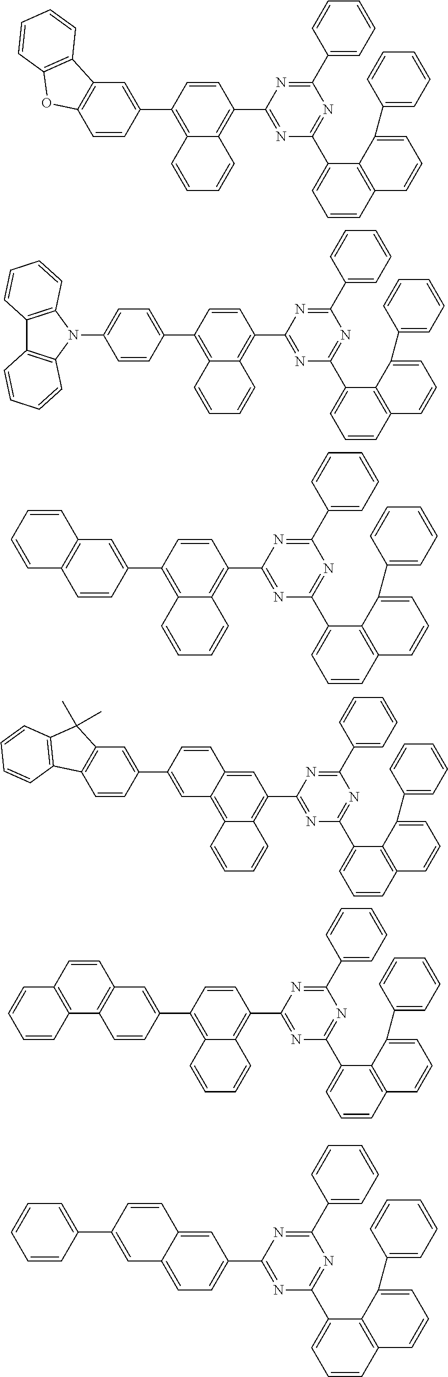

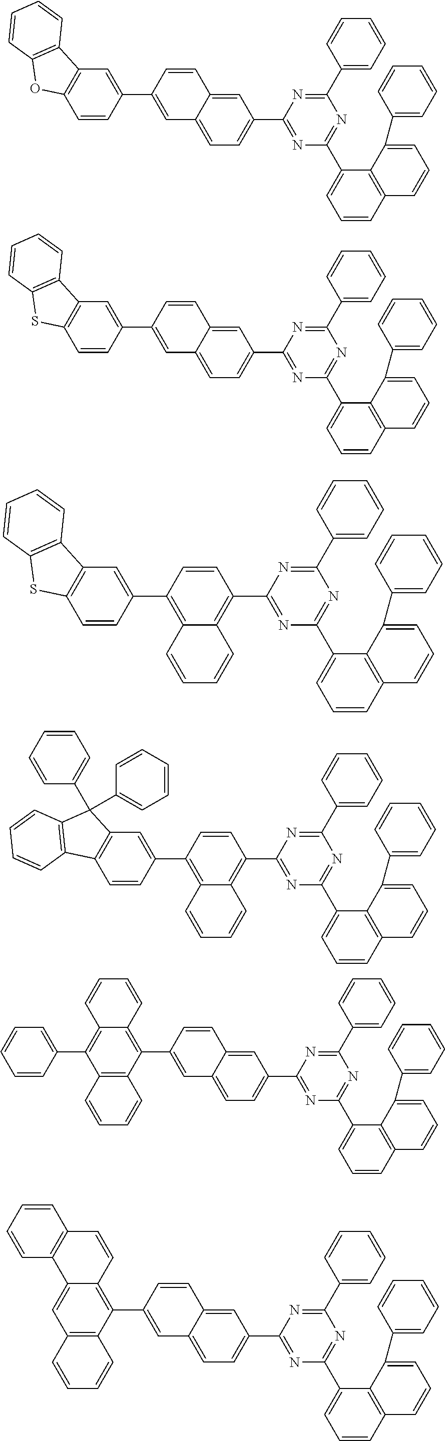

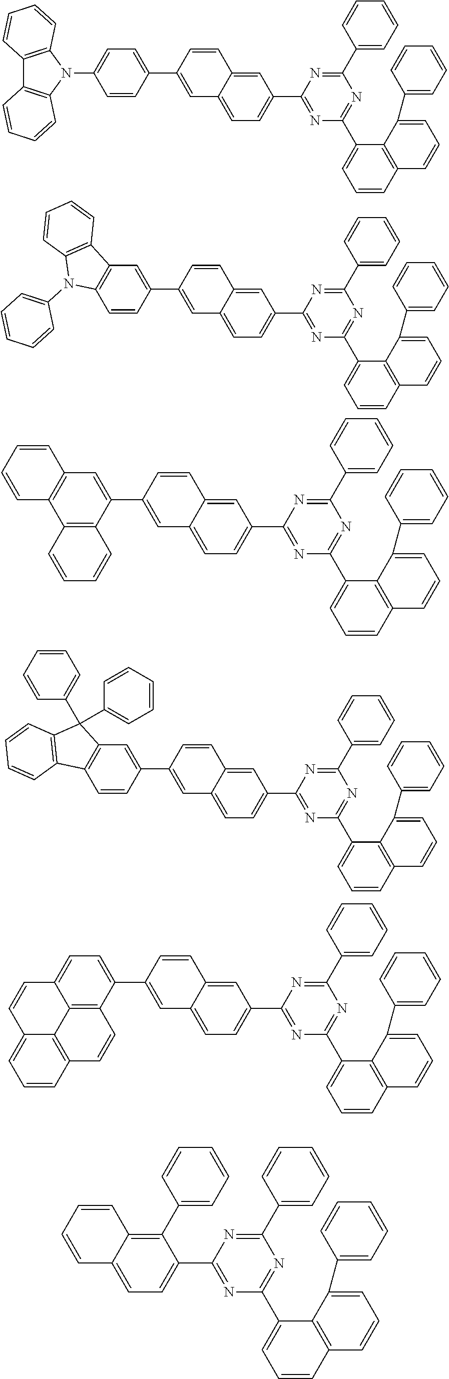

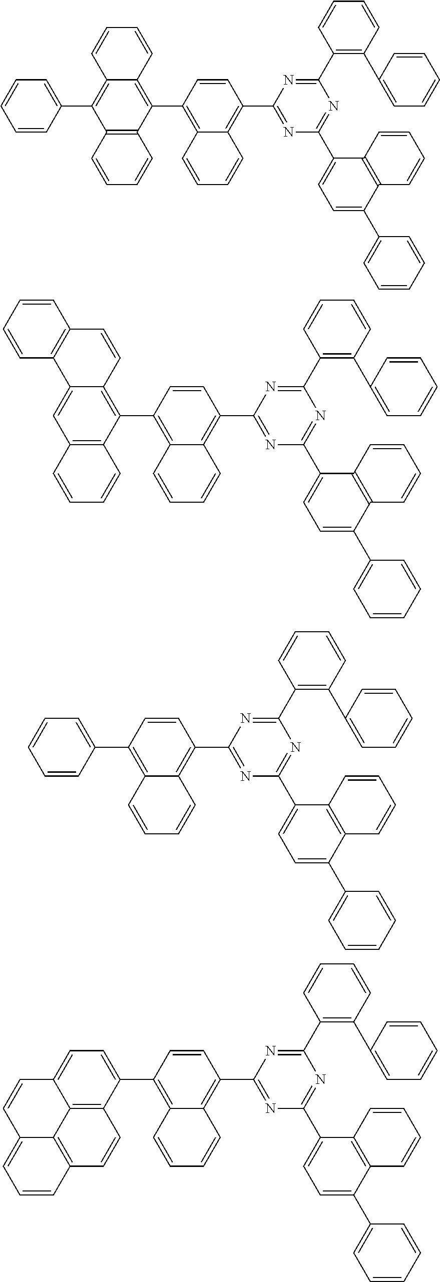

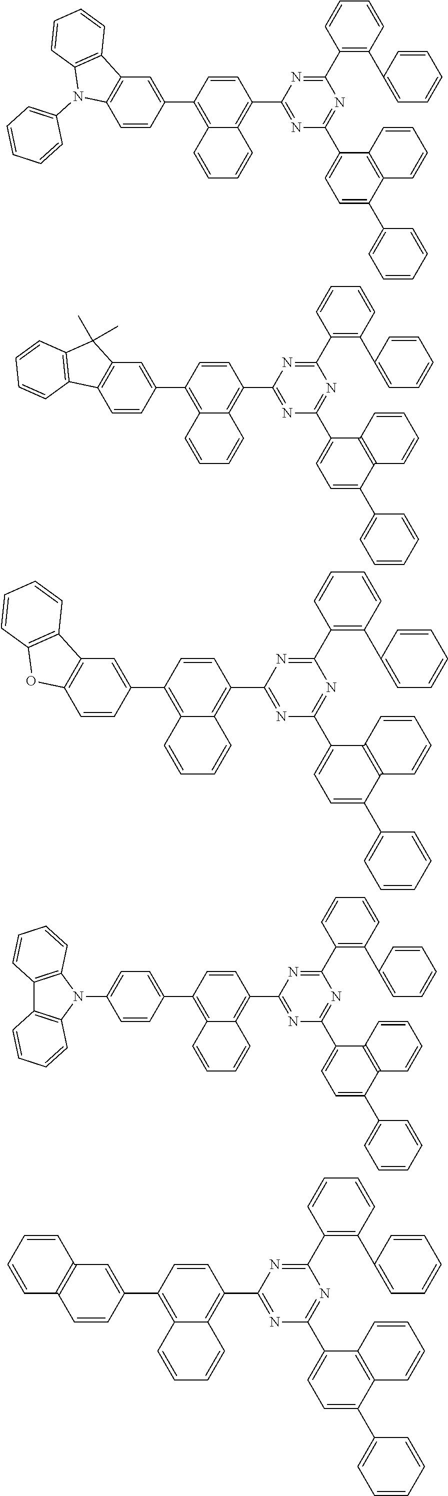

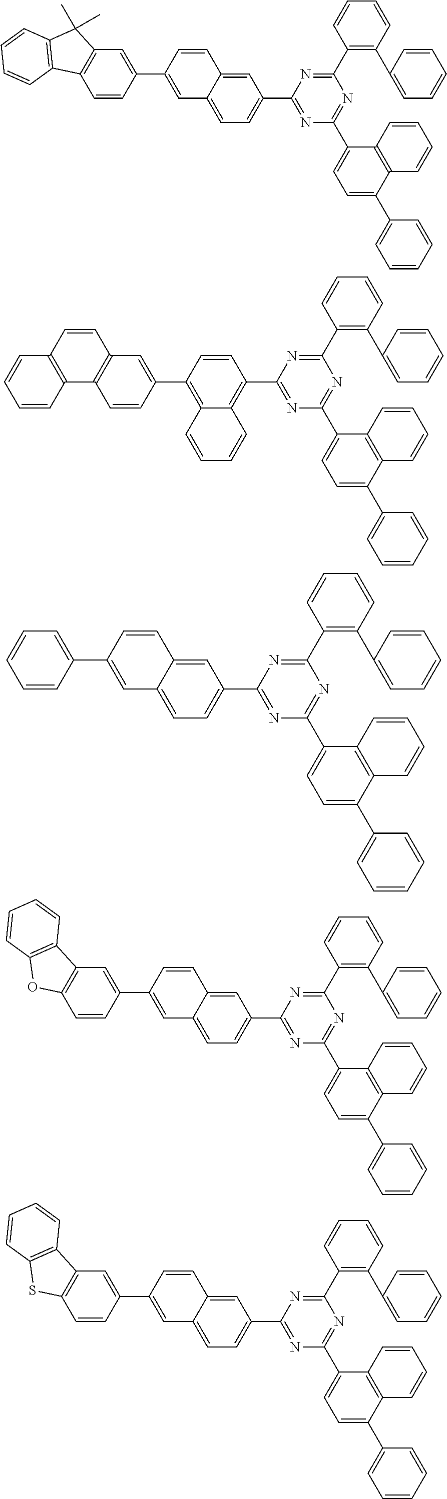

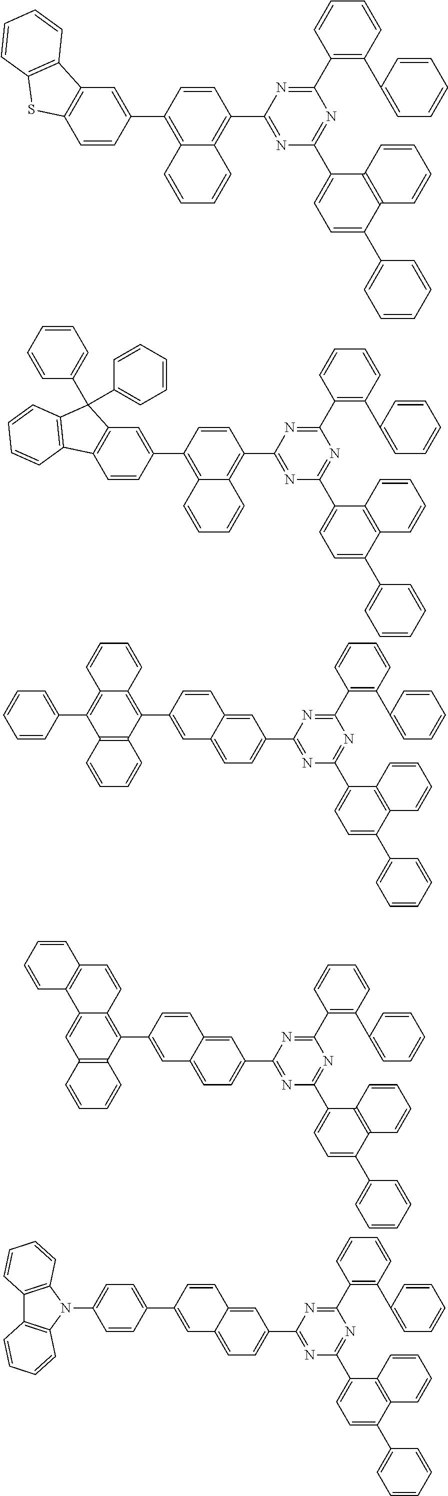

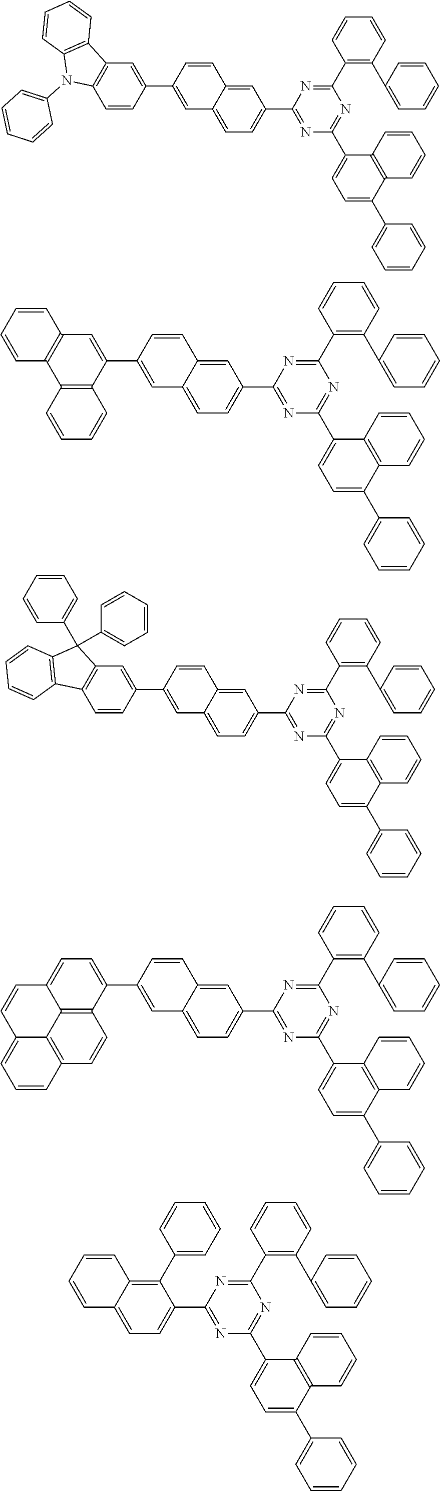

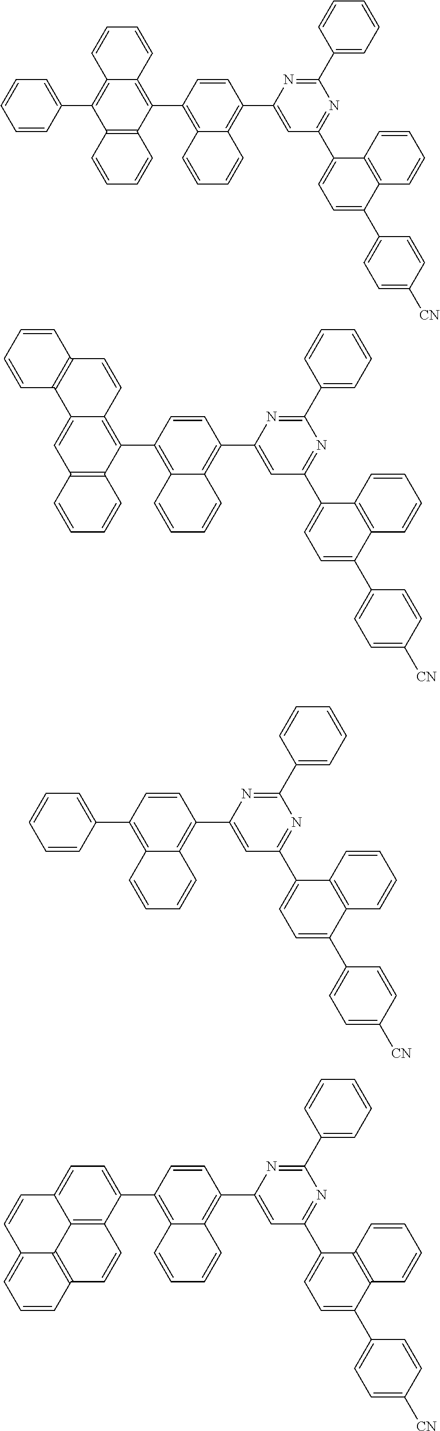 [SMILES: N#Cc1ccc(-c2ccc(-c3cc(-c4ccc(-c5c6ccccc6c(-c6ccccc6)c6ccccc56)c5ccccc45)nc(-c4ccccc4)n3)c3ccccc23)cc1 ; N#Cc1ccc(-c2ccc(-c3cc(-c4ccc(-c5c6ccccc6cc6c5ccc5ccccc56)c5ccccc45)nc(-c4ccccc4)n3)c3ccccc23)cc1 ; N#Cc1ccc(-c2ccc(-c3cc(-c4ccc(-c5ccccc5)c5ccccc45)nc(-c4ccccc4)n3)c3ccccc23)cc1 ; N#Cc1ccc(-c2ccc(-c3cc(-c4ccc(-c5ccc6ccc7cccc8ccc5c6c78)c5ccccc45)nc(-c4ccccc4)n3)c3ccccc23)cc1]

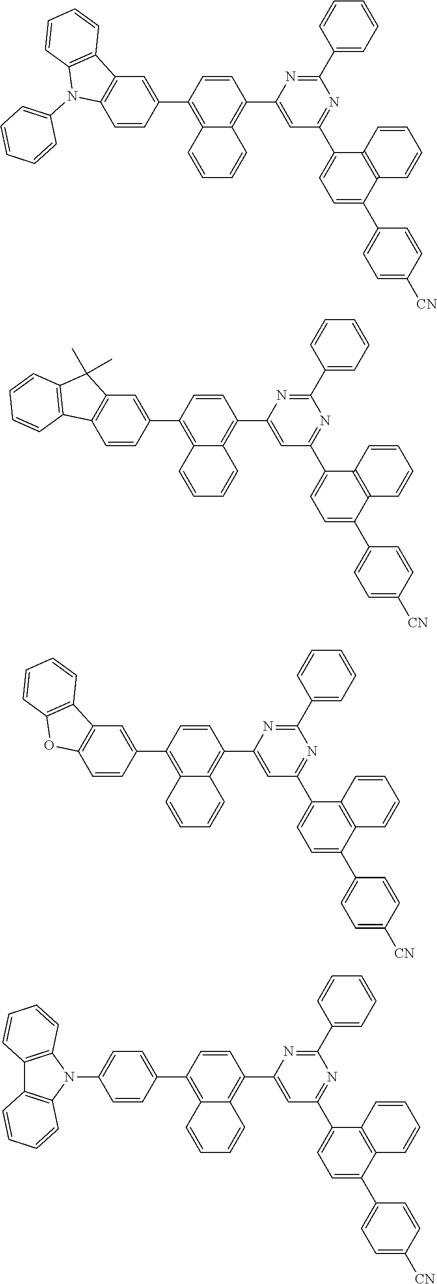 [SMILES: N#Cc1ccc(-c2ccc(-c3cc(-c4ccc(-c5ccc6c(c5)c5ccccc5n6-c5ccccc5)c5ccccc45)nc(-c4ccccc4)n3)c3ccccc23)cc1 ; CC1(C)c2ccccc2-c2ccc(-c3ccc(-c4cc(-c5ccc(-c6ccc(C#N)cc6)c6ccccc56)nc(-c5ccccc5)n4)c4ccccc34)cc21 ; N#Cc1ccc(-c2ccc(-c3cc(-c4ccc(-c5ccc6oc7ccccc7c6c5)c5ccccc45)nc(-c4ccccc4)n3)c3ccccc23)cc1 ; N#Cc1ccc(-c2ccc(-c3cc(-c4ccc(-c5ccc(-n6c7ccccc7c7ccccc76)cc5)c5ccccc45)nc(-c4ccccc4)n3)c3ccccc23)cc1]

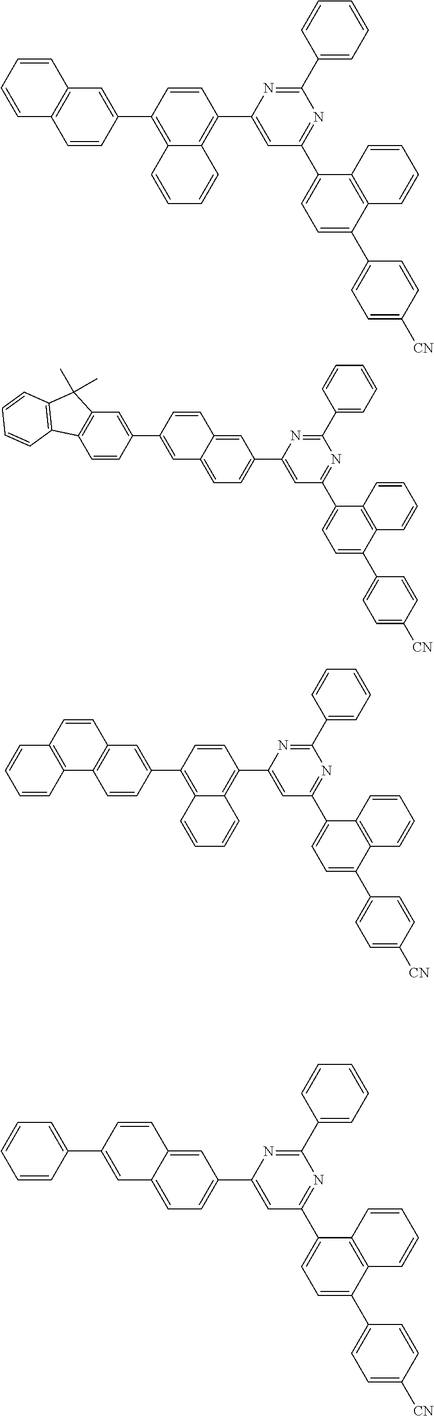 [SMILES: N#Cc1ccc(-c2ccc(-c3cc(-c4ccc(-c5ccc6ccccc6c5)c5ccccc45)nc(-c4ccccc4)n3)c3ccccc23)cc1 ; CC1(C)c2ccccc2-c2ccc(-c3ccc4cc(-c5cc(-c6ccc(-c7ccc(C#N)cc7)c7ccccc67)nc(-c6ccccc6)n5)ccc4c3)cc21 ; N#Cc1ccc(-c2ccc(-c3cc(-c4ccc(-c5ccc6c(ccc7ccccc76)c5)c5ccccc45)nc(-c4ccccc4)n3)c3ccccc23)cc1 ; N#Cc1ccc(-c2ccc(-c3cc(-c4ccc5cc(-c6ccccc6)ccc5c4)nc(-c4ccccc4)n3)c3ccccc23)cc1]

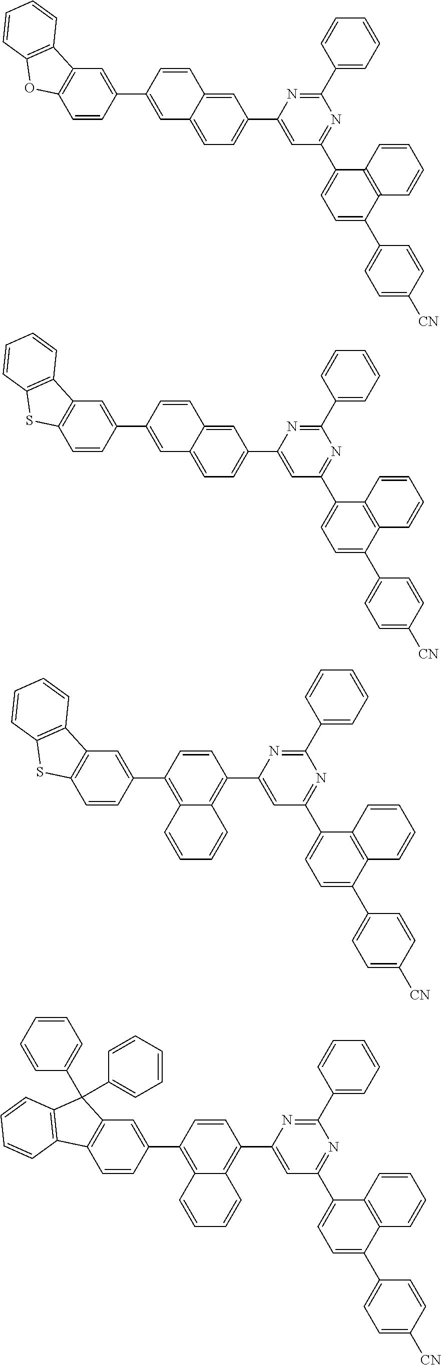 [SMILES: N#Cc1ccc(-c2ccc(-c3cc(-c4ccc5cc(-c6ccc7oc8ccccc8c7c6)ccc5c4)nc(-c4ccccc4)n3)c3ccccc23)cc1 ; N#Cc1ccc(-c2ccc(-c3cc(-c4ccc5cc(-c6ccc7sc8ccccc8c7c6)ccc5c4)nc(-c4ccccc4)n3)c3ccccc23)cc1 ; N#Cc1ccc(-c2ccc(-c3cc(-c4ccc(-c5ccc6sc7ccccc7c6c5)c5ccccc45)nc(-c4ccccc4)n3)c3ccccc23)cc1 ; N#Cc1ccc(-c2ccc(-c3cc(-c4ccc(-c5ccc6c(c5)C(c5ccccc5)(c5ccccc5)c5ccccc5-6)c5ccccc45)nc(-c4ccccc4)n3)c3ccccc23)cc1]

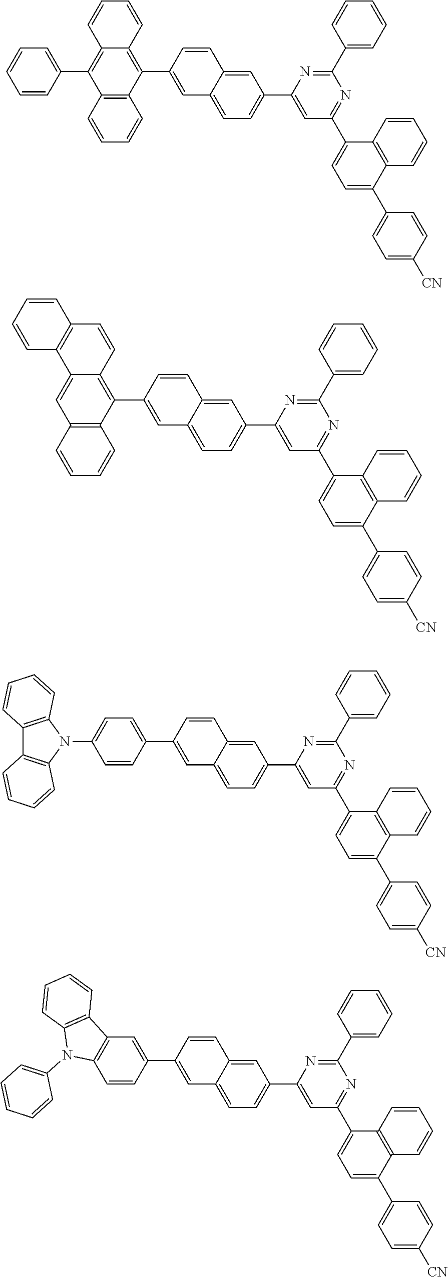 [SMILES: N#Cc1ccc(-c2ccc(-c3cc(-c4ccc5cc(-c6c7ccccc7c(-c7ccccc7)c7ccccc67)ccc5c4)nc(-c4ccccc4)n3)c3ccccc23)cc1 ; N#Cc1ccc(-c2ccc(-c3cc(-c4ccc5cc(-c6c7ccccc7cc7c6ccc6ccccc67)ccc5c4)nc(-c4ccccc4)n3)c3ccccc23)cc1 ; N#Cc1ccc(-c2ccc(-c3cc(-c4ccc5cc(-c6ccc(-n7c8ccccc8c8ccccc87)cc6)ccc5c4)nc(-c4ccccc4)n3)c3ccccc23)cc1 ; N#Cc1ccc(-c2ccc(-c3cc(-c4ccc5cc(-c6ccc7c(c6)c6ccccc6n7-c6ccccc6)ccc5c4)nc(-c4ccccc4)n3)c3ccccc23)cc1]

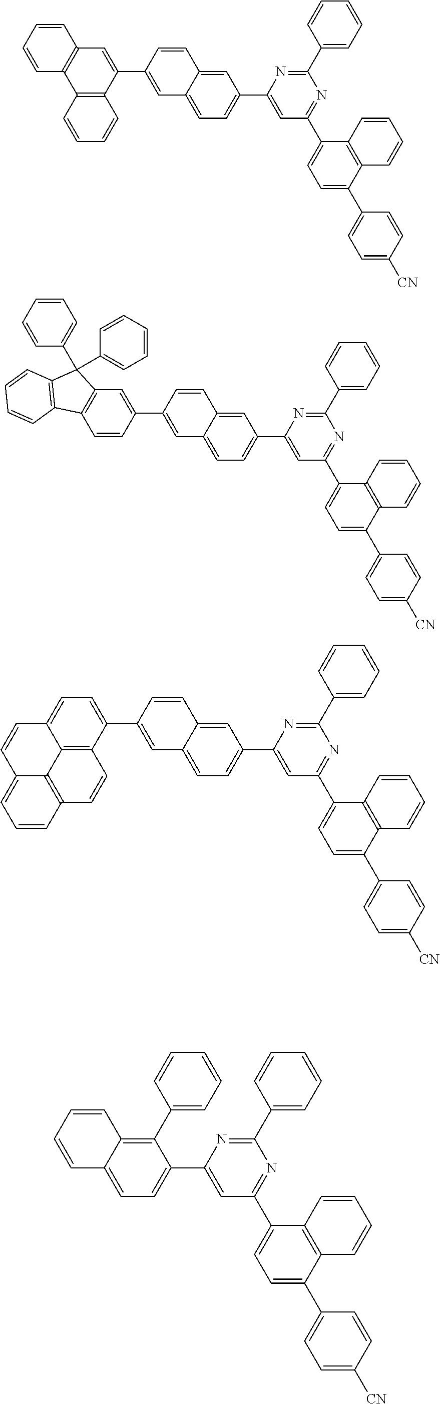 [SMILES: N#Cc1ccc(-c2ccc(-c3cc(-c4ccc5cc(-c6cc7ccccc7c7ccccc67)ccc5c4)nc(-c4ccccc4)n3)c3ccccc23)cc1 ; N#Cc1ccc(-c2ccc(-c3cc(-c4ccc5cc(-c6ccc7c(c6)C(c6ccccc6)(c6ccccc6)c6ccccc6-7)ccc5c4)nc(-c4ccccc4)n3)c3ccccc23)cc1 ; N#Cc1ccc(-c2ccc(-c3cc(-c4ccc5cc(-c6ccc7ccc8cccc9ccc6c7c89)ccc5c4)nc(-c4ccccc4)n3)c3ccccc23)cc1 ; N#Cc1ccc(-c2ccc(-c3cc(-c4ccc5ccccc5c4-c4ccccc4)nc(-c4ccccc4)n3)c3ccccc23)cc1]

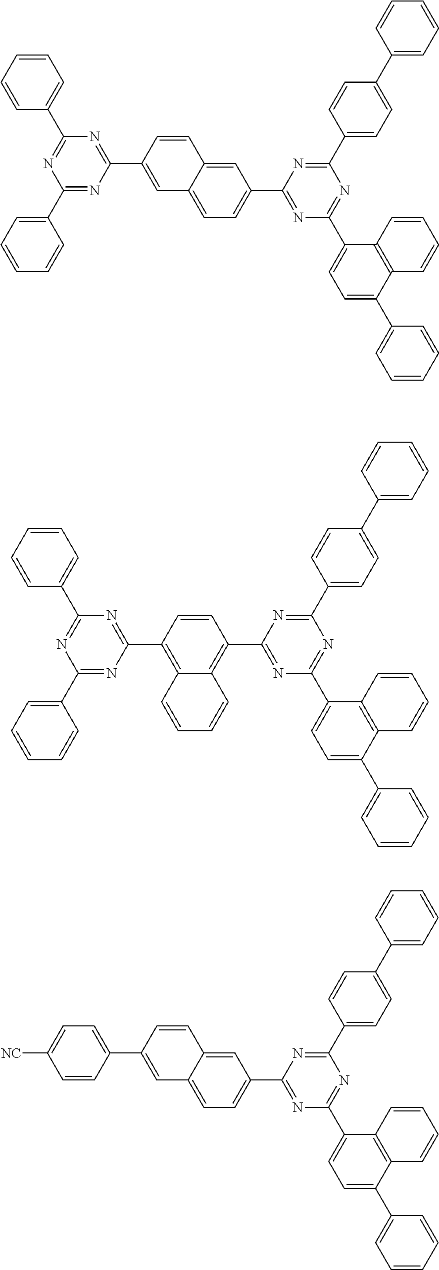 [SMILES: c1ccc(-c2ccc(-c3nc(-c4ccc5cc(-c6nc(-c7ccccc7)nc(-c7ccccc7)n6)ccc5c4)nc(-c4ccc(-c5ccccc5)c5ccccc45)n3)cc2)cc1 ; c1ccc(-c2ccc(-c3nc(-c4ccc(-c5ccccc5)c5ccccc45)nc(-c4ccc(-c5nc(-c6ccccc6)nc(-c6ccccc6)n5)c5ccccc45)n3)cc2)cc1 ; N#Cc1ccc(-c2ccc3cc(-c4nc(-c5ccc(-c6ccccc6)cc5)nc(-c5ccc(-c6ccccc6)c6ccccc56)n4)ccc3c2)cc1]

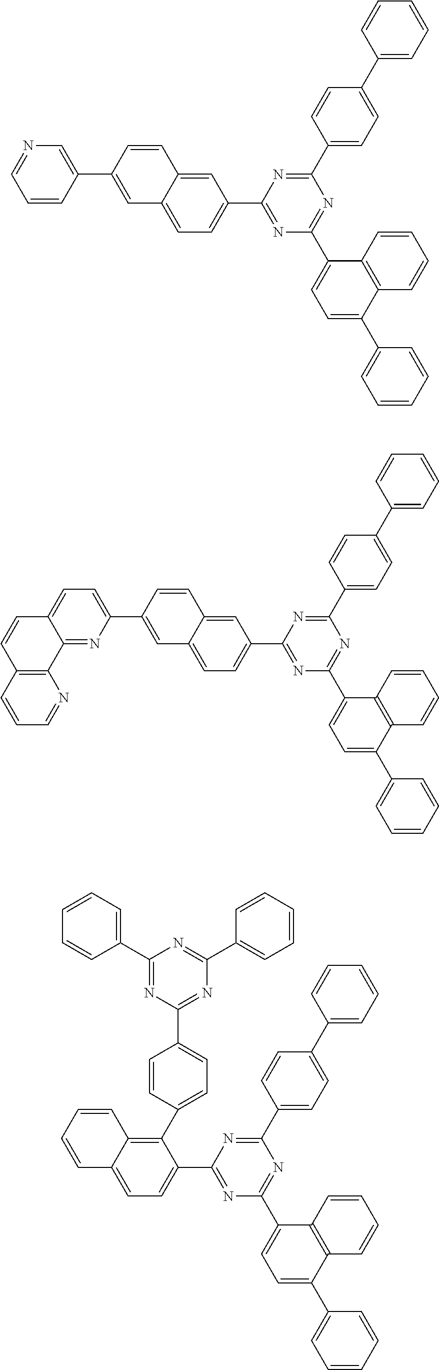 [SMILES: c1ccc(-c2ccc(-c3nc(-c4ccc5cc(-c6cccnc6)ccc5c4)nc(-c4ccc(-c5ccccc5)c5ccccc45)n3)cc2)cc1 ; c1ccc(-c2ccc(-c3nc(-c4ccc5cc(-c6ccc7ccc8cccnc8c7n6)ccc5c4)nc(-c4ccc(-c5ccccc5)c5ccccc45)n3)cc2)cc1 ; c1ccc(-c2ccc(-c3nc(-c4ccc5ccccc5c4-c4ccc(-c5nc(-c6ccccc6)nc(-c6ccccc6)n5)cc4)nc(-c4ccc(-c5ccccc5)c5ccccc45)n3)cc2)cc1]

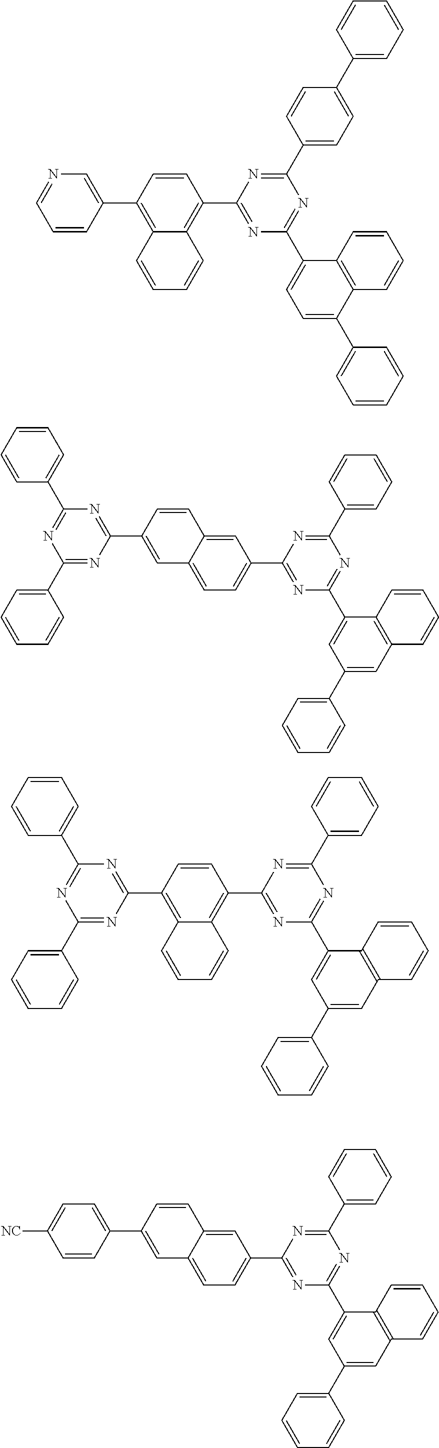

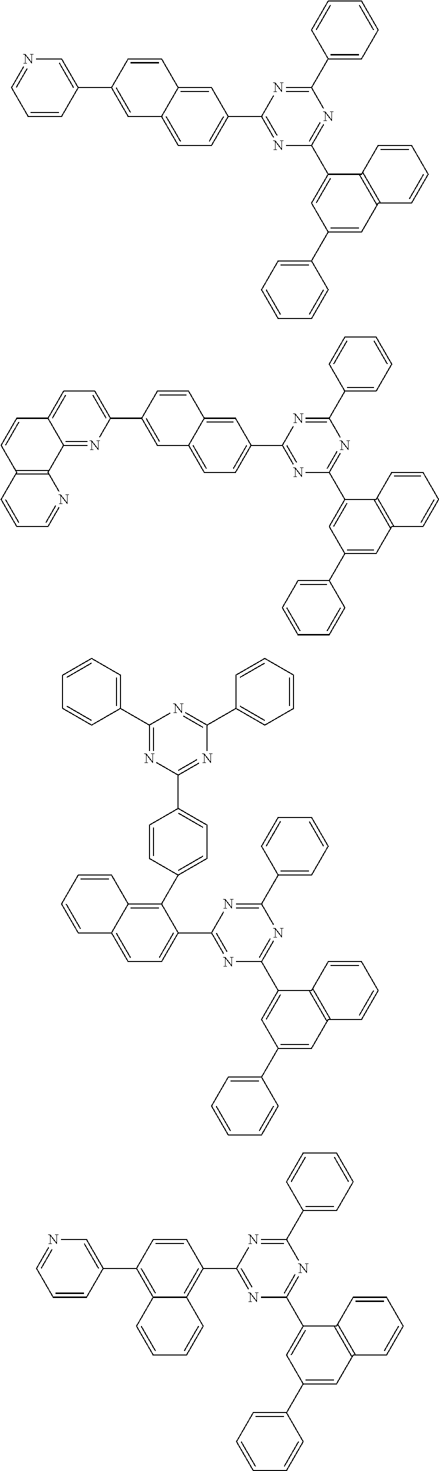

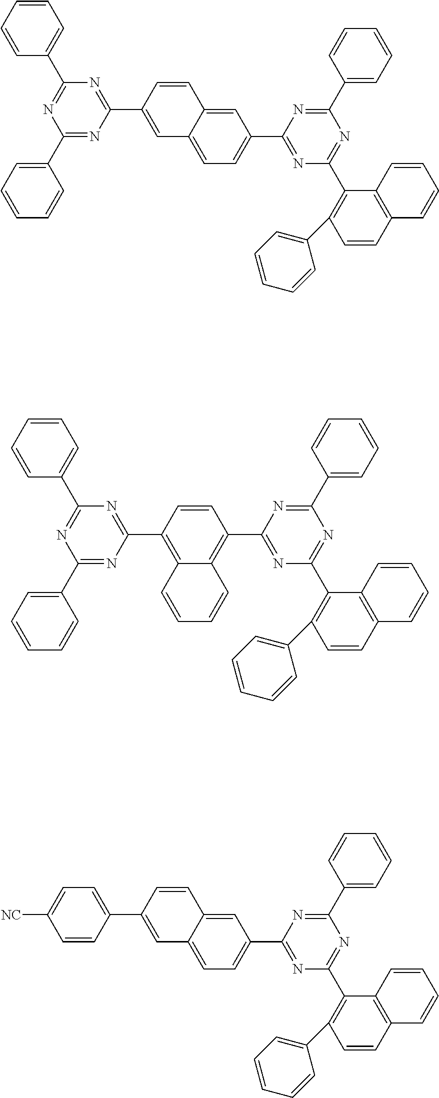 [SMILES: c1ccc(-c2nc(-c3ccccc3)nc(-c3ccc4cc(-c5nc(-c6ccccc6)nc(-c6c(-c7ccccc7)ccc7ccccc67)n5)ccc4c3)n2)cc1 ; c1ccc(-c2nc(-c3ccccc3)nc(-c3ccc(-c4nc(-c5ccccc5)nc(-c5c(-c6ccccc6)ccc6ccccc56)n4)c4ccccc34)n2)cc1 ; N#Cc1ccc(-c2ccc3cc(-c4nc(-c5ccccc5)nc(-c5c(-c6ccccc6)ccc6ccccc56)n4)ccc3c2)cc1]

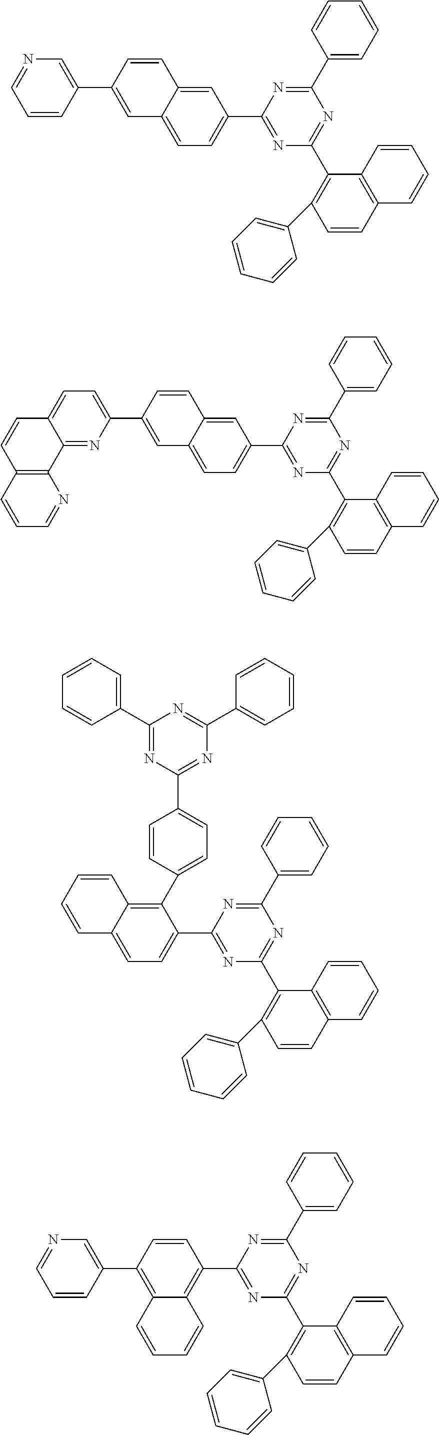 [SMILES: c1ccc(-c2nc(-c3ccc4cc(-c5cccnc5)ccc4c3)nc(-c3c(-c4ccccc4)ccc4ccccc34)n2)cc1 ; c1ccc(-c2nc(-c3ccc4cc(-c5ccc6ccc7cccnc7c6n5)ccc4c3)nc(-c3c(-c4ccccc4)ccc4ccccc34)n2)cc1 ; c1ccc(-c2nc(-c3ccccc3)nc(-c3ccc(-c4c(-c5nc(-c6ccccc6)nc(-c6c(-c7ccccc7)ccc7ccccc67)n5)ccc5ccccc45)cc3)n2)cc1 ; c1ccc(-c2nc(-c3c(-c4ccccc4)ccc4ccccc34)nc(-c3ccc(-c4cccnc4)c4ccccc34)n2)cc1]

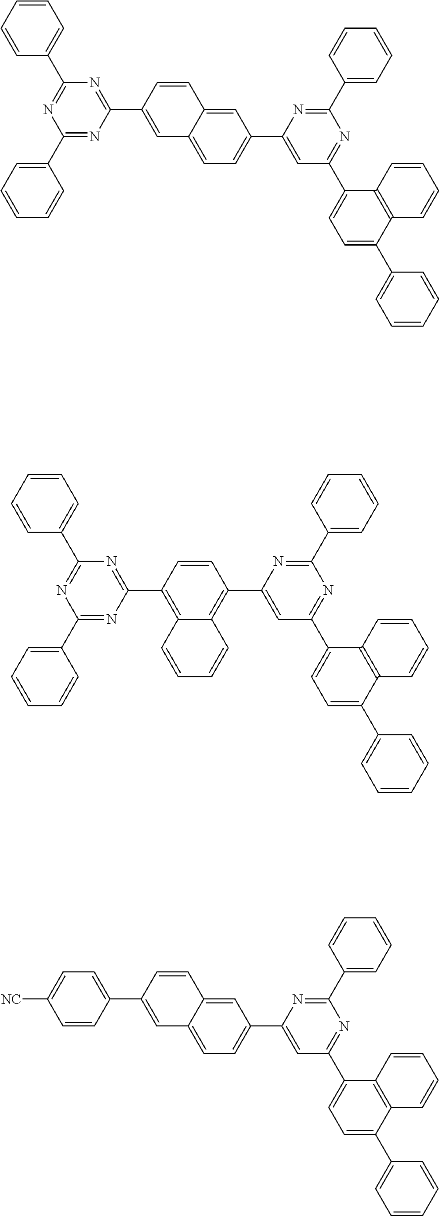 [SMILES: c1ccc(-c2nc(-c3ccc4cc(-c5nc(-c6ccccc6)nc(-c6ccccc6)n5)ccc4c3)cc(-c3ccc(-c4ccccc4)c4ccccc34)n2)cc1 ; c1ccc(-c2nc(-c3ccc(-c4ccccc4)c4ccccc34)cc(-c3ccc(-c4nc(-c5ccccc5)nc(-c5ccccc5)n4)c4ccccc34)n2)cc1 ; N#Cc1ccc(-c2ccc3cc(-c4cc(-c5ccc(-c6ccccc6)c6ccccc56)nc(-c5ccccc5)n4)ccc3c2)cc1]

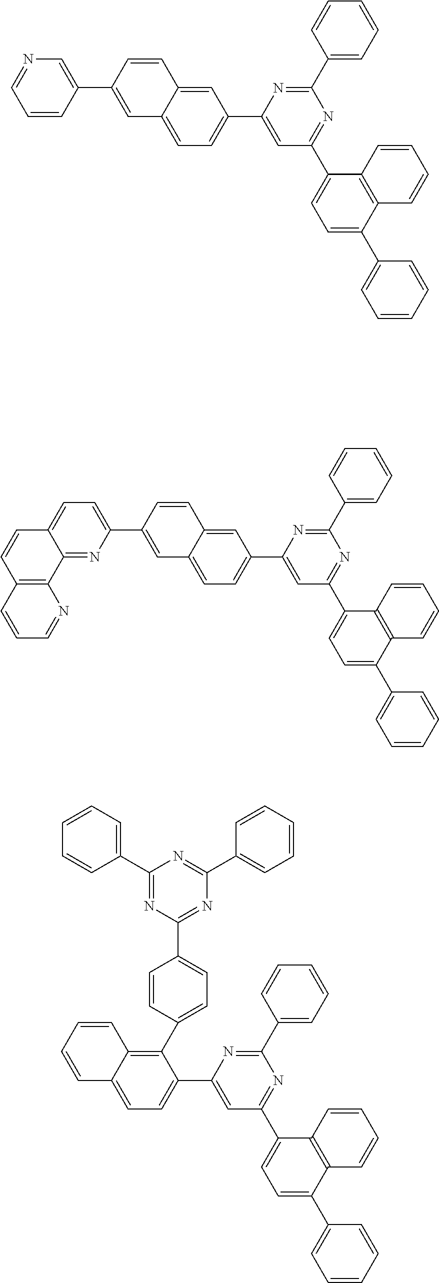 [SMILES: c1ccc(-c2nc(-c3ccc4cc(-c5cccnc5)ccc4c3)cc(-c3ccc(-c4ccccc4)c4ccccc34)n2)cc1 ; c1ccc(-c2nc(-c3ccc4cc(-c5ccc6ccc7cccnc7c6n5)ccc4c3)cc(-c3ccc(-c4ccccc4)c4ccccc34)n2)cc1 ; c1ccc(-c2nc(-c3ccc4ccccc4c3-c3ccc(-c4nc(-c5ccccc5)nc(-c5ccccc5)n4)cc3)cc(-c3ccc(-c4ccccc4)c4ccccc34)n2)cc1]

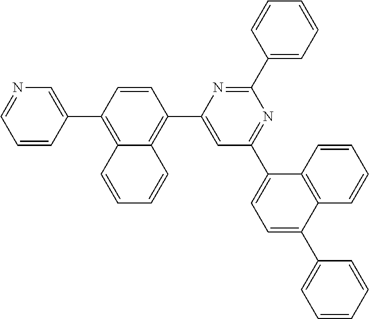 [SMILES: c1ccc(-c2nc(-c3ccc(-c4ccccc4)c4ccccc34)cc(-c3ccc(-c4cccnc4)c4ccccc34)n2)cc1]

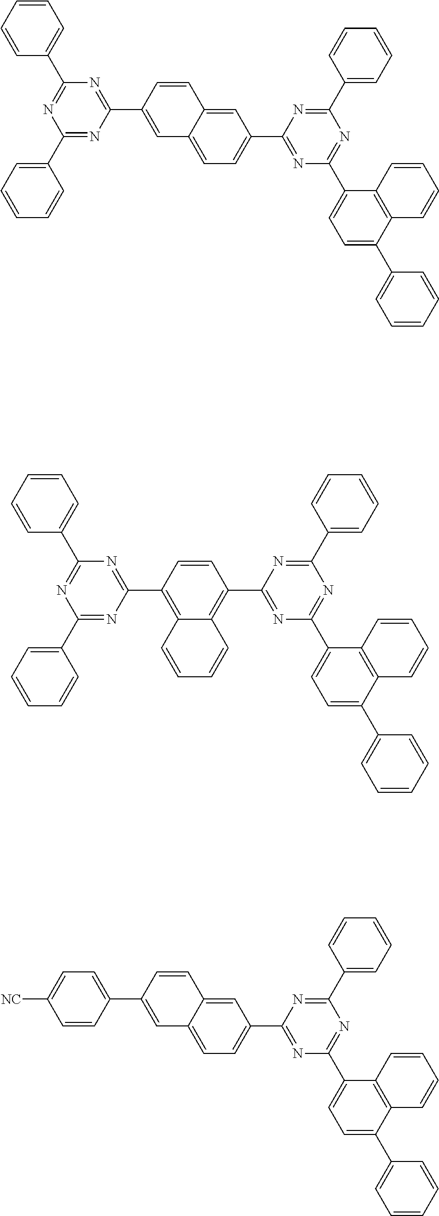 [SMILES: c1ccc(-c2nc(-c3ccccc3)nc(-c3ccc4cc(-c5nc(-c6ccccc6)nc(-c6ccc(-c7ccccc7)c7ccccc67)n5)ccc4c3)n2)cc1 ; c1ccc(-c2nc(-c3ccccc3)nc(-c3ccc(-c4nc(-c5ccccc5)nc(-c5ccc(-c6ccccc6)c6ccccc56)n4)c4ccccc34)n2)cc1 ; N#Cc1ccc(-c2ccc3cc(-c4nc(-c5ccccc5)nc(-c5ccc(-c6ccccc6)c6ccccc56)n4)ccc3c2)cc1]

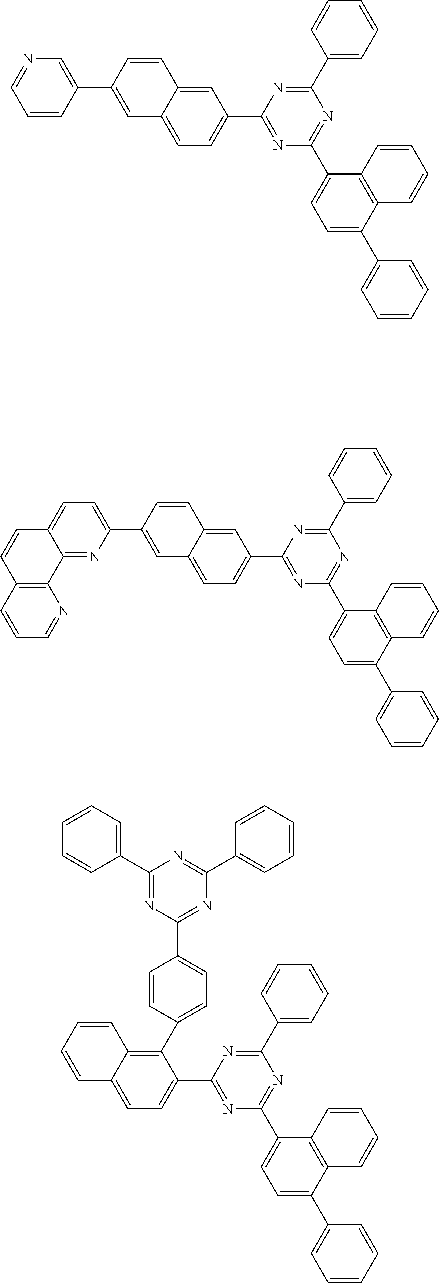 [SMILES: c1ccc(-c2nc(-c3ccc4cc(-c5cccnc5)ccc4c3)nc(-c3ccc(-c4ccccc4)c4ccccc34)n2)cc1 ; c1ccc(-c2nc(-c3ccc4cc(-c5ccc6ccc7cccnc7c6n5)ccc4c3)nc(-c3ccc(-c4ccccc4)c4ccccc34)n2)cc1 ; c1ccc(-c2nc(-c3ccccc3)nc(-c3ccc(-c4c(-c5nc(-c6ccccc6)nc(-c6ccc(-c7ccccc7)c7ccccc67)n5)ccc5ccccc45)cc3)n2)cc1]

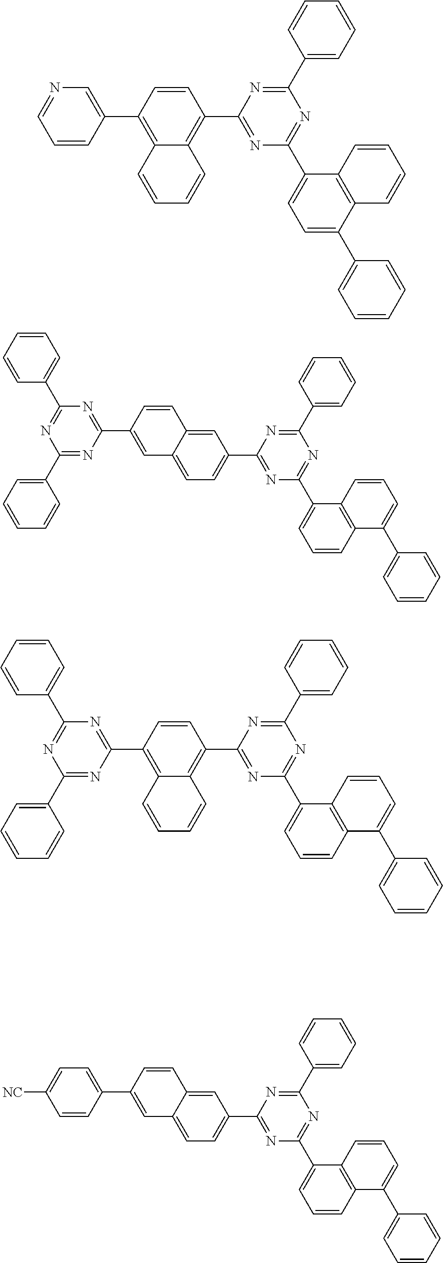 [SMILES: c1ccc(-c2nc(-c3ccc(-c4ccccc4)c4ccccc34)nc(-c3ccc(-c4cccnc4)c4ccccc34)n2)cc1 ; c1ccc(-c2nc(-c3ccccc3)nc(-c3ccc4cc(-c5nc(-c6ccccc6)nc(-c6cccc7c(-c8ccccc8)cccc67)n5)ccc4c3)n2)cc1 ; c1ccc(-c2nc(-c3ccccc3)nc(-c3ccc(-c4nc(-c5ccccc5)nc(-c5cccc6c(-c7ccccc7)cccc56)n4)c4ccccc34)n2)cc1 ; N#Cc1ccc(-c2ccc3cc(-c4nc(-c5ccccc5)nc(-c5cccc6c(-c7ccccc7)cccc56)n4)ccc3c2)cc1]

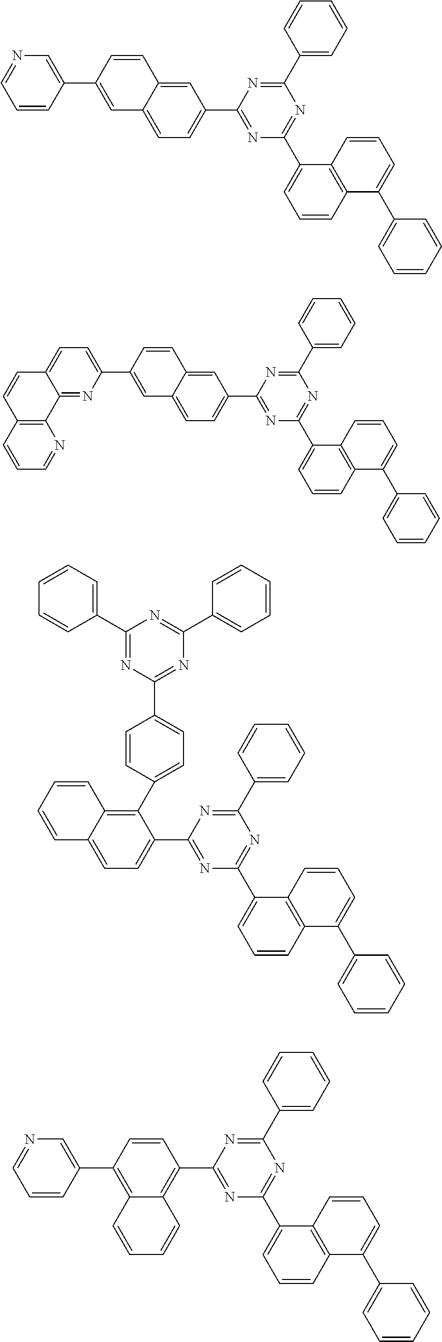 [SMILES: c1ccc(-c2nc(-c3ccc4cc(-c5cccnc5)ccc4c3)nc(-c3cccc4c(-c5ccccc5)cccc34)n2)cc1 ; c1ccc(-c2nc(-c3ccc4cc(-c5ccc6ccc7cccnc7c6n5)ccc4c3)nc(-c3cccc4c(-c5ccccc5)cccc34)n2)cc1 ; c1ccc(-c2nc(-c3ccccc3)nc(-c3ccc(-c4c(-c5nc(-c6ccccc6)nc(-c6cccc7c(-c8ccccc8)cccc67)n5)ccc5ccccc45)cc3)n2)cc1 ; c1ccc(-c2nc(-c3cccc4c(-c5ccccc5)cccc34)nc(-c3ccc(-c4cccnc4)c4ccccc34)n2)cc1]

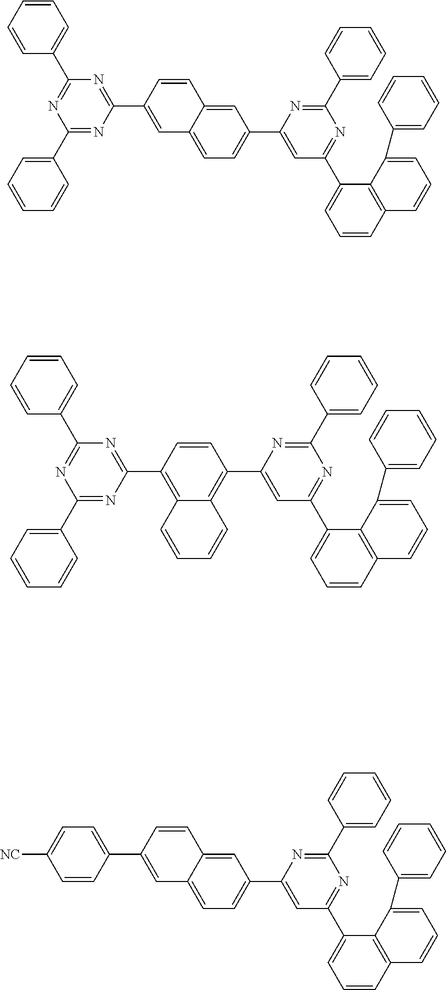 [SMILES: c1ccc(-c2nc(-c3ccc4cc(-c5nc(-c6ccccc6)nc(-c6ccccc6)n5)ccc4c3)cc(-c3cccc4cccc(-c5ccccc5)c34)n2)cc1 ; c1ccc(-c2nc(-c3ccc(-c4nc(-c5ccccc5)nc(-c5ccccc5)n4)c4ccccc34)cc(-c3cccc4cccc(-c5ccccc5)c34)n2)cc1 ; N#Cc1ccc(-c2ccc3cc(-c4cc(-c5cccc6cccc(-c7ccccc7)c56)nc(-c5ccccc5)n4)ccc3c2)cc1]

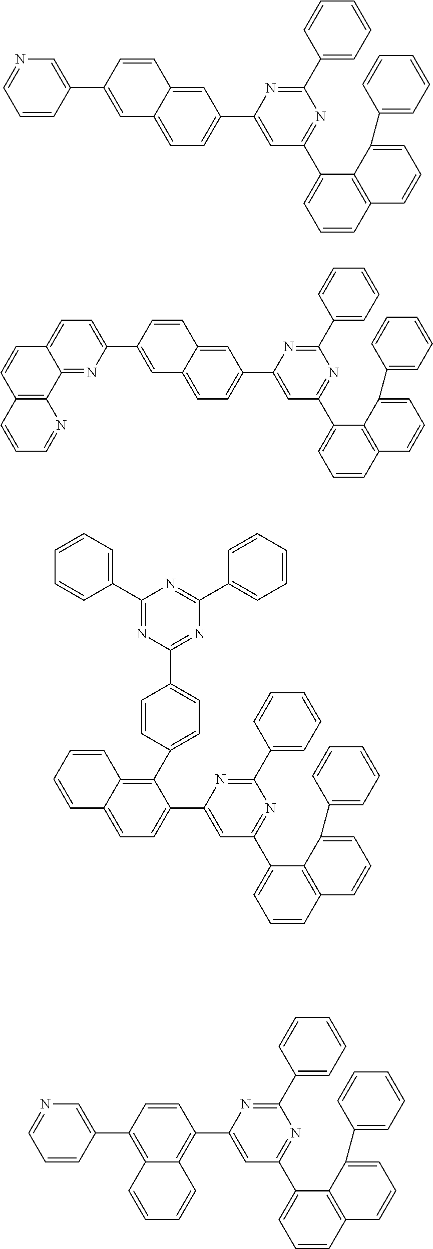

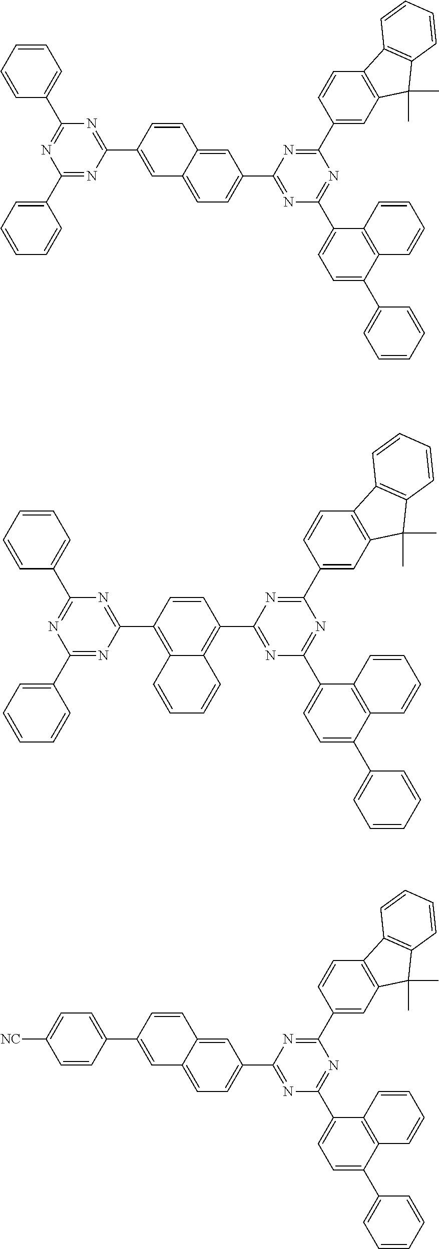 [SMILES: CC1(C)c2ccccc2-c2ccc(-c3nc(-c4ccc5cc(-c6nc(-c7ccccc7)nc(-c7ccccc7)n6)ccc5c4)nc(-c4ccc(-c5ccccc5)c5ccccc45)n3)cc21 ; CC1(C)c2ccccc2-c2ccc(-c3nc(-c4ccc(-c5ccccc5)c5ccccc45)nc(-c4ccc(-c5nc(-c6ccccc6)nc(-c6ccccc6)n5)c5ccccc45)n3)cc21 ; CC1(C)c2ccccc2-c2ccc(-c3nc(-c4ccc5cc(-c6ccc(C#N)cc6)ccc5c4)nc(-c4ccc(-c5ccccc5)c5ccccc45)n3)cc21]

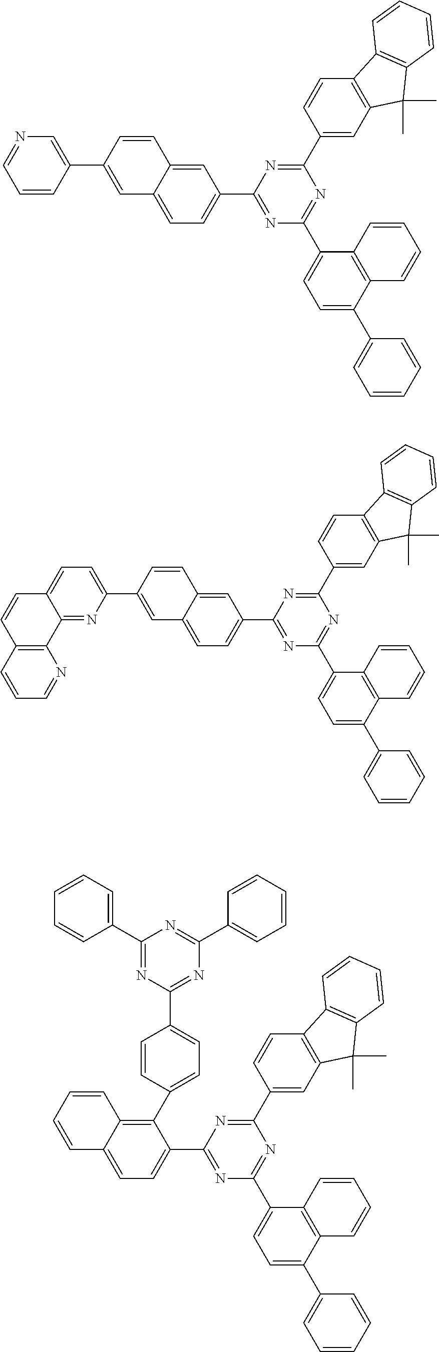 [SMILES: CC1(C)c2ccccc2-c2ccc(-c3nc(-c4ccc5cc(-c6cccnc6)ccc5c4)nc(-c4ccc(-c5ccccc5)c5ccccc45)n3)cc21 ; CC1(C)c2ccccc2-c2ccc(-c3nc(-c4ccc5cc(-c6ccc7ccc8cccnc8c7n6)ccc5c4)nc(-c4ccc(-c5ccccc5)c5ccccc45)n3)cc21 ; CC1(C)c2ccccc2-c2ccc(-c3nc(-c4ccc5ccccc5c4-c4ccc(-c5nc(-c6ccccc6)nc(-c6ccccc6)n5)cc4)nc(-c4ccc(-c5ccccc5)c5ccccc45)n3)cc21]

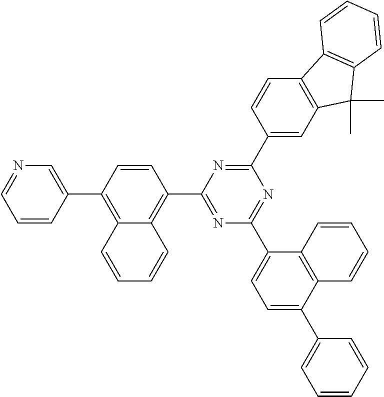 [SMILES: CC1(C)c2ccccc2-c2ccc(-c3nc(-c4ccc(-c5ccccc5)c5ccccc45)nc(-c4ccc(-c5cccnc5)c5ccccc45)n3)cc21]

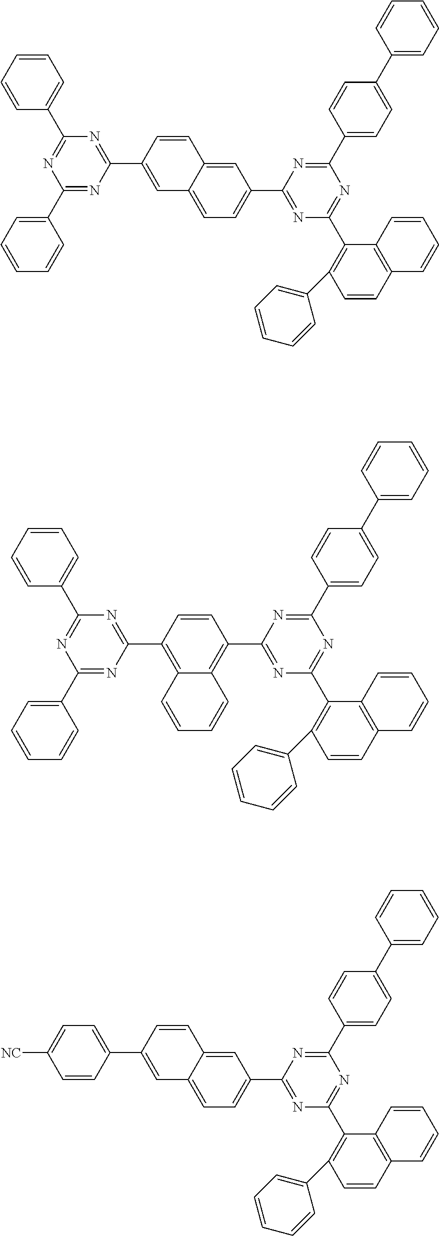 [SMILES: c1ccc(-c2ccc(-c3nc(-c4ccc5cc(-c6nc(-c7ccccc7)nc(-c7ccccc7)n6)ccc5c4)nc(-c4c(-c5ccccc5)ccc5ccccc45)n3)cc2)cc1 ; c1ccc(-c2ccc(-c3nc(-c4c(-c5ccccc5)ccc5ccccc45)nc(-c4ccc(-c5nc(-c6ccccc6)nc(-c6ccccc6)n5)c5ccccc45)n3)cc2)cc1 ; N#Cc1ccc(-c2ccc3cc(-c4nc(-c5ccc(-c6ccccc6)cc5)nc(-c5c(-c6ccccc6)ccc6ccccc56)n4)ccc3c2)cc1]

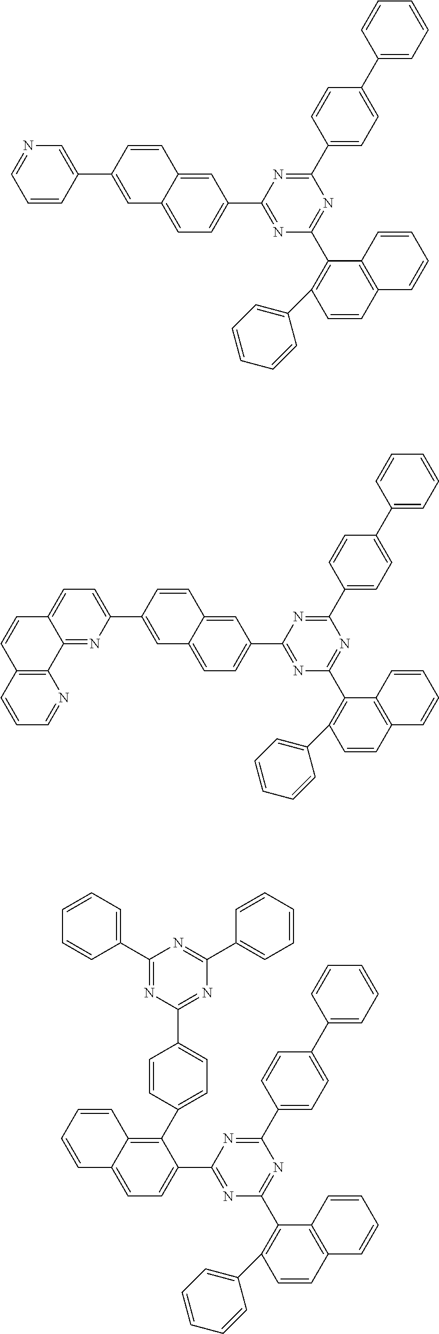 [SMILES: c1ccc(-c2ccc(-c3nc(-c4ccc5cc(-c6cccnc6)ccc5c4)nc(-c4c(-c5ccccc5)ccc5ccccc45)n3)cc2)cc1 ; c1ccc(-c2ccc(-c3nc(-c4ccc5cc(-c6ccc7ccc8cccnc8c7n6)ccc5c4)nc(-c4c(-c5ccccc5)ccc5ccccc45)n3)cc2)cc1 ; c1ccc(-c2ccc(-c3nc(-c4ccc5ccccc5c4-c4ccc(-c5nc(-c6ccccc6)nc(-c6ccccc6)n5)cc4)nc(-c4c(-c5ccccc5)ccc5ccccc45)n3)cc2)cc1]

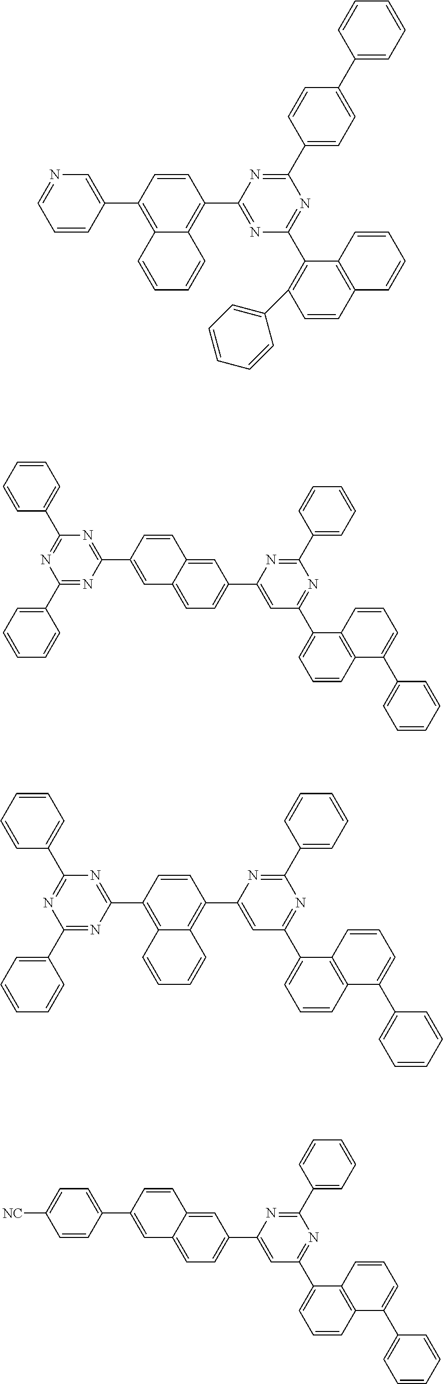 [SMILES: c1ccc(-c2ccc(-c3nc(-c4c(-c5ccccc5)ccc5ccccc45)nc(-c4ccc(-c5cccnc5)c5ccccc45)n3)cc2)cc1 ; c1ccc(-c2nc(-c3ccc4cc(-c5nc(-c6ccccc6)nc(-c6ccccc6)n5)ccc4c3)cc(-c3cccc4c(-c5ccccc5)cccc34)n2)cc1 ; c1ccc(-c2nc(-c3cccc4c(-c5ccccc5)cccc34)cc(-c3ccc(-c4nc(-c5ccccc5)nc(-c5ccccc5)n4)c4ccccc34)n2)cc1 ; N#Cc1ccc(-c2ccc3cc(-c4cc(-c5cccc6c(-c7ccccc7)cccc56)nc(-c5ccccc5)n4)ccc3c2)cc1]

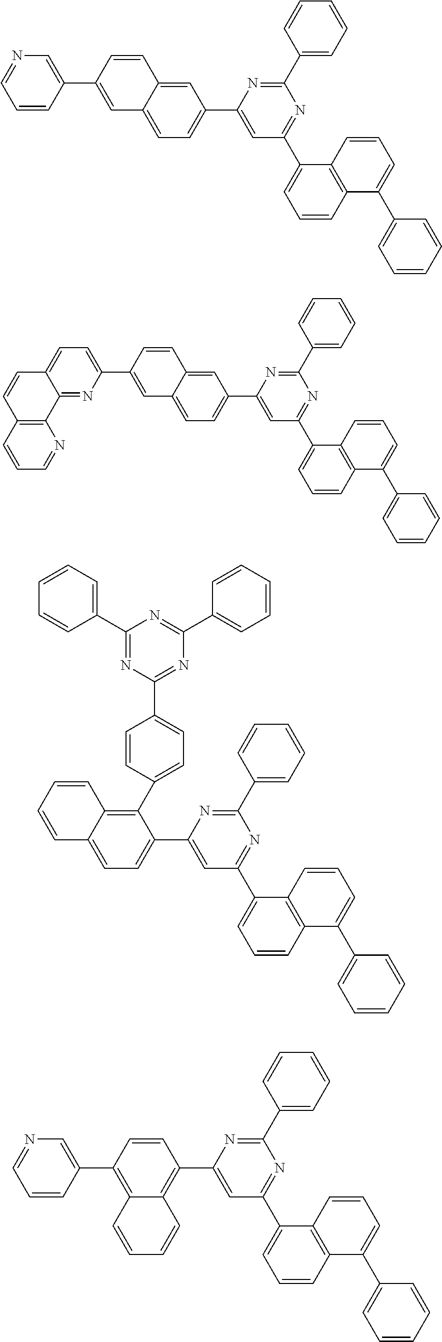 [SMILES: c1ccc(-c2nc(-c3ccc4cc(-c5cccnc5)ccc4c3)cc(-c3cccc4c(-c5ccccc5)cccc34)n2)cc1 ; c1ccc(-c2nc(-c3ccc4cc(-c5ccc6ccc7cccnc7c6n5)ccc4c3)cc(-c3cccc4c(-c5ccccc5)cccc34)n2)cc1 ; c1ccc(-c2nc(-c3ccc4ccccc4c3-c3ccc(-c4nc(-c5ccccc5)nc(-c5ccccc5)n4)cc3)cc(-c3cccc4c(-c5ccccc5)cccc34)n2)cc1 ; c1ccc(-c2nc(-c3cccc4c(-c5ccccc5)cccc34)cc(-c3ccc(-c4cccnc4)c4ccccc34)n2)cc1]

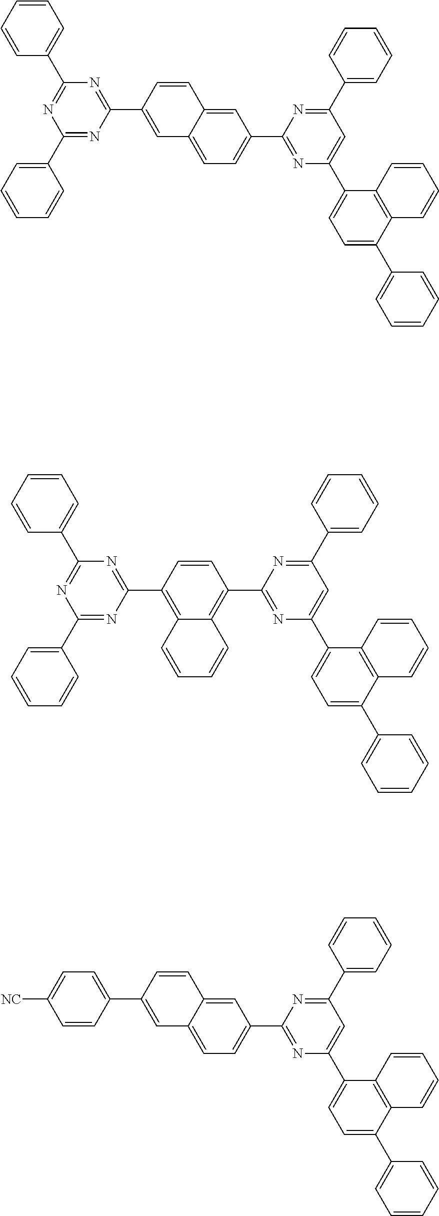 [SMILES: c1ccc(-c2cc(-c3ccc(-c4ccccc4)c4ccccc34)nc(-c3ccc4cc(-c5nc(-c6ccccc6)nc(-c6ccccc6)n5)ccc4c3)n2)cc1 ; c1ccc(-c2cc(-c3ccc(-c4ccccc4)c4ccccc34)nc(-c3ccc(-c4nc(-c5ccccc5)nc(-c5ccccc5)n4)c4ccccc34)n2)cc1 ; N#Cc1ccc(-c2ccc3cc(-c4nc(-c5ccccc5)cc(-c5ccc(-c6ccccc6)c6ccccc56)n4)ccc3c2)cc1]

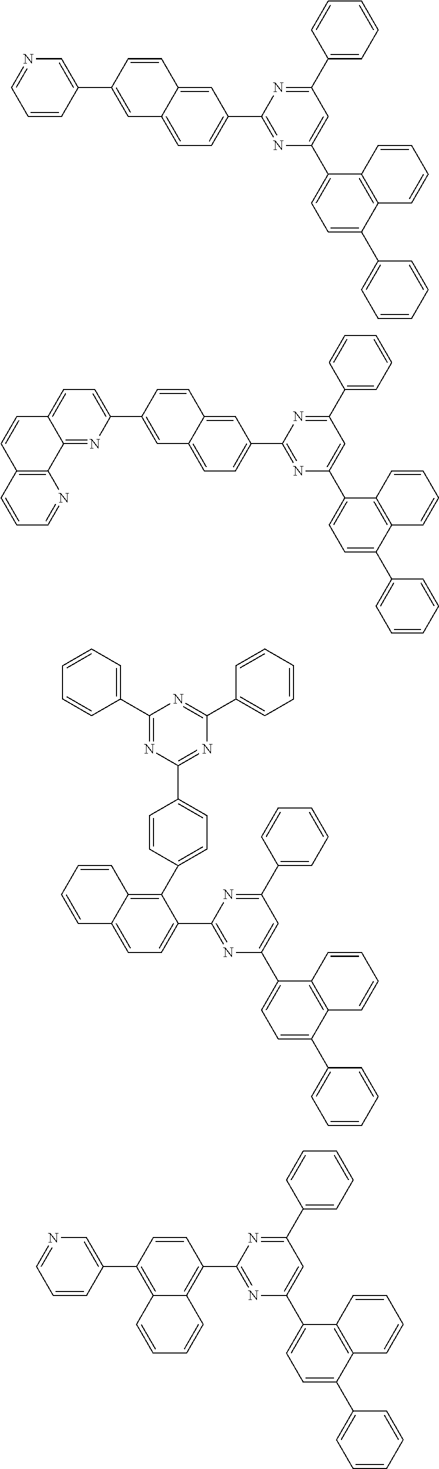

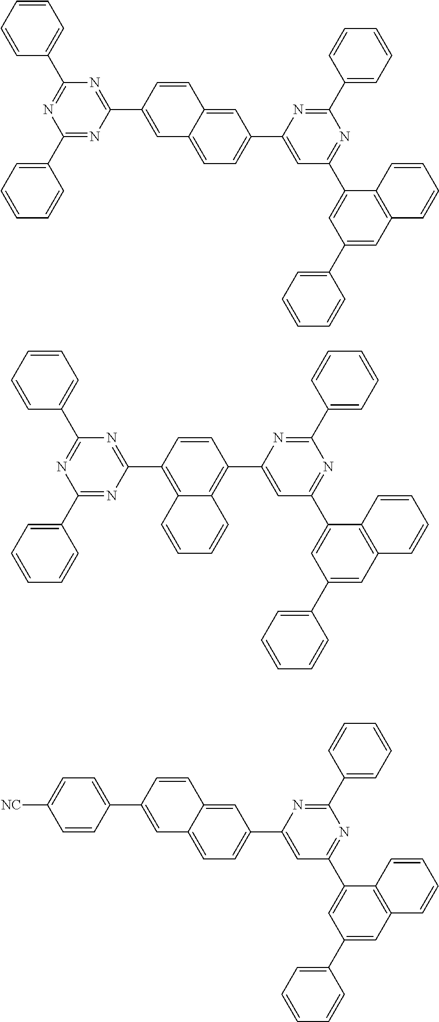

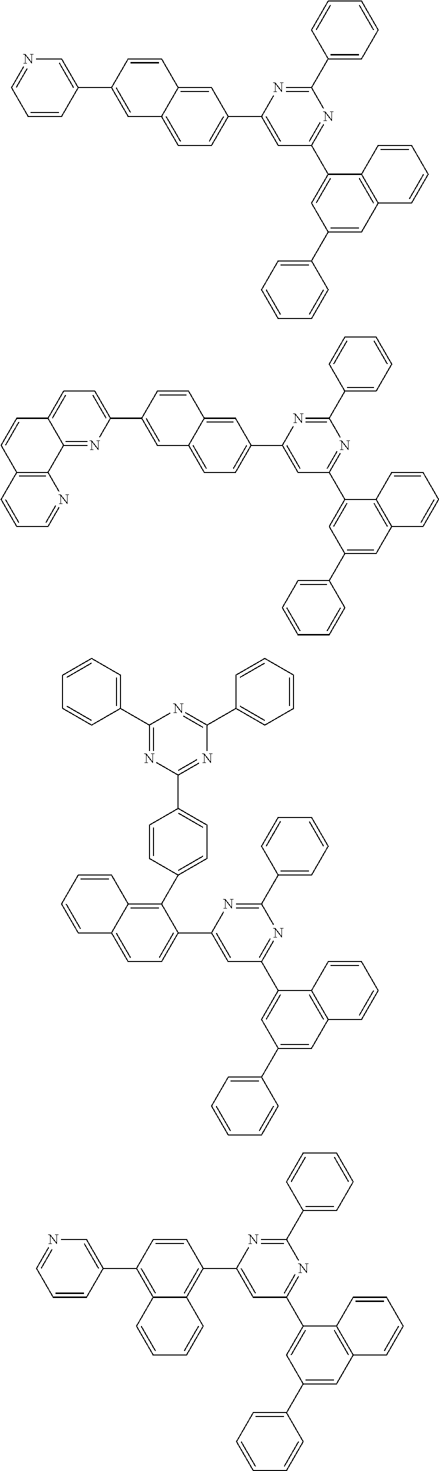

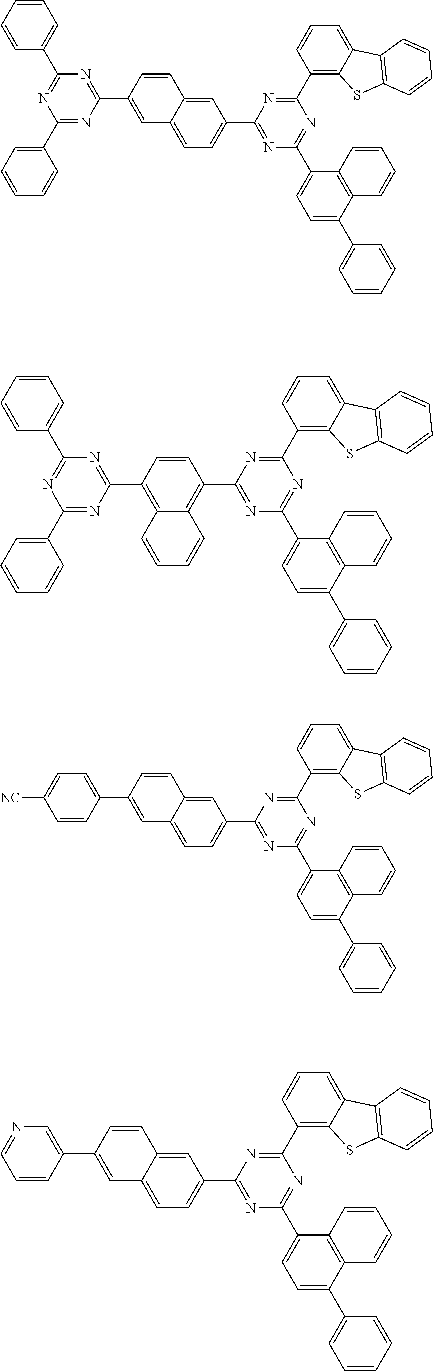 [SMILES: c1ccc(-c2nc(-c3ccccc3)nc(-c3ccc4cc(-c5nc(-c6ccc(-c7ccccc7)c7ccccc67)nc(-c6cccc7c6sc6ccccc67)n5)ccc4c3)n2)cc1 ; c1ccc(-c2nc(-c3ccccc3)nc(-c3ccc(-c4nc(-c5ccc(-c6ccccc6)c6ccccc56)nc(-c5cccc6c5sc5ccccc56)n4)c4ccccc34)n2)cc1 ; N#Cc1ccc(-c2ccc3cc(-c4nc(-c5ccc(-c6ccccc6)c6ccccc56)nc(-c5cccc6c5sc5ccccc56)n4)ccc3c2)cc1 ; c1ccc(-c2ccc(-c3nc(-c4ccc5cc(-c6cccnc6)ccc5c4)nc(-c4cccc5c4sc4ccccc45)n3)c3ccccc23)cc1]

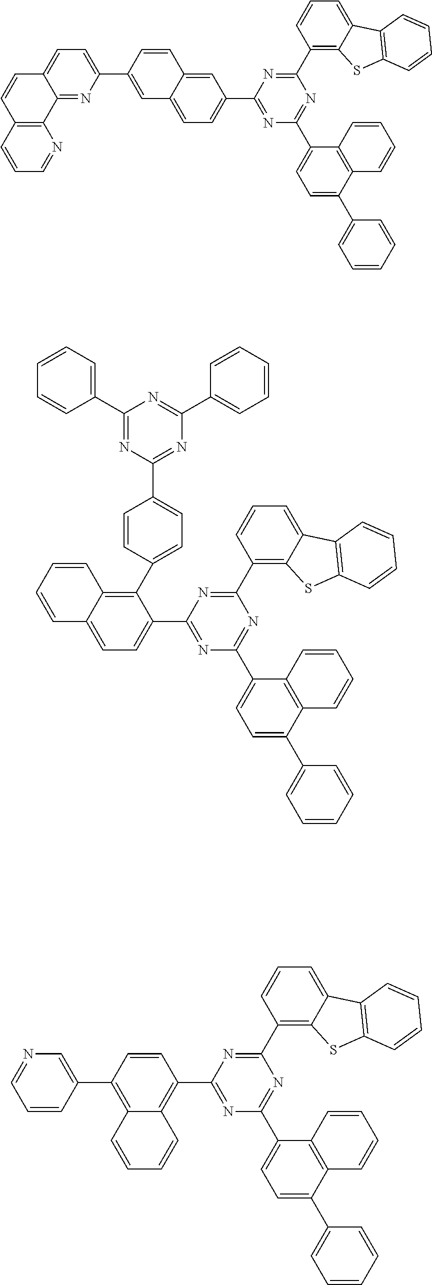 [SMILES: c1ccc(-c2ccc(-c3nc(-c4ccc5cc(-c6ccc7ccc8cccnc8c7n6)ccc5c4)nc(-c4cccc5c4sc4ccccc45)n3)c3ccccc23)cc1 ; c1ccc(-c2nc(-c3ccccc3)nc(-c3ccc(-c4c(-c5nc(-c6ccc(-c7ccccc7)c7ccccc67)nc(-c6cccc7c6sc6ccccc67)n5)ccc5ccccc45)cc3)n2)cc1 ; c1ccc(-c2ccc(-c3nc(-c4ccc(-c5cccnc5)c5ccccc45)nc(-c4cccc5c4sc4ccccc45)n3)c3ccccc23)cc1]

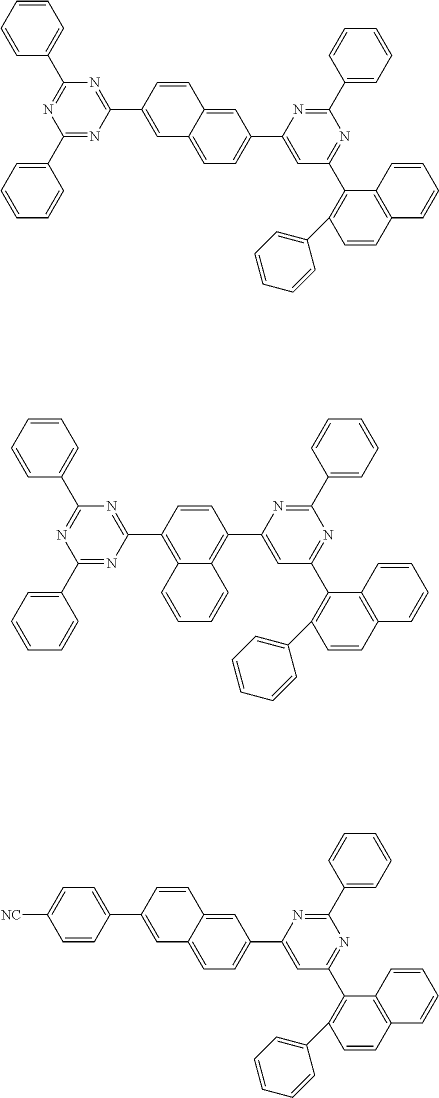 [SMILES: c1ccc(-c2nc(-c3ccc4cc(-c5nc(-c6ccccc6)nc(-c6ccccc6)n5)ccc4c3)cc(-c3c(-c4ccccc4)ccc4ccccc34)n2)cc1 ; c1ccc(-c2nc(-c3c(-c4ccccc4)ccc4ccccc34)cc(-c3ccc(-c4nc(-c5ccccc5)nc(-c5ccccc5)n4)c4ccccc34)n2)cc1 ; N#Cc1ccc(-c2ccc3cc(-c4cc(-c5c(-c6ccccc6)ccc6ccccc56)nc(-c5ccccc5)n4)ccc3c2)cc1]

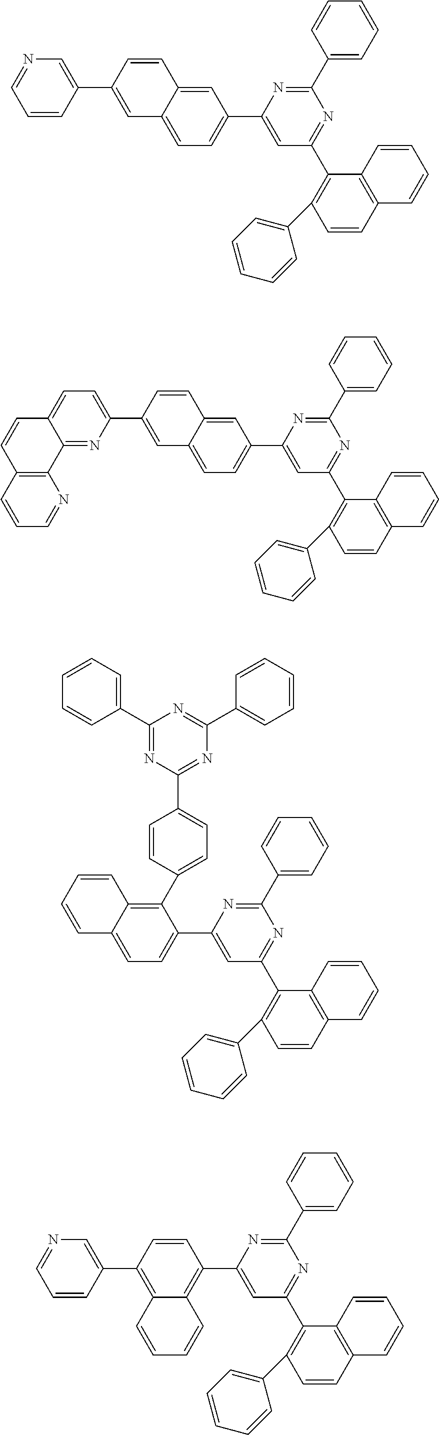 [SMILES: c1ccc(-c2nc(-c3ccc4cc(-c5cccnc5)ccc4c3)cc(-c3c(-c4ccccc4)ccc4ccccc34)n2)cc1 ; c1ccc(-c2nc(-c3ccc4cc(-c5ccc6ccc7cccnc7c6n5)ccc4c3)cc(-c3c(-c4ccccc4)ccc4ccccc34)n2)cc1 ; c1ccc(-c2nc(-c3ccc4ccccc4c3-c3ccc(-c4nc(-c5ccccc5)nc(-c5ccccc5)n4)cc3)cc(-c3c(-c4ccccc4)ccc4ccccc34)n2)cc1 ; c1ccc(-c2nc(-c3c(-c4ccccc4)ccc4ccccc34)cc(-c3ccc(-c4cccnc4)c4ccccc34)n2)cc1]

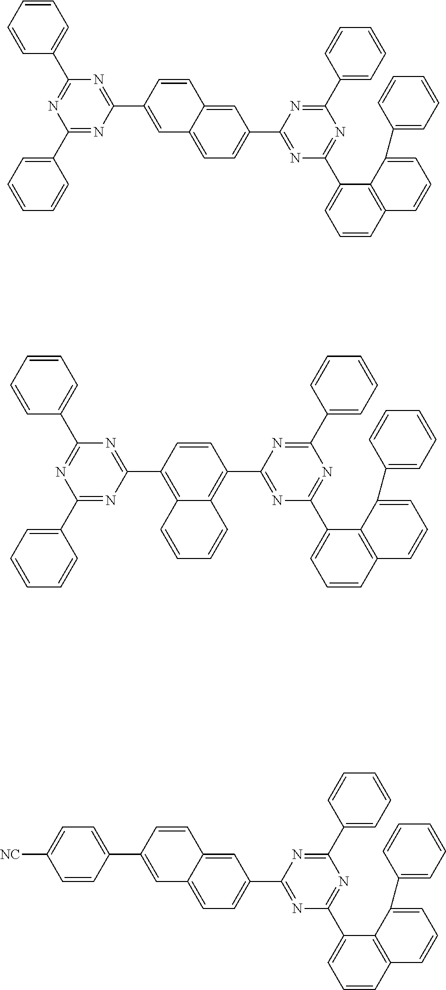 [SMILES: c1ccc(-c2nc(-c3ccccc3)nc(-c3ccc4cc(-c5nc(-c6ccccc6)nc(-c6cccc7cccc(-c8ccccc8)c67)n5)ccc4c3)n2)cc1 ; c1ccc(-c2nc(-c3ccccc3)nc(-c3ccc(-c4nc(-c5ccccc5)nc(-c5cccc6cccc(-c7ccccc7)c56)n4)c4ccccc34)n2)cc1 ; N#Cc1ccc(-c2ccc3cc(-c4nc(-c5ccccc5)nc(-c5cccc6cccc(-c7ccccc7)c56)n4)ccc3c2)cc1]

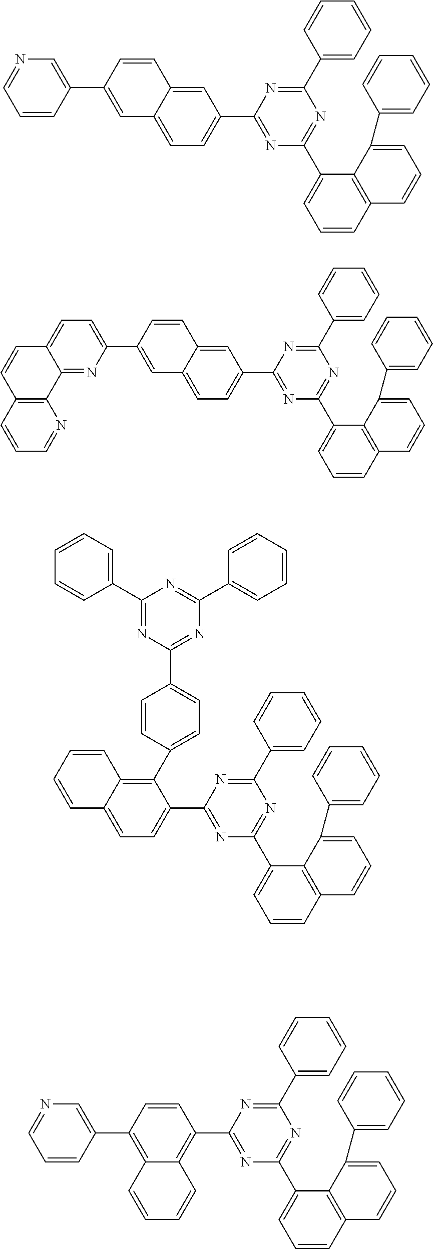

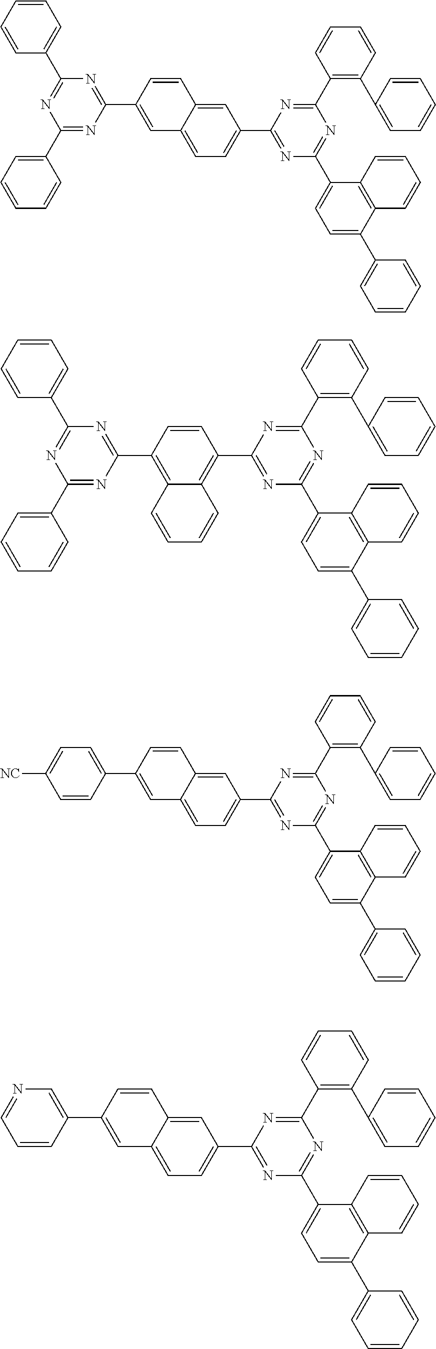

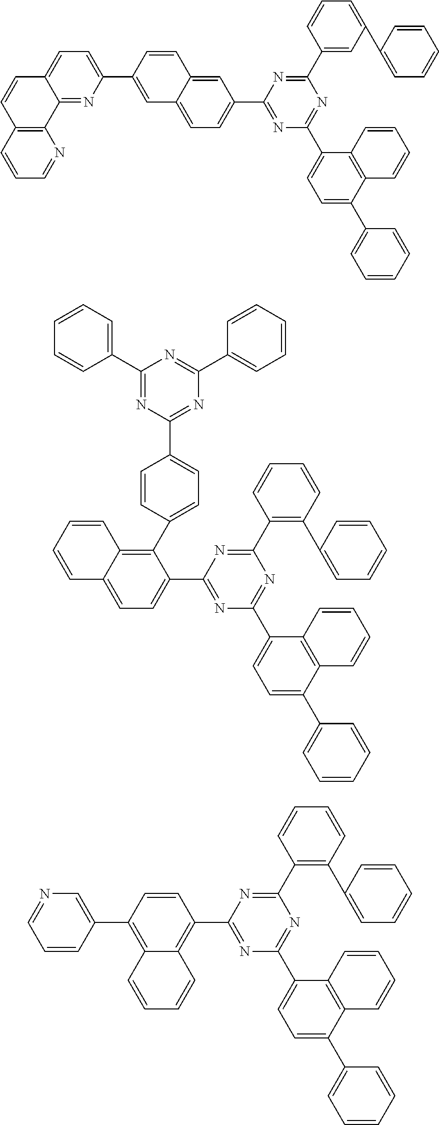

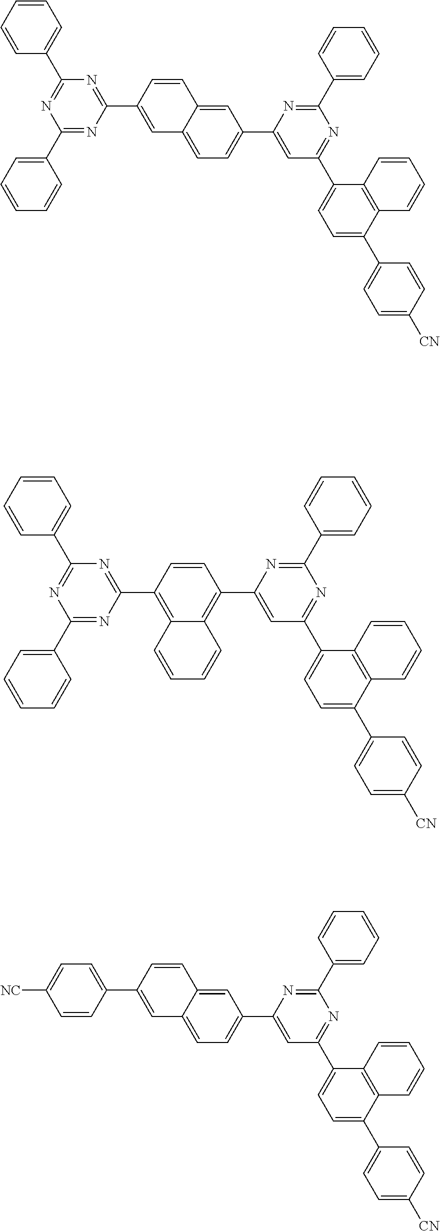 [SMILES: N#Cc1ccc(-c2ccc(-c3cc(-c4ccc5cc(-c6nc(-c7ccccc7)nc(-c7ccccc7)n6)ccc5c4)nc(-c4ccccc4)n3)c3ccccc23)cc1 ; N#Cc1ccc(-c2ccc(-c3cc(-c4ccc(-c5nc(-c6ccccc6)nc(-c6ccccc6)n5)c5ccccc45)nc(-c4ccccc4)n3)c3ccccc23)cc1 ; N#Cc1ccc(-c2ccc3cc(-c4cc(-c5ccc(-c6ccc(C#N)cc6)c6ccccc56)nc(-c5ccccc5)n4)ccc3c2)cc1]

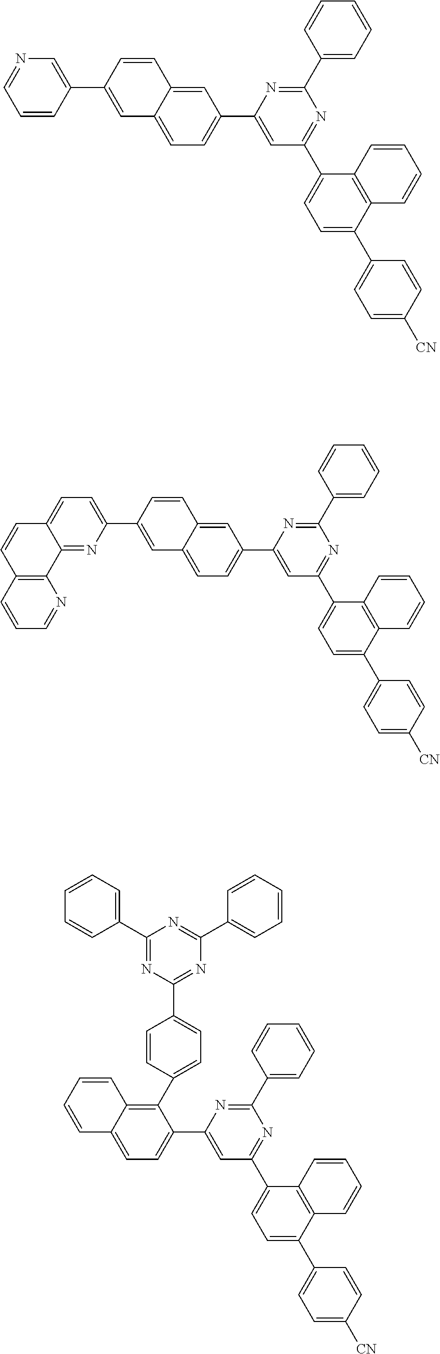 [SMILES: N#Cc1ccc(-c2ccc(-c3cc(-c4ccc5cc(-c6cccnc6)ccc5c4)nc(-c4ccccc4)n3)c3ccccc23)cc1 ; N#Cc1ccc(-c2ccc(-c3cc(-c4ccc5cc(-c6ccc7ccc8cccnc8c7n6)ccc5c4)nc(-c4ccccc4)n3)c3ccccc23)cc1 ; N#Cc1ccc(-c2ccc(-c3cc(-c4ccc5ccccc5c4-c4ccc(-c5nc(-c6ccccc6)nc(-c6ccccc6)n5)cc4)nc(-c4ccccc4)n3)c3ccccc23)cc1]

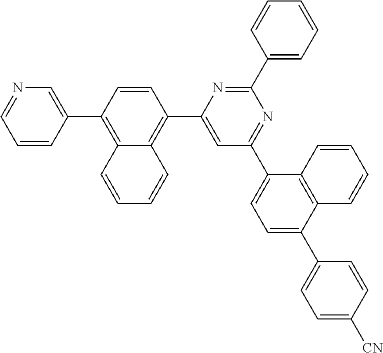 [SMILES: N#Cc1ccc(-c2ccc(-c3cc(-c4ccc(-c5cccnc5)c5ccccc45)nc(-c4ccccc4)n3)c3ccccc23)cc1]

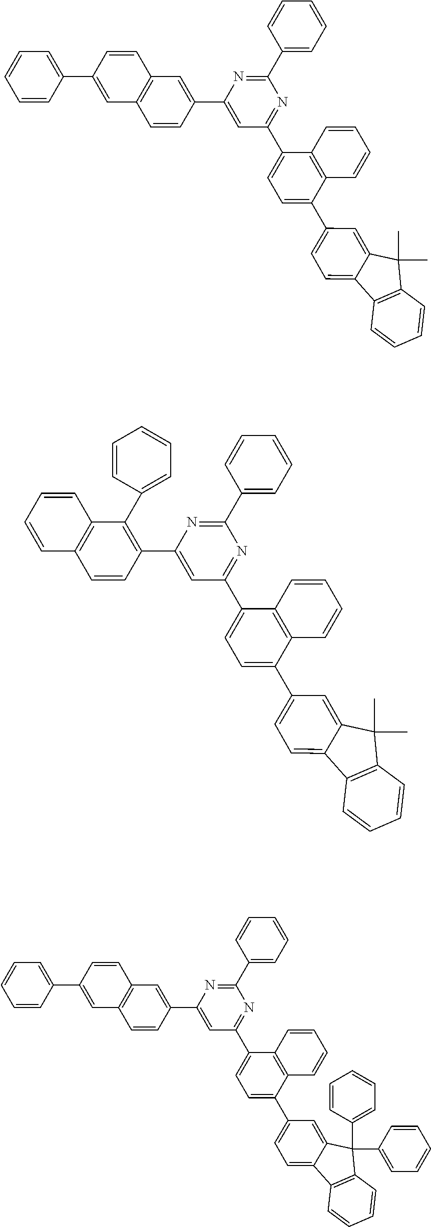 [SMILES: CC1(C)c2ccccc2-c2ccc(-c3ccc(-c4cc(-c5ccc6cc(-c7ccccc7)ccc6c5)nc(-c5ccccc5)n4)c4ccccc34)cc21 ; CC1(C)c2ccccc2-c2ccc(-c3ccc(-c4cc(-c5ccc6ccccc6c5-c5ccccc5)nc(-c5ccccc5)n4)c4ccccc34)cc21 ; c1ccc(-c2ccc3cc(-c4cc(-c5ccc(-c6ccc7c(c6)C(c6ccccc6)(c6ccccc6)c6ccccc6-7)c6ccccc56)nc(-c5ccccc5)n4)ccc3c2)cc1]

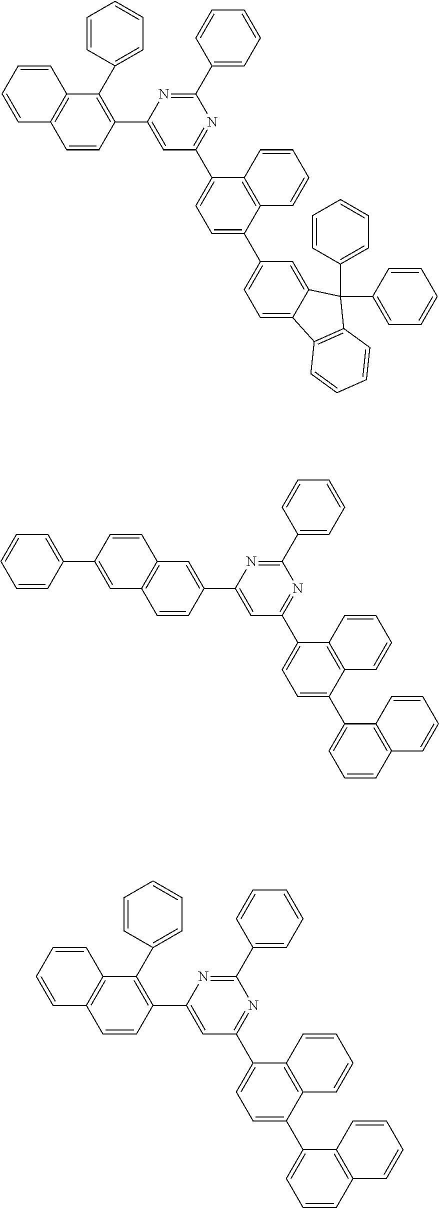 [SMILES: c1ccc(-c2nc(-c3ccc4ccccc4c3-c3ccccc3)cc(-c3ccc(-c4ccc5c(c4)C(c4ccccc4)(c4ccccc4)c4ccccc4-5)c4ccccc34)n2)cc1 ; c1ccc(-c2ccc3cc(-c4cc(-c5ccc(-c6cccc7ccccc67)c6ccccc56)nc(-c5ccccc5)n4)ccc3c2)cc1 ; c1ccc(-c2nc(-c3ccc4ccccc4c3-c3ccccc3)cc(-c3ccc(-c4cccc5ccccc45)c4ccccc34)n2)cc1]

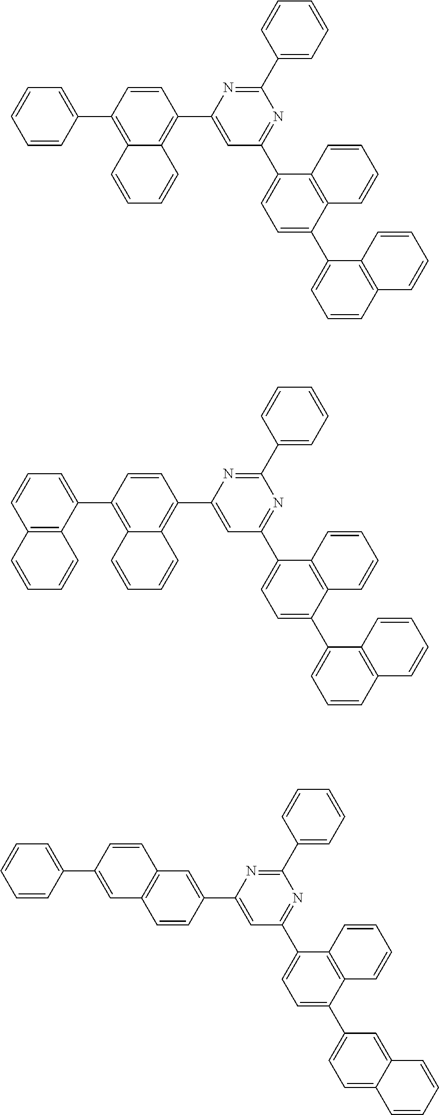 [SMILES: c1ccc(-c2nc(-c3ccc(-c4ccccc4)c4ccccc34)cc(-c3ccc(-c4cccc5ccccc45)c4ccccc34)n2)cc1 ; c1ccc(-c2nc(-c3ccc(-c4cccc5ccccc45)c4ccccc34)cc(-c3ccc(-c4cccc5ccccc45)c4ccccc34)n2)cc1 ; c1ccc(-c2ccc3cc(-c4cc(-c5ccc(-c6ccc7ccccc7c6)c6ccccc56)nc(-c5ccccc5)n4)ccc3c2)cc1]

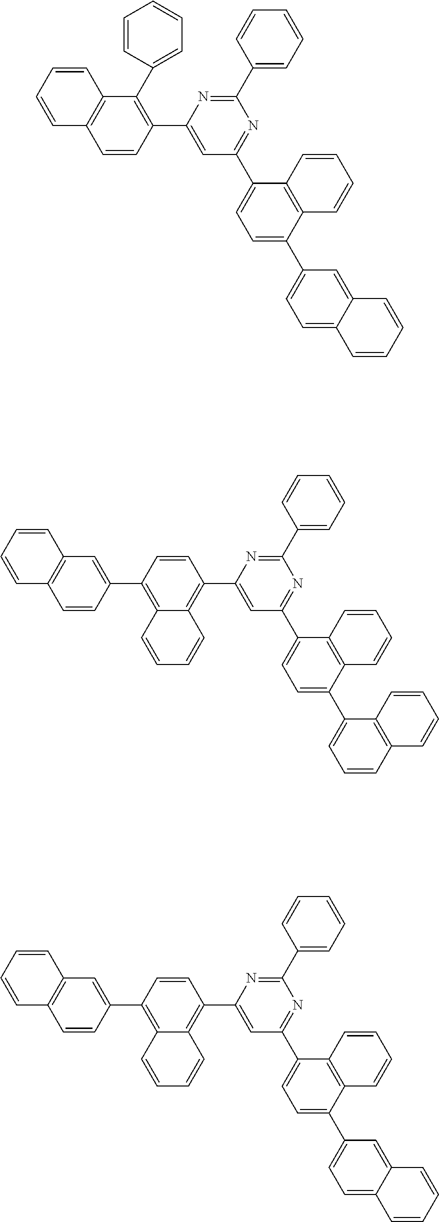 [SMILES: c1ccc(-c2nc(-c3ccc4ccccc4c3-c3ccccc3)cc(-c3ccc(-c4ccc5ccccc5c4)c4ccccc34)n2)cc1 ; c1ccc(-c2nc(-c3ccc(-c4ccc5ccccc5c4)c4ccccc34)cc(-c3ccc(-c4cccc5ccccc45)c4ccccc34)n2)cc1 ; c1ccc(-c2nc(-c3ccc(-c4ccc5ccccc5c4)c4ccccc34)cc(-c3ccc(-c4ccc5ccccc5c4)c4ccccc34)n2)cc1]

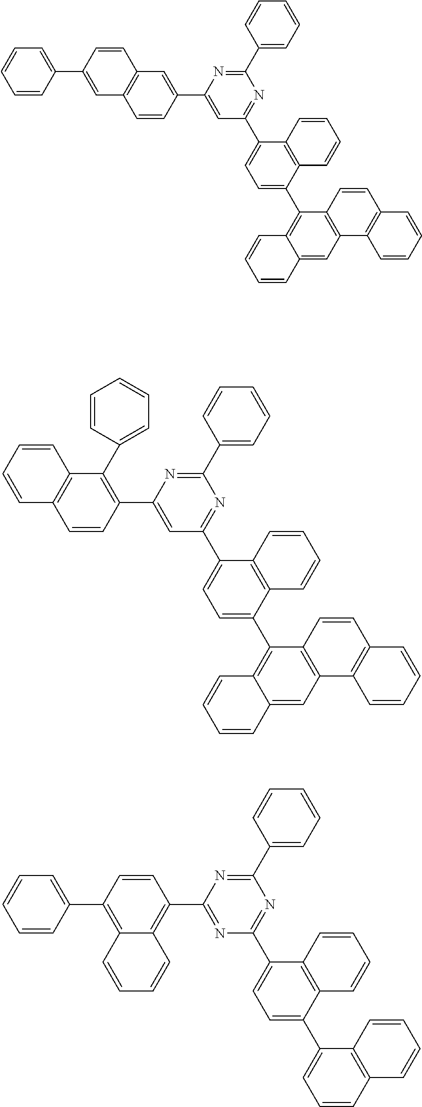 [SMILES: c1ccc(-c2ccc3cc(-c4cc(-c5ccc(-c6c7ccccc7cc7c6ccc6ccccc67)c6ccccc56)nc(-c5ccccc5)n4)ccc3c2)cc1 ; c1ccc(-c2nc(-c3ccc4ccccc4c3-c3ccccc3)cc(-c3ccc(-c4c5ccccc5cc5c4ccc4ccccc45)c4ccccc34)n2)cc1 ; c1ccc(-c2nc(-c3ccc(-c4ccccc4)c4ccccc34)nc(-c3ccc(-c4cccc5ccccc45)c4ccccc34)n2)cc1]

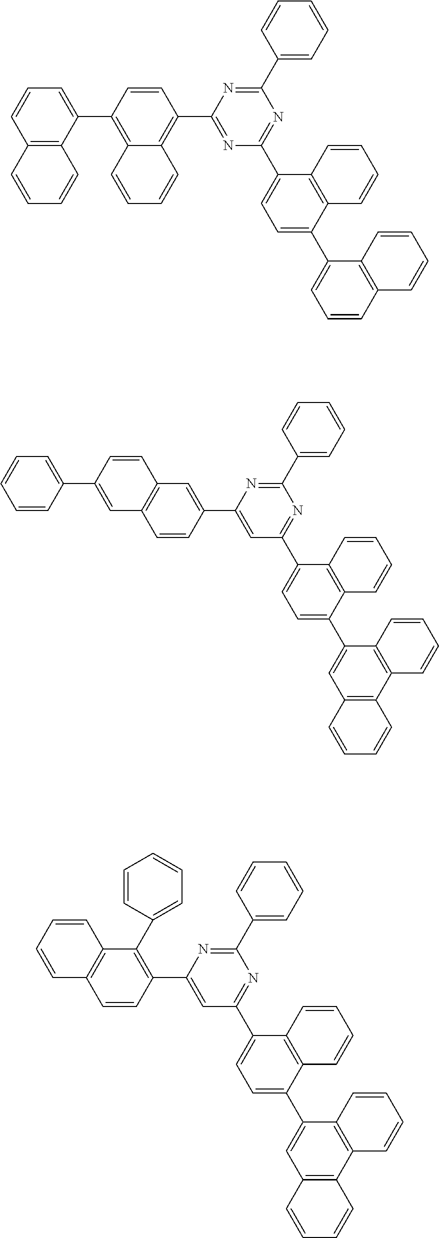 [SMILES: c1ccc(-c2nc(-c3ccc(-c4cccc5ccccc45)c4ccccc34)nc(-c3ccc(-c4cccc5ccccc45)c4ccccc34)n2)cc1 ; c1ccc(-c2ccc3cc(-c4cc(-c5ccc(-c6cc7ccccc7c7ccccc67)c6ccccc56)nc(-c5ccccc5)n4)ccc3c2)cc1 ; c1ccc(-c2nc(-c3ccc4ccccc4c3-c3ccccc3)cc(-c3ccc(-c4cc5ccccc5c5ccccc45)c4ccccc34)n2)cc1]

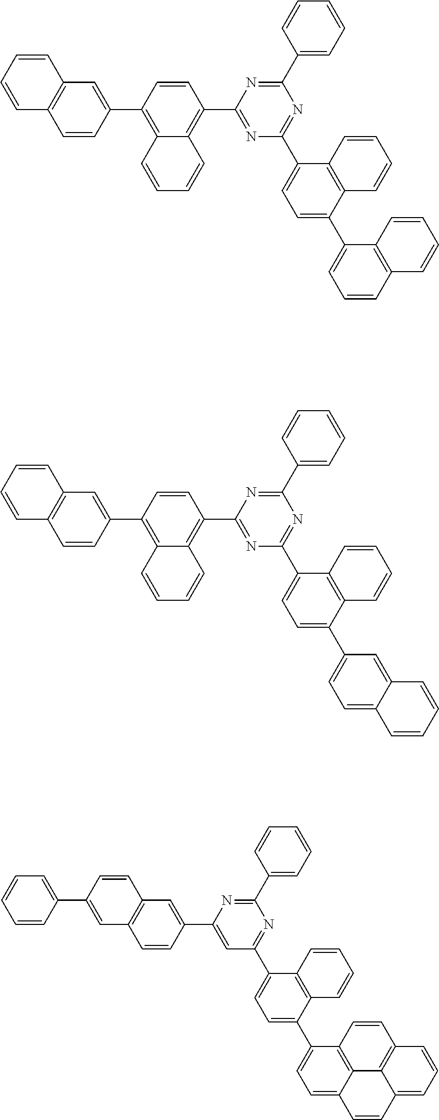 [SMILES: c1ccc(-c2nc(-c3ccc(-c4ccc5ccccc5c4)c4ccccc34)nc(-c3ccc(-c4cccc5ccccc45)c4ccccc34)n2)cc1 ; c1ccc(-c2nc(-c3ccc(-c4ccc5ccccc5c4)c4ccccc34)nc(-c3ccc(-c4ccc5ccccc5c4)c4ccccc34)n2)cc1 ; c1ccc(-c2ccc3cc(-c4cc(-c5ccc(-c6ccc7ccc8cccc9ccc6c7c89)c6ccccc56)nc(-c5ccccc5)n4)ccc3c2)cc1]

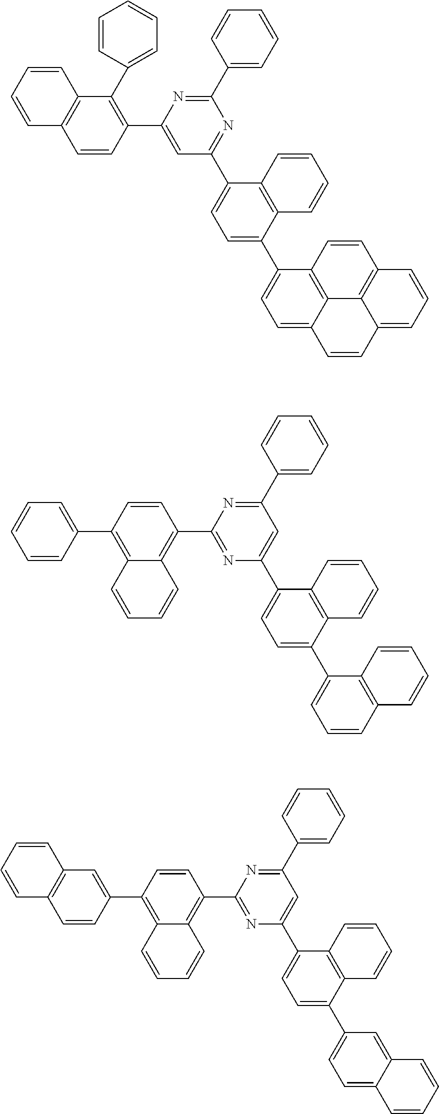 [SMILES: c1ccc(-c2nc(-c3ccc4ccccc4c3-c3ccccc3)cc(-c3ccc(-c4ccc5ccc6cccc7ccc4c5c67)c4ccccc34)n2)cc1 ; c1ccc(-c2cc(-c3ccc(-c4cccc5ccccc45)c4ccccc34)nc(-c3ccc(-c4ccccc4)c4ccccc34)n2)cc1 ; c1ccc(-c2cc(-c3ccc(-c4ccc5ccccc5c4)c4ccccc34)nc(-c3ccc(-c4ccc5ccccc5c4)c4ccccc34)n2)cc1]

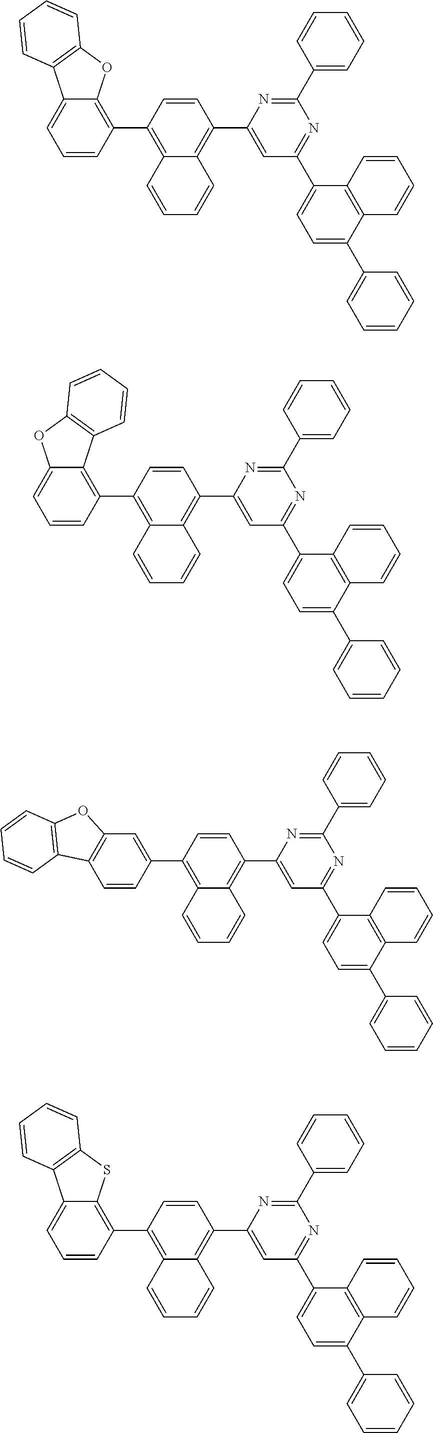 [SMILES: c1ccc(-c2nc(-c3ccc(-c4ccccc4)c4ccccc34)cc(-c3ccc(-c4cccc5c4oc4ccccc45)c4ccccc34)n2)cc1 ; c1ccc(-c2nc(-c3ccc(-c4ccccc4)c4ccccc34)cc(-c3ccc(-c4cccc5oc6ccccc6c45)c4ccccc34)n2)cc1 ; c1ccc(-c2nc(-c3ccc(-c4ccccc4)c4ccccc34)cc(-c3ccc(-c4ccc5c(c4)oc4ccccc45)c4ccccc34)n2)cc1 ; c1ccc(-c2nc(-c3ccc(-c4ccccc4)c4ccccc34)cc(-c3ccc(-c4cccc5c4sc4ccccc45)c4ccccc34)n2)cc1]

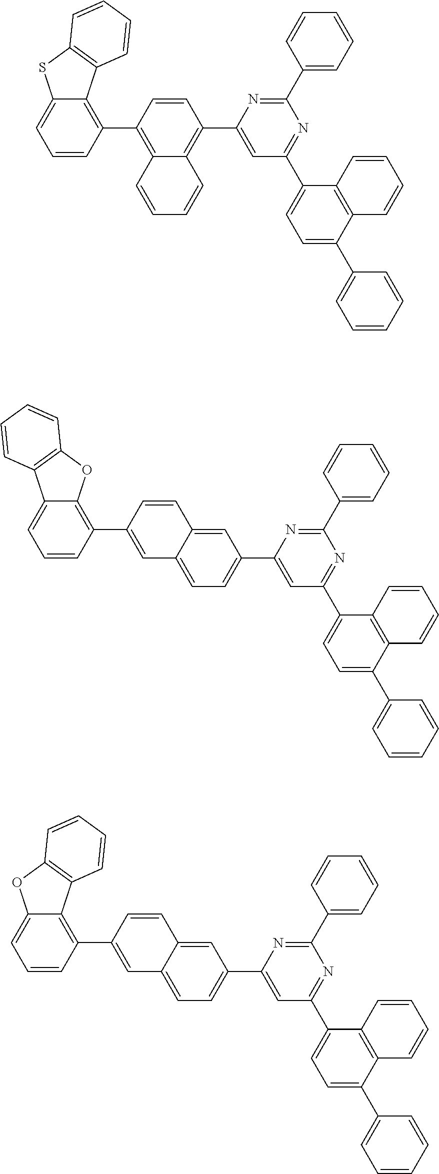 [SMILES: c1ccc(-c2nc(-c3ccc(-c4ccccc4)c4ccccc34)cc(-c3ccc(-c4cccc5sc6ccccc6c45)c4ccccc34)n2)cc1 ; c1ccc(-c2nc(-c3ccc4cc(-c5cccc6c5oc5ccccc56)ccc4c3)cc(-c3ccc(-c4ccccc4)c4ccccc34)n2)cc1 ; c1ccc(-c2nc(-c3ccc4cc(-c5cccc6oc7ccccc7c56)ccc4c3)cc(-c3ccc(-c4ccccc4)c4ccccc34)n2)cc1]

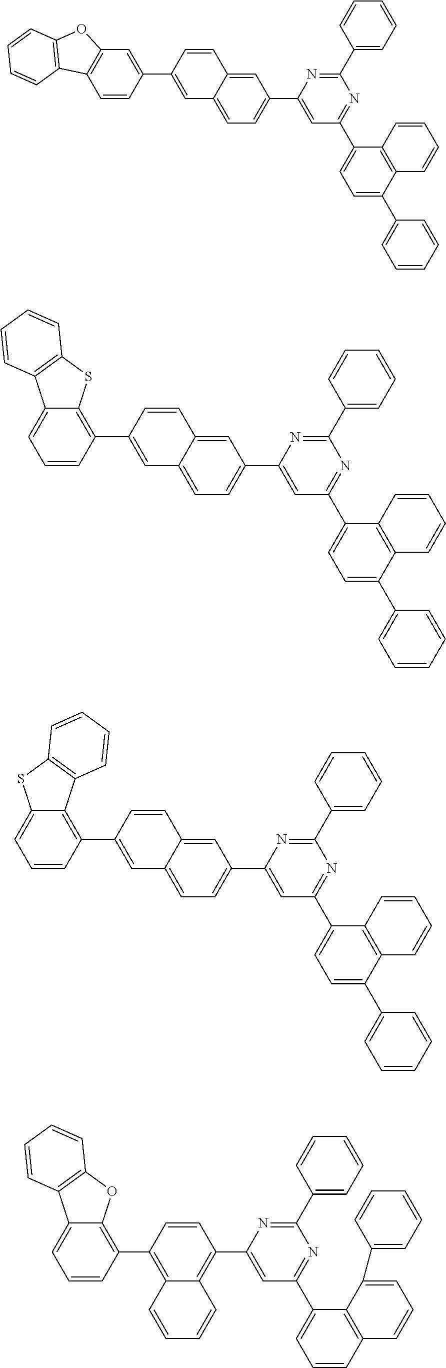 [SMILES: c1ccc(-c2nc(-c3ccc4cc(-c5ccc6c(c5)oc5ccccc56)ccc4c3)cc(-c3ccc(-c4ccccc4)c4ccccc34)n2)cc1 ; c1ccc(-c2nc(-c3ccc4cc(-c5cccc6c5sc5ccccc56)ccc4c3)cc(-c3ccc(-c4ccccc4)c4ccccc34)n2)cc1 ; c1ccc(-c2nc(-c3ccc4cc(-c5cccc6sc7ccccc7c56)ccc4c3)cc(-c3ccc(-c4ccccc4)c4ccccc34)n2)cc1 ; c1ccc(-c2nc(-c3ccc(-c4cccc5c4oc4ccccc45)c4ccccc34)cc(-c3cccc4cccc(-c5ccccc5)c34)n2)cc1]

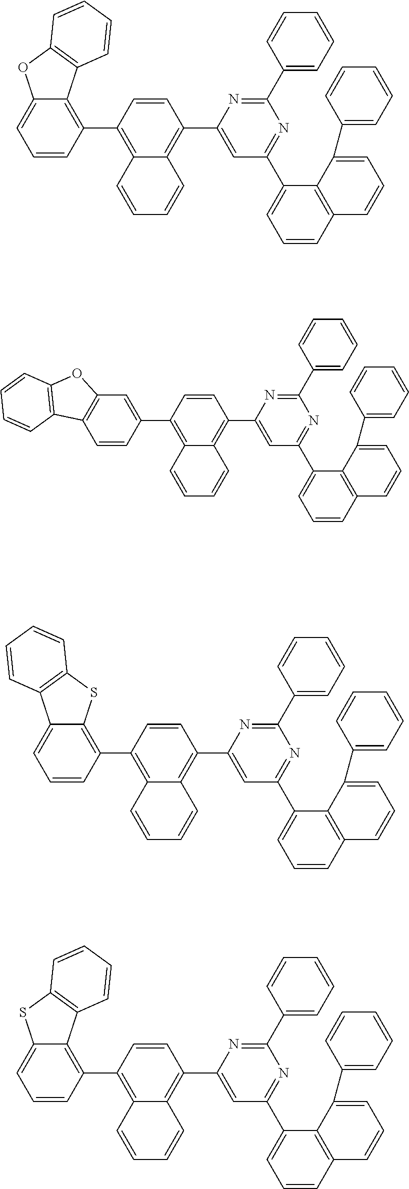 [SMILES: c1ccc(-c2nc(-c3ccc(-c4cccc5oc6ccccc6c45)c4ccccc34)cc(-c3cccc4cccc(-c5ccccc5)c34)n2)cc1 ; c1ccc(-c2nc(-c3ccc(-c4ccc5c(c4)oc4ccccc45)c4ccccc34)cc(-c3cccc4cccc(-c5ccccc5)c34)n2)cc1 ; c1ccc(-c2nc(-c3ccc(-c4cccc5c4sc4ccccc45)c4ccccc34)cc(-c3cccc4cccc(-c5ccccc5)c34)n2)cc1 ; c1ccc(-c2nc(-c3ccc(-c4cccc5sc6ccccc6c45)c4ccccc34)cc(-c3cccc4cccc(-c5ccccc5)c34)n2)cc1]

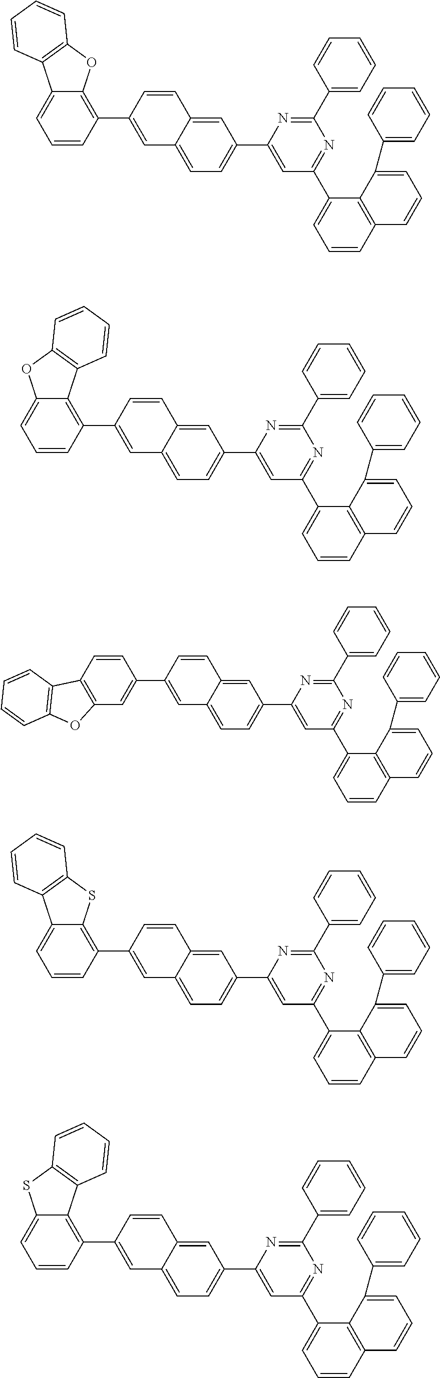 [SMILES: c1ccc(-c2nc(-c3ccc4cc(-c5cccc6c5oc5ccccc56)ccc4c3)cc(-c3cccc4cccc(-c5ccccc5)c34)n2)cc1 ; c1ccc(-c2nc(-c3ccc4cc(-c5cccc6oc7ccccc7c56)ccc4c3)cc(-c3cccc4cccc(-c5ccccc5)c34)n2)cc1 ; c1ccc(-c2nc(-c3ccc4cc(-c5ccc6c(c5)oc5ccccc56)ccc4c3)cc(-c3cccc4cccc(-c5ccccc5)c34)n2)cc1 ; c1ccc(-c2nc(-c3ccc4cc(-c5cccc6c5sc5ccccc56)ccc4c3)cc(-c3cccc4cccc(-c5ccccc5)c34)n2)cc1 ; c1ccc(-c2nc(-c3ccc4cc(-c5cccc6sc7ccccc7c56)ccc4c3)cc(-c3cccc4cccc(-c5ccccc5)c34)n2)cc1]

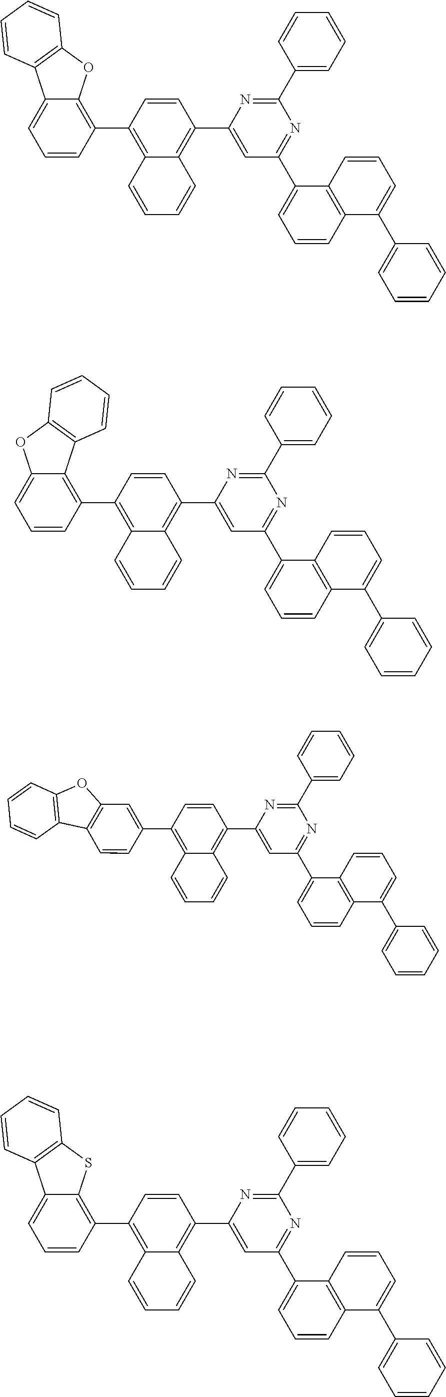 [SMILES: c1ccc(-c2nc(-c3cccc4c(-c5ccccc5)cccc34)cc(-c3ccc(-c4cccc5c4oc4ccccc45)c4ccccc34)n2)cc1 ; c1ccc(-c2nc(-c3cccc4c(-c5ccccc5)cccc34)cc(-c3ccc(-c4cccc5oc6ccccc6c45)c4ccccc34)n2)cc1 ; c1ccc(-c2nc(-c3cccc4c(-c5ccccc5)cccc34)cc(-c3ccc(-c4ccc5c(c4)oc4ccccc45)c4ccccc34)n2)cc1 ; c1ccc(-c2nc(-c3cccc4c(-c5ccccc5)cccc34)cc(-c3ccc(-c4cccc5c4sc4ccccc45)c4ccccc34)n2)cc1]

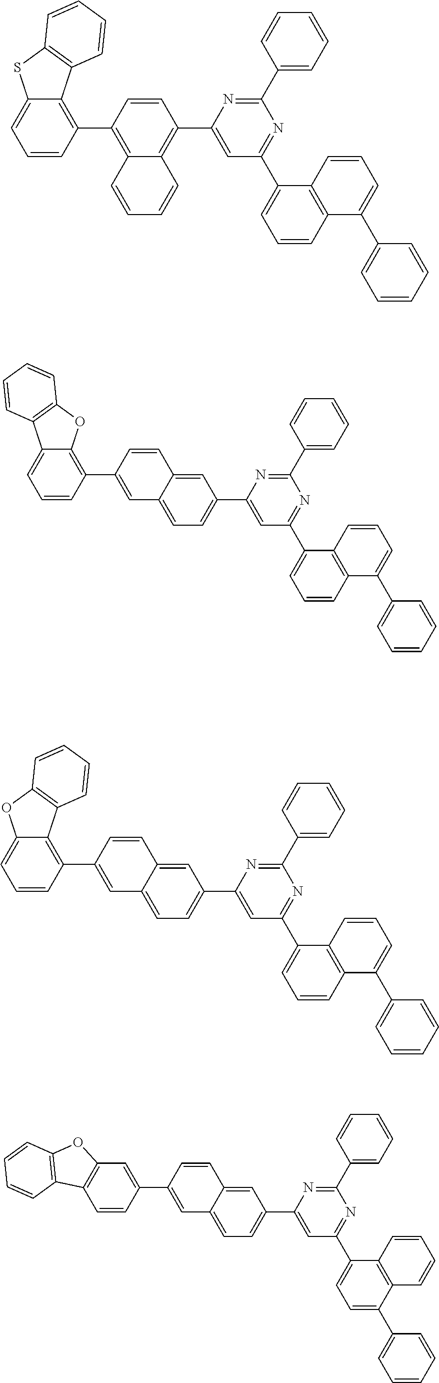 [SMILES: c1ccc(-c2nc(-c3cccc4c(-c5ccccc5)cccc34)cc(-c3ccc(-c4cccc5sc6ccccc6c45)c4ccccc34)n2)cc1 ; c1ccc(-c2nc(-c3ccc4cc(-c5cccc6c5oc5ccccc56)ccc4c3)cc(-c3cccc4c(-c5ccccc5)cccc34)n2)cc1 ; c1ccc(-c2nc(-c3ccc4cc(-c5cccc6oc7ccccc7c56)ccc4c3)cc(-c3cccc4c(-c5ccccc5)cccc34)n2)cc1 ; c1ccc(-c2nc(-c3ccc4cc(-c5ccc6c(c5)oc5ccccc56)ccc4c3)cc(-c3ccc(-c4ccccc4)c4ccccc34)n2)cc1]

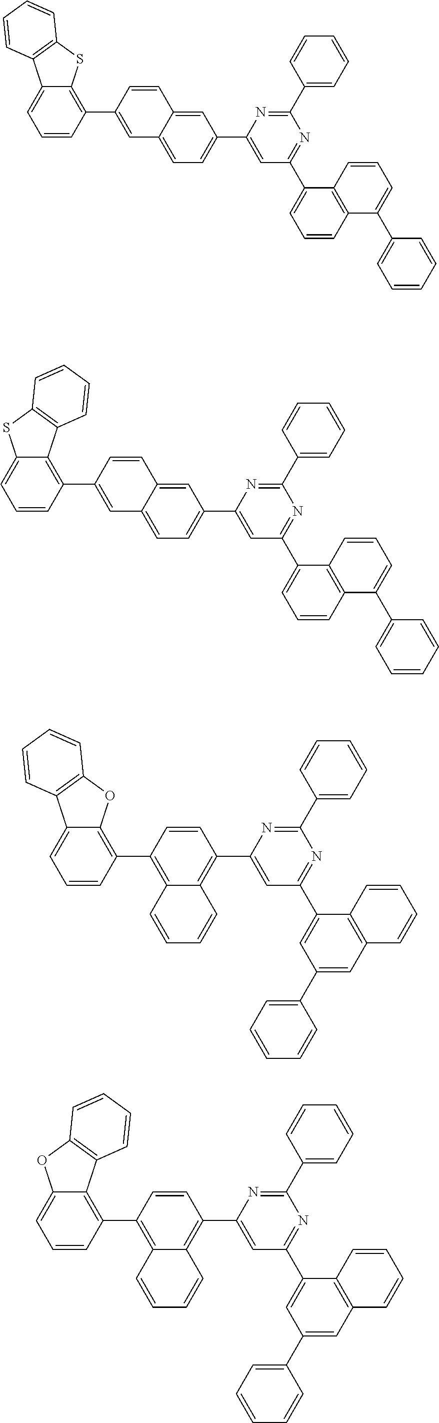 [SMILES: c1ccc(-c2nc(-c3ccc4cc(-c5cccc6c5sc5ccccc56)ccc4c3)cc(-c3cccc4c(-c5ccccc5)cccc34)n2)cc1 ; c1ccc(-c2nc(-c3ccc4cc(-c5cccc6sc7ccccc7c56)ccc4c3)cc(-c3cccc4c(-c5ccccc5)cccc34)n2)cc1 ; c1ccc(-c2cc(-c3cc(-c4ccc(-c5cccc6c5oc5ccccc56)c5ccccc45)nc(-c4ccccc4)n3)c3ccccc3c2)cc1 ; c1ccc(-c2cc(-c3cc(-c4ccc(-c5cccc6oc7ccccc7c56)c5ccccc45)nc(-c4ccccc4)n3)c3ccccc3c2)cc1]

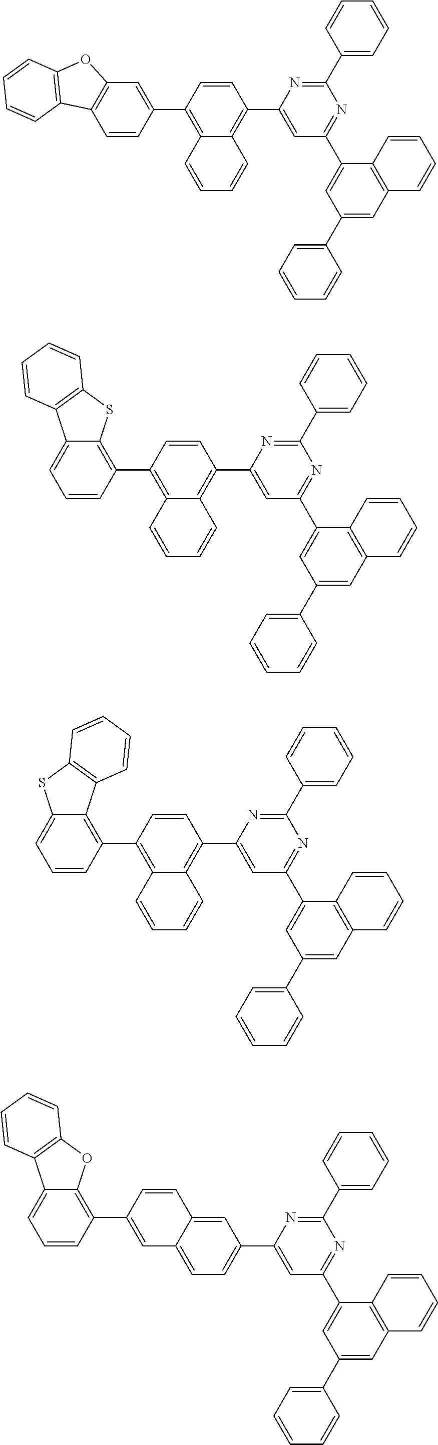 [SMILES: c1ccc(-c2cc(-c3cc(-c4ccc(-c5ccc6c(c5)oc5ccccc56)c5ccccc45)nc(-c4ccccc4)n3)c3ccccc3c2)cc1 ; c1ccc(-c2cc(-c3cc(-c4ccc(-c5cccc6c5sc5ccccc56)c5ccccc45)nc(-c4ccccc4)n3)c3ccccc3c2)cc1 ; c1ccc(-c2cc(-c3cc(-c4ccc(-c5cccc6sc7ccccc7c56)c5ccccc45)nc(-c4ccccc4)n3)c3ccccc3c2)cc1 ; c1ccc(-c2cc(-c3cc(-c4ccc5cc(-c6cccc7c6oc6ccccc67)ccc5c4)nc(-c4ccccc4)n3)c3ccccc3c2)cc1]

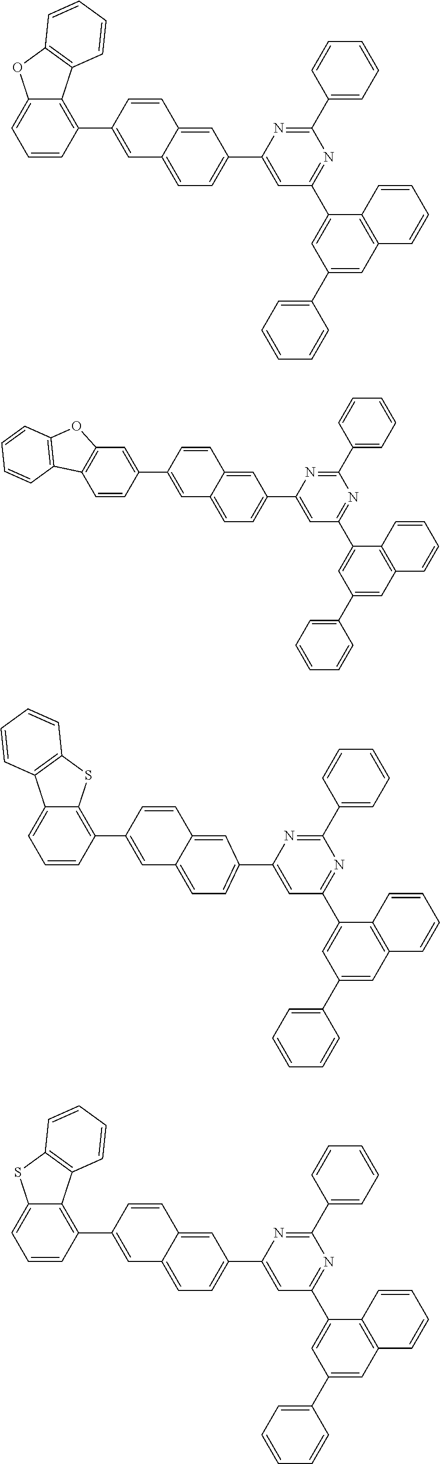 [SMILES: c1ccc(-c2cc(-c3cc(-c4ccc5cc(-c6cccc7oc8ccccc8c67)ccc5c4)nc(-c4ccccc4)n3)c3ccccc3c2)cc1 ; c1ccc(-c2cc(-c3cc(-c4ccc5cc(-c6ccc7c(c6)oc6ccccc67)ccc5c4)nc(-c4ccccc4)n3)c3ccccc3c2)cc1 ; c1ccc(-c2cc(-c3cc(-c4ccc5cc(-c6cccc7c6sc6ccccc67)ccc5c4)nc(-c4ccccc4)n3)c3ccccc3c2)cc1 ; c1ccc(-c2cc(-c3cc(-c4ccc5cc(-c6cccc7sc8ccccc8c67)ccc5c4)nc(-c4ccccc4)n3)c3ccccc3c2)cc1]

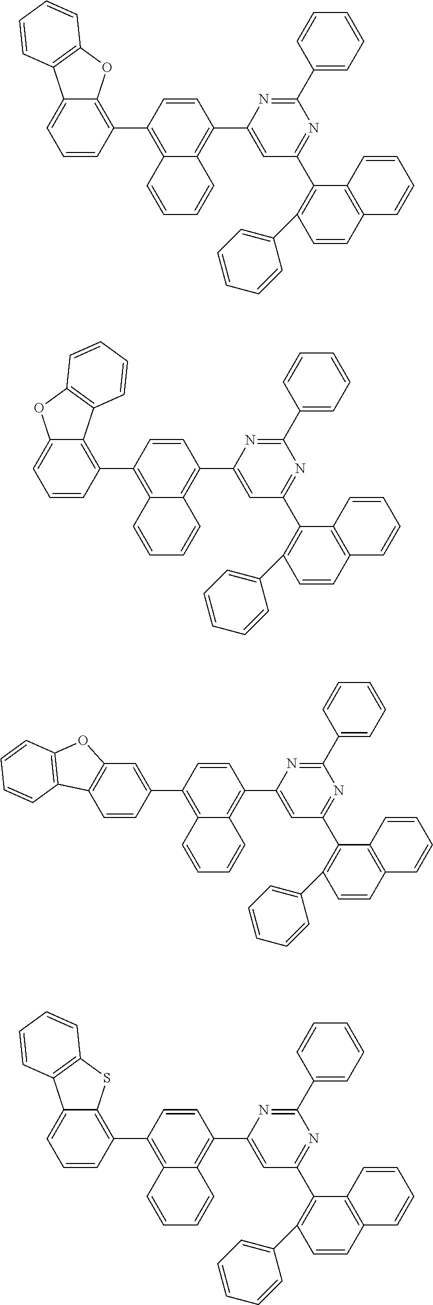 [SMILES: c1ccc(-c2nc(-c3c(-c4ccccc4)ccc4ccccc34)cc(-c3ccc(-c4cccc5c4oc4ccccc45)c4ccccc34)n2)cc1 ; c1ccc(-c2nc(-c3c(-c4ccccc4)ccc4ccccc34)cc(-c3ccc(-c4cccc5oc6ccccc6c45)c4ccccc34)n2)cc1 ; c1ccc(-c2nc(-c3c(-c4ccccc4)ccc4ccccc34)cc(-c3ccc(-c4ccc5c(c4)oc4ccccc45)c4ccccc34)n2)cc1 ; c1ccc(-c2nc(-c3c(-c4ccccc4)ccc4ccccc34)cc(-c3ccc(-c4cccc5c4sc4ccccc45)c4ccccc34)n2)cc1]

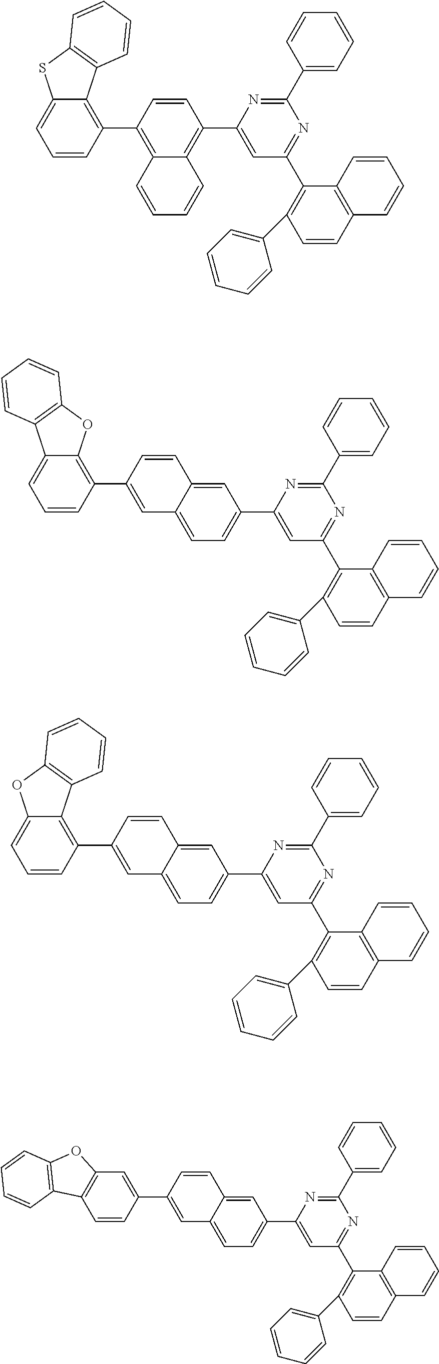

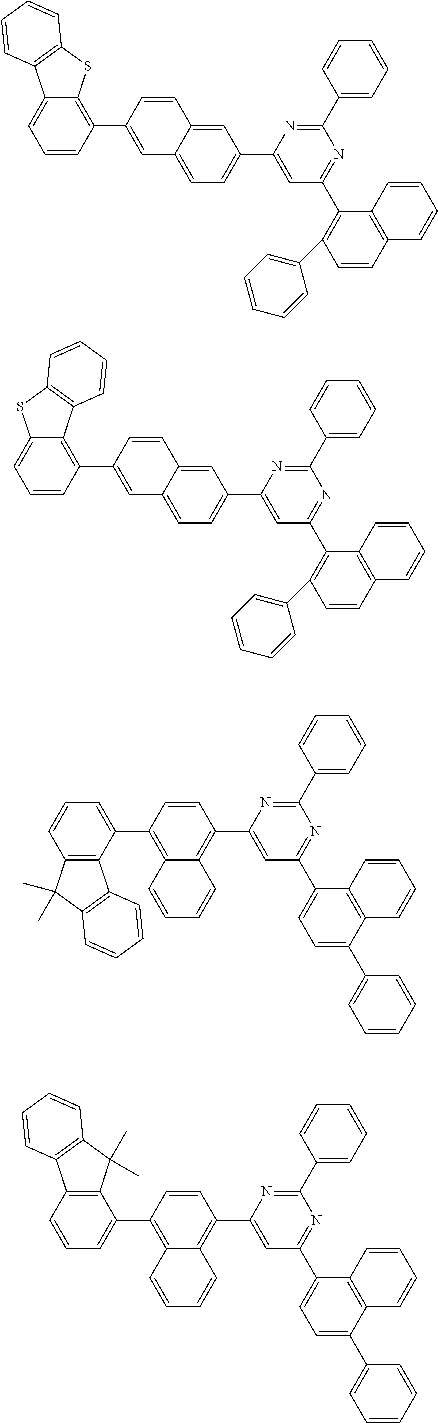 [SMILES: c1ccc(-c2nc(-c3ccc4cc(-c5cccc6c5sc5ccccc56)ccc4c3)cc(-c3c(-c4ccccc4)ccc4ccccc34)n2)cc1 ; c1ccc(-c2nc(-c3ccc4cc(-c5cccc6sc7ccccc7c56)ccc4c3)cc(-c3c(-c4ccccc4)ccc4ccccc34)n2)cc1 ; CC1(C)c2ccccc2-c2c(-c3ccc(-c4cc(-c5ccc(-c6ccccc6)c6ccccc56)nc(-c5ccccc5)n4)c4ccccc34)cccc21 ; CC1(C)c2ccccc2-c2cccc(-c3ccc(-c4cc(-c5ccc(-c6ccccc6)c6ccccc56)nc(-c5ccccc5)n4)c4ccccc34)c21]

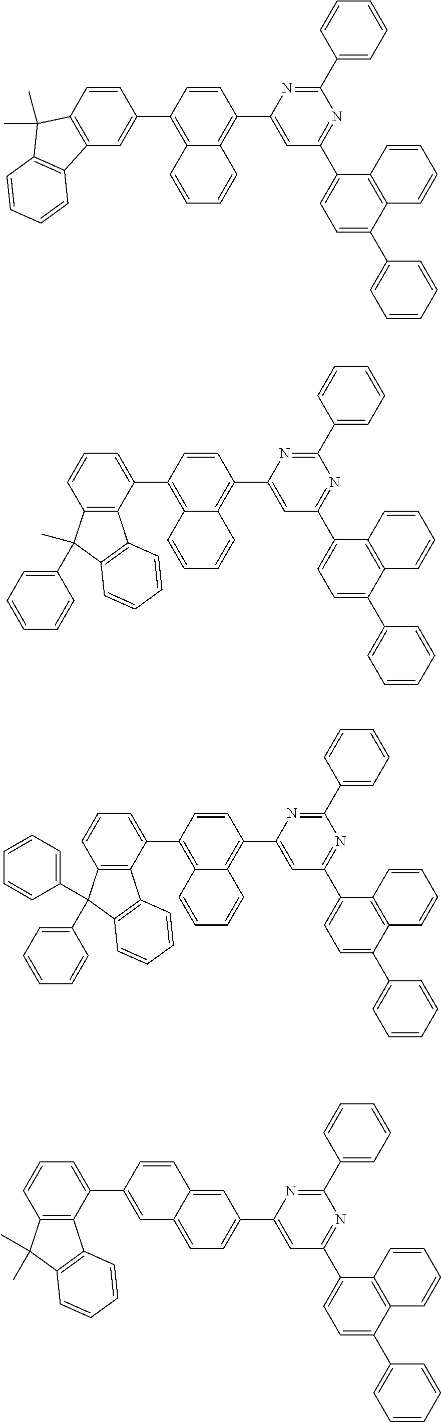

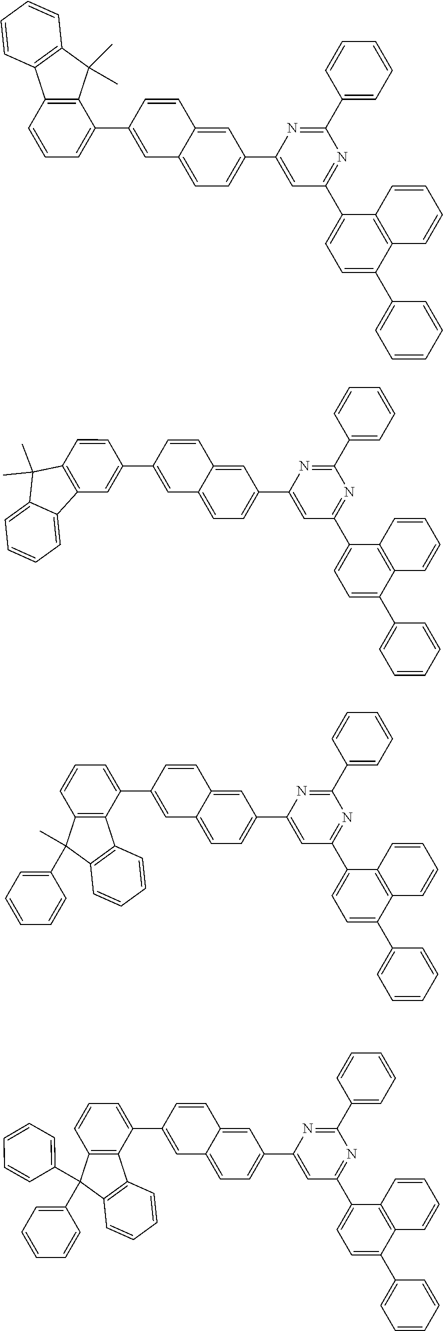

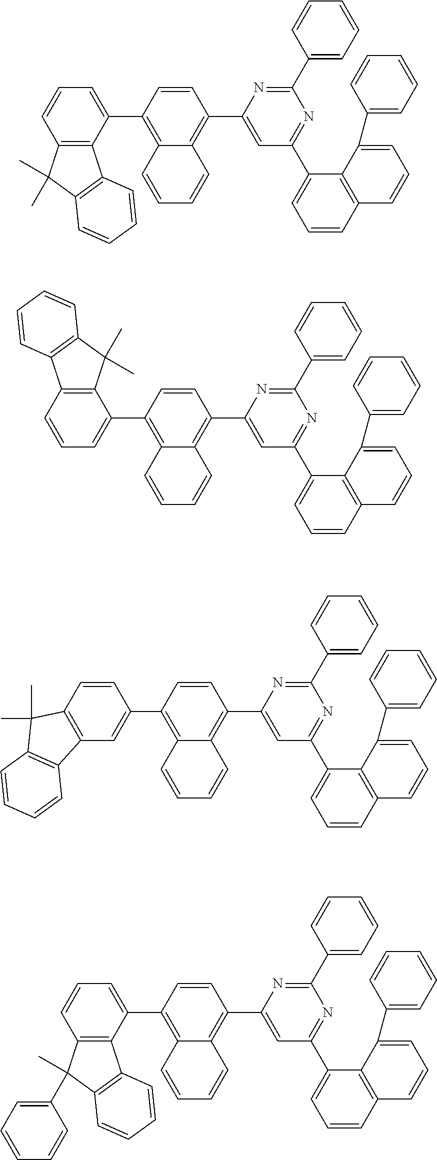 [SMILES: CC1(C)c2ccccc2-c2c(-c3ccc(-c4cc(-c5cccc6cccc(-c7ccccc7)c56)nc(-c5ccccc5)n4)c4ccccc34)cccc21 ; CC1(C)c2ccccc2-c2cccc(-c3ccc(-c4cc(-c5cccc6cccc(-c7ccccc7)c56)nc(-c5ccccc5)n4)c4ccccc34)c21 ; CC1(C)c2ccccc2-c2cc(-c3ccc(-c4cc(-c5cccc6cccc(-c7ccccc7)c56)nc(-c5ccccc5)n4)c4ccccc34)ccc21 ; CC1(c2ccccc2)c2ccccc2-c2c(-c3ccc(-c4cc(-c5cccc6cccc(-c7ccccc7)c56)nc(-c5ccccc5)n4)c4ccccc34)cccc21]

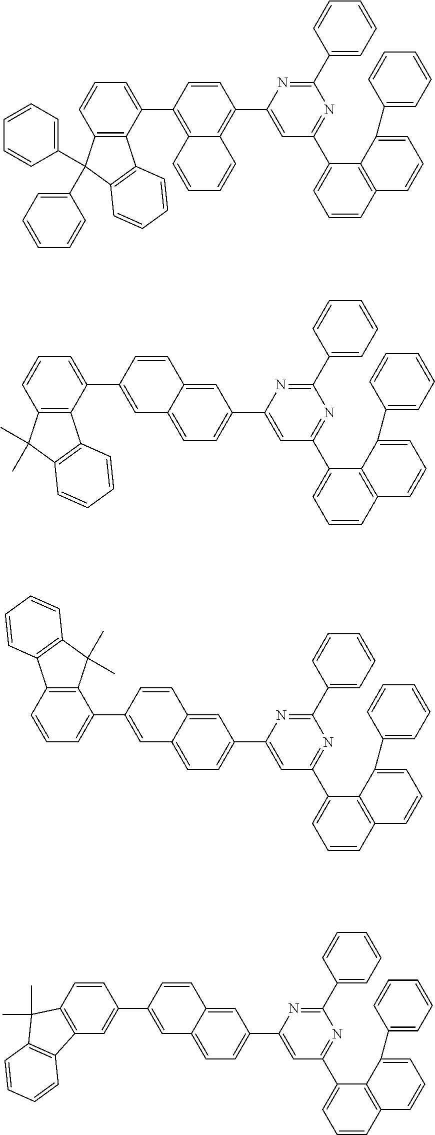 [SMILES: c1ccc(-c2nc(-c3ccc(-c4cccc5c4-c4ccccc4C5(c4ccccc4)c4ccccc4)c4ccccc34)cc(-c3cccc4cccc(-c5ccccc5)c34)n2)cc1 ; CC1(C)c2ccccc2-c2c(-c3ccc4cc(-c5cc(-c6cccc7cccc(-c8ccccc8)c67)nc(-c6ccccc6)n5)ccc4c3)cccc21 ; CC1(C)c2ccccc2-c2cccc(-c3ccc4cc(-c5cc(-c6cccc7cccc(-c8ccccc8)c67)nc(-c6ccccc6)n5)ccc4c3)c21 ; CC1(C)c2ccccc2-c2cc(-c3ccc4cc(-c5cc(-c6cccc7cccc(-c8ccccc8)c67)nc(-c6ccccc6)n5)ccc4c3)ccc21]

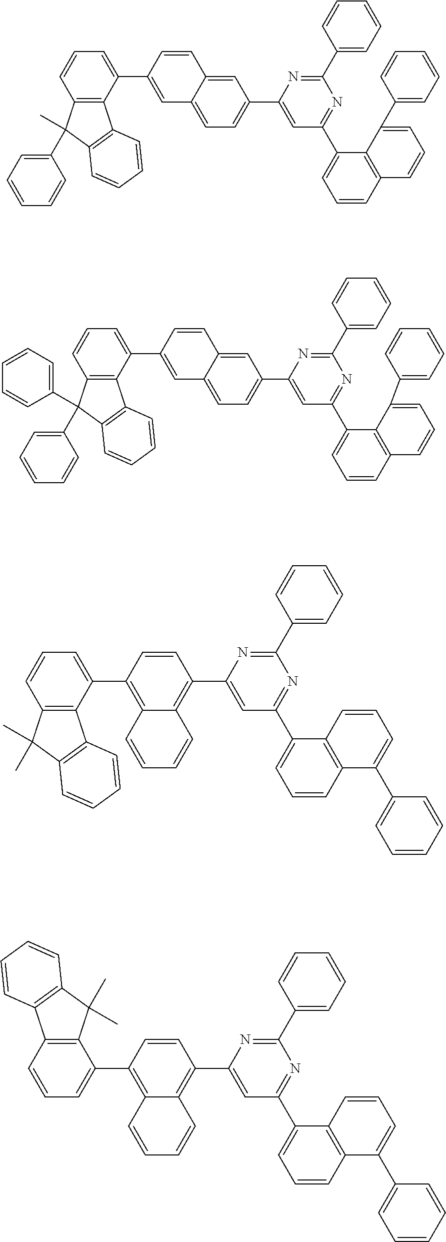 [SMILES: CC1(c2ccccc2)c2ccccc2-c2c(-c3ccc4cc(-c5cc(-c6cccc7cccc(-c8ccccc8)c67)nc(-c6ccccc6)n5)ccc4c3)cccc21 ; c1ccc(-c2nc(-c3ccc4cc(-c5cccc6c5-c5ccccc5C6(c5ccccc5)c5ccccc5)ccc4c3)cc(-c3cccc4cccc(-c5ccccc5)c34)n2)cc1 ; CC1(C)c2ccccc2-c2c(-c3ccc(-c4cc(-c5cccc6c(-c7ccccc7)cccc56)nc(-c5ccccc5)n4)c4ccccc34)cccc21 ; CC1(C)c2ccccc2-c2cccc(-c3ccc(-c4cc(-c5cccc6c(-c7ccccc7)cccc56)nc(-c5ccccc5)n4)c4ccccc34)c21]

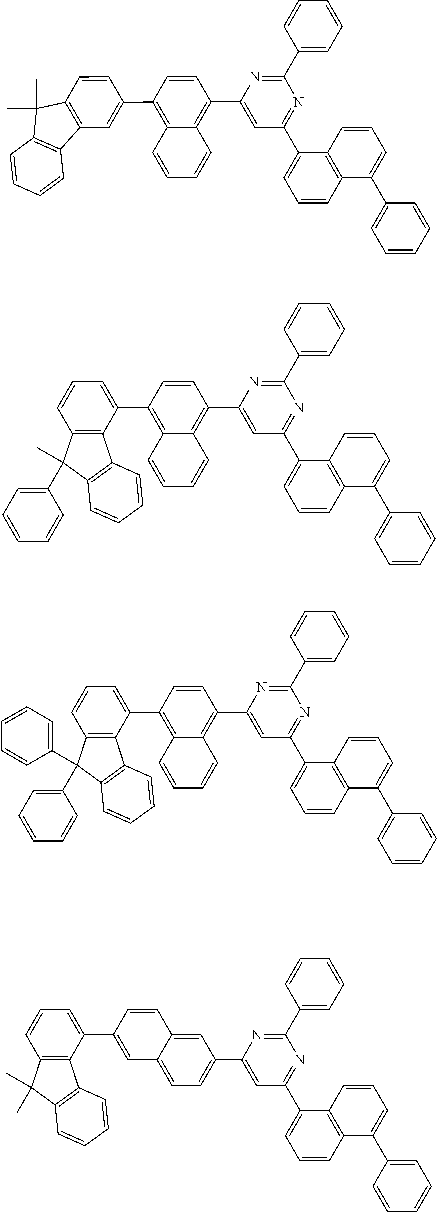 [SMILES: CC1(C)c2ccccc2-c2cc(-c3ccc(-c4cc(-c5cccc6c(-c7ccccc7)cccc56)nc(-c5ccccc5)n4)c4ccccc34)ccc21 ; CC1(c2ccccc2)c2ccccc2-c2c(-c3ccc(-c4cc(-c5cccc6c(-c7ccccc7)cccc56)nc(-c5ccccc5)n4)c4ccccc34)cccc21 ; c1ccc(-c2nc(-c3cccc4c(-c5ccccc5)cccc34)cc(-c3ccc(-c4cccc5c4-c4ccccc4C5(c4ccccc4)c4ccccc4)c4ccccc34)n2)cc1 ; CC1(C)c2ccccc2-c2c(-c3ccc4cc(-c5cc(-c6cccc7c(-c8ccccc8)cccc67)nc(-c6ccccc6)n5)ccc4c3)cccc21]

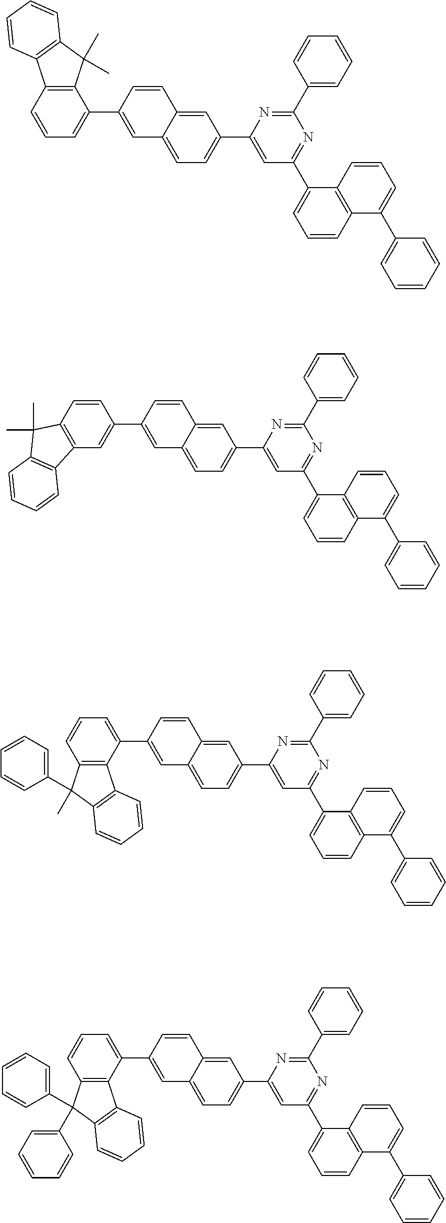 [SMILES: CC1(C)c2ccccc2-c2cccc(-c3ccc4cc(-c5cc(-c6cccc7c(-c8ccccc8)cccc67)nc(-c6ccccc6)n5)ccc4c3)c21 ; CC1(C)c2ccccc2-c2cc(-c3ccc4cc(-c5cc(-c6cccc7c(-c8ccccc8)cccc67)nc(-c6ccccc6)n5)ccc4c3)ccc21 ; CC1(c2ccccc2)c2ccccc2-c2c(-c3ccc4cc(-c5cc(-c6cccc7c(-c8ccccc8)cccc67)nc(-c6ccccc6)n5)ccc4c3)cccc21 ; c1ccc(-c2nc(-c3ccc4cc(-c5cccc6c5-c5ccccc5C6(c5ccccc5)c5ccccc5)ccc4c3)cc(-c3cccc4c(-c5ccccc5)cccc34)n2)cc1]

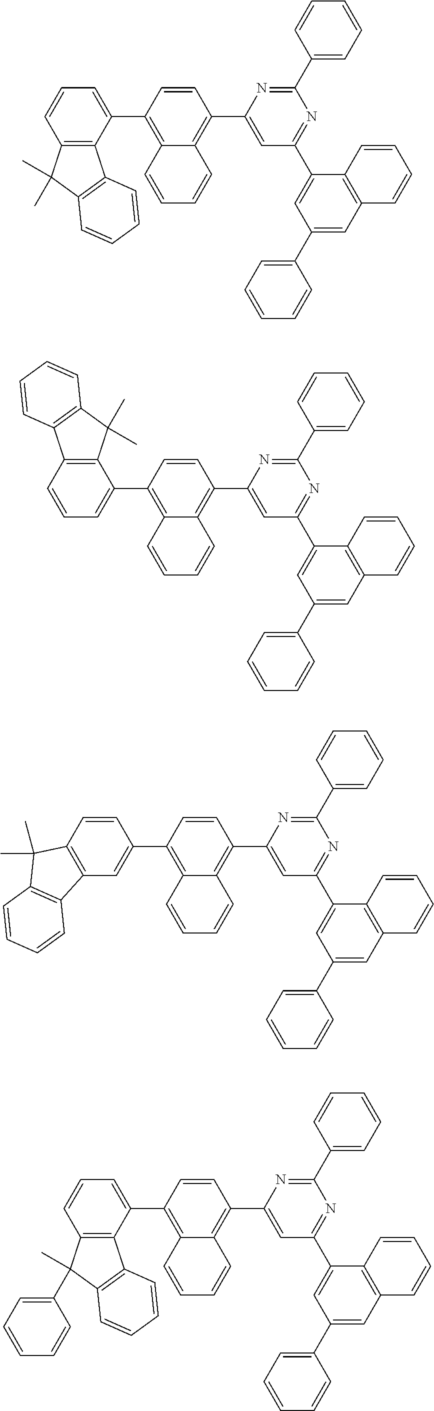 [SMILES: CC1(C)c2ccccc2-c2c(-c3ccc(-c4cc(-c5cc(-c6ccccc6)cc6ccccc56)nc(-c5ccccc5)n4)c4ccccc34)cccc21 ; CC1(C)c2ccccc2-c2cccc(-c3ccc(-c4cc(-c5cc(-c6ccccc6)cc6ccccc56)nc(-c5ccccc5)n4)c4ccccc34)c21 ; CC1(C)c2ccccc2-c2cc(-c3ccc(-c4cc(-c5cc(-c6ccccc6)cc6ccccc56)nc(-c5ccccc5)n4)c4ccccc34)ccc21 ; CC1(c2ccccc2)c2ccccc2-c2c(-c3ccc(-c4cc(-c5cc(-c6ccccc6)cc6ccccc56)nc(-c5ccccc5)n4)c4ccccc34)cccc21]

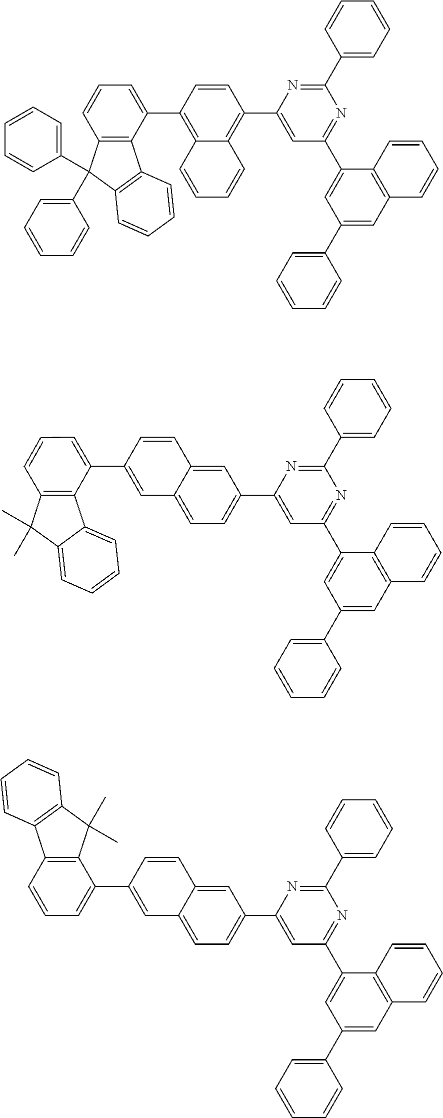 [SMILES: c1ccc(-c2cc(-c3cc(-c4ccc(-c5cccc6c5-c5ccccc5C6(c5ccccc5)c5ccccc5)c5ccccc45)nc(-c4ccccc4)n3)c3ccccc3c2)cc1 ; CC1(C)c2ccccc2-c2c(-c3ccc4cc(-c5cc(-c6cc(-c7ccccc7)cc7ccccc67)nc(-c6ccccc6)n5)ccc4c3)cccc21 ; CC1(C)c2ccccc2-c2cccc(-c3ccc4cc(-c5cc(-c6cc(-c7ccccc7)cc7ccccc67)nc(-c6ccccc6)n5)ccc4c3)c21]

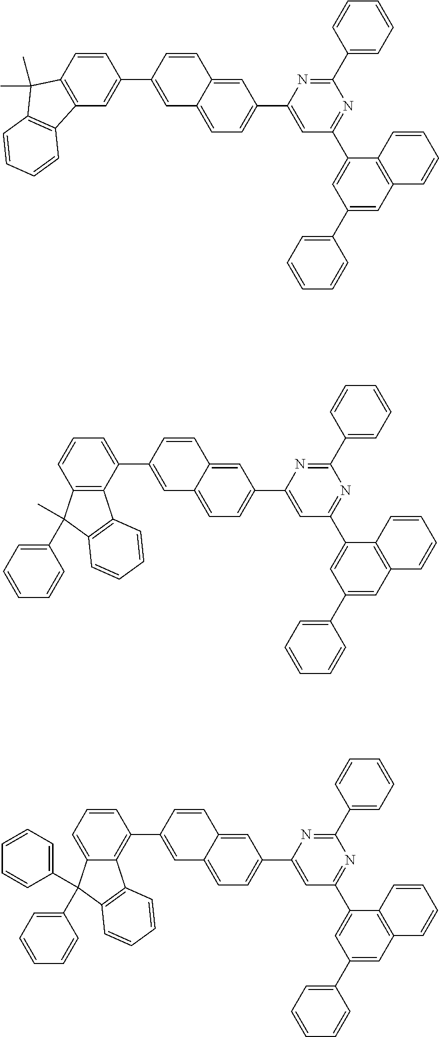 [SMILES: CC1(C)c2ccccc2-c2cc(-c3ccc4cc(-c5cc(-c6cc(-c7ccccc7)cc7ccccc67)nc(-c6ccccc6)n5)ccc4c3)ccc21 ; CC1(c2ccccc2)c2ccccc2-c2c(-c3ccc4cc(-c5cc(-c6cc(-c7ccccc7)cc7ccccc67)nc(-c6ccccc6)n5)ccc4c3)cccc21 ; c1ccc(-c2cc(-c3cc(-c4ccc5cc(-c6cccc7c6-c6ccccc6C7(c6ccccc6)c6ccccc6)ccc5c4)nc(-c4ccccc4)n3)c3ccccc3c2)cc1]

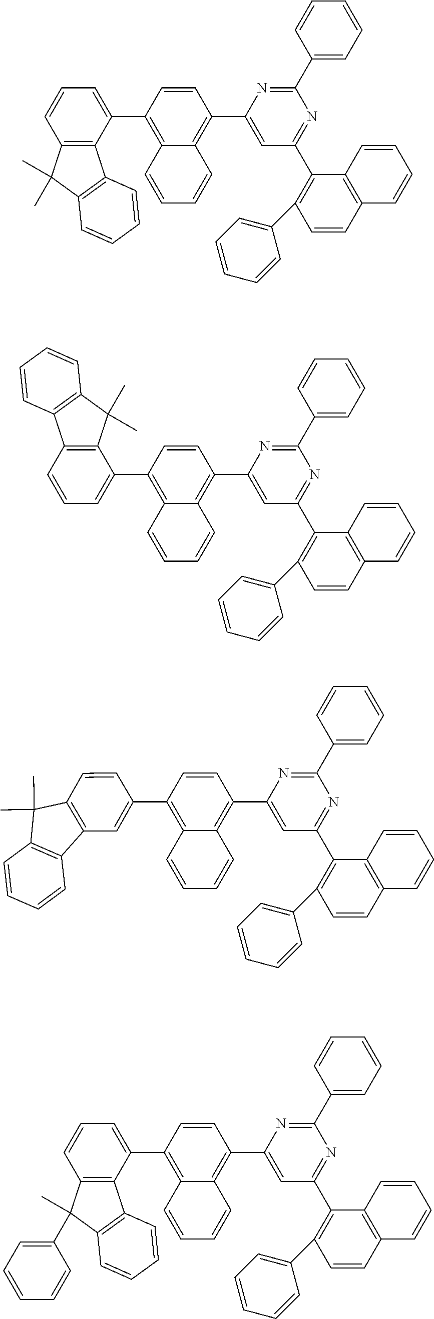 [SMILES: CC1(C)c2ccccc2-c2c(-c3ccc(-c4cc(-c5c(-c6ccccc6)ccc6ccccc56)nc(-c5ccccc5)n4)c4ccccc34)cccc21 ; CC1(C)c2ccccc2-c2cccc(-c3ccc(-c4cc(-c5c(-c6ccccc6)ccc6ccccc56)nc(-c5ccccc5)n4)c4ccccc34)c21 ; CC1(C)c2ccccc2-c2cc(-c3ccc(-c4cc(-c5c(-c6ccccc6)ccc6ccccc56)nc(-c5ccccc5)n4)c4ccccc34)ccc21 ; CC1(c2ccccc2)c2ccccc2-c2c(-c3ccc(-c4cc(-c5c(-c6ccccc6)ccc6ccccc56)nc(-c5ccccc5)n4)c4ccccc34)cccc21]

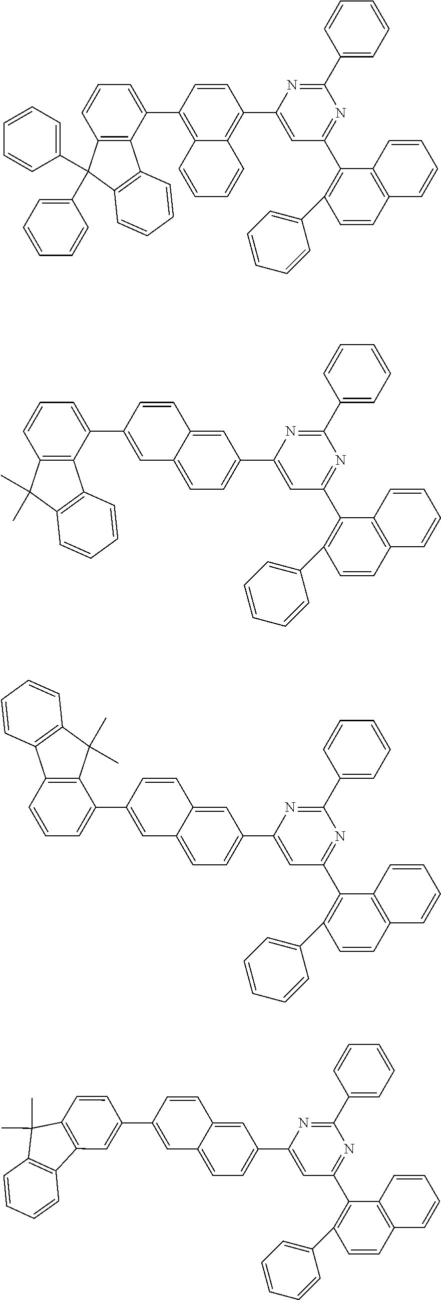 [SMILES: c1ccc(-c2nc(-c3c(-c4ccccc4)ccc4ccccc34)cc(-c3ccc(-c4cccc5c4-c4ccccc4C5(c4ccccc4)c4ccccc4)c4ccccc34)n2)cc1 ; CC1(C)c2ccccc2-c2c(-c3ccc4cc(-c5cc(-c6c(-c7ccccc7)ccc7ccccc67)nc(-c6ccccc6)n5)ccc4c3)cccc21 ; CC1(C)c2ccccc2-c2cccc(-c3ccc4cc(-c5cc(-c6c(-c7ccccc7)ccc7ccccc67)nc(-c6ccccc6)n5)ccc4c3)c21 ; CC1(C)c2ccccc2-c2cc(-c3ccc4cc(-c5cc(-c6c(-c7ccccc7)ccc7ccccc67)nc(-c6ccccc6)n5)ccc4c3)ccc21]

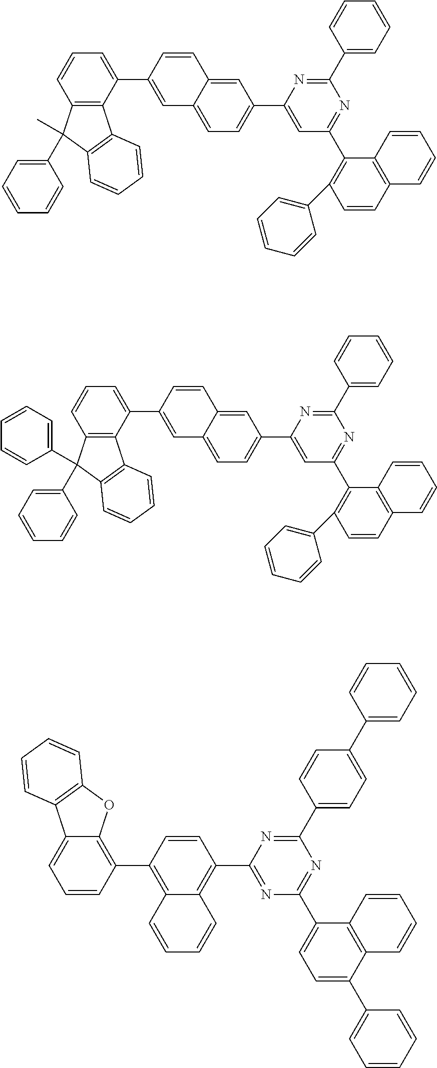 [SMILES: CC1(c2ccccc2)c2ccccc2-c2c(-c3ccc4cc(-c5cc(-c6c(-c7ccccc7)ccc7ccccc67)nc(-c6ccccc6)n5)ccc4c3)cccc21 ; c1ccc(-c2nc(-c3ccc4cc(-c5cccc6c5-c5ccccc5C6(c5ccccc5)c5ccccc5)ccc4c3)cc(-c3c(-c4ccccc4)ccc4ccccc34)n2)cc1 ; c1ccc(-c2ccc(-c3nc(-c4ccc(-c5ccccc5)c5ccccc45)nc(-c4ccc(-c5cccc6c5oc5ccccc56)c5ccccc45)n3)cc2)cc1]

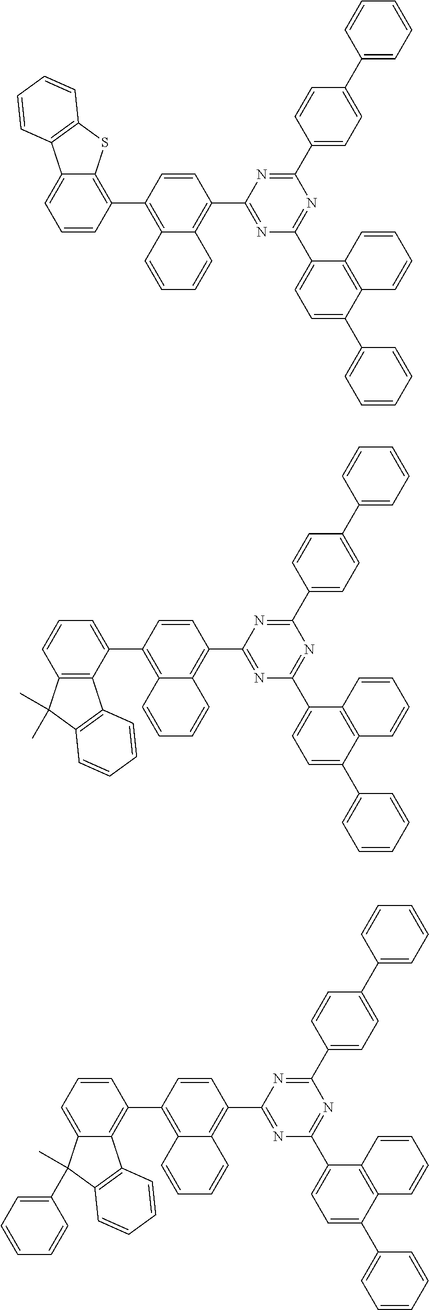

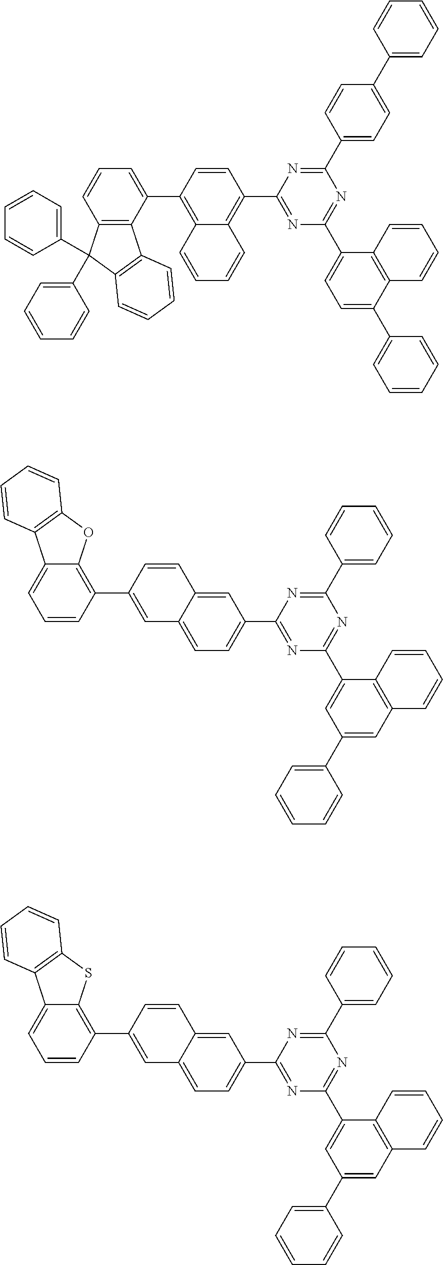 [SMILES: c1ccc(-c2ccc(-c3nc(-c4ccc(-c5ccccc5)c5ccccc45)nc(-c4ccc(-c5cccc6c5-c5ccccc5C6(c5ccccc5)c5ccccc5)c5ccccc45)n3)cc2)cc1 ; c1ccc(-c2cc(-c3nc(-c4ccccc4)nc(-c4ccc5cc(-c6cccc7c6oc6ccccc67)ccc5c4)n3)c3ccccc3c2)cc1 ; c1ccc(-c2cc(-c3nc(-c4ccccc4)nc(-c4ccc5cc(-c6cccc7c6sc6ccccc67)ccc5c4)n3)c3ccccc3c2)cc1]

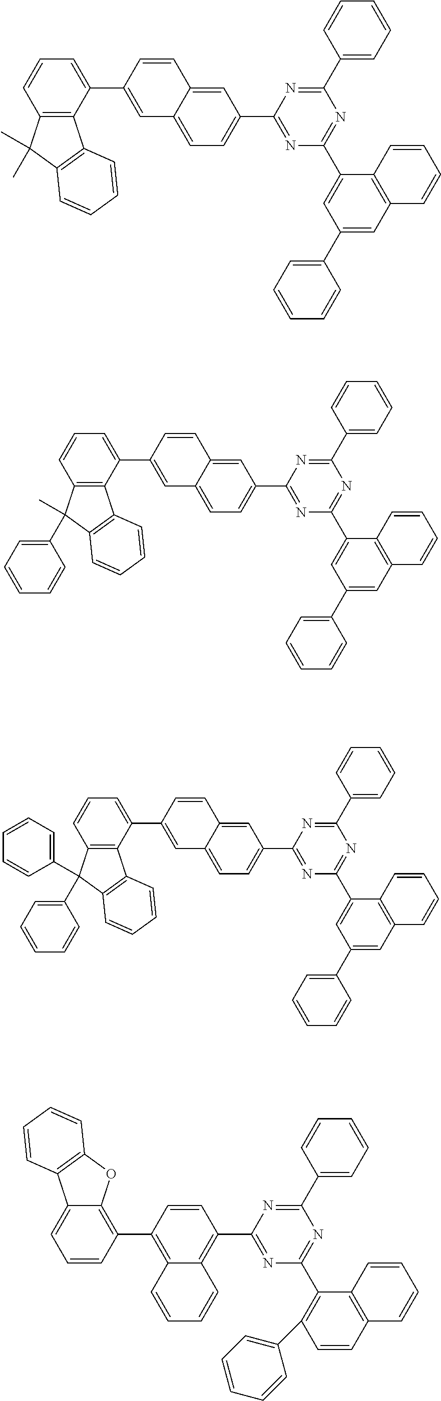 [SMILES: CC1(C)c2ccccc2-c2c(-c3ccc4cc(-c5nc(-c6ccccc6)nc(-c6cc(-c7ccccc7)cc7ccccc67)n5)ccc4c3)cccc21 ; CC1(c2ccccc2)c2ccccc2-c2c(-c3ccc4cc(-c5nc(-c6ccccc6)nc(-c6cc(-c7ccccc7)cc7ccccc67)n5)ccc4c3)cccc21 ; c1ccc(-c2cc(-c3nc(-c4ccccc4)nc(-c4ccc5cc(-c6cccc7c6-c6ccccc6C7(c6ccccc6)c6ccccc6)ccc5c4)n3)c3ccccc3c2)cc1 ; c1ccc(-c2nc(-c3c(-c4ccccc4)ccc4ccccc34)nc(-c3ccc(-c4cccc5c4oc4ccccc45)c4ccccc34)n2)cc1]

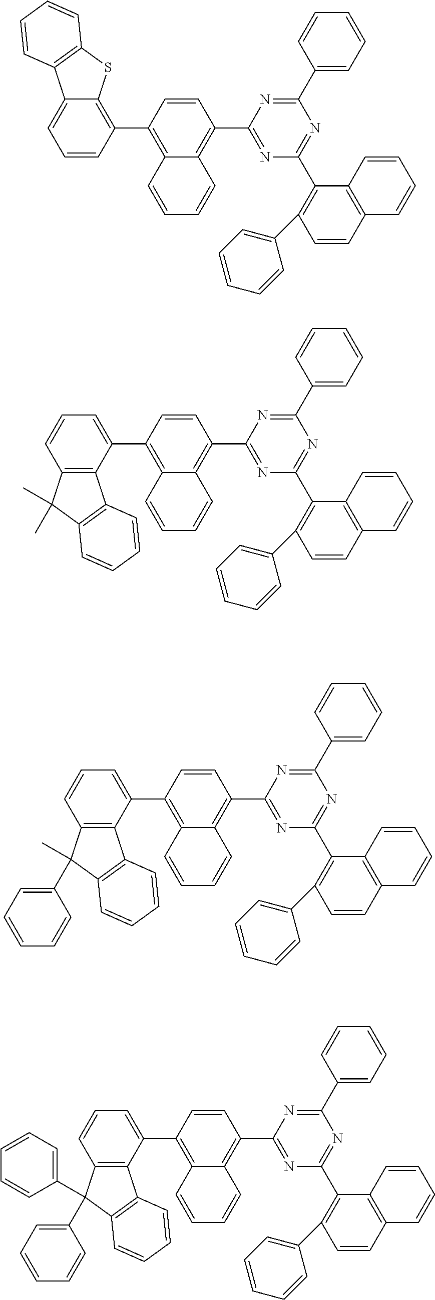 [SMILES: c1ccc(-c2nc(-c3c(-c4ccccc4)ccc4ccccc34)nc(-c3ccc(-c4cccc5c4sc4ccccc45)c4ccccc34)n2)cc1 ; CC1(C)c2ccccc2-c2c(-c3ccc(-c4nc(-c5ccccc5)nc(-c5c(-c6ccccc6)ccc6ccccc56)n4)c4ccccc34)cccc21 ; CC1(c2ccccc2)c2ccccc2-c2c(-c3ccc(-c4nc(-c5ccccc5)nc(-c5c(-c6ccccc6)ccc6ccccc56)n4)c4ccccc34)cccc21 ; c1ccc(-c2nc(-c3c(-c4ccccc4)ccc4ccccc34)nc(-c3ccc(-c4cccc5c4-c4ccccc4C5(c4ccccc4)c4ccccc4)c4ccccc34)n2)cc1]

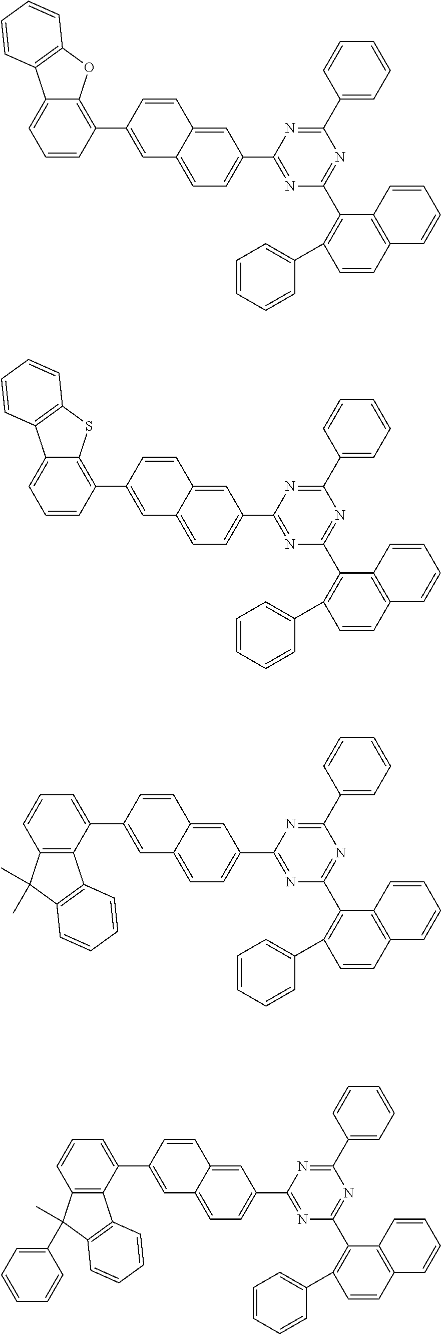 [SMILES: c1ccc(-c2nc(-c3ccc4cc(-c5cccc6c5oc5ccccc56)ccc4c3)nc(-c3c(-c4ccccc4)ccc4ccccc34)n2)cc1 ; c1ccc(-c2nc(-c3ccc4cc(-c5cccc6c5sc5ccccc56)ccc4c3)nc(-c3c(-c4ccccc4)ccc4ccccc34)n2)cc1 ; CC1(C)c2ccccc2-c2c(-c3ccc4cc(-c5nc(-c6ccccc6)nc(-c6c(-c7ccccc7)ccc7ccccc67)n5)ccc4c3)cccc21 ; CC1(c2ccccc2)c2ccccc2-c2c(-c3ccc4cc(-c5nc(-c6ccccc6)nc(-c6c(-c7ccccc7)ccc7ccccc67)n5)ccc4c3)cccc21]

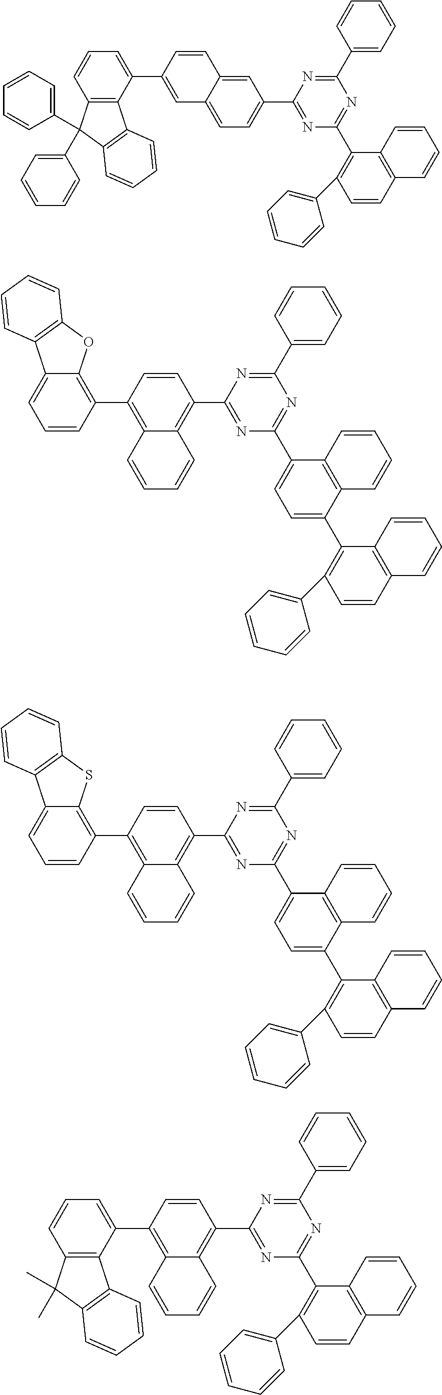

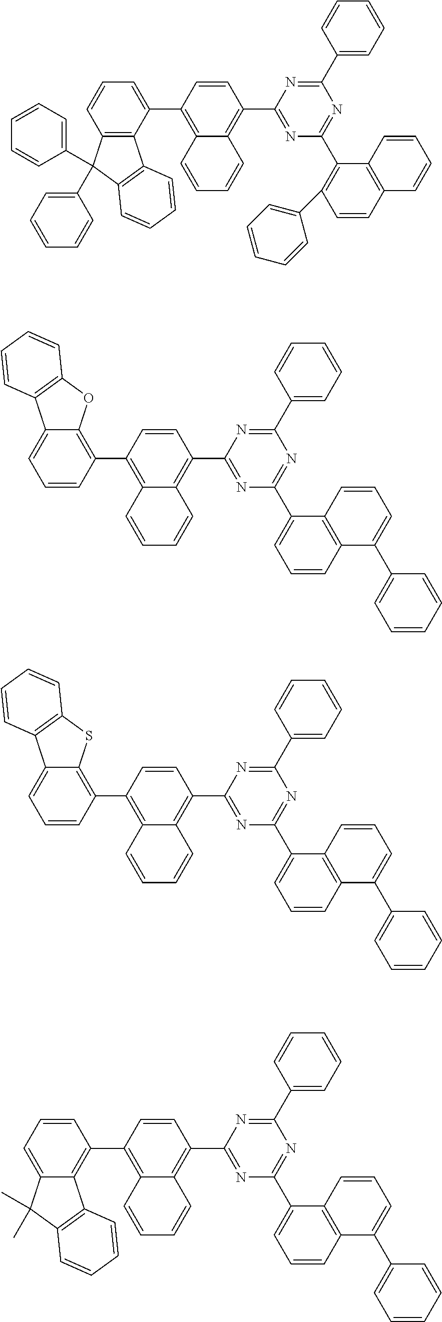 [SMILES: c1ccc(-c2nc(-c3c(-c4ccccc4)ccc4ccccc34)nc(-c3ccc(-c4cccc5c4-c4ccccc4C5(c4ccccc4)c4ccccc4)c4ccccc34)n2)cc1 ; c1ccc(-c2nc(-c3cccc4c(-c5ccccc5)cccc34)nc(-c3ccc(-c4cccc5c4oc4ccccc45)c4ccccc34)n2)cc1 ; c1ccc(-c2nc(-c3cccc4c(-c5ccccc5)cccc34)nc(-c3ccc(-c4cccc5c4sc4ccccc45)c4ccccc34)n2)cc1 ; CC1(C)c2ccccc2-c2c(-c3ccc(-c4nc(-c5ccccc5)nc(-c5cccc6c(-c7ccccc7)cccc56)n4)c4ccccc34)cccc21]

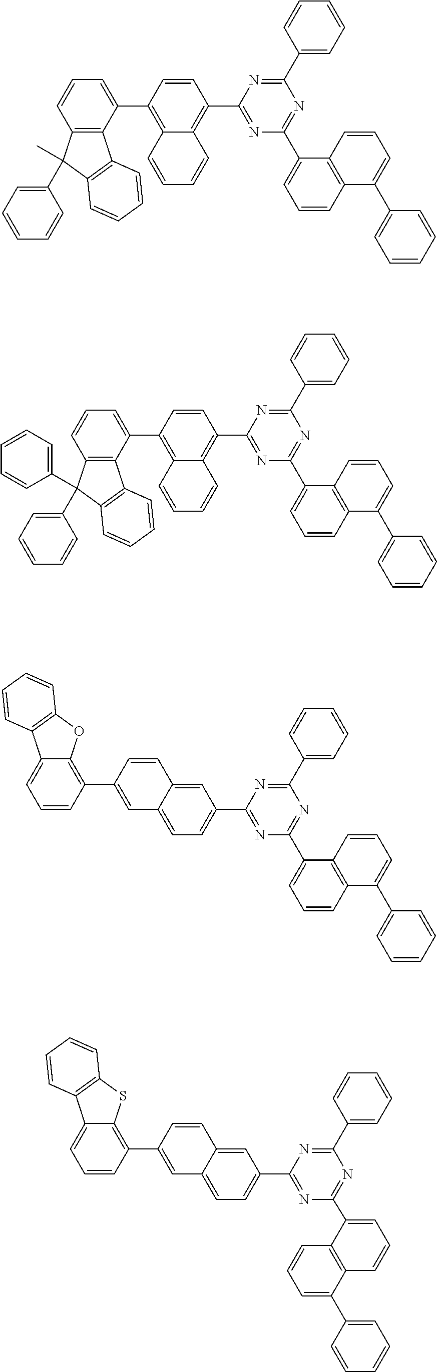 [SMILES: CC1(c2ccccc2)c2ccccc2-c2c(-c3ccc(-c4nc(-c5ccccc5)nc(-c5cccc6c(-c7ccccc7)cccc56)n4)c4ccccc34)cccc21 ; c1ccc(-c2nc(-c3cccc4c(-c5ccccc5)cccc34)nc(-c3ccc(-c4cccc5c4-c4ccccc4C5(c4ccccc4)c4ccccc4)c4ccccc34)n2)cc1 ; c1ccc(-c2nc(-c3ccc4cc(-c5cccc6c5oc5ccccc56)ccc4c3)nc(-c3cccc4c(-c5ccccc5)cccc34)n2)cc1 ; c1ccc(-c2nc(-c3ccc4cc(-c5cccc6c5sc5ccccc56)ccc4c3)nc(-c3cccc4c(-c5ccccc5)cccc34)n2)cc1]

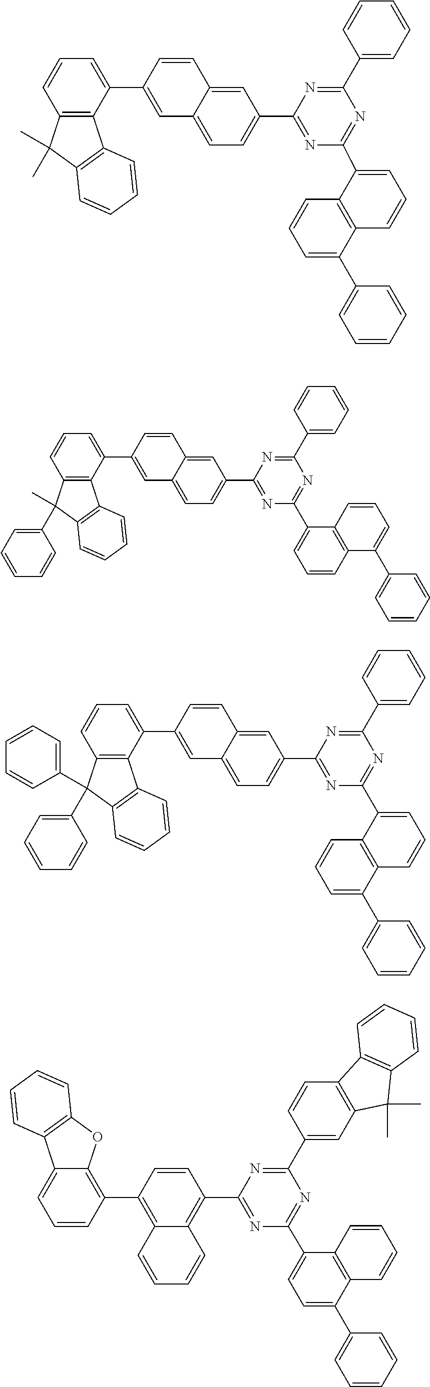

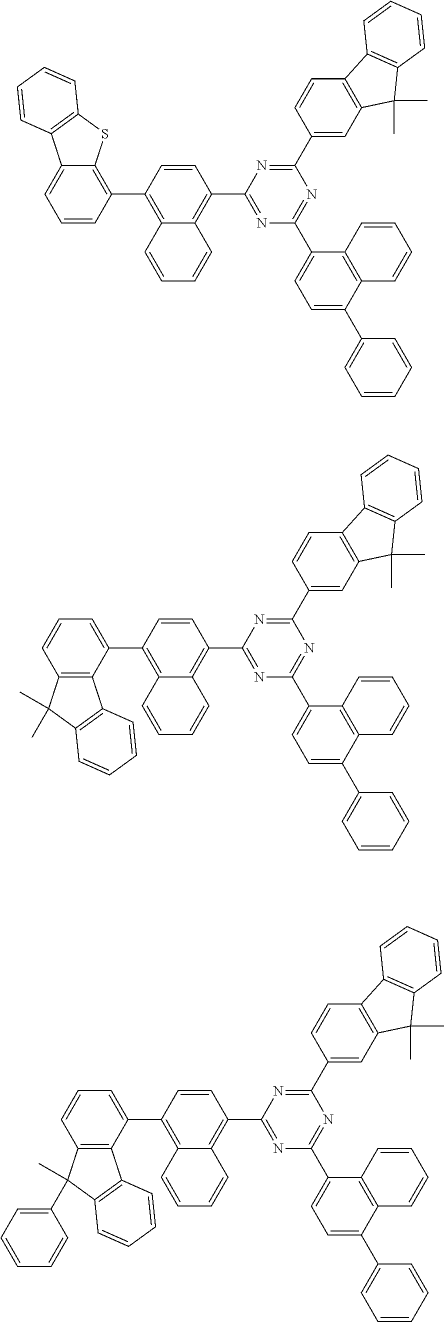

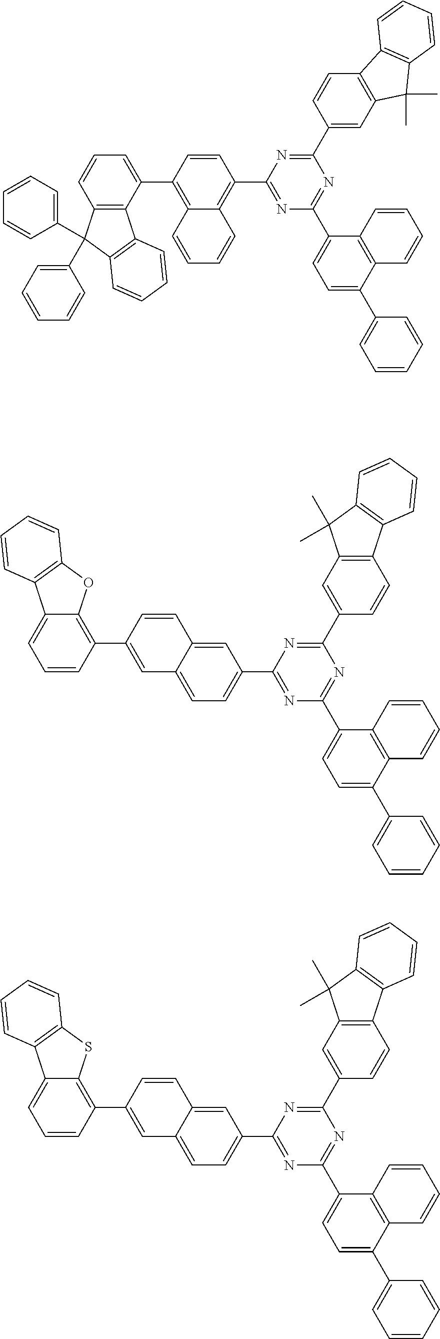 [SMILES: CC1(C)c2ccccc2-c2ccc(-c3nc(-c4ccc(-c5ccccc5)c5ccccc45)nc(-c4ccc(-c5cccc6c5-c5ccccc5C6(c5ccccc5)c5ccccc5)c5ccccc45)n3)cc21 ; CC1(C)c2ccccc2-c2ccc(-c3nc(-c4ccc5cc(-c6cccc7c6oc6ccccc67)ccc5c4)nc(-c4ccc(-c5ccccc5)c5ccccc45)n3)cc21 ; CC1(C)c2ccccc2-c2ccc(-c3nc(-c4ccc5cc(-c6cccc7c6sc6ccccc67)ccc5c4)nc(-c4ccc(-c5ccccc5)c5ccccc45)n3)cc21]

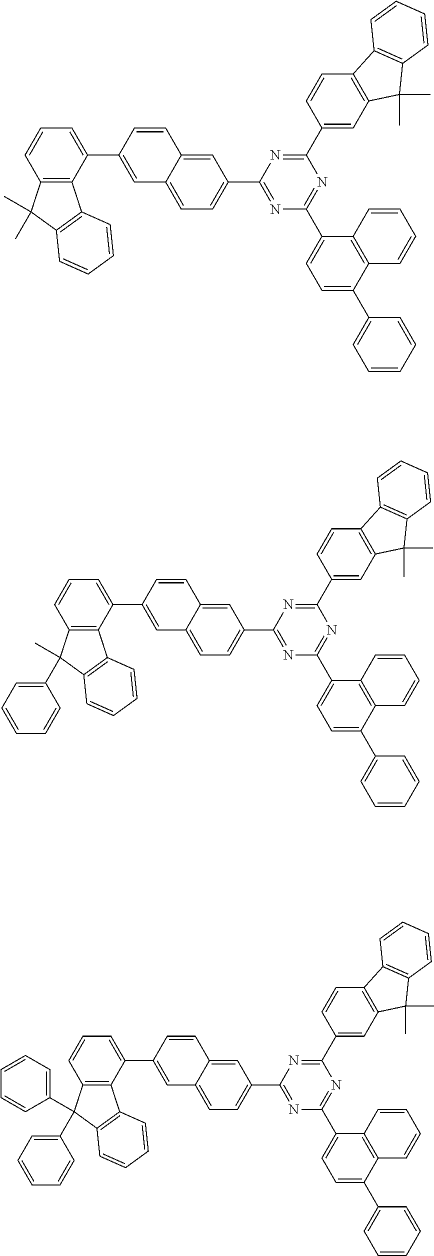

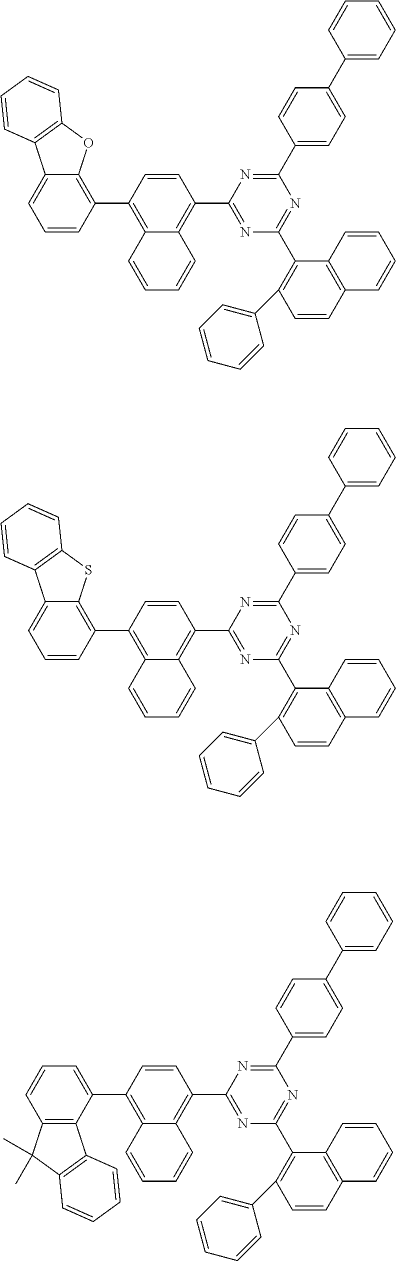 [SMILES: c1ccc(-c2ccc(-c3nc(-c4c(-c5ccccc5)ccc5ccccc45)nc(-c4ccc(-c5cccc6c5oc5ccccc56)c5ccccc45)n3)cc2)cc1 ; c1ccc(-c2ccc(-c3nc(-c4c(-c5ccccc5)ccc5ccccc45)nc(-c4ccc(-c5cccc6c5sc5ccccc56)c5ccccc45)n3)cc2)cc1 ; CC1(C)c2ccccc2-c2c(-c3ccc(-c4nc(-c5ccc(-c6ccccc6)cc5)nc(-c5c(-c6ccccc6)ccc6ccccc56)n4)c4ccccc34)cccc21]

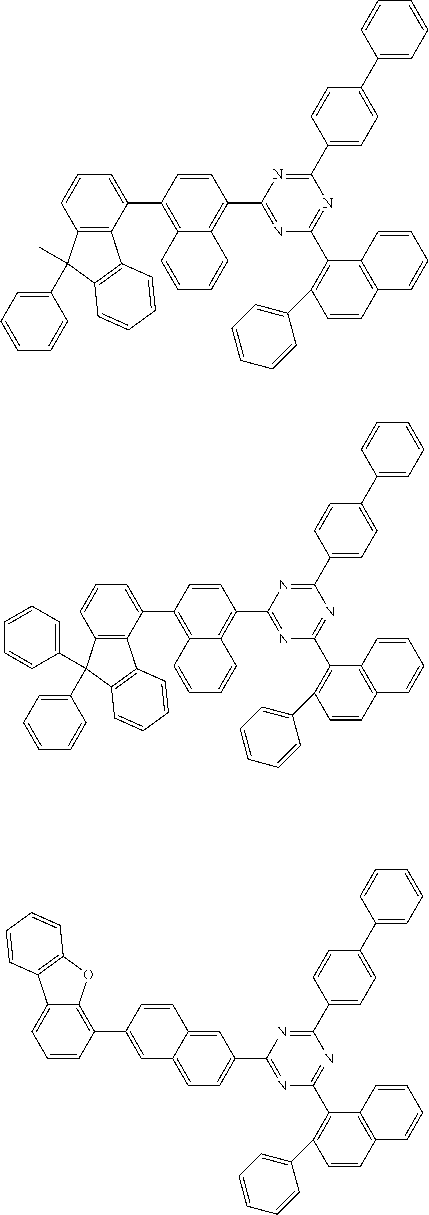 [SMILES: CC1(c2ccccc2)c2ccccc2-c2c(-c3ccc(-c4nc(-c5ccc(-c6ccccc6)cc5)nc(-c5c(-c6ccccc6)ccc6ccccc56)n4)c4ccccc34)cccc21 ; c1ccc(-c2ccc(-c3nc(-c4c(-c5ccccc5)ccc5ccccc45)nc(-c4ccc(-c5cccc6c5-c5ccccc5C6(c5ccccc5)c5ccccc5)c5ccccc45)n3)cc2)cc1 ; c1ccc(-c2ccc(-c3nc(-c4ccc5cc(-c6cccc7c6oc6ccccc67)ccc5c4)nc(-c4c(-c5ccccc5)ccc5ccccc45)n3)cc2)cc1]

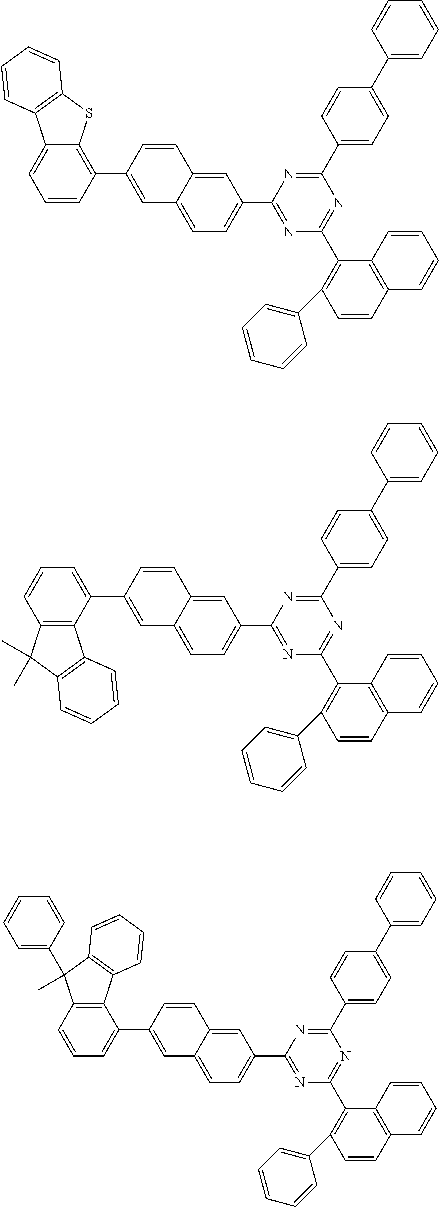 [SMILES: c1ccc(-c2ccc(-c3nc(-c4ccc5cc(-c6cccc7c6sc6ccccc67)ccc5c4)nc(-c4c(-c5ccccc5)ccc5ccccc45)n3)cc2)cc1 ; CC1(C)c2ccccc2-c2c(-c3ccc4cc(-c5nc(-c6ccc(-c7ccccc7)cc6)nc(-c6c(-c7ccccc7)ccc7ccccc67)n5)ccc4c3)cccc21 ; CC1(c2ccccc2)c2ccccc2-c2c(-c3ccc4cc(-c5nc(-c6ccc(-c7ccccc7)cc6)nc(-c6c(-c7ccccc7)ccc7ccccc67)n5)ccc4c3)cccc21]

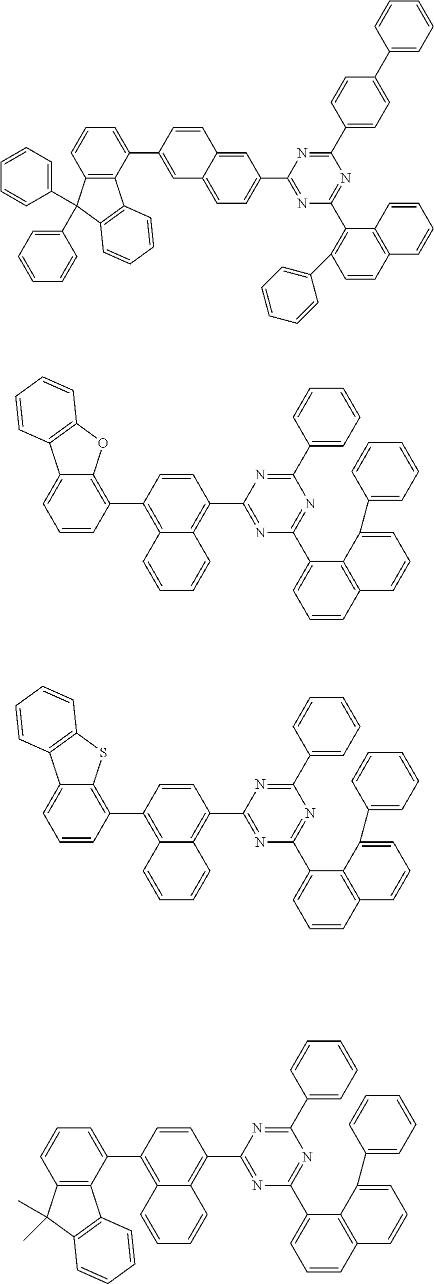 [SMILES: c1ccc(-c2ccc(-c3nc(-c4ccc5cc(-c6cccc7c6-c6ccccc6C7(c6ccccc6)c6ccccc6)ccc5c4)nc(-c4c(-c5ccccc5)ccc5ccccc45)n3)cc2)cc1 ; c1ccc(-c2nc(-c3ccc(-c4cccc5c4oc4ccccc45)c4ccccc34)nc(-c3cccc4cccc(-c5ccccc5)c34)n2)cc1 ; c1ccc(-c2nc(-c3ccc(-c4cccc5c4sc4ccccc45)c4ccccc34)nc(-c3cccc4cccc(-c5ccccc5)c34)n2)cc1 ; CC1(C)c2ccccc2-c2c(-c3ccc(-c4nc(-c5ccccc5)nc(-c5cccc6cccc(-c7ccccc7)c56)n4)c4ccccc34)cccc21]

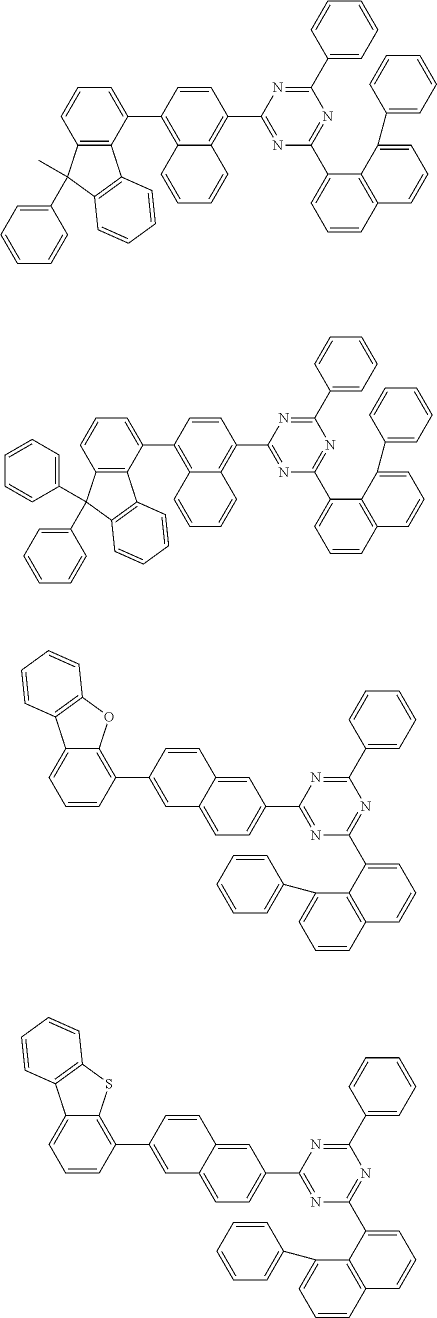 [SMILES: CC1(c2ccccc2)c2ccccc2-c2c(-c3ccc(-c4nc(-c5ccccc5)nc(-c5cccc6cccc(-c7ccccc7)c56)n4)c4ccccc34)cccc21 ; c1ccc(-c2nc(-c3ccc(-c4cccc5c4-c4ccccc4C5(c4ccccc4)c4ccccc4)c4ccccc34)nc(-c3cccc4cccc(-c5ccccc5)c34)n2)cc1 ; c1ccc(-c2nc(-c3ccc4cc(-c5cccc6c5oc5ccccc56)ccc4c3)nc(-c3cccc4cccc(-c5ccccc5)c34)n2)cc1 ; c1ccc(-c2nc(-c3ccc4cc(-c5cccc6c5sc5ccccc56)ccc4c3)nc(-c3cccc4cccc(-c5ccccc5)c34)n2)cc1]

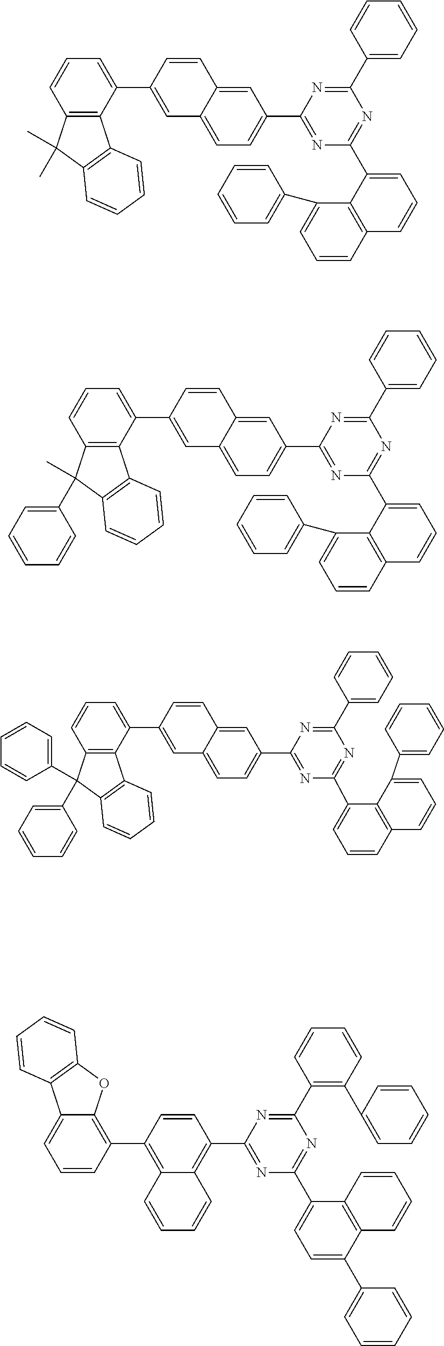 [SMILES: CC1(C)c2ccccc2-c2c(-c3ccc4cc(-c5nc(-c6ccccc6)nc(-c6cccc7cccc(-c8ccccc8)c67)n5)ccc4c3)cccc21 ; CC1(c2ccccc2)c2ccccc2-c2c(-c3ccc4cc(-c5nc(-c6ccccc6)nc(-c6cccc7cccc(-c8ccccc8)c67)n5)ccc4c3)cccc21 ; c1ccc(-c2nc(-c3ccc4cc(-c5cccc6c5-c5ccccc5C6(c5ccccc5)c5ccccc5)ccc4c3)nc(-c3cccc4cccc(-c5ccccc5)c34)n2)cc1 ; c1ccc(-c2ccccc2-c2nc(-c3ccc(-c4ccccc4)c4ccccc34)nc(-c3ccc(-c4cccc5c4oc4ccccc45)c4ccccc34)n2)cc1]

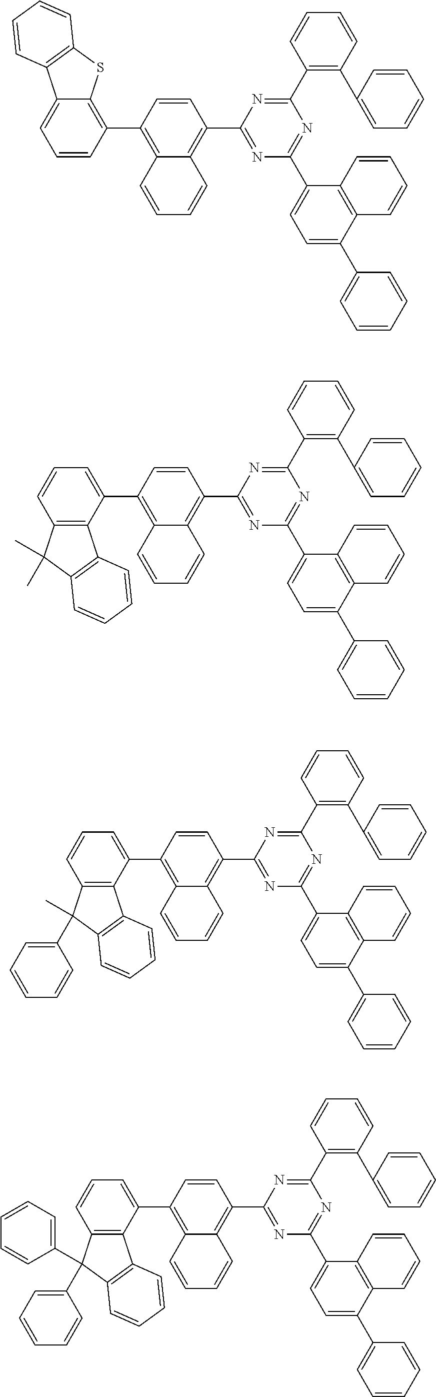 [SMILES: c1ccc(-c2ccccc2-c2nc(-c3ccc(-c4ccccc4)c4ccccc34)nc(-c3ccc(-c4cccc5c4sc4ccccc45)c4ccccc34)n2)cc1 ; CC1(C)c2ccccc2-c2c(-c3ccc(-c4nc(-c5ccccc5-c5ccccc5)nc(-c5ccc(-c6ccccc6)c6ccccc56)n4)c4ccccc34)cccc21 ; CC1(c2ccccc2)c2ccccc2-c2c(-c3ccc(-c4nc(-c5ccccc5-c5ccccc5)nc(-c5ccc(-c6ccccc6)c6ccccc56)n4)c4ccccc34)cccc21 ; c1ccc(-c2ccccc2-c2nc(-c3ccc(-c4ccccc4)c4ccccc34)nc(-c3ccc(-c4cccc5c4-c4ccccc4C5(c4ccccc4)c4ccccc4)c4ccccc34)n2)cc1]

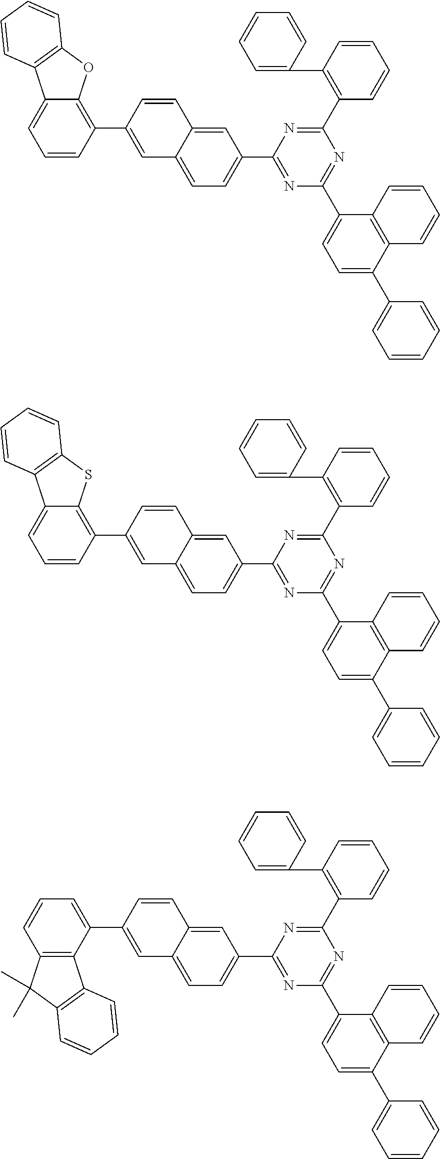 [SMILES: c1ccc(-c2ccccc2-c2nc(-c3ccc4cc(-c5cccc6c5oc5ccccc56)ccc4c3)nc(-c3ccc(-c4ccccc4)c4ccccc34)n2)cc1 ; c1ccc(-c2ccccc2-c2nc(-c3ccc4cc(-c5cccc6c5sc5ccccc56)ccc4c3)nc(-c3ccc(-c4ccccc4)c4ccccc34)n2)cc1 ; CC1(C)c2ccccc2-c2c(-c3ccc4cc(-c5nc(-c6ccccc6-c6ccccc6)nc(-c6ccc(-c7ccccc7)c7ccccc67)n5)ccc4c3)cccc21]

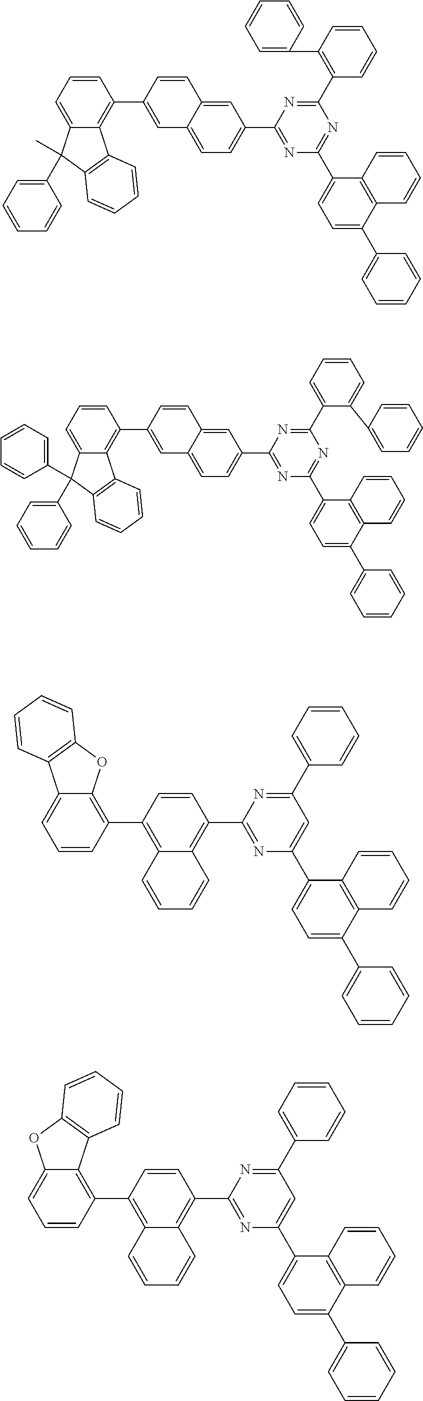 [SMILES: CC1(c2ccccc2)c2ccccc2-c2c(-c3ccc4cc(-c5nc(-c6ccccc6-c6ccccc6)nc(-c6ccc(-c7ccccc7)c7ccccc67)n5)ccc4c3)cccc21 ; c1ccc(-c2ccccc2-c2nc(-c3ccc4cc(-c5cccc6c5-c5ccccc5C6(c5ccccc5)c5ccccc5)ccc4c3)nc(-c3ccc(-c4ccccc4)c4ccccc34)n2)cc1 ; c1ccc(-c2cc(-c3ccc(-c4ccccc4)c4ccccc34)nc(-c3ccc(-c4cccc5c4oc4ccccc45)c4ccccc34)n2)cc1 ; c1ccc(-c2cc(-c3ccc(-c4ccccc4)c4ccccc34)nc(-c3ccc(-c4cccc5oc6ccccc6c45)c4ccccc34)n2)cc1]

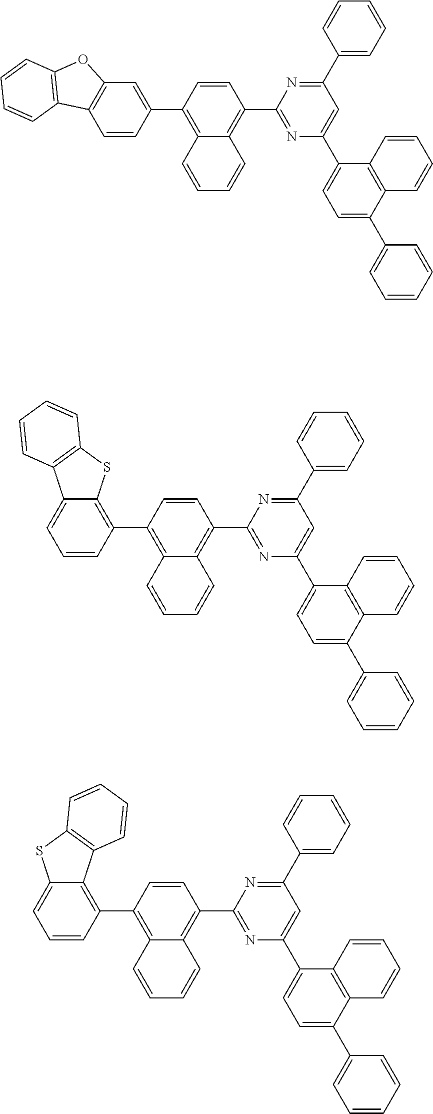 [SMILES: c1ccc(-c2cc(-c3ccc(-c4ccccc4)c4ccccc34)nc(-c3ccc(-c4ccc5c(c4)oc4ccccc45)c4ccccc34)n2)cc1 ; c1ccc(-c2cc(-c3ccc(-c4ccccc4)c4ccccc34)nc(-c3ccc(-c4cccc5c4sc4ccccc45)c4ccccc34)n2)cc1 ; c1ccc(-c2cc(-c3ccc(-c4ccccc4)c4ccccc34)nc(-c3ccc(-c4cccc5sc6ccccc6c45)c4ccccc34)n2)cc1]

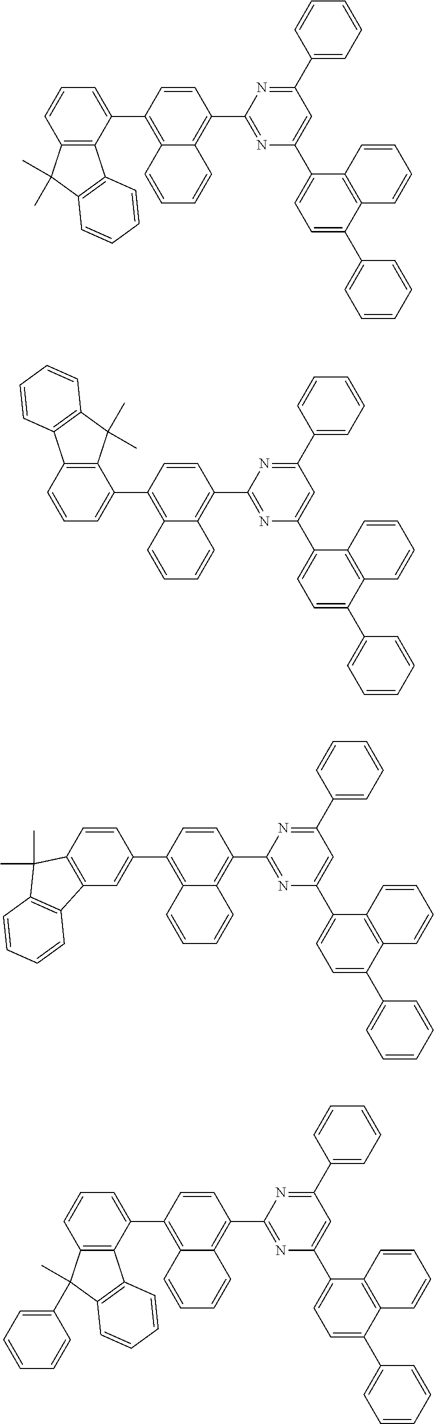 [SMILES: CC1(C)c2ccccc2-c2c(-c3ccc(-c4nc(-c5ccccc5)cc(-c5ccc(-c6ccccc6)c6ccccc56)n4)c4ccccc34)cccc21 ; CC1(C)c2ccccc2-c2cccc(-c3ccc(-c4nc(-c5ccccc5)cc(-c5ccc(-c6ccccc6)c6ccccc56)n4)c4ccccc34)c21 ; CC1(C)c2ccccc2-c2cc(-c3ccc(-c4nc(-c5ccccc5)cc(-c5ccc(-c6ccccc6)c6ccccc56)n4)c4ccccc34)ccc21 ; CC1(c2ccccc2)c2ccccc2-c2c(-c3ccc(-c4nc(-c5ccccc5)cc(-c5ccc(-c6ccccc6)c6ccccc56)n4)c4ccccc34)cccc21]

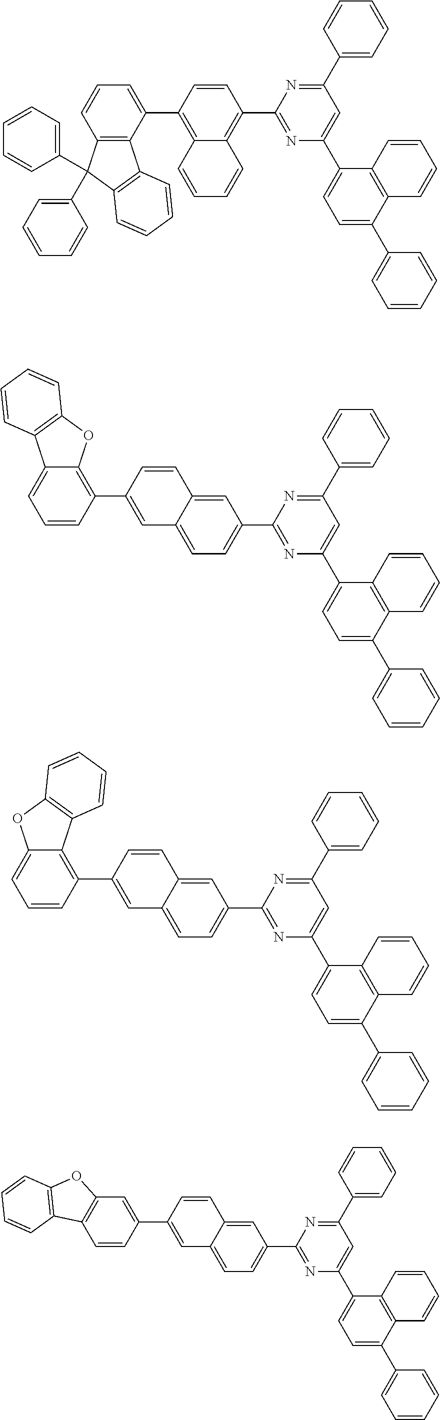

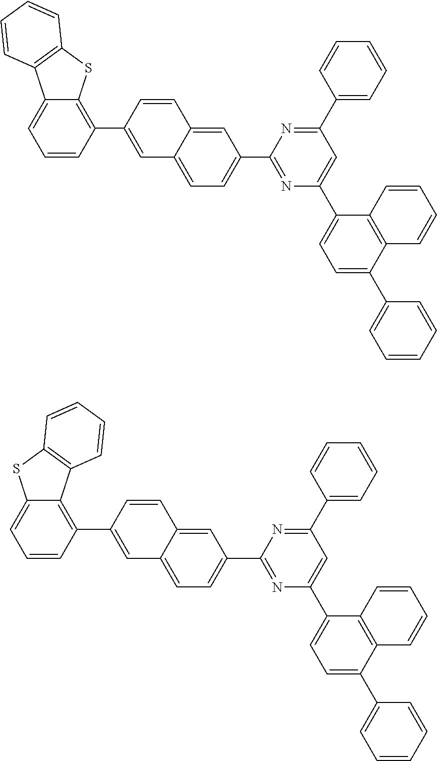 [SMILES: c1ccc(-c2cc(-c3ccc(-c4ccccc4)c4ccccc34)nc(-c3ccc4cc(-c5cccc6c5sc5ccccc56)ccc4c3)n2)cc1 ; c1ccc(-c2cc(-c3ccc(-c4ccccc4)c4ccccc34)nc(-c3ccc4cc(-c5cccc6sc7ccccc7c56)ccc4c3)n2)cc1]

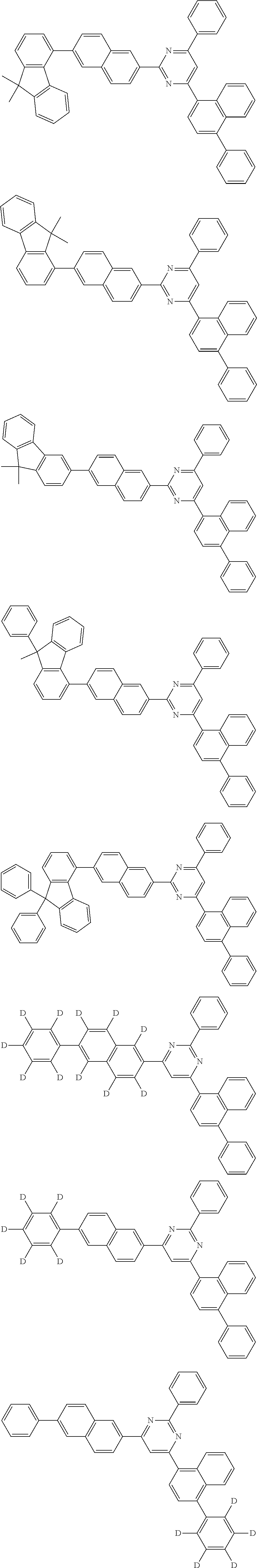

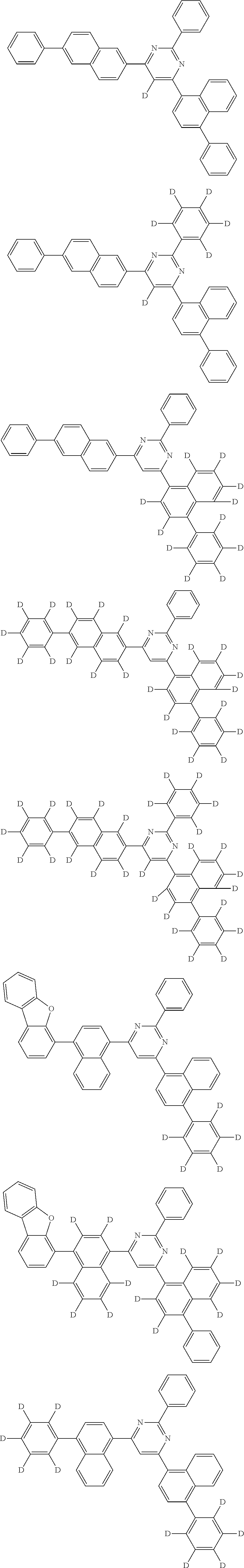

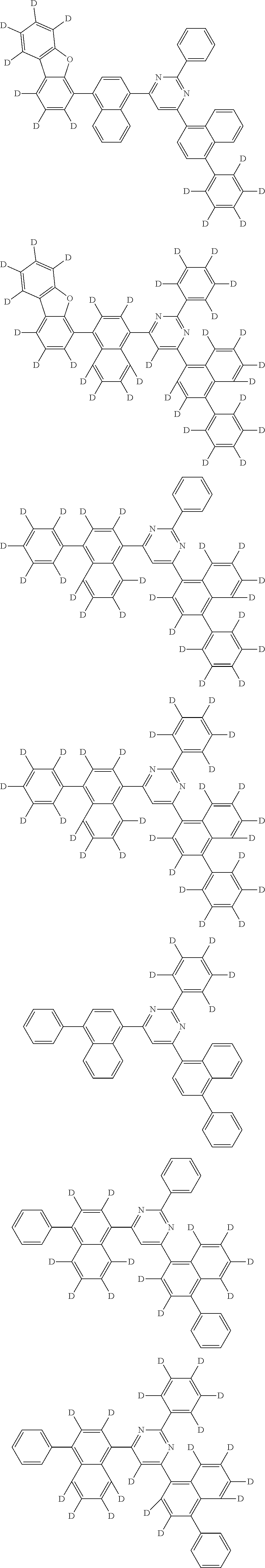

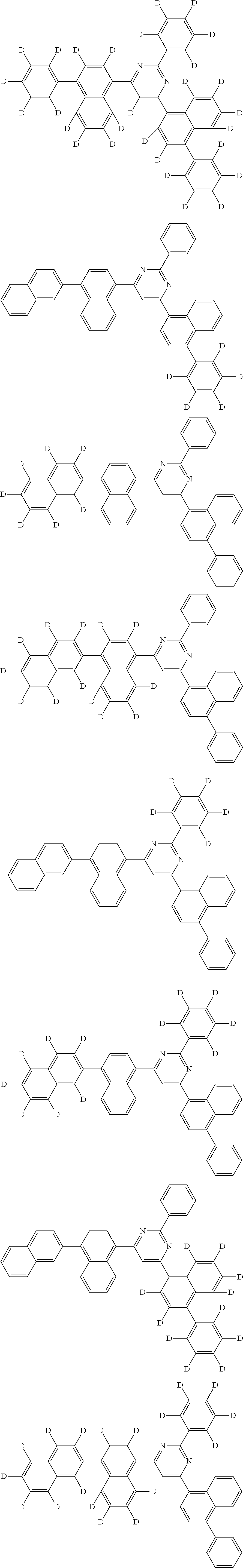

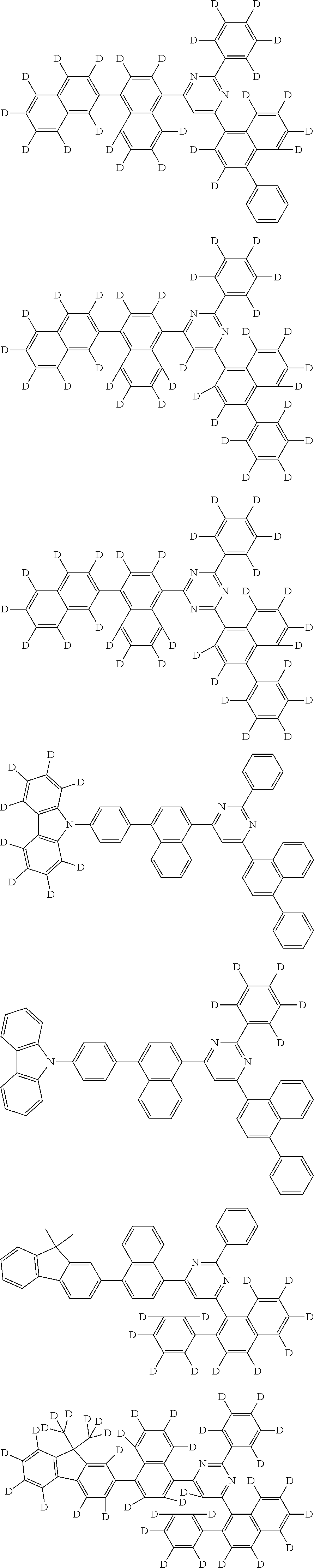

### [Material for Organic Electroluminescence Device]

The compound according to an aspect of the present invention is useful as a material for an organic EL device, and for example, is useful as a material used for an electron-transporting region of an organic EL device.

### [Organic Electroluminescence Device]

An organic EL device according to an aspect of the present invention is described.

The organic EL device according to an aspect of the present invention includes a cathode, an anode, and one or more organic layers arranged between the cathode and the anode, wherein at least one layer of the one or more organic layers includes the compound (compound represented by the formula (1)) according to an aspect of the present invention.

In one embodiment, the organic EL device according to an aspect of the present invention includes an anode, an emitting layer, an electron-transporting region, and a cathode in this order, wherein the electron-transporting region includes the compound (compound represented by the formula (1)) according to an aspect of the present invention.

In one embodiment, the electron-transporting region includes at least a first layer and a second layer in this order from the emitting layer side, and the first layer includes the compound.

In one embodiment, the organic EL device according to an aspect of the present invention includes a hole-transporting region between the anode and the emitting layer.

As a representative device configuration of the organic EL device, structures in which the following structures are stacked on a substrate can be given as examples:
(1) an anode/an emitting layer/an electron-transporting region/a cathode,
(2) an anode/a hole-transporting region/an emitting layer/an electron-transporting region/a cathode,
   wherein “/” indicates that the layers are stacked adjacent to each other.

The electron-transporting region is generally composed of one or more layers selected from an electron-injecting layer and an electron-transporting layer. The hole-transporting region is generally composed of one or more layers selected from a hole-injecting layer and a hole-transporting layer.

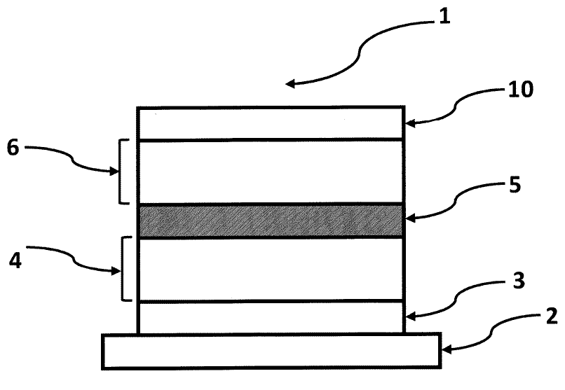

The schematic configuration of the organic EL device according to an aspect of the present invention is described with reference to FIG. 1.

An organic EL device 1 according to an aspect of the present invention includes a substrate 2, an anode 3, an emitting layer 5, a cathode 10, a hole-transporting region 4 between the anode 3 and the emitting layer 5, and an electron-transporting region 6 between the emitting layer 5 and the cathode 10.

Members which can be used in the organic EL device according to an aspect of the present invention, materials for forming each layer, other than the above-mentioned compounds, and the like are described below.

### (Substrate)

The substrate is used as a support of an emitting device. As the substrate, glass, quartz, plastic, or the like can be used, for example. In addition, a flexible substrate may be used. The term “flexible substrate” means a bendable (flexible) substrate, and a specific example thereof is a plastic substrate formed of polycarbonate or polyvinyl chloride.

### (Anode)

For the anode formed on the substrate, metals, alloys, electrically conductive compounds, mixtures thereof, and the like which have large work function (specifically 4.0 eV or more) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, and graphene. In addition thereto, specific examples thereof include gold (Au), platinum (Pt), and a nitride of a metallic material (e.g., titanium nitride).

### (Hole-Injecting Layer)

The hole-injecting layer is a layer containing a substance having high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (e.g., an oligomer, a dendrimer, or a polymer), or the like can be used.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound, such as poly(N-vinylcarbazole) (abbreviation: PVK) or poly(4-vinyltriphenylamine) (abbreviation: PVTPA), can also be used. Provided that a substance other than the above-described substances may be used as long as the substance has higher hole-transporting property than electron-transporting property. The layer containing the substance having high hole-transporting property may be not only a single layer, but also layers in which two or more layers formed of the above-described substances are stacked.

### (Guest (Dopant) Material of Emitting Layer)

The emitting layer is a layer containing a substance having high luminous property, and various materials can be used. For example, as the substance having high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives, and the like can be used.

As a blue phosphorescent emitting material which can be used for the emitting layer, metal complexes, such as iridium complexes, osmium complexes, and platinum complexes, are used. As a green phosphorescent emitting material which can be used for the emitting layer, iridium complexes and the like are used. As a red phosphorescent emitting material which can be used for the emitting layer, metal complexes, such as iridium complexes, platinum complexes, terbium complexes, and europium complexes, are used.

### (Host Material for Emitting Layer)

The emitting layer may have a constitution in which the substance having high emitting property (guest material) is dispersed in another substance (host material). As a substance for dispersing the substance having high emitting property, a variety of substances can be used, and it is preferred to use a substance having a higher lowest unoccupied molecular orbital level (LUMO level) and a lower highest occupied molecular orbital level (HOMO level) than a substance having high emitting property.

As a substance (host material) for dispersing the substance having high emitting property, 1) a metal complex, such as an aluminum complex, a beryllium complex, or a zinc complex, 2) a heterocyclic compound, such as an oxadiazole derivative, a benzimidazole derivative, or a phenanthroline derivative, 3) a fused aromatic compound, such as a carbazole derivative, an anthracene derivative, a phenanthrene derivative, a pyrene derivative, or a chrysene derivative, or 4) an aromatic amine compound, such as a triarylamine derivative or a fused polycyclic aromatic amine derivative, is used.

### (Electron-Transporting Layer)

The electron-transporting layer is a layer containing a substance having high electron-transporting property. For the electron-transporting layer, 1) a metal complex, such as an aluminum complex, a beryllium complex, or a zinc complex, 2) a heteroaromatic complex, such as an imidazole derivative, a benzimidazole derivative, an azine derivative, a carbazole derivative, or a phenanthroline derivative, or 3) a polymer compound can be used.

In one aspect of the present invention, the electron-transporting layer may include or may not include another substance described above in addition to the compound (compound represented by the formula (1)) according to an aspect of the present invention.

### (Electron-Injecting Layer)

The electron-injecting layer is a layer containing a substance having high electron-injecting property. For the electron-injecting layer, lithium (Li), ytterbium (Yb), lithium fluoride (LiF), cesium fluoride (CsF), calcium fluoride (CaF₂), a metal complex compound such as 8-hydroxyquinolinolato-lithium (Liq), or an alkali metal, an alkaline earth metal, or a compound thereof, such as lithium oxide (LiOₓ), can be used.

### (Cathode)

For the cathode, metals, alloys, electrically conductive compounds, mixtures thereof, and the like which have small work function (specifically 3.8 eV or less) are preferably used. Specific examples of such a cathode material include: elements each belonging to Group 1 or Group 2 of the Periodic Table of the Elements, i.e., alkali metals, such as lithium (Li) and cesium (Cs), and alkaline earth metals, such as magnesium (Mg), calcium (Ca), and strontium (Sr), and alloys containing these elements (e.g., MgAg and AILi); and rare earth metals, such as europium (Eu) and ytterbium (Yb), and alloys containing these elements.

The cathode is usually formed by a vacuum deposition method or a sputtering method. In addition, when silver paste or the like is used, a coating method, an inkjet method, or the like can be used.

In addition, when the electron-injecting layer is provided, the cathode can be formed using various conductive materials, such as aluminum, silver, ITO, graphene, and indium oxide-tin oxide containing silicon or silicon oxide, regardless of the work function value.

In the organic EL device according to an aspect of the present invention, the thickness of each layer is not particularly limited, but normally preferably falls within the range of from several nm to 1 μm generally in order to suppress defects such as pinholes, to suppress applied voltages to be low, and to improve luminous efficiency.

### (Method of Fabricating Organic EL Device)

In the organic EL device according to an aspect of the present invention, the method of forming each layer is not particularly limited. A conventionally-known method of forming each layer, such as a vacuum deposition process and a spin coating process, can be used. Each layer such as the emitting layer can be formed by a known method, such as a vacuum deposition process, a molecular beam deposition process (MBE process), or an application process, such as a dipping process, a spin coating process, a casting process, a bar coating process, or a roll coating process, using a solution prepared by dissolving the material in a solvent.

### [Electronic Apparatus]

An electronic apparatus according to an aspect of the present invention is characterized by including the organic EL device according to an aspect of the present invention.

Specific examples of the electronic apparatus include: display components such as an organic EL panel module; display devices, such as a television, a cellular phone, and a personal computer; and emitting devices, such as a light and a vehicular lamp.

### EXAMPLES

### <Compound>

Compounds represented by the formula (1) used in the fabrication of organic EL devices of Examples are shown below.

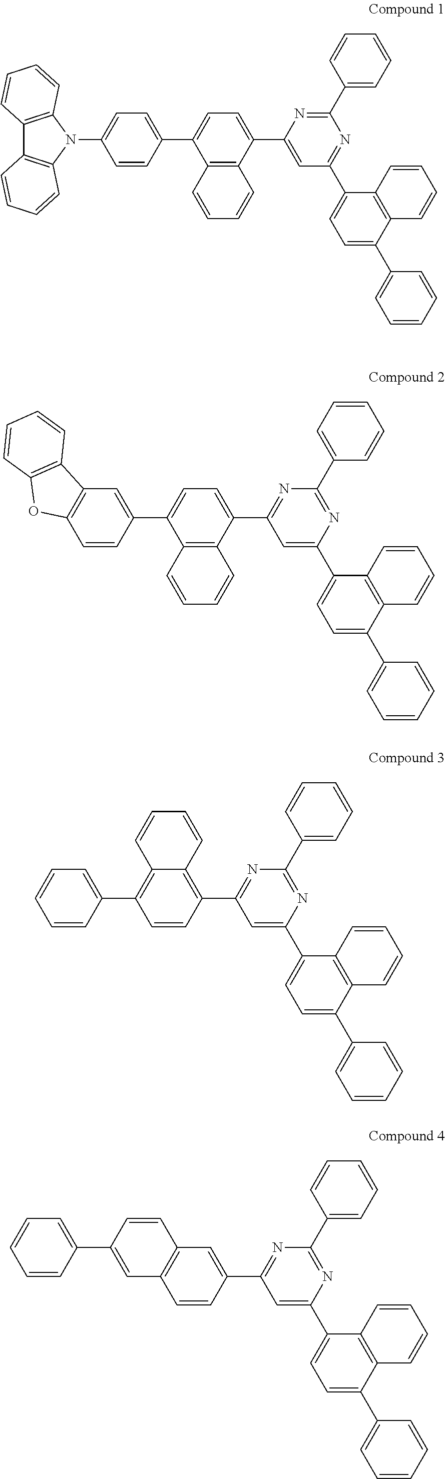 [SMILES: c1ccc(-c2nc(-c3ccc(-c4ccccc4)c4ccccc34)cc(-c3ccc(-c4ccc(-n5c6ccccc6c6ccccc65)cc4)c4ccccc34)n2)cc1 ; c1ccc(-c2nc(-c3ccc(-c4ccccc4)c4ccccc34)cc(-c3ccc(-c4ccc5oc6ccccc6c5c4)c4ccccc34)n2)cc1 ; c1ccc(-c2nc(-c3ccc(-c4ccccc4)c4ccccc34)cc(-c3ccc(-c4ccccc4)c4ccccc34)n2)cc1 ; c1ccc(-c2ccc3cc(-c4cc(-c5ccc(-c6ccccc6)c6ccccc56)nc(-c5ccccc5)n4)ccc3c2)cc1]

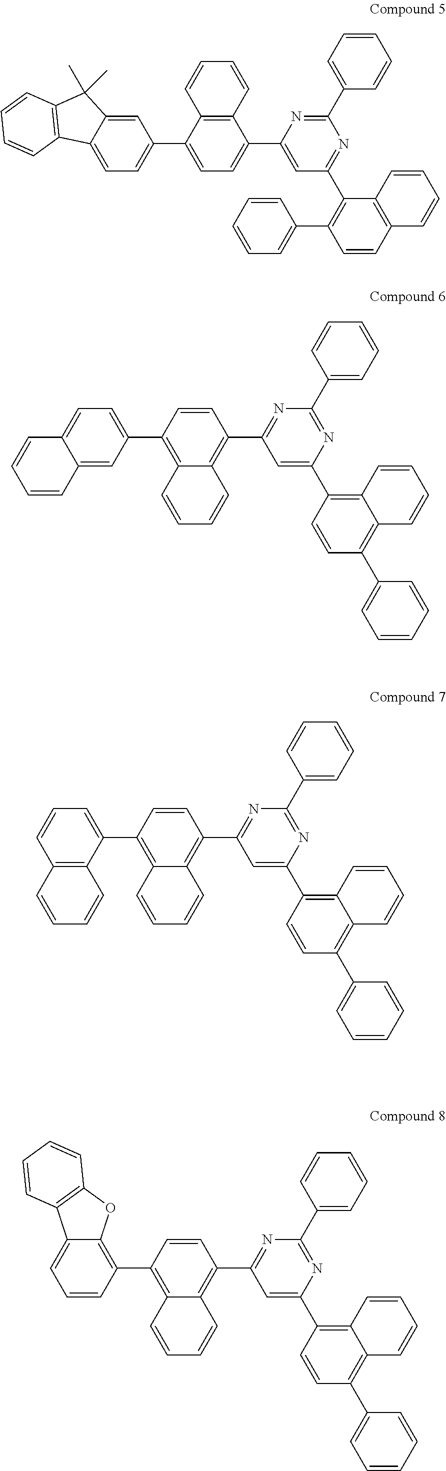 [SMILES: CC1(C)c2ccccc2-c2ccc(-c3ccc(-c4cc(-c5c(-c6ccccc6)ccc6ccccc56)nc(-c5ccccc5)n4)c4ccccc34)cc21 ; c1ccc(-c2nc(-c3ccc(-c4ccccc4)c4ccccc34)cc(-c3ccc(-c4ccc5ccccc5c4)c4ccccc34)n2)cc1 ; c1ccc(-c2nc(-c3ccc(-c4ccccc4)c4ccccc34)cc(-c3ccc(-c4cccc5ccccc45)c4ccccc34)n2)cc1 ; c1ccc(-c2nc(-c3ccc(-c4ccccc4)c4ccccc34)cc(-c3ccc(-c4cccc5c4oc4ccccc45)c4ccccc34)n2)cc1]

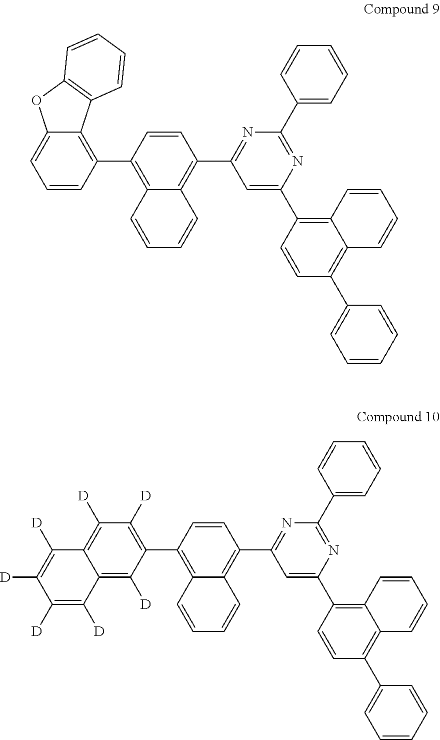 [SMILES: c1ccc(-c2nc(-c3ccc(-c4ccccc4)c4ccccc34)cc(-c3ccc(-c4cccc5oc6ccccc6c45)c4ccccc34)n2)cc1 ; [2H]c1c([2H])c([2H])c2c([2H])c(-c3ccc(-c4cc(-c5ccc(-c6ccccc6)c6ccccc56)nc(-c5ccccc5)n4)c4ccccc34)c([2H])c([2H])c2c1[2H]]

Comparative compounds used in the fabrication of organic EL devices of Comparative Examples are shown below.

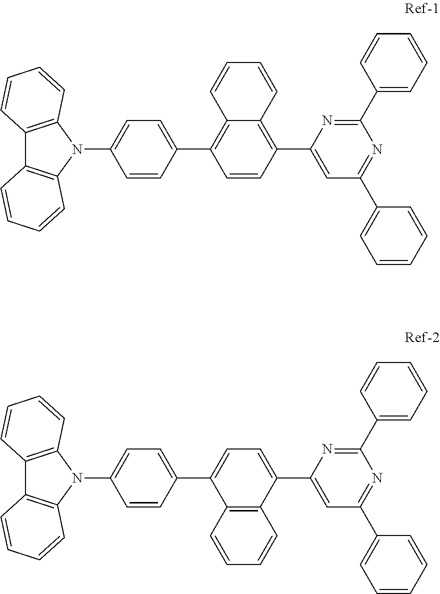 [SMILES: c1ccc(-c2cc(-c3ccc(-c4ccc(-n5c6ccccc6c6ccccc65)cc4)c4ccccc34)nc(-c3ccccc3)n2)cc1 ; c1ccc(-c2cc(-c3ccc(-c4ccc(-n5c6ccccc6c6ccccc65)cc4)c4ccccc34)nc(-c3ccccc3)n2)cc1]

Other compound structures used in the fabrication of the organic EL devices of Examples and Comparative Examples are shown below.

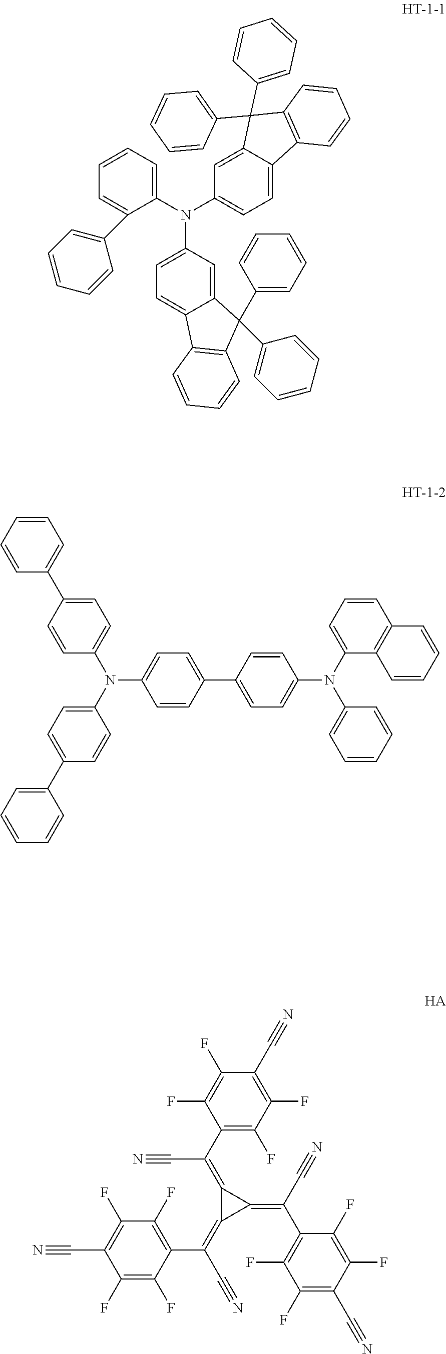 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

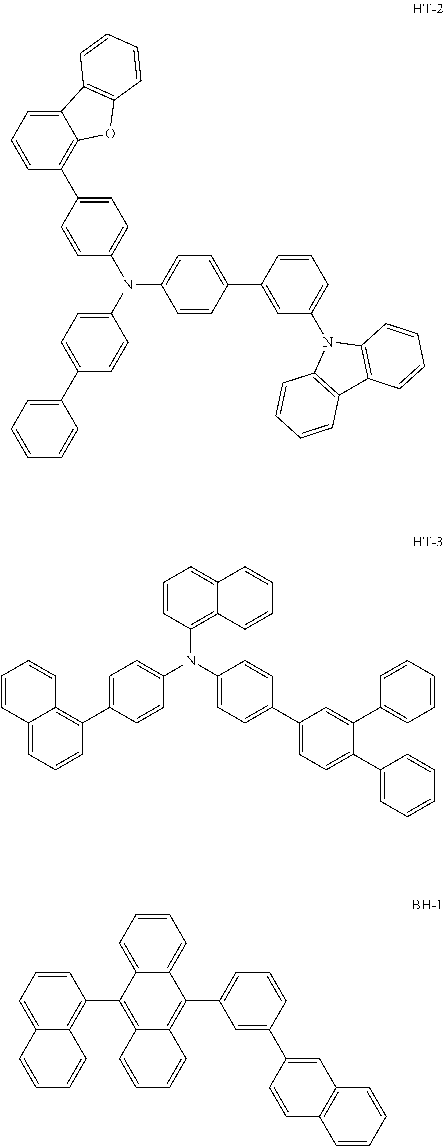

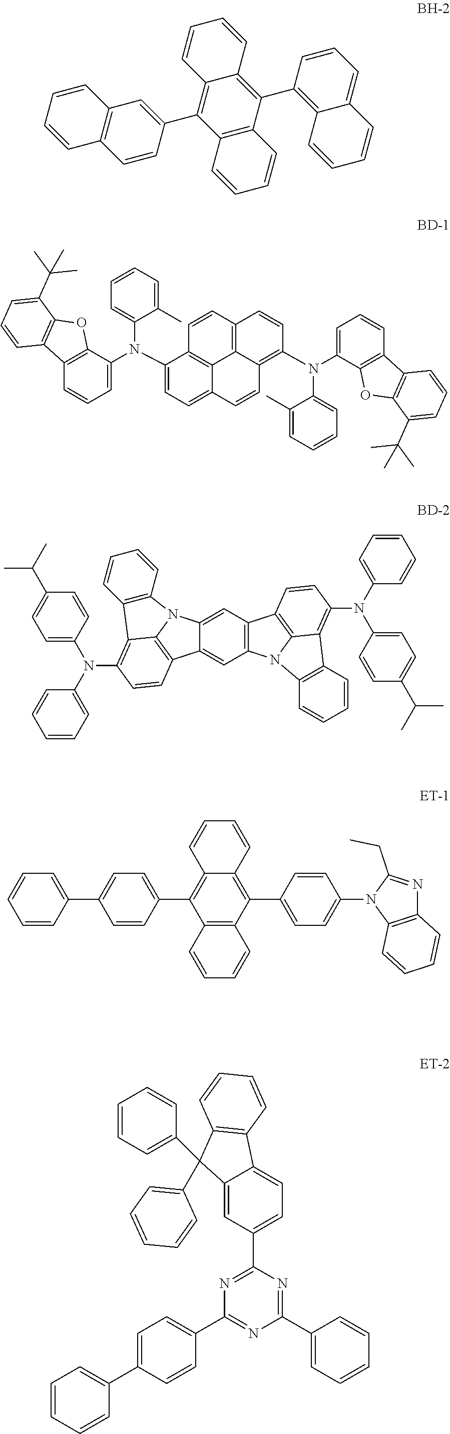

### <Fabrication of Organic EL Device>

An organic EL device was fabricated as described below.

### Example 1

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and was then subjected to UV-ozone cleaning for 30 minutes. The thickness of the ITO was set to 130 nm.

The glass substrate with the transparent electrode after the cleaning was mounted onto the substrate holder of a vacuum vapor deposition apparatus. First, the compounds HT-1-1 and HA were co-deposited on the surface of the substrate on the side having formed thereon the transparent electrode so that the compounds covered the transparent electrode, and so that the proportion of the compound HA became 3% by mass. Thus, a first hole-transporting layer having a thickness of 10 nm was formed.

The compound HT-1-1 was deposited on the first hole-transporting layer to form a second hole-transporting layer having a thickness of 80 nm.

The compound HT-2 was deposited on the second hole-transporting layer to form a third hole-transporting layer (also referred to as “electron barrier layer”) having a thickness of 10 nm.

The compound BH-1 (host material) and the compound BD-1 (dopant material) were co-deposited on the third hole-transporting layer so that the proportion of the compound BD-1 became 4% by mass. Thus, an emitting layer having a thickness of 25 nm was formed.

The compound 1 was deposited on the emitting layer to form a first electron-transporting layer having a thickness of 10 nm.

The compound ET-1 was deposited on the first electron-transporting layer to form a second electron-transporting layer having a thickness of 15 nm.

Lithium fluoride (LiF) was deposited on the second electron-transporting layer to form an electron-injecting layer having a thickness of 1 nm.

Metal Al was deposited on the electron-injecting layer to form a cathode having a thickness of 50 nm.

The device configuration of the organic EL device of Example 1 is schematically described as follows.

$$ITO ( 130 ) / HT - 1 - 1 : HA ( 10 : 3 \% ) / HT - 1 - 1 ( 80 ) / HT - 2 ( 10 ) / BH - 1 : BD - 1 ( 25 : 4 \% ) / compound 1 ( 15 ) : LiF ( 1 ) / Al ( 50 )$$

The numerical values in parentheses indicate thickness (unit: nm). In addition, the numerical values represented by percent in parentheses each indicate the proportion (% by mass) of the latter compound in the corresponding layer.

### Examples 2 to 5

Organic EL devices were each fabricated in the same manner as in Example 1 except that in the formation of the first electron-transporting layer, a compound shown in Table 1 was used.

### Comparative Example 1

An organic EL device was fabricated in the same manner as in Example 1 except that in the formation of the first electron-transporting layer, the compound Ref-1 was used.

### Comparative Example 2

An organic EL device was fabricated in the same manner as in Example 1 except that in the formation of the first electron-transporting layer, the compound Ref-2 was used.

### <Evaluation of Organic EL Device>

The organic EL devices fabricated in Examples 1 to 5, and Comparative Examples 1 and 2 were evaluated for their device lifetimes as described below. The results are shown in Table 1.

### Device Lifetime

Regarding the obtained organic EL device, a voltage was applied to the organic EL device at room temperature so that the current density became 50 mA/cm², and the time until the luminance became 95% of the initial luminance (LT95 (unit: h)) was measured.

TABLE 1

| | First electron- | Device lifetime |
|---|---|---|
| | transporting layer | [h] |
| Example 1 | Compound 1 | 166 |
| Example 2 | Compound 2 | 150 |
| Example 3 | Compound 3 | 112 |
| Example 4 | Compound 4 | 145 |
| Example 5 | Compound 5 | 134 |
| Comparative Example 1 | Compound Ref-1 | 85 |
| Comparative Example 2 | Compound Ref-2 | 102 |

### <Fabrication 2 of Organic EL Device>

An organic EL device was fabricated as described below.

### Example 6

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and was then subjected to UV-ozone cleaning for 30 minutes. The thickness of the ITO was set to 130 nm.

The glass substrate with the transparent electrode after the cleaning was mounted on the substrate holder of a vacuum vapor deposition apparatus. First, the compounds HT-1-2 and HA were co-deposited on the surface of the substrate on the side having formed thereon the transparent electrode so that the compounds covered the transparent electrode, and so that the proportion of the compound HA became 3% by mass. Thus, a first hole-transporting layer having a thickness of 10 nm was formed.

The compound HT-1-2 was deposited on the first hole-transporting layer to form a second hole-transporting layer having a thickness of 80 nm.

The compound HT-3 was deposited on the second hole-transporting layer to form a third hole-transporting layer (also referred to as “electron barrier layer”) having a thickness of 5 nm.

The compound BH-2 (host material) and the compound BD-2 (dopant material) were co-deposited on the third hole-transporting layer so that the proportion of the compound BD-2 became 1% by mass. Thus, an emitting layer having a thickness of 20 nm was formed.

The compound 1 was deposited on the emitting layer to form a first electron-transporting layer having a thickness of 5 nm.

The compound ET-2 and Liq were co-deposited on the first electron-transporting layer so that the proportion of Liq became 50% by mass. Thus, a second electron-transporting layer having a thickness of 25 nm was formed.

Metal Yb was deposited on the second electron-transporting layer to form an electron-injecting layer having a thickness of 1 nm.

Metal Al was deposited on the electron-injecting layer to form a cathode having a thickness of 50 nm.

The device configuration of the organic EL device of Example 6 is schematically described as follows.

$$ITO ( 130 ) / HT - 1 - 2 : HA ( 10 : 3 \% ) / HT - 1 - 2 ( 80 ) / HT - 3 ( 5 ) / BH - 2 : BD - 2 ( 20 : 1 \% ) / compound 1 ( 5 ) / ET - 2 : Liq ( 25 : 50 \% ) / Yb ( 1 ) / Al ( 50 )$$

The numerical values in parentheses indicate thicknesses (unit: nm). In addition, the numerical values represented by percent in parentheses each indicate the proportion (% by mass) of the latter compound in the corresponding layer.

### Examples 7 to 11

Organic EL devices were each fabricated in the same manner as in Example 6 except that in the formation of the first electron-transporting layer, a compound shown in Table 2 was used.

### Comparative Example 3

An organic EL device was fabricated in the same manner as in Example 6 except that in the formation of the first electron-transporting layer, the compound Ref-2 was used.

### <Evaluation of Organic EL Device>

The organic EL devices fabricated in Examples 6 to 11 and Comparative Example 3 were evaluated for their device lifetimes in the same manner as in Example 1. The results are shown in Table 2. In Table 2, the device lifetimes are shown as relative values when the device lifetime of Comparative Example 3 is defined as 100.

TABLE 2

| | First electron- | Device lifetime |
|---|---|---|
| | transporting layer | [h] |
| Example 6 | Compound 1 | 131 |
| Example 7 | Compound 6 | 146 |
| Example 8 | Compound 7 | 117 |
| Example 9 | Compound 8 | 144 |
| Example 10 | Compound 9 | 114 |
| Example 11 | Compound 10 | 134 |
| Comparative Example 3 | Compound Ref-2 | 100 |

### <Synthesis of Compound>

### (Synthesis Example 1) Synthesis of Compound 1

The compound 1 was synthesized through the following synthetic route.

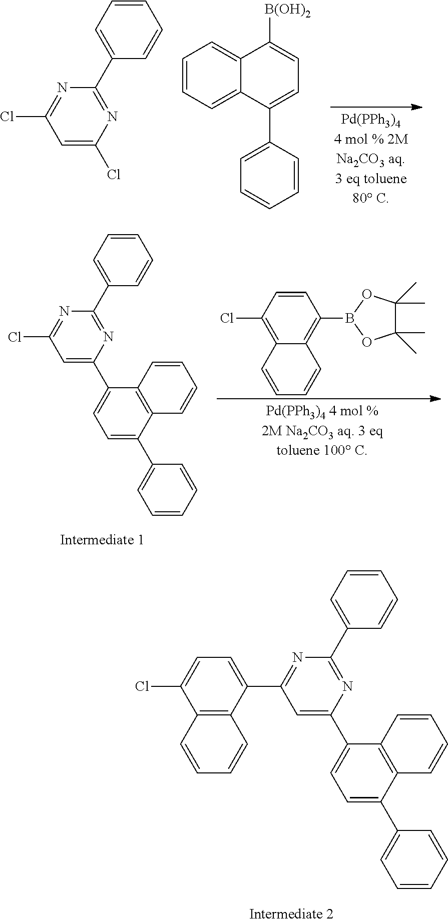

### (1) Synthesis of Intermediate 1

Under an argon atmosphere, toluene (420 mL) and a 2 M aqueous solution of sodium carbonate (63 mL) were added to 2-phenyl-4,6-dichloropyrimidine (11.3 g, 50.3 mmol), (4-phenylnaphthalen-1-yl)boronic acid (10.4 g, 41.9 mmol), and tetrakis(triphenylphosphine)palladium(0) (1.94 g, 1.68 mmol), and the mixture was stirred at 80° C. for 15 hours. After the completion of the reaction, the resultant was extracted with toluene, and the organic phase was concentrated, followed by the purification of the residue by column chromatography. Thus, a white solid (11.4 g) was obtained. The resultant solid was the Intermediate 1 which was an intended product, and as a result of its mass spectral analysis, its ratio m/e was 393, which was a value coinciding with its molecular weight, that is, 392.9.

### (2) Synthesis of Intermediate 2

Under an argon atmosphere, toluene (200 mL) and a 2 M aqueous solution of sodium carbonate (34 mL) were added to the Intermediate 1 (8.91 g, 22.7 mmol), 1-(4,4,5,5-tetramethyl-1,3,2-dioxaborolan-2-yl)-4-chloronaphthalene (6.54 g, 22.7 mmol), and tetrakis(triphenylphosphine)palladium(0) (1.05 g, 0.907 mmol), and the mixture was stirred at 100° C. for 24 hours. After the completion of the reaction, the resultant was passed through a short-path silica gel column chromatograph so that the solvent was concentrated. The resultant solid was recrystallized with toluene to provide a white solid (9.93 g, 84% yield). The resultant solid was the Intermediate 2 which was an intended product, and as a result of its mass spectral analysis, its ratio m/e was 519, which was a value coinciding with its molecular weight, that is, 519.0.

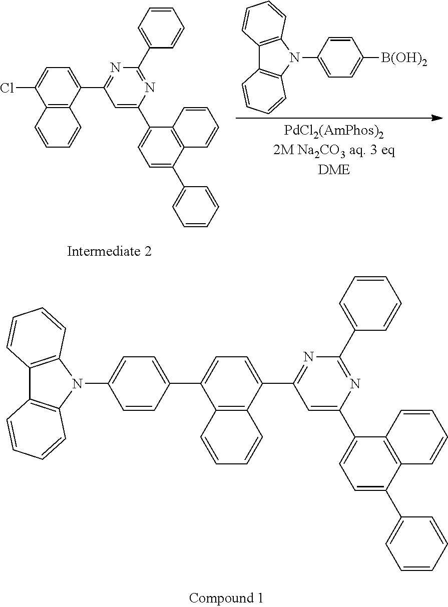 [SMILES: Clc1ccc(-c2cc(-c3ccc(-c4ccccc4)c4ccccc34)nc(-c3ccccc3)n2)c2ccccc12 ; OB(O)c1ccc(-n2c3ccccc3c3ccccc32)cc1 ; c1ccc(-c2nc(-c3ccc(-c4ccccc4)c4ccccc34)cc(-c3ccc(-c4ccc(-n5c6ccccc6c6ccccc65)cc4)c4ccccc34)n2)cc1]

### (3) Synthesis of Compound 1

Under an argon atmosphere, 1,2-dimethoxyethane (60 mL) and a 2 M aqueous solution of sodium carbonate (8.6 mL) were added to the Intermediate 2 (3.00 g, 5.78 mmol), (4-(9H-carbazol-9-yl)phenyl)boronic acid (1.83 g, 6.36 mmol), and PdCl₂(AmPhos)₂ (164 mg, 0.231 mmol), and the mixture was heated and stirred at 70° C. for 15 hours. After the completion of the reaction, the resultant was extracted with toluene, and the solvent was concentrated. The resultant solid was purified by column chromatography to provide a white solid (2.22 g, 53% yield). The resultant solid was the compound 1 which was an intended product, and as a result of its mass spectral analysis, its ratio m/e was 726, which was a value coinciding with its molecular weight, that is, 725.9.

### (Synthesis Example 2) Synthesis of Compound 2

The compound 2 was synthesized through the following synthetic route.

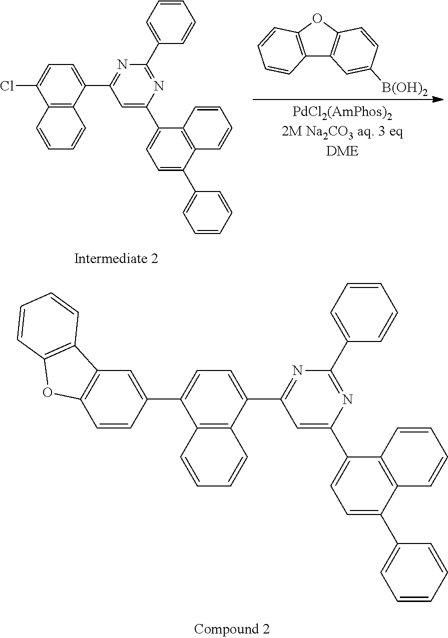 [SMILES: Clc1ccc(-c2cc(-c3ccc(-c4ccccc4)c4ccccc34)nc(-c3ccccc3)n2)c2ccccc12 ; OB(O)c1ccc2oc3ccccc3c2c1 ; c1ccc(-c2nc(-c3ccc(-c4ccccc4)c4ccccc34)cc(-c3ccc(-c4ccc5oc6ccccc6c5c4)c4ccccc34)n2)cc1]

Under an argon atmosphere, 1,2-dimethoxyethane (48 mL) and a 2 M aqueous solution of sodium carbonate (7.2 mL) were added to the Intermediate 2 (2.50 g, 4.82 mmol), dibenzo[b,d]furan-2-ylboronic acid (1.123 g, 5.30 mmol), and PdCl₂(AmPhos)₂ (136 mg, 0.193 mmol), and the mixture was heated and stirred at 70° C. for 15 hours. After the completion of the reaction, the resultant was purified by silica gel column chromatography to provide a white solid (1.5 g, 48% yield). The resultant solid was the compound 2 which was an intended product, and as a result of its mass spectral analysis, its ratio m/e was 651, which was a value coinciding with its molecular weight, that is, 650.8.

### (Synthesis Example 3) Synthesis of Compound 3

The compound 3 was synthesized through the following synthetic route.

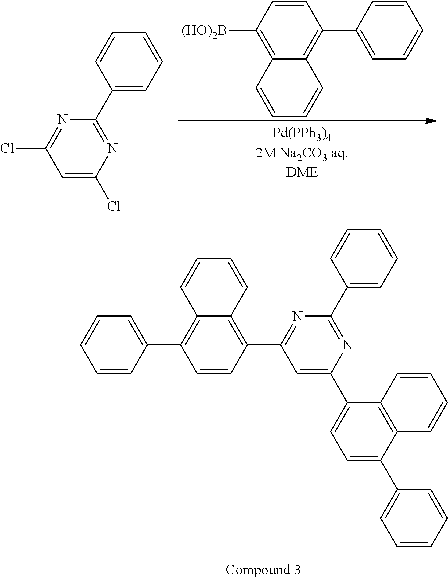 [SMILES: Clc1cc(Cl)nc(-c2ccccc2)n1 ; OB(O)c1ccc(-c2ccccc2)c2ccccc12 ; c1ccc(-c2nc(-c3ccc(-c4ccccc4)c4ccccc34)cc(-c3ccc(-c4ccccc4)c4ccccc34)n2)cc1]

Under an argon atmosphere, 1,2-dimethoxyethane (500 mL) and a 2 M aqueous solution of sodium carbonate (100 mL) were added to 2-phenyl-4,6-dichloropyrimidine (15.0 g, 66.6 mmol), (4-phenylnaphthalen-1-yl)boronic acid (39.7 g, 160 mmol), and tetrakis(triphenylphosphine)palladium(0) (3.85 g, 3.33 mmol), and the mixture was stirred at 80° C. for 15 hours. After the completion of the reaction, the resultant was extracted with toluene, and then the organic phase was concentrated, followed by the purification of the residue by column chromatography. Thus, a white solid (23.2 g, 62% yield) was obtained. The resultant solid was the compound 3 which was an intended product, and as a result of its mass spectral analysis, its ratio m/e was 561, which was a value coinciding with its molecular weight, that is, 560.7.

### (Synthesis Example 4) Synthesis of Compound 4

The compound 4 was synthesized through the following synthetic route.

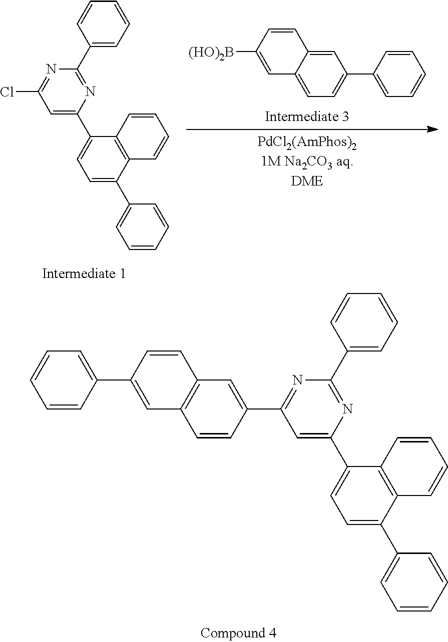 [SMILES: Clc1cc(-c2ccc(-c3ccccc3)c3ccccc23)nc(-c2ccccc2)n1 ; OB(O)c1ccc2cc(-c3ccccc3)ccc2c1 ; c1ccc(-c2ccc3cc(-c4cc(-c5ccc(-c6ccccc6)c6ccccc56)nc(-c5ccccc5)n4)ccc3c2)cc1]

Under an argon atmosphere, 1,2-dimethoxyethane (60 mL) and a 1 M aqueous solution of sodium carbonate (19 mL) were added to the Intermediate 1 (2.50 g, 6.36 mmol), the Intermediate 3 (1.74 g, 7.00 mmol), and PdCl₂(AmPhos)₂ (180 mg, 0.255 mmol), and the mixture was heated and stirred at 70° C. for 15 hours. After the completion of the reaction, the resultant was extracted with toluene, and the solvent was concentrated. The resultant solid was purified by column chromatography to provide a white solid (3.39 g, 95% yield). The resultant white solid was the compound 4 which was an intended product, and as a result of its mass spectral analysis, its ratio m/e was 561, which was a value coinciding with its molecular weight, that is, 560.7.

### (Synthesis Example 5) Synthesis of Compound 5

The compound 5 was synthesized through the following synthetic route.

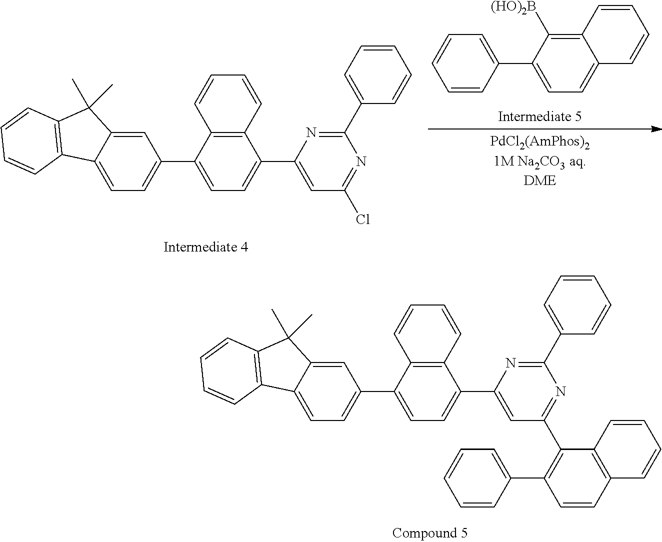

Under an argon atmosphere, 1,2-dimethoxyethane (50 mL) and a 1 M aqueous solution of sodium carbonate (16 mL) were added to the Intermediate 4 (2.70 g, 5.30 mmol), the Intermediate 5 (1.45 g, 5.83 mmol), and PdCl₂(AmPhos)₂ (150 mg, 0.212 mmol), and the mixture was heated and stirred at 70° C. for 15 hours. After the completion of the reaction, the resultant was extracted with toluene, and the solvent was concentrated. The resultant solid was purified by column chromatography to provide a white solid (2.60 g, 72% yield). The resultant white solid was the compound 5 which was an intended product, and as a result of its mass spectral analysis, its ratio m/e was 677, which was a value coinciding with its molecular weight, that is, 676.9.

### (Synthesis Example 6) Synthesis of Compound 6

The compound 6 was synthesized through the following synthetic route.

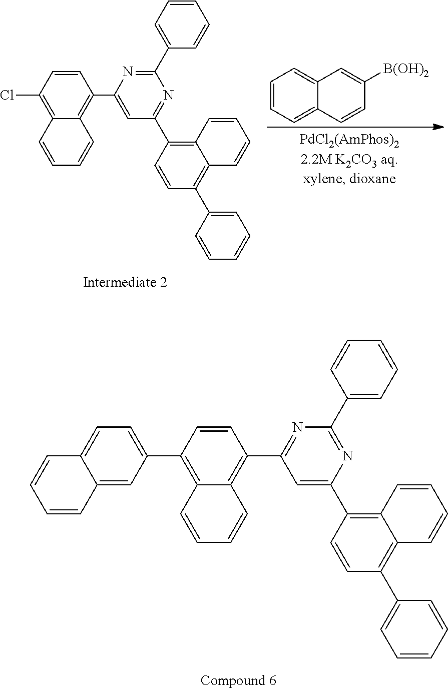

Under an argon atmosphere, xylene (50 mL), dioxane (30 mL), and a 2.2 M aqueous solution of potassium carbonate (18 mL) were added to the Intermediate 2 (8.00 g, 15.4 mmol), (naphthalen-2-yl)boronic acid (2.92 g, 16.95 mmol), and PdCl₂(AmPhos)₂ (327 mg, 0.462 mmol), and the mixture was heated and stirred at 95° C. for an hour. After the completion of the reaction, the resultant was filtered with Celite, and the organic phase was washed with water and a saturated saline solution, followed by the concentration of the solvent. The resultant solid was purified by column chromatography to provide a white solid (8.2 g, 87% yield). The resultant white solid was the compound 6 which was an intended product, and as a result of its mass spectral analysis, its ratio m/e was 611, which was a value coinciding with its molecular weight, that is, 610.8.

### (Synthesis Example 7) Synthesis of Compound 7

The compound 7 was synthesized through the following synthetic route.

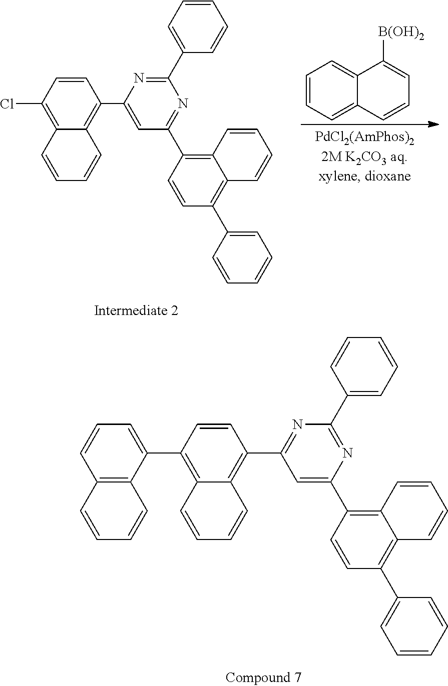 [SMILES: Clc1ccc(-c2cc(-c3ccc(-c4ccccc4)c4ccccc34)nc(-c3ccccc3)n2)c2ccccc12 ; OB(O)c1cccc2ccccc12 ; c1ccc(-c2nc(-c3ccc(-c4ccccc4)c4ccccc34)cc(-c3ccc(-c4cccc5ccccc45)c4ccccc34)n2)cc1]

Under an argon atmosphere, xylene (50 mL), dioxane (30 mL), and a 2 M aqueous solution of potassium carbonate (19 mL) were added to the Intermediate 2 (8.00 g, 15.4 mmol), (naphthalen-1-yl)boronic acid (2.92 g, 16.95 mmol), and PdCl₂(AmPhos)₂ (437 mg, 0.616 mmol), and the mixture was heated and stirred at 95° C. for 2.5 hours. After the completion of the reaction, the resultant was filtered with Celite, and the organic phase was washed with water and a saturated saline solution, followed by the concentration of the solvent. The resultant solid was purified by column chromatography to provide a white solid (7.53 g, 79% yield). The resultant white solid was the compound 7 which was an intended product, and as a result of its mass spectral analysis, its ratio m/e was 611, which was a value coinciding with its molecular weight, that is, 610.8.

### (Synthesis Example 8) Synthesis of Compound 8

The compound 8 was synthesized through the following synthetic route.

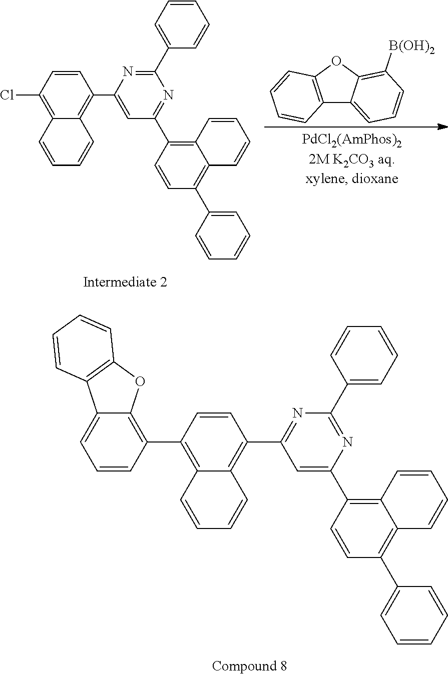 [SMILES: Clc1ccc(-c2cc(-c3ccc(-c4ccccc4)c4ccccc34)nc(-c3ccccc3)n2)c2ccccc12 ; OB(O)c1cccc2c1oc1ccccc12 ; c1ccc(-c2nc(-c3ccc(-c4ccccc4)c4ccccc34)cc(-c3ccc(-c4cccc5c4oc4ccccc45)c4ccccc34)n2)cc1]

Under an argon atmosphere, xylene (60 mL), dioxane (30 mL), and a 2 M aqueous solution of potassium carbonate (19 mL) were added to the Intermediate 2 (8.00 g, 15.4 mmol), dibenzo[b,d]furan-4-ylboronic acid (3.59 g, 16.95 mmol), and PdCl₂(AmPhos)₂ (327 mg, 0.462 mmol), and the mixture was heated and stirred at 95° C. for 2.5 hours. After the completion of the reaction, the resultant was filtered with Celite, and the organic phase was washed with water and a saturated saline solution, followed by the concentration of the solvent. The resultant solid was purified by column chromatography to provide a white solid (7.91 g, 78% yield). The resultant white solid was the compound 8 which was an intended product, and as a result of its mass spectral analysis, its ratio m/e was 651, which was a value coinciding with its molecular weight, that is, 650.8.

### (Synthesis Example 9) Synthesis of Compound 9

The compound 9 was synthesized through the following synthetic route.

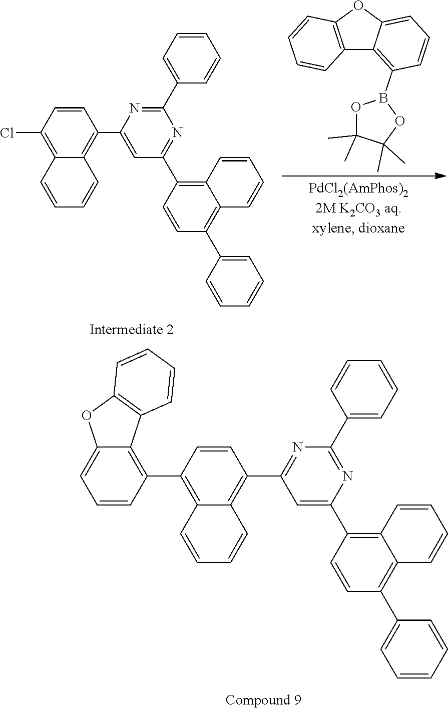 [SMILES: Clc1ccc(-c2cc(-c3ccc(-c4ccccc4)c4ccccc34)nc(-c3ccccc3)n2)c2ccccc12 ; CC1(C)OB(c2cccc3oc4ccccc4c23)OC1(C)C ; c1ccc(-c2nc(-c3ccc(-c4ccccc4)c4ccccc34)cc(-c3ccc(-c4cccc5oc6ccccc6c45)c4ccccc34)n2)cc1]

Under an argon atmosphere, xylene (60 mL), dioxane (30 mL), and a 2 M aqueous solution of potassium carbonate (19 mL) were added to the Intermediate 2 (8.00 g, 15.4 mmol), 2-(dibenzo[b,d]furan-1-yl)-4,4,5,5-tetramethyl-1,3,2-dioxaborolane (4.99 g, 16.95 mmol), and PdCl₂(AmPhos)₂ (327 mg, 0.462 mmol), and the mixture was heated and stirred at 95° C. for 4 hours. After the completion of the reaction, the resultant was filtered with Celite, and the organic phase was washed with water and a saturated saline solution, followed by the concentration of the solvent. The resultant solid was purified by column chromatography to provide a white solid (8.16 g, 81% yield). The resultant white solid was the compound 9 which was an intended product, and as a result of its mass spectral analysis, its ratio m/e was 651, which was a value coinciding with its molecular weight, that is, 650.8.

### (Synthesis Example 10) Synthesis of Compound 10

The compound 10 was synthesized through the following synthetic route.

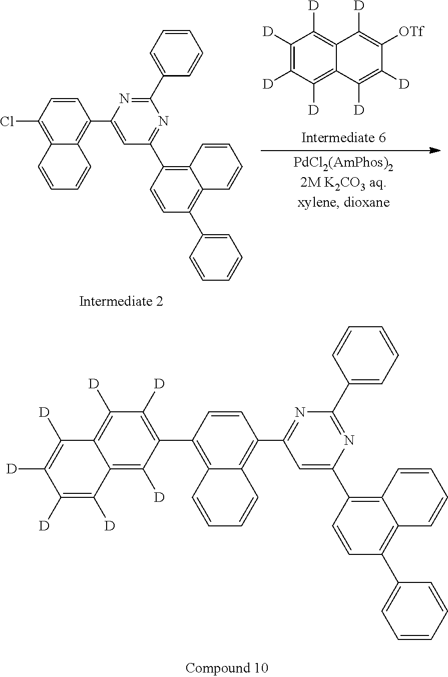 [SMILES: Clc1ccc(-c2cc(-c3ccc(-c4ccccc4)c4ccccc34)nc(-c3ccccc3)n2)c2ccccc12 ; [2H]c1c([2H])c([2H])c2c([2H])c(OS(=O)(=O)C(F)(F)F)c([2H])c([2H])c2c1[2H] ; [2H]c1c([2H])c([2H])c2c([2H])c(-c3ccc(-c4cc(-c5ccc(-c6ccccc6)c6ccccc56)nc(-c5ccccc5)n4)c4ccccc34)c([2H])c([2H])c2c1[2H]]

Under an argon atmosphere, xylene (30 mL), dioxane (15 mL), and a 2 M aqueous solution of potassium carbonate (10 mL) were added to the Intermediate 2 (4.00 g, 7.7 mmol), the Intermediate 6 (2.40 g, 8.5 mmol), and PdCl₂(AmPhos)₂ (164 mg, 0.231 mmol), and the mixture was heated and stirred at 95° C. for two hours. After the completion of the reaction, the resultant was filtered with Celite, and the organic phase was washed with water and a saturated saline solution, followed by the concentration of the solvent. The resultant solid was purified by column chromatography to provide a white solid (3.4 g, 71% yield). The resultant white solid was the compound 10 which was an intended product, and as a result of its mass spectral analysis, its ratio m/e was 618, which was a value coinciding with its molecular weight, that is, 617.8.

### (Synthesis Example 11) Synthesis of Compound 11

The compound 11 was synthesized through the following synthetic route.

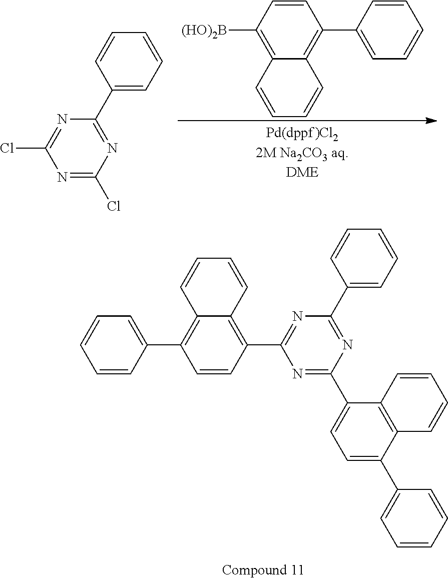 [SMILES: Clc1nc(Cl)nc(-c2ccccc2)n1 ; OB(O)c1ccc(-c2ccccc2)c2ccccc12 ; c1ccc(-c2nc(-c3ccc(-c4ccccc4)c4ccccc34)nc(-c3ccc(-c4ccccc4)c4ccccc34)n2)cc1]

Under an argon atmosphere, 1,2-dimethoxyethane (44 mL) and a 1 M aqueous solution of sodium carbonate (20 mL) were added to 2,4-dichloro-6-phenyl-1,3,5-triazine (1.50 g, 6.6 mmol), (4-phenylnaphthalen-1-yl)boronic acid (3.62 g, 14.6 mmol), and Pd(dppf)Cl₂ (194 mg, 0.265 mmol), and the mixture was stirred at 80° C. for 18 hours. After the completion of the reaction, the resultant was extracted with toluene, and then the organic phase was concentrated, followed by the purification of the residue by column chromatography. Thus, a white solid (2.57 g, 69% yield) was obtained. The resultant white solid was the compound 11 which was an intended product, and as a result of its mass spectral analysis, its ratio m/e was 562, which was a value coinciding with its molecular weight, that is, 561.7.

Although some embodiments and/or Examples of the present invention have been described in detail above, those skilled in the art will readily appreciate that many modifications are possible in the exemplary embodiments and/or Examples without materially departing from the novel teachings and effects of the present invention. Accordingly, the many modifications are included in the scope of the present invention.

The literatures described in this description and the contents of the application(s) on the basis of which the present application claims Paris convention priority are incorporated herein by reference in their entirety.

1. A compound represented by the following formula (1): 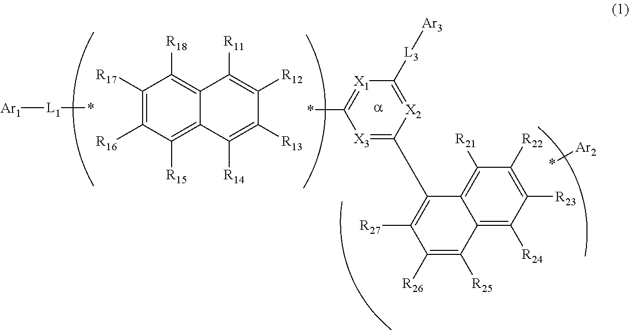
wherein in the formula (1),
X₁ to X₃ are independently C(R₁) or N, and at least two of X₁ to X₃ are N,
R₁ is a hydrogen atom or a substituent R,
Ar₁ and Ar₃ are independently
a substituted or unsubstituted aryl group having 6 to 50 ring carbon atoms, or
a substituted or unsubstituted monovalent heterocyclic group having 5 to 50 ring atoms,
Ar₂ is
a substituted or unsubstituted aryl group having 6 to 50 ring carbon atoms,
L₁ and L₃ are
a single bond,
a substituted or unsubstituted arylene group having 6 to 30 ring carbon atoms,
a substituted or unsubstituted divalent heterocyclic group having 5 to 30 ring atoms, or
a divalent group obtained by combining two or three of a substituted or unsubstituted arylene group having 6 to 30 ring carbon atoms, and a substituted or unsubstituted divalent heterocyclic group having 5 to 30 ring atoms,
one or more sets of adjacent two or more of R₁₁ to R₁₈ do not bond with each other,
any one of R₁₁ to R₁₈ represents a bond with a six-membered ring α,
any one of R₁₁ to R₁₈ which do not represent bonds with the six-membered ring α represents a bond with L₁, and when L₁ is a single bond, any one of R₁₁ to R₁₈ which do not represent bonds with the six-membered ring α is directly bonded with Ar₁,
R₁₁ to R₁₈ which do not represent bonds with the six-membered ring α and which do not represent bonds with L₁ are independently a hydrogen atom or a substituent R,
one or more sets of adjacent two or more of R₂₁ to R₂₇ do not bond with each other,
any one of R₂₁ to R₂₇ represents a bond with Ar₂,
R₂₁ to R₂₇ which do not represent bonds with Ar₂ are independently a hydrogen atom or a substituent R,
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇):
wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms, and when two or more of each of R₉₀₁ to R₉₀₇ are present, the two or more of each of R₉₀₁ to R₉₀₇ may be the same as or different from each other;
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms, and
when the two or more substituents R are present, the two or more substituents R may be the same as or different from each other.

2. The compound according to claim 1, wherein the compound represented by the formula (1) is a compound represented by the following formula (1-1) or (1-2): 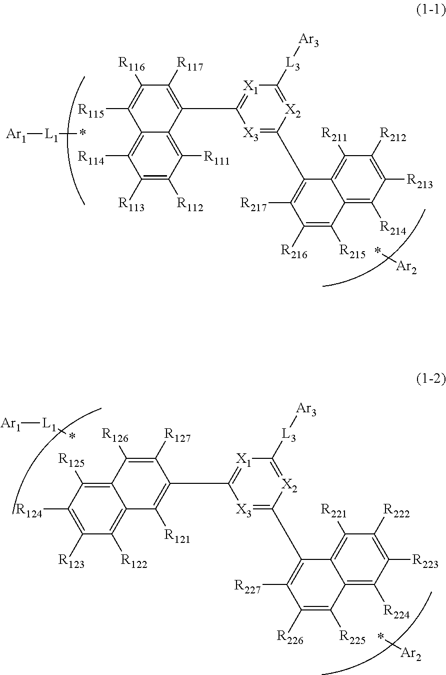
wherein in the formulas (1-1) to (1-2), X₁ to X₃, Ar₁ to Ar₃, L₁, and L₃ are the same as defined in the formula (1),
in the formula (1-1),
one or more sets of adjacent two or more of R₁₁₁ to R₁₁₇ and R₂₁₁ to R₂₁₇ do not bond with each other,
any one of R₁₁₁ to R₁₁₇ represents a bond with L₁, and when L₁ is a single bond, any one of R₁₁₁ to R₁₁₇ is directly bonded with Ar₁,
R₁₁₁ to R₁₁₇ which do not represent bonds with L₁ are independently a hydrogen atom or a substituent R,
any one of R₂₁₁ to R₂₁₇ represents a bond with Ar₂, and
R₂₁₁ to R₂₁₇ which do not represent bonds with Ar₂ are independently a hydrogen atom or a substituent R, and
in the formula (1-2),
one or more sets of adjacent two or more of R₁₂₁ to R₁₂₇ and R₂₂₁ to R₂₂₇ do not bond with each other,
any one of R₁₂₁ to R₁₂₇ represents a bond with L₁, and when L₁ is a single bond, any one of R₁₂₁ to R₁₂₇ is directly bonded with Ar₁,
R₁₂₁ to R₁₂₇ which do not represent bonds with L₁ are independently a hydrogen atom or a substituent R,
any one of R₂₂₁ to R₂₂₇ represents a bond with Ar₂,
R₂₂₁ to R₂₂₇ which do not represent bonds with Ar₂ are independently a hydrogen atom or a substituent R, and
the substituent R is the same as defined in the formula (1).

3. The compound according to claim 1, wherein two of X₁ to X₃ are N, and another one thereof is C(R₁).

4. The compound according to claim 1, wherein X₁ and X₂ are N, and X₃ is C(R₁).

5. The compound according to claim 1, wherein R₁ is a hydrogen atom.

6. The compound according to claim 1, wherein the compound represented by the formula (1) is selected from the group consisting of compounds represented by the following formulas (1-11) to (1-31): 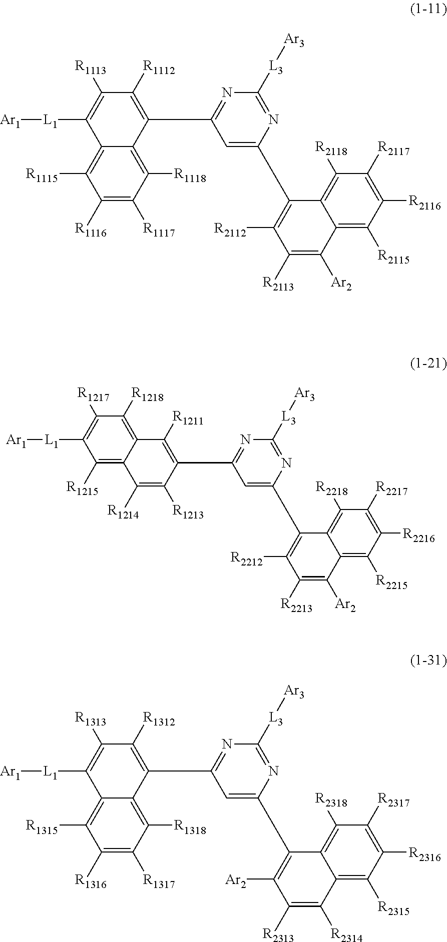
wherein in the formulas (1-11) to (1-31), Ar₁ to Ar₃, L₁, and L₃ are the same as defined in the formula (1),
one or more sets of adjacent two or more of R₁₁₁₂, R₁₁₁₃, R₁₁₁₅ to R₁₁₁₈, R₂₁₁₂, R₂₁₁₃, R₂₁₁₅ to R₂₁₁₈, R₁₂₁₁, R₁₂₁₃ to R₁₂₁₅, R₁₂₁₇ to R₁₂₁₈, R₂₂₁₂ to R₂₂₁₃, R₂₂₁₅ to R₂₂₁₈, R₁₃₁₂ to R₁₃₁₃, R₁₃₁₅ to R₁₃₁₈, and R₂₃₁₃ to R₂₃₁₈ do not bond with each other,
R₁₁₁₂, R₁₁₁₃, R₁₁₁₅ to R₁₁₁₈, R₂₁₁₂, R₂₁₁₃, R₂₁₁₅ to R₂₁₁₈, R₁₂₁₁, R₁₂₁₃ to R₁₂₁₅, R₁₂₁₇ to R₁₂₁₈, R₂₂₁₂ to R₂₂₁₃, R₂₂₁₅ to R₂₂₁₈, R₁₃₁₂ to R₁₃₁₃, R₁₃₁₅ to R₁₃₁₈, and R₂₃₁₃ to R₂₃₁₈ are independently a hydrogen atom or a substituent R, and
the substituent R is the same as defined in the formula (1).

7. The compound according to claim 1, wherein Ar₁ is a group represented by the following formula (1A): 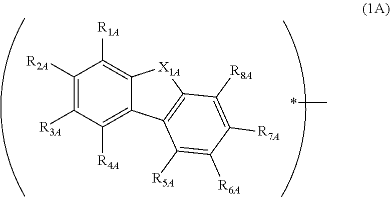
wherein in the formula (1A),
X_{1A} is O, S, N(R_{9A}), or C(R_{10A})(R_{11A}),
any one of R_{9A} in a case where X_{1A} is N(R_{9A}), and R_{1A} to R_{8A} represents a bond with L₁ with which Ar₁ is bonded, and when L₁ is a single bond, any one of R_{9A} in the case where X_{1A} is N(R_{9A}), and R_{1A} to R_{8A} is directly bonded with a naphthalene skeleton with which Ar₁ is bonded,
one or more sets of adjacent two or more of R_{10A} and R_{11A} in a case where X_{1A} is C(R_{10A})(R_{11A}), and R_{1A} to R_{8A} which do not represent bonds with L₁
form a substituted or unsubstituted single ring by bonding with each other,
form a substituted or unsubstituted fused ring by bonding with each other, or
do not bond with each other,
R_{9A} which does not represent a bond with L₁, R_{10A} and R_{11A} which do not form the substituted or unsubstituted single ring and which do not form the substituted or unsubstituted fused ring, and R_{1A} to R_{8A} which do not represent bonds with L₁, which do not form the substituted or unsubstituted single ring, and which do not form the substituted or unsubstituted fused ring are independently a hydrogen atom or a substituent R, and
the substituent R is the same as defined in the formula (1).

8. The compound according to claim 1, wherein Ar₁ is
a substituted or unsubstituted phenyl group,
a substituted or unsubstituted 1-naphthyl group, or
a substituted or unsubstituted 2-naphthyl group.

9. The compound according to claim 1, wherein Ar₂ is
a substituted or unsubstituted phenyl group,
a substituted or unsubstituted 1-naphthyl group, or
a substituted or unsubstituted 2-naphthyl group.

10. The compound according to claim 1, wherein Ar₃ is a substituted or unsubstituted aryl group having 6 to 50 ring carbon atoms.

11. The compound according to claim 1, wherein Ar₃ is
a substituted or unsubstituted phenyl group,
a substituted or unsubstituted 1-naphthyl group, or
a substituted or unsubstituted 2-naphthyl group.

12. The compound according to claim 1, wherein L₃ is a single bond.

13. The compound according to claim 1, wherein in the formula (1), R₁₁ to R₁₈ which do not represent bonds with the six-membered ring α and which do not represent bonds with Ar₁, and R₂₁ to R₂₇ which do not represent bonds with Ar₂ are hydrogen atoms.

14. The compound according to claim 1, wherein a substituent in a case of “substituted or unsubstituted” is selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇): wherein R₉₀₁ to R₉₀₇ are independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms, and when two or more of each of R₉₀₁ to R₉₀₇ are present, the two or more of each of R₉₀₁ to R₉₀₇ may be the same as or different from each other;
a halogen atom, a cyano group, a nitro group,
an unsubstituted aryl group having 6 to 50 ring carbon atoms, and
an unsubstituted heterocyclic group having 5 to 50 ring atoms.

15. The compound according to claim 1, wherein a substituent in a case of “substituted or unsubstituted” is selected from the group consisting of
an alkyl group having 1 to 18 carbon atoms,
an aryl group having 6 to 18 ring carbon atoms, and
a monovalent heterocyclic group having 5 to 18 ring atoms.

16. (canceled)

17. An organic electroluminescence device comprising
a cathode,
an anode, and
one or more organic layers arranged between the cathode and the anode,
wherein at least one layer of the one or more organic layers comprises the compound according to claim 1.

18. The organic electroluminescence device according to claim 17, which comprises an anode, an emitting layer, an electron-transporting region, and a cathode in this order, wherein the electron-transporting region comprises the compound.

19. The organic electroluminescence device according to claim 18, wherein the electron-transporting region comprises at least a first layer and a second layer in this order from the emitting layer side, and
the first layer comprises the compound.

20. The organic electroluminescence device according to claim 18, which comprises a hole-transporting region between the anode and the emitting layer.

21. An electronic apparatus comprising the organic electroluminescence device according to claim 17.
